(19) United States
(12) Patent Application Publication
(10) Pub. No.: US 2025/0188135 A1
(43) Pub. Date: Jun. 12, 2025

(54) A MULTIPLE ANTIGEN PRESENTING SYSTEM (MAPS)-BASED STAPHYLOCOCCUS AUREUS VACCINE COMPRISING B- AND T-CELL ANTIGENS, IMMUNOGENIC COMPOSITION, AND USES THEREOF

(71) Applicant: THE CHILDREN'S MEDICAL CENTER CORPORATION, Boston, MA (US)

(72) Inventors: Richard MALLEY, Beverly, MA (US); Yingjie LU, West Roxbury, MA (US); Fan ZHANG, West Roxbury, MA (US); Kristin MOFFITT, Woburn, MA (US)

(73) Assignee: THE CHILDREN'S MEDICAL CENTER CORPORATION, Boston, MA (US)

(21) Appl. No.: 18/846,176

(22) PCT Filed: Mar. 10, 2023
(86) PCT No.: PCT/US2023/014993

§ 371 Date: Sep. 11, 2024

Related U.S. Application Data
(60) Provisional application No. 63/318,951, filed on Mar. 11, 2022.

(51) Int. Cl. C07K 14/31; A61K 39/00

(52) U.S. Cl. CPC C07K 14/31; A61K 39/00; A61K 2039/6068; A61K 2039/625

(57) ABSTRACT

The present embodiments provide for an S. aureus (SA). Multiple Antigen Presenting System (MAPS) immunogenic composition comprising an immunogenic polysaccharide which induces a B-cell and T-cell immune response, where at least one S. aureus (SA) peptide or polypeptide antigen is associated to the immunogenic polysaccharide by complementary affinity molecules. The present SA-MAPS immunogenic composition herein generates an immune response in a subject, preferably an antibody response and a B-cell and/or T-cell response, and can comprise at least one B-cell SA antigen and at least one T-cell SA antigen, and can elicit both humoral and cellular immune responses to the immunogenic polysaccharide and one, or multiple SA antigens at the same time.

### CROSS-REFERENCED TO RELATED APPLICATIONS

This application is a 371 National Phase Entry of International Patent Application which claims benefit under 35 U.S.C. § 119(e) of U.S. Provisional Application No. 63/318,951 filed Mar. 11, 2022, the contents of which is incorporated herein by reference in its entirety.

The present invention relates to molecular genetics, immunology, and microbiology. The present application is generally directed to compositions and methods for preparation of immunogenic compositions. More specifically, an embodiment of the present invention provides for an immunogenic composition comprising at least two immunogenic Staphylococcus aureus peptide antigens attached to an immunogenic polysaccharide. In some embodiments, this complex can be used as an immunogenic composition, such as a vaccine, to confer a synergistic humoral and cellular immune response; and in some embodiments, elicits synergistic antibody and/or B-cell response and also in some embodiments, a T-cell mediated protection against S. aureus infection and colonization and carriage.

### BACKGROUND OF INVENTION

Staphylococcus aureus (SA) is an important Gram-positive bacterium that causes a wide range of infections in both healthy and compromised individuals. SA is one of the leading causes of community- and hospital-acquired bacterial infections and postsurgical wound infections, resulting in prolonged hospital stay and significantly increased healthcare cost. Staphylococcal bacteremia is associated with high mortality (about 20-40% in adults) even after appropriate antibiotic treatment. Skin and soft tissue infection (SSTI) is a common chronic SA infection with frequent recurrence. Depending on the severity and depth of the infection, SSTI may represent as scalded skin syndrome, boils, impetigo, cellulitis, abscess, fasciitis or myonecrosis. SA is also a cause of invasive disease, including meningitis, endocarditis, osteomyelitis, pneumonia, sepsis and toxic shock syndrome. SA colonizes about 20% of the human population persistently and up to 80% transiently, serving as a reservoir for future infection and transmission. The treatment of SA infection includes surgical procedure, antibiotics, or a combination of both. However, the effectiveness of antibiotic treatment has been severely impacted by the rapid emergence of multi-drug resistant strains (Methicillin-resistant SA, MRSA, as well as Vancomycin-intermediate strains, or VISA) in both community-acquired (CA-) and hospital-acquired (HA-) infections in the past two decades.

Humans are the natural reservoirs for Staphylococcus aureus (S. aureus). Healthy individuals can be colonized by S. aureus on the skin, in the nares and the throat either persistently (10-35%), intermittently (20-75%) or be in a non-carriage state (5-70%) with no associated disease. See Vandenbergh et al, J. Clin. Micro. 37:3133-3140 (1999). Disease subsequently occurs when individuals become immunocompromised due to breaches in immune barriers, such as during surgery, placement of indwelling catheters or other devices, trauma, or wounds. The resulting S. aureus infection can cause a wide range of diseases that range from mild skin infections to endocarditis, osteomyelitis, bacteremia, sepsis, and other forms of disease with accompanying high mortality rates. The large human reservoir enhances opportunity for evolution and spread of adapted pathogenic clonal types.

Invasive staphylococcal infections from the Gram positive cocci S. aureus and S. epidermidis are of particular concern because they are an increasing public health problem worldwide. Specifically, S. aureus is responsible for the majority of hospital-acquired (nosocomial) infections, and its prevalence in community-onset infections is increasing. For example, the incidence of invasive methicillin-resistant S. aureus (MRSA) was estimated at 31.8 per 100,000 persons, including 18,650 deaths in the United States in 2005. See Klevens R. M. et al, JAMA, 298: 1763-71 (2007). Staphylococcal diseases have seen a dramatic increase in the last 20 years; this increase parallels the use of intravascular devices and invasive procedures. The rise in disease incidence is made more troubling because of the parallel rise of antibiotic resistance; therefore, there is an urgent need for immunogenic compositions for use in vaccines or to elicit polyclonal or monoclonal antibodies to confer passive immunity as a means to prevent or treat staphylococcal infection and associated diseases.

A vaccine against SA would represent a very attractive alternative. Vaccines provide prevention of and treatment for a variety of diseases, including microorganism infection, viral infection, and cancers. Success of polysaccharide-based vaccines and passive immunization for the prevention of colonization or disease has demonstrated the importance of capsular antibodies, in particular in controlling disease caused by S. pneumoniae. Further, studies in both animals and humans demonstrate that antibodies elicited from pneumococcal vaccination can protect against nasopharyngeal (NP) pneumococcal colonization, which precedes pneumococcal disease.

If successful, a SA vaccine could provide broad, long-term benefit to the population via both direct and herd immunities. Efforts in the early SA vaccine development have focused on generating antibodies to various polysaccharide or protein antigens, including the capsular polysaccharides, the extracellular polysaccharides, the toxins and the surface proteins. The strategy of taking a combination of capsular polysaccharides and/or proteins has been successfully used against many human pathogens, such as Haemophilus influenzae type b, Streptococcus pneumoniae, Neisseria meningitidis (including most recently serogroup B), pertussis. The same approach has been attempted for vaccines for SA. However, prior vaccine candidates for SA to date—which include use of SA polysaccharides and proteins in vaccines, or antibodies directed against these antigens, have failed in clinical trials. This is not expected considering that there was clear demonstration of efficacy of these vaccines in various animal models of invasive SA infections.

Given this failure, there remains a need to improve the efficacy of SA vaccines, particularly to prevent infection and/or colonization and carriage.

### SUMMARY OF THE INVENTION

The present invention provides for an improved immunogenic multiple antigen presenting system (MAPS) comprising a biotinylated immunogenic polysaccharide, and at least one Staphylococcus aureus (SA) antigen selected from a specific group of antigens (Group A) attached to the immunogenic polysaccharide, where the specific SA antigen is fused to a biotin-binding protein. Such a Staphylococcus aureus-MAPS composition, referred to herein as “SA-MAPS” as disclosed herein is useful for the production of immunogenic compositions, such as those useful in vaccines, as well as for treatment.

Staphylococcus aureus (SA) is a major cause of morbidity and mortality worldwide. Vaccine development against SA has been challenging, likely due to the complexity of pathogenesis and an incomplete understanding of protective immune mechanisms. The inventors previously developed a vaccine platform referred to the Multiple-Antigen-Presenting-System (MAPS), as disclosed in US patent Application 2014/0154287, which is incorporated herein in its entirety by reference, which enables the induction of broad adaptive immune responses.

Herein, the inventors have developed and optimized the system for the treatment and prevention of infection from Staphylococcus aureus. Herein, the inventors have identified and used novel SA-specific polypeptide antigens for use in a SA-MAPS immunogenic composition which demonstrate that B- and T-cell mediated immune mechanisms contribute differentially to host defense against SA in models of skin necrosis, skin abscess, invasive disease or mucosal colonization.

The inventors have previously described in US application US2021/0008192 (″192 application) an immunogenic MAPS composition comprising at least one Staphylococcus aureus (SA) peptide antigens (SA-MAPS). In the '192 application, which is incorporated herein by reference in its entirety, the SA peptide antigens are selected from any of: hemolysin (Hl) (e.g., hemolysin α or Hla), Clumping factor A (ClfA), Clumping factor B (ClfB), serine-aspirate repeat protein D (SdrD), Iron regulator surface protein A (IsdA) and Iron regulator surface protein B (IsdB). The '192 application discloses a specific SA-MAPS immunogenic composition.

Herein, the inventors have improved on the SA-MAPS composition disclosed in the '192 application. In particular, the inventors have discovered additional Staphylococcus aureus (SA) peptide antigens to those disclosed in the '192 application. In some embodiments, at least one of these newly discovered SA polypeptide antigens can used alone, or added to a selection of at least 1, or at least 2, or at least 3 or at least 4 or more SA polypeptide antigens disclosed in the '192 application for in an improved SA-MAPS immunogenic composition. In some embodiments, these newly discovered SA peptide antigens that can be used in the improved SA-MAPS immunogenic composition as disclosed herein are selected at least one of SA peptide antigens selected from any of: SA1739 (B2), SA1720 (B1), SA1890 (B3), SA0103 (TI), SA0377 (T2), SA0693 (T3) and SA2105 (T4).

In one embodiment, the SA-MAPS composition useful for the method and compositions as disclosed herein comprises at least one B-cell antigen, selected from SA1739 (B2), SA1720 (B1), SA1890 (B3), and at least one T-cell SA antigen, selected from any of SA0103 (TI), SA0377 (T2), SA0693 (T3) and SA2105 (T4). In some embodiments, a SA-MAPS composition useful for the method and compositions as disclosed herein comprises at least one B-cell antigen, selected from SA1739 (B2), SA1720 (B1), SA1890 (B3), and at least one T-cell SA antigen, selected from any of SA0103 (T1), SA0377 (T2), SA0693 (T3) and SA2105 (T4), and at least one, or at least 2, or at least 3, or at least 4 SA antigens disclosed in the '192 application, or at least 1, or at least 2, or at least 3 or at least 4 or more SA-MAPS peptide antigens selected from any of: hemolysin (Hl) (e.g., hemolysin a or Hla), Clumping factor A (ClfA), Clumping factor B (ClfB), serine-aspirate repeat protein D (SdrD), Iron regulator surface protein A (IsdA) and Iron regulator surface protein B (IsdB).

In some embodiments, the SA-MAPS composition as disclosed herein can comprise at least one, or at least two, or more than two SA peptide antigens selected from SA1739 (B2), SA1720 (B1), SA1890 (B3), SA0103 (T1), SA0377 (T2), SA0693 (T3) and SA2105 (T4).

In some embodiments, the SA-MAPS composition as disclosed herein can comprise at least one, or at least two, or more than two SA peptide antigens selected from SA1739 (B2), SA1720 (B1), SA1890 (B3), SA0103 (T1), SA0377 (T2), SA0693 (T3) and SA2105 (T4), and at least 1, or at least 2, or at least 3 or at least 4 or more SA-MAPS peptide antigens disclosed in '192 application. For example, and not wishing to be limited by theory, a SA-MAPS composition as disclosed herein can comprise at least one, or at least two SA peptide antigens selected from SA1739 (B2), SA1720 (B1), SA1890 (B3), SA0103 (T1), SA0377 (T2), SA0693 (T3) and SA2105 (T4), and at least 1, or at least 2, or at least 3 or at least 4 or more SA-MAPS peptide antigens selected from any of: hemolysin (Hl) (e.g., hemolysin α or Hla), Clumping factor A (ClfA), Clumping factor B (ClfB), serine-aspirate repeat protein D (SdrD), Iron regulator surface protein A (IsdA) and Iron regulator surface protein B (IsdB).

In some embodiments, a SA-MAPS composition as disclosed herein can comprise at least one, or at least two SA peptide antigens, or more than two SA antigens selected from SA1739 (B2), SA1720 (B1), SA1890 (B3), SA0103 (T1), SA0377 (T2), SA0693 (T3) and SA2105 (T4), and at least 1, or at least 2, or at least 3 or at least all 4 SA-MAPS peptide antigens selected from any of: hemolysin (Hl) (e.g., hemolysin a or Hla), Clumping factor A (ClfA), Clumping factor B (ClfB), serine-aspirate repeat protein D (SdrD). An immunogenic SA-MAPS composition comprising four (4) SA-MAPS peptide antigens of: hemolysin (Hl) (e.g., hemolysin α or Hla), Clumping factor A (ClfA), Clumping factor B (ClfB), serine-aspirate repeat protein D (SdrD) is referred to herein as “MAPS4”.

In some embodiments, the SA-MAPS immunogenic composition as disclosed herein generates an immune response in a subject, preferably an antibody response and a B-cell and/or T-cell response. In some embodiments, the SA-MAPS immunogenic composition as disclosed herein generates a CD8+ T-cell response, a CD4+ T-cell response or a CD8+/CD4+ T-cell response. The inventors demonstrate that mice immunized with or administered a SA-MAPS immunogenic composition as disclosed responded to SA antigens and produced significant amount of IFN-γ, IL-17A and IL-22, demonstrating that the SA-MAPS composition can generate of Th1, Th2, Th17 and Th22 responses. Accordingly, in some embodiments, a SA-MAPS immunogenic composition as disclosed herein generates a T-cell response and, more specifically, any one or more of a Th1, Th2, Th17 and Th22 response to a SA peptide or protein present in the SA-MAPS composition. In some embodiments, a SA-MAPS immunogenic composition as disclosed herein generates an anti-polysaccharide antibody response and/or a B-cell and/or T-cell, e.g., Th1/Th2/Th17/Th22 response. In some embodiments, the immune response elicited by the SA-MAPS immunogenic composition as disclosed herein is an antibody or B cell response to at least one antigenic polysaccharide, and an antibody or B cell response and a CD4+ and/or CD8+ T cell response, including Th1, Th2, Th17 or Th22 responses, or a CD8+ T cell response.

In some embodiments, a SA-MAPS immunogenic composition as disclosed herein elicits an immune response that results in activation of INF-γ, IL-17A, IL-17F, IL-21 or IL-22 producing cells, or produces INF-γ, IL-17A and IL-22 producing cells. This is important in that the SA-MAPS immunogenic composition presents a major advantage by eliciting two forms of immunity—that is, a conventional humoral (B-cell dependent) immune response to an immunogenic polysaccharide and SA-antigens, as well as a T-cell response and, more specifically, any one or more of Th17, Th1, Th2 or Th22 responses to a SA peptide or protein present in the SA-MAPS composition. Moreover, in some embodiments, the SA-MAPS immunogenic composition as disclosed herein can enhance specific B-cell or T-cell responses by modifying the protein/polysaccharide ratio, complex size, or by incorporating specific co-stimulatory factor, such as TLR2/4 ligands, etc., into the composition.

In particular, the present invention is relates to compositions comprising an immunogenic polysaccharide, at least one Staphylococcus aureus peptide antigen as disclosed herein; and at least one complementary affinity-molecule pair comprising (i) a first affinity molecule that associates with the immunogenic polysaccharide, and (ii) a complementary affinity molecule that associates with the Staphylococcus aureus polypeptide antigen, such that the first and complementary affinity molecules serve as an indirect link between the immunogenic polysaccharide and SA polypeptide antigens. In some embodiments, the SA-MAPS immunogenic composition comprises (i) at least one immunogenic polysaccharide, (ii) at least two S. aureus polypeptide antigens, and (iii) at least one pair of affinity molecules, wherein the at least one pair of affinity molecules comprises (a) a first affinity molecule comprising a biotin, and (b) a second affinity molecule comprising a biotin-binding protein, wherein the first affinity molecule is associated with the at least one immunogenic polysaccharide, and the second affinity molecule is associated with the at least S. aureus polypeptide antigens, and wherein the first affinity molecule non-covalently associates with the second affinity molecule to link the S. aureus polypeptide antigen(s) and the immunogenic polysaccharide, wherein the at least one of the S. aureus polypeptide antigen is selected from any of: SA1739 (B2), SA1720 (B1), SA1890 (B3), SA0103 (T1), SA0377 (T2), SA0693 (T3) and SA2105 (T4). In some embodiments, the at least one immunogenic polysaccharide comprises type 1 capsular polysaccharide of Streptococcus pneumoniae, a type 5 capsular polysaccharide of S. aureus, and/or a type 8 capsular polysaccharide of S. aureus.

In some embodiments, a SA-MAPS composition as disclosed herein refers to, in brief, an immunogenic composition comprising (i) at least one immunogenic polysaccharide which has a first affinity molecule attached (e.g., biotin attached), and (ii) at least one S. aureus polypeptide antigen, where the S. aureus polypeptide antigen is a SA-antigen as disclosed herein, e.g., at least one Group A SA-antigen, and is fused to a second affinity molecule, (e.g., a biotin-binding moiety, such as, but not limited to Rhizavidin comprising the amino acid sequence of SEQ ID NO: 1), wherein the first affinity molecule non-covalently associates with the second affinity molecule, thereby linking the S. aureus polypeptide antigen(s) and the immunogenic polysaccharide.

In another embodiment, a SA-MAPS composition useful in the methods and compositions as disclosed herein refers to, in brief, an immunogenic composition comprising, (i) a biotinylated immunogenic polysaccharide and (ii) at least one fusion protein, the fusion protein comprising a biotin-binding protein fused to at least one SA-antigen as disclosed herein, e.g., at least one Group A SA-antigen as disclosed herein, and where the biotinylated polysaccharide antigen is non-covalently associated with the biotin-binding moiety of the fusion protein to form an immunogenic complex. In some embodiments, the immunogenic polysaccharide is selected from any of: type 1 capsular polysaccharide of Streptococcus pneumoniae, a type 5 capsular polysaccharide of S. aureus, and/or a type 8 capsular polysaccharide of S. aureus.

Such a system allows for a modular immunogenic composition, where one or more SA polypeptide antigens can be attached to the immunogenic polysaccharide in a modular fashion, allowing for flexibility in the number and type of SA antigens attached to immunogenic polysaccharide. Accordingly, the immunogenic polysaccharide can attach at least 1, or at least 2, or a plurality of the same, or different SA polypeptide antigens. In some embodiments, the immunogenic polysaccharide is antigenic, and in some embodiments, the immunogenic polysaccharide is Type 5 (CP5) or Type 8 (CP8), or a combination of Type 5 or Type 8 capsular polysaccharide from Staphylococcus aureus, or can be a pneumococcal capsular polysaccharide, e.g., Type 1 (CP1) capsular polysaccharide from S. pneumoniae.

In some embodiments, the SA-MAPS comprises at least one or more SA antigens, where at least one SA antigen is an antigenic polypeptide selected from any of the group of: SA1739 (B2), SA1720 (B1), SA1890 (B3), SA0103 (T1), SA0377 (T2), SA0693 (T3) and SA2105 (T4). In some embodiments, the SA-MAPS comprises at least one SA antigen is an antigenic polypeptide selected from any of the group of: SA1739 (B2), SA1720 (B1), SA1890 (B3), SA0103 (T1), SA0377 (T2), SA0693 (T3) and SA2105 (T4), and at least one SA antigenic polypeptide selected from any of the group consisting of: hemolysin (Hl) (e.g., hemolysin α or Hla), Clumping factor A (ClfA), Clumping factor B (ClfB), serine-aspirate repeat protein D (SdrD), Iron regulator surface protein A (IsdA) and Iron regulator surface protein B (IsdB).

In some embodiments, the SA-MAPS comprises at least one SA antigenic polypeptide selected from any of the group of: SA1739 (B2), SA1720 (B1), SA1890 (B3), SA0103 (T1), SA0377 (T2), SA0693 (T3) and SA2105 (T4), and one or more peptide or polypeptide fragments of these proteins, as long as the fragment is antigenic, and/or comprises one or more epitopes to induce an immune response.

In some embodiments, the SA-MAPS comprises at least one SA antigenic polypeptide selected from any of the group of: SA1739 (B2), SA1720 (B1), SA1890 (B3), SA0103 (T1), SA0377 (T2), SA0693 (T3) and SA2105 (T4), and one or more peptide or polypeptide fragments selected from, for example, but are not limited to Hla209(27-319), ClfA(221-559), ClfB (203-542), SdrD (246-682), IsdA (47-324), IsdB (48-447). In some embodiments, a SA-MAPS immunogenic composition as disclosed herein comprises at least one, or at least 2 or more SA antigenic polypeptide selected from any of the group of: SA1739 (B2), SA1720 (B1), SA1890 (B3), SA0103 (T1), SA0377 (T2), SA0693 (T3) and SA2105 (T4), and at least 2, or at least 3, or at least 4, or at least 5, or all 6 peptide or polypeptide SA-antigens of Hla209(27-319), ClfA(221-559), ClfB (203-542), SdrD (246-682), IsdA (47-324), IsdB (48-447), or proteins or peptides of at least 85% sequence identity thereto. In some embodiments, any of the above listed SA antigens can be substituted for a different SA peptide or polypeptide antigen known to one of ordinary skill in the art. Exemplary SA antigens can be any peptide or polypeptide comprising at least part of the serine-aspirate repeat protein E (SdrE) protein, Leukotoxin D (LukD) protein, or Leukotoxin E (LukE) protein, provided that the any peptide or polypeptide is immunogenic, or is antigenic. Other SA antigens can be used, and are disclosed herein.

The SA-MAPS immunogenic composition as disclosed herein can elicit both humoral and cellular responses to one or multiple SA antigens at the same time. The SA-MAPS immunogenic compositions provide for a long-lasting memory response, potentially protecting a subject from future infection. This allows for a single SA-MAPS immunogenic composition that raise a high titer of functional anti-SA polysaccharide antibodies, and is similar or compares favorably with the antibody level induced by conventional conjugate vaccine. Moreover, there is no restriction to specific immunogenic polysaccharide used in the MAPS construct, which is typically a SA capsular polysaccharide or other bacterial capsular or noncapsular polysaccharide, or the various SA antigen peptide or polypeptides used in SA-MAPS conjugate to generate a robust anti-polysaccharide antibody response. Additionally, the strong antibody response as well as Th17/Th1 and/or Th22 responses are specific to multiple SA protein antigens presented via the SA-MAPS composition. This is important in that the SA-MAPS immunogenic composition presents a major advantage by eliciting two forms of immunity—that is, a conventional immune response to an immunogenic polysaccharide and SA-antigens, as well as a T-cell response and, more specifically, any one or more of Th17, Th1, Th2 or Th22 responses to a SA peptide or protein present in the SA-MAPS composition. Moreover, the SA-MAPS immunogenic composition as disclosed herein provides a potential to enhance specific B-cell or T-cell responses by modifying the protein/polysaccharide ratio, complex size, or by incorporating specific co-stimulatory factor, such as TLR2/4 ligands, etc., into the composition.

Accordingly, the SA-MAPS immunogenic composition as disclosed herein uses an affinity-pair method to conjugate the SA antigens to the immunogenic polysaccharide, therefore enabling a modular approach that is easy and highly flexible for the preparation of a Staphylococcus aureus vaccine composition. The SA-MAPS immunogenic composition is highly specific and stable; it can remain in the cold for months and retain its potency. The assembly process is simple enough to ensure high reproducibility; there are only a few steps required, which reduces the risk of lot-to-lot variation, of great industrial advantage. The SA-MAPS immunogenic composition assembly is highly efficient (over 95%), even at low concentrations of protein and polysaccharide (such as 0.1 mg/ml); this is a major advantage, because inefficiencies in conjugate manufacture (typically efficiencies are in the <50% range) represent a major hurdle and reason for the high cost of vaccines. For formulation: it is easy to adjust the composition and physical properties of the final product. The protein: polysaccharide ratio in the complex is adjustable; with moderate biotinylation of polymer, protein: polysaccharide can be 10:1 (w/w) or more; conversely, the ratio can be 1:10 or less if such is the interest based on immunological goals. Additionally, the size of the immunogenic SA-MAPS composition can be adjusted by the choice of immunogenic polysaccharide size. The methods of making the SA-MAPS provide for ease in combining SA protein antigens and immunogenic polysaccharide with little modification, and allows the generation of a multivalent SA-MAPS composition by loading multiple SA peptide or protein antigens onto single immunogenic construct. As such, the SA-MAPS immunogenic composition as disclosed herein can be used to decrease the number of vaccines required to immunize a subject against Staphylococcus aureus, in particular, different strains of Staphylococcus aureus.

In some embodiments, the SA-MAPS immunogenic compositions as disclosed herein can be used to protect or treat a human susceptible to S. aureus infection, by means of administering the immunogenic compositions via a systemic, dermal or mucosal route or be used to generate a polyclonal or monoclonal antibody preparation that could be used to confer passive immunity on another subject. These administrations can include injection via the intramuscular, intraperitoneal, intradermal or subcutaneous routes; or via mucosal administration to the oral/alimentary, respiratory or genitourinary tracts. In one embodiment, intranasal administration is used for the treatment or prevention of nasopharyngeal carriage of S. aureus, thus attenuating infection at its earliest stage. In some embodiments, the SA-MAPS immunogenic compositions as disclosed herein may also be used to generate antibodies that are functional as measured by the killing of bacteria in either an animal efficacy model or via an opsonophagocytic killing assay.

In some embodiments, aspects of the invention disclosed herein relate to a SA-MAPS immunogenic composition comprising an immunogenic polysaccharide, at least one S. aureus peptide or polypeptide antigen, and at least one complementary affinity-molecule pair comprising: (a) a first affinity molecule associated with the immunogenic polysaccharide, and (b) a complementary affinity molecule associated with the at least S. aureus peptide or polypeptide antigen, where the first affinity molecule associates with the complementary affinity molecule to link the S. aureus peptide or polypeptide antigen and the immunogenic polysaccharide.

In some embodiments, a SA-MAPS composition as disclosed herein is used to elicit an immune response to S. aureus in a subject, for example, where the immune response is any of or a combination of: (i) an antibody or B-cell response, (ii) an antibody or B-cell response and T-cell response, (iii) an immune response to at least one immunogenic polysaccharide and at least one peptide or polypeptide S. aureus antigen, (iv) a CD4+ T cell response, including Th1, Th2, or Th17 or Th22 response, or a CD8+ T cell response, or CD4+ and CD8+ T cell response, (v) an antibody or B cell response to at least one antigenic polysaccharide and a CD4+ T cell response, including Th1, Th2, or Th17 or Th22 response, or a CD8+ T cell response, or CD4+/CD8+ T cell response to at least one peptide or polypeptide antigen, (vi) an antibody or B cell response to at least one antigenic polysaccharide, and an antibody or B cell response and a CD4+ T cell response, including Th1, Th2, Th17 or Th22 responses, or a CD8+ T cell response, or CD4+/CD8+ T cell response to at least one peptide or polypeptide antigen, (vii) results in activation of INF-γ, IL-17A or IL-22 producing cells, or INF-γ, IL-17A and IL-22 producing cells, (viii) an antibody or B-cell response against the S. aureus antigen which associates with the immunogenic polysaccharide.

In some embodiments, a SA-MAPS composition as disclosed herein, further comprises at least one adjuvant.

In some embodiments, a SA-MAPS composition as disclosed herein is used in a diagnostic for exposure to a pathogen or immune threat. In some embodiments, a SA-MAPS composition as disclosed herein is used in preventing infection by S. aureus. In some embodiments, a SA-MAPS composition as disclosed herein is used for preventing colonization of a subject by S. aureus.

Another aspect of the technology disclosed herein relates to a method for inducing an immune response in a subject to S. aureus, comprising administering to the subject a SA-MAPS composition as disclosed herein. For example, the SA-MAPS composition as disclosed herein is used to induce an immune response in a subject to S. aureus, where the immune response is, for example, any of or a combination of: (i) an antibody or B-cell response, (ii) an antibody or B-cell response and T-cell response, (iii) an immune response to at least one immunogenic polysaccharide and at least one peptide or polypeptide S. aureus antigen, (iv) a CD4+ T cell response, including Th1, Th2, or Th17 or Th22 response, or a CD8+ T cell response, or CD4+ and CD8+ T cell response, (v) an antibody or B cell response to at least one antigenic polysaccharide and a CD4+ T cell response, including Th1, Th2, or Th17 or Th22 response, or a CD8+ T cell response, or CD4+/CD8+ T cell response to at least one peptide or polypeptide antigen, (vi) an antibody or B cell response to at least one antigenic polysaccharide, and an antibody or B cell response and a CD4+ T cell response, including Th1, Th2, Th17 or Th22 responses, or a CD8+ T cell response, or CD4+/CD8+ T cell response to at least one peptide or polypeptide antigen, (vii) results in activation of INF-γ, IL-17A or IL-22 producing cells, or INF-γ, IL-17A and IL-22 producing cells, (viii) an antibody or B-cell response against the S. aureus antigen which associates with the immunogenic polysaccharide.

Another aspect of the technology disclosed herein relates to a method of vaccinating a mammal against at least one antigen-bearing pathogen, the method comprising administering to the mammal a SA-MAPS composition as disclosed herein. In some embodiments, the subject or mammal is a human. In alternative embodiments, the subject or mammal is an agricultural or non-domestic animal, or a domestic animal.

In some embodiments, a SA-MAPS composition as disclosed herein is administered via subcutaneous, intranasal, intradermal, or intra muscular injection, or via transdermal skin patch.

Another aspect of the technology disclosed herein relates to a kit comprising: (a) a container comprising an immunogenic polysaccharide cross-linked with a plurality of first affinity molecules; and (b) a container comprising a complementary affinity molecule which associates with the first affinity molecule, wherein the complementary affinity molecule associates with at least one S. aureus antigen. In some embodiments, a kit can further comprise any one or more of: (i) a means or agent to attach the complementary affinity molecule to the antigen, (ii) at least one co-stimulation factor, (iii) a cross-linking reagent which can be selected from the group consisting of: CDAP (1-cyano-4-dimethylaminopyridinium tetrafluoroborate), EDC (1-Ethyl-3-[3-dimethylaminopropyl]carbodiimide hydrochloride), sodium cyanoborohydride, cyanogen bromide, or ammonium bicarbonate/iodoacetic acid for linking the co-factor to the polysaccharide, (iv) a container comprising an expression vector for expressing an antigen-affinity molecule fusion protein, for example, an expression vector that can optionally comprise a sequence for a linker peptide, wherein the expression vector can expresses an antigen-affinity molecule fusion protein comprising a linker peptide between the antigen and the affinity molecule, and/or (v) one or more of a fusion protein as disclosed herein, wherein the fusion protein is selected from any of: (i) a protein comprising SEQ ID NO: 1 (or a protein of at least 80% or 85% or more sequence identity thereto) is fused to any of: SA1739 (B2), SA1720 (B1), SA1890 (B3), SA0103 (T1), SA0377 (T2), SA0693 (T3) and SA2105 (T4), or fragments thereof, or (ii) a fusion protein comprising a the C-terminal of SEQ ID NO: 1 (or a protein of at least 80% or 85% or more sequence identity thereto) fused to any one of: SA 1739 (B2) (SEQ ID NO: 51), SA1720 (B1) (SEQ ID NO: 50), SA1890 (B3) (SEQ ID NO: 52), SA0103 (T1) (SEQ ID NO: 53), SA0377 (T2) (SEQ ID NO: 54), SA0693 (T3) (SEQ ID NO: 55) and SA2105 (T4) (SEQ ID NO: 56), or proteins or peptides having at least 85% sequence identity thereto, or (iii) a fusion protein selected from any of Rhavi-SA1739, Rhavi-SA1720, Rhavi-SA1890, Rhavi-SA0103, Rhavi-SA0377, Rhavi-SA0693, Rhavi-SA2105, Rhavi-SA0103-SA0377, Rhivi-SA0103-SA0693, Rhivi-SA0103-SA2105, Rhavi-SA0103-SA1739, Rhavi-SA0103-SA1720, Rhavi-SA0103-SA1890, Rhivi-SA0377-SA0693, Rhivi-SA0377-SA2105, Rhavi-SA0377-SA1739, Rhavi-SA0377-SA1720, Rhavi-SA0377-SA1890, Rhivi-SA0693-SA2105, Rhavi-SA0693-SA1739, Rhavi-SA0693-SA1720, Rhavi-SA0693-SA1890, Rhavi-SA2105-SA1739, Rhavi-SA2105-SA1720, Rhavi-SA2105-SA1890, Rhavi-SA1739-SA1720, Rhavi-SA1739-SA1890, Rhavi-SA1720-SA1890.

Accordingly, one aspect of the present invention relates to an immunogenic composition comprising a polymer, e.g., immunogenic polysaccharide, at least one SA-polypeptide antigen as disclosed herein (e.g., a Group A SA-antigen), and at least one complementary affinity-molecule pair, where the complementary affinity-molecule pair comprises a first affinity molecule (e.g., biotin) that associates with the immunogenic polysaccharide and a complementary affinity molecule (e.g., biotin-binding protein) that associates with the SA-polypeptide antigen, so that when the first affinity molecule associates with the complementary affinity molecule, it indirectly links the SA-polypeptide antigen to the immunogenic polysaccharide.

Provided herein also is a method of vaccinating a subject, e.g., a mammal, e.g., a human with the SA-MAPS immunogenic compositions as disclosed herein, the method comprising administering a vaccine composition comprising a SA-MAPS composition as disclosed herein to the subject.

Provided herein also are pharmaceutical compositions comprising the SA-MAPS immunogenic compositions as disclosed herein. Also provided herein are fusion proteins comprising a biotin-binding protein (e.g., Rhavi) and at least one SA-antigen as disclosed herein. Also provided herein are nucleic acid sequence encoding the fusion proteins disclosed herein, comprising a biotin-binding protein (e.g., Rhavi) and at least one SA-antigen as disclosed herein.

### DESCRIPTION OF THE DRAWINGS

This patent or application file contains at least one drawing executed in color. Copies of this patent or patent application publication with color drawing(s) will be provided by the Office upon request and payment of the necessary fee.

FIG. 1 shows a schematic of the surface protein library construction used in a method to identify SA antigens that elicit a B-cell response for use in the SA-MAPS vaccine compositions as disclosed herein. A surface protein library was constructed from S. aureus USA300 genome (2564 genes), and screened through a number of parameters, including (i) selection of genes with signal peptides or transmembrane domains (789 genes), (ii) selection of genes with >100 amino acids in the extracellular domain (266 genes), (iii) selection of genes convered in all sequences strains (231 genes), (iv) construction of vectors and screening of constructs expressing the genes (231 genes), and (v) expression and purification of SA antigens in E. coli (195 proteins).

FIG. 2 shows results of the OPA killing assay, which showed highest percentage killing by the antigens in the group 3. From about 195 SA proteins in the surface protein library, the inventors excluded toxins. The remaining 159 candidate SA surface proteins were allocated into 8 groups of 20 and used for immunization of 5 Balb/c mice per group (each mouse group received a combination of 20 proteins at 5 μg per protein). Mice were bled two weeks after the second immunization for initial test and terminally bled at 5 weeks post-second immunization. Serum was used in OPA killing assays with neutrophils derived from HL-60 cells.

FIG. 3A-3F shows the results of the OPA killing assay, showing the percent inhibition of killing by selected purified SA proteins. FIG. 3A shows a significant percentage inhibition of killing HL-60 cells by SA proteins 419, 579, 629, 641, 693 and 717. FIG. 3B shows a significant percentage inhibition of killing HL-60 cells by SA proteins 1002, 1018 and 1030. FIG. 3C shows a significant percentage inhibition of killing HL-60 cells by SA proteins 1313, 1341 and 1441. FIG. 3D shows a significant percentage inhibition of killing HL-60 cells by SA proteins 1682, 1720, 1739 and 1890. FIG. 3E shows a significant percentage inhibition of killing HL-60 cells by SA proteins 2256 and 2309. FIG. 3F shows a significant percentage inhibition of killing HL-60 cells by SA protein 2448 and 2572.

FIG. 4 is a table of the SA proteins selected for further analysis.

FIG. 5 shows in vitro Assessment of killing activity by 14 candidate SA antigens proteins in the sera. 14 candidate SA proteins were assessed by immunization of Balb/c mice and the serum from immunized mice was used in the OPA killing assay. Briefly, Balb/C were immunized with one of the 14 selected proteins twice at two-week interval, and the mice were bled two weeks after the second immunization for evaluation of killing activity of sera and boosted again at 4 weeks after the second immunization. Sera were used in OPA killing assays with neutrophils derived from HL-60 cells. Mice were infected two weeks after last immunization.

FIG. 6A-6D shows the results of the In vivo assessment of SA antigens to protect Balb/C or B16 mice from S. aureus infection. FIG. 6A shows a Kaplien Mier plot showing the percent survival of the mice of Balb/C mice immunized with individual candidate SA antigens, and shows results of single SA protein protection of Balb/C mice infected with S. aureus. FIG. 6B shows that 100% of Balb/C mice innoculated with SA1216 survived by day 14, and 60% of Balb/C mice innoculated with SA1890, SA1739 survived by 14 days after infection with S. aureus. FIG. 6B shows the percent survival of the mice of B16 mice immunized with individual candidate SA antigens SA1720, SA1739, SA1890 and SA2256. Mice immunized with SA1890, or SA1739 or SA1720 showed a 50%, 30% and 20% survival rate, respectively, 14-days after infection with S. aureus. FIG. 6C shows the percent survival of the Balb/C mice immunized with a combination of SA antigens after i.v. infection with S. aureus, and shows that B16 mice immunized with all three SA proteins (SA1720, SA1739 and SA1890), or with two SA proteins (SA1739 and SA1890) had a 60% survival 14 days after infection with S. aureus, or B16 mice immunized with two SA proteins (SA1720 and SA1739) or SA1739 alone had a 50% survival 14 days after infection with S. aureus. FIG. 6D shows the percent survival of the mice of B16 mice immunized with SA antigens (SA1739 or SA1739 and SA1720), with a combination with known SA antigens previously disclosed in US2021/0008192 (″192 application). Two SA proteins (SA1739 or SA1730) in combination with a MAPS4 complex (an immunogenic MAPS composition comprising a pneumococcal type 1 polysacharide, and comprising 4 SA proteins: hemolysin (Hl) (e.g., Hla209AA), Clumping factor A (ClfA), Clumping factor B (ClfB) and serine-aspirate repeat protein D (SdrD)). FIG. 6D shows that B16 mice immunized with MAPS4 complex alone had a 20% survival rate 14-days after infection with s. aureus, whereas when B16 mice were immunized with MAPS4 and SA1739 or MAPS and both SA1720 and SA1739, had a 40% or 20% survival rate, respectively, 14-days after infection with S. aureus, demonstrating that at least SA1739 can enhance the protective effect of the MAPS4 complex.

FIG. 7 show the clinical study overview to identify SA antigens eliciting a T-cell response, and details of the clinical enrollment and sampling schedules where serum was obtained from human subjects (n=31). Screening of the T-cell surface protein library against human immune cells was performed to identify antigens that specifically induce Th1 and Th17 responses in the resistant population, which could be candidates for inclusion in a vaccine.

FIG. 8 shows the categorization of subject in the clinical study (n=31) where the T-cell surface protein library was screened against human immune cells to identify antigens that specifically induce Th1 and Th17 responses in the resistant population, which could be candidates for inclusion in a vaccine.

FIG. 9 shows identified T-cell inducing SA antigens which were assessed. Antigens that have superantigen potential (e.g. Eap) were not selected for potential safety concerns; nor were antigens that had a high stimulatory in every subject, making them unlikely to be good antigen targets (e.g. SA protein 1588).

FIG. 10 shows a table often T-cell candidate SA antigens selected for purification and further analysis.

FIG. 11 shows a table of the four T-cell antigens SA0103, SA0377, SA0693 and SA2105 selected for fusion to a biotin-binding protein (e.g., Rhavi) for use in a SA-MAPS complex.

FIG. 12 shows results of in vivo protection against skin abscess by four T-cell SA antigens. SA antigens SA0103 (T1), SA0377 (T2), SA0693 (T3) and SA2105 (T4) were purified as Rhavi fusion proteins and complexed to pneumococcal CPS1. Mice were immunized three times with two-weeks apart and challenged with S. aureus (USA300 TCH959 strain) subcutaneously, and bacterial CFU in the abscess was determined 4 days after infection. FIG. 12 shows the significant protection against abscess by T cell antigens SA0103 (T1), SA0377 (T2), SA0693 (T3) and SA2105 (T4), when they present as part of a MAPS composition, or when both SA0103 (T1) and SA2105 (T4) are present in a MAPS composition.

FIG. 13 shows a Kaplien Mier plot showing protection (the percent survival) after a sepsis challenge of the mice of B16 mice immunized with MAPS4 or MAPS4 plus additional candidate T-cell or B-cell SA antigens, and shows results of protection of B16 mice infected with S. aureus. Mice were immunized two times with two-weeks apart and challenged IV with 29213 strain. T1T2 is a fusion of SA0103, SA0377 and Rhavi (Rhavi-SA0103-SA0377). T1T3 is a fusion of SA0103, SA0693 and Rhavi (Rhavi-SA0103-SA0693). FIG. 13 shows MAPS4 plus SA1739 (B2) shows the highest percent survival against sepsis, and MAPS4 plus three different SA antigens selected from the groups of: (i) SA0103 (T1)+SA0377 (T2)+SA1739 (B2) or (ii) SA0103 (T1), +SA0693 (T3)+SA1739 (B2) results in significant survival benefit from sepsis. MAPS4 is an immunogenic MAPS composition comprising a pneumococcal type 1 polysacharide, and comprising 4 SA proteins: hemolysin (Hl) (e.g., Hla209AA), Clumping factor A (ClfA), Clumping factor B (ClfB) and serine-aspirate repeat protein D (SdrD).

FIG. 14 shows enhanced in vivo protection against abscess by addition of T-cell and B-cell SA antigens as part of a MAPS4 complex. Mice were immunized two times with two-weeks apart and challenged with USA300 TCH959 SA strain subcutaneously, and bacterial CFU in the abscess was determined 4 days after infection. FIG. 14 shows a comparison of the protection by (i) MAPS4 alone (ii) MAPS4 plus a B-cell SA antigen (e.g., MAPS4+SA1739 (B2)) or (iii) MAPS4 plus both a B-cell and T cell SA antigen (e.g., MAPS4+ TI(SA0103)-T2(SA0377) fusion+B2 (SA1739)) or (iv) MAPS4+ T1(SA0103)-T3(SA0693) fusion+B3 (SA1739), where T1-T2 (SA0103-SA0377) and T1-T3 (SA0103)-T3(SA0693) are T-cell SA antigen fusion proteins. A significant improvement in protection against sepsis was detected when the combination of both a B-cell antigen (e.g., SA1739 (B2)) and a T-cell antigen (e.g., SA0103-SA0377 or T1(SA0103)-T3(SA0693)) was added to the MAPS4 complex as compared to MAPS4 alone. Pneumococcal CPS1 was used as backbone of MAPS4. T1T2 is a fusion of SA0103, SA0377 and Rhavi (e.g., Rhavi-SA0103-SA0377 fusion protein). T1T3 is a fusion of SA0103, SA0693 and Rhavi (e.g. Rhavi-SA0103-SA0693 fusion protein).

### DETAILED DESCRIPTION OF THE INVENTION

The present invention relates immunogenic compositions and compositions comprising an improved immunogenic complex that comprises at least two Staphylococcus aureus antigen, attached to an immunogenic polysaccharide scaffold for use in eliciting an immune response (both a cellular and humoral immune response) to each of the SA polypeptide antigens attached to the immunogenic polysaccharide and to the immunogenic polysaccharide, when administered to a subject.

More specifically, disclosed herein is an immunogenic Multiple Antigen Presenting System (MAPS) comprising an immunogenic polysaccharide, and attached to the immunogenic polysaccharide via an affinity binding pair, at least one Staphylococcus aureus (SA) antigen. Such a Staphylococcus aureus-MAPS (SA-MAPS) composition as disclosed herein comprises at least one newly discovered SA polypeptide antigen alone, or in addition to those disclosed in US application US2021/0008192 (the '192 application), which is incorporated herein in its entirety by reference. Such a SA-MAPS composition as disclosed herein is useful for the production of immunogenic compositions, which are useful in vaccines, and/or for treatment. The SA-MAPS immunogenic composition as disclosed herein stimulates a humoral and cellular immune response: it can generate anti-polysaccharide antibody and the B-cell and T-cell, e.g., Th1/Th17 responses to multiple Staphylococcus aureus (SA) antigen using single SA-MAPS immunogenic construct. A combination of B- and T-cell immunity to Staphylococcus aureus will be a useful vaccine strategy against Staphylococcus aureus invasive diseases, as well as from mild skin infections to endocarditis, dermonecrosis, osteomyelitis, bacteremia, sepsis, and other forms of disease associated with Staphylococcus aureus.

The inventors previously developed a vaccine platform referred to the Multiple-Antigen-Presenting-System (MAPS), as disclosed in US patent Application 2014/0154287, which is incorporated herein in its entirety by reference, which enables the induction of broad adaptive immune responses. Herein, the inventors have developed and optimized the system for the treatment and prevention of infection from Staphylococcus aureus. Additionally, the inventors previously described in US application US2021/0008192 (″192 application) an immunogenic MAPS composition comprising at least one Staphylococcus aureus (SA) peptide antigens (SA-MAPS). In the '192 application, which is incorporated herein by reference in its entirety, the SA peptide antigens are selected from any of: hemolysin (Hl) (e.g., hemolysin α or Hla), Clumping factor A (ClfA), Clumping factor B (ClfB), serine-aspirate repeat protein D (SdrD), Iron regulator surface protein A (IsdA) and Iron regulator surface protein B (IsdB). The '192 application discloses a specific SA-MAPS immunogenic composition.

Without wishing to be bound by theory, a SA-MAPS composition as disclosed herein refers to, in brief, an immunogenic composition comprising (i) at least one immunogenic polysaccharide which has a first affinity molecule attached (e.g., biotin attached), and (ii) at least one S. aureus polypeptide antigen, where the S. aureus polypeptide antigen is a SA-antigen as disclosed herein, e.g., at least one Group A SA-antigen, and is fused to a second affinity molecule, (e.g., a biotin-binding moiety, such as, but not limited to Rhizavidin comprising the amino acid sequence of SEQ ID NO: 1), wherein the first affinity molecule non-covalently associates with the second affinity molecule, thereby linking the S. aureus polypeptide antigen(s) and the immunogenic polysaccharide.

Stated differently, in one embodiment, the SA-MAPS composition as disclosed herein refers to, in brief, an immunogenic composition comprising, (i) a biotinylated immunogenic polysaccharide and (ii) at least one fusion protein, the fusion protein comprising a biotin-binding protein fused to at least one SA-antigen as disclosed herein, e.g., at least one Group A SA-antigen as disclosed herein, and where the biotinylated polysaccharide antigen is non-covalently associated with the biotin-binding moiety of the fusion protein to form an immunogenic complex. In some embodiments, the immunogenic polysaccharide is selected from any of: type 1 capsular polysaccharide of Streptococcus pneumoniae, a type 5 capsular polysaccharide of S. aureus, and/or a type 8 capsular polysaccharide of S. aureus.

Herein, the technology relates to an improved SA-MAPS composition, where the immunogenic SA polypeptide antigens are selected from at least one SA polypeptide antigen from “Group A” SA polypeptide antigens, where Group A SA polypeptide antigens comprise: SA1739 (B2), SA1720 (B1), SA1890 (B3), SA0103 (T1), SA0377 (T2), SA0693 (T3) and SA2105 (T4). In some embodiments, the SA-MAPS immunogenic composition as disclosed herein comprises one or more SA polypeptide antigens of Group A, or fragments of these proteins, as long as the fragment is antigenic, and/or comprises one or more epitopes to induce an immune response.

Herein, the technology relates to an improved SA-MAPS composition, where the immunogenic SA polypeptide antigens are selected from at least one SA polypeptide antigen from “Group A(i)” SA polypeptide antigens, where Group A(i) is a subset of Group A, and are B-cell SA polypeptide antigens and comprise: SA1739 (B2), SA1720 (B1), SA1890 (B3). Herein, the technology relates to an improved SA-MAPS composition, where the immunogenic SA polypeptide antigens are selected from at least one SA polypeptide antigen from “Group A(ii)” SA polypeptide antigens, where Group A(ii) is a subset of Group A, and are T-cell SA polypeptide antigens and comprise: SA0103 (T1), SA0377 (T2), SA0693 (T3) and SA2105 (T4).

Herein, the technology relates to an improved SA-MAPS composition, where the immunogenic SA polypeptide antigens are selected from at least one SA polypeptide antigen from Group A, Group A(i) or Group A(ii) SA polypeptide antigens, and at least one SA polypeptide antigen from “Group B” SA polypeptide antigens, where Group B(i) comprise: hemolysin (Hl) (e.g., hemolysin α or Hla), Clumping factor A (ClfA), Clumping factor B (ClfB), serine-aspirate repeat protein D (SdrD), Iron regulator surface protein A (IsdA) and Iron regulator surface protein B (IsdB), or fragments thereof. In some embodiments, the technology relates to a SA-MAPS composition, where the immunogenic SA polypeptide antigens are selected from at least one SA polypeptide antigen from Group A, Group A(i) or Group A(ii) or fragments thereof, and at least one SA polypeptide antigen from “Group B(i)”, where Group B(i) are fragments of Group B SA antigens and comprises: Hla209(27-319), ClfA(221-559), ClfB (203-542), SdrD (246-682), IsdA (47-324), IsdB (48-447), or proteins or peptides of at least 85% sequence identity thereto. In some embodiments, the technology relates to a SA-MAPS composition, where the immunogenic SA polypeptide antigens are selected from at least one SA polypeptide antigen from Group A, Group A(i) or Group A(ii) or fragments thereof, and at least one SA polypeptide antigen from “Group B(ii)”, where Group B(ii) is a subgroup of Group B SA antigens and comprises: Hla209(27-319), ClfA(221-559), ClfB (203-542), SdrD (246-682), IsdA (47-324), IsdB (48-447), or fragments or proteins or peptides of at least 85% sequence identity thereto. In some embodiments, the technology relates to a SA-MAPS composition, where the immunogenic SA polypeptide antigens are selected from at least one SA polypeptide antigen from Group A, Group A(i) or Group A(ii) or fragments thereof, and at least one SA polypeptide antigen from “Group B(iii)”, where Group B(iii) is a subgroup of Group B(i) SA antigens and comprises: Hla209(27-319), ClfA(221-559), ClfB (203-542), SdrD (246-682) or fragments or proteins or peptides of at least 85% sequence identity thereto.

Exemplary SA polypeptide antigens of Group A, Group A(i) and Group A(ii) for use in a SA-MAPS immunogenic composition are disclosed in Table 1A. Exemplary SA polypeptide antigens of Group B, Group B(i), Group B(ii) and Group B(iii) for use in a SA-MAPS immunogenic composition are disclosed in Table 1B.

TABLE 1A

| | SA polypeptide antigens |
|---|---|
| Group A | SA1739 (B2), SA1720 (B1), SA1890 (B3), SA0103 |
| | (T1), SA0377 (T2), SA0693 (T3) and SA2105 (T4). |
| Group A(i) | SA1739 (B2), SA1720 (B1), SA1890 (B3), |
| Group A(ii) | SA0103 (T1), SA0377 (T2), SA0693 (T3) and |
| | SA2105 (T4). |

TABLE 1B

| | SA polypeptide antigens |
|---|---|
| Group B | hemolysin (Hl) (e.g., hemolysin α or Hla), Clumping |
| | factor A (ClfA), Clumping factor B (ClfB), serine- |
| | spirate repeat protein D (SdrD), Iron regulator |
| | surface protein A (IsdA) and Iron regulator surface |
| | protein B (IsdB), or fragments thereof |
| Group B(i) | Hla209(27-319), ClfA(221-559), ClfB (203-542), |
| | SdrD (246-682), IsdA (47-324), IsdB (48-447) |
| Group B (ii) | hemolysin (Hl) (e.g., hemolysin α or Hla), Clumping |
| (“MAPS4”) | factor A (ClfA), Clumping factor B (ClfB), serine- |
| | aspirate repeat protein D (SdrD) |
| Group B(iii) | Hla209(27-319), ClfA(221-559), ClfB (203-542), SdrD |
| | (246-682) |

In particular, the inventors have generated a SA-MAPS immunogenic composition comprising an immunogenic polysaccharide (typically SA CP5, CP8 or S. pneumoniae CP1, or other PS or variants or combinations thereof), at least one Staphylococcus aureus polypeptide antigen selected from the SA polypeptide antigens of Group A; and at least one complementary affinity-molecule pair comprising (i) a first affinity molecule that associates with the immunogenic polysaccharide, where in some embodiments the affinity molecule is biotin, and (ii) a complementary affinity molecule that associates with the Staphylococcus aureus polypeptide or peptide antigen, where the complementary affinity molecule is a biotin-binding moiety (e.g., Rhavi), such that the first and complementary affinity molecules serve as an indirect link between the immunogenic polysaccharide and SA polypeptide antigens. Such a system allows for a modular immunogenic composition, where one or more SA polypeptide antigens can be attached to the immunogenic polysaccharide in a modular fashion, allowing for flexibility in the number and type of SA antigens attached to immunogenic polysaccharide. Accordingly, the immunogenic polysaccharide can attach at least 1, or at least 2, or a plurality of the same or different SA polypeptide antigens. In some embodiments, the immunogenic polysaccharide is antigenic, and in some embodiments, the immunogenic polysaccharide is Type 5 (CP5) or Type 8 (CP8), or a combination of Type 5 or Type 8 capsular polysaccharide from Staphylococcus aureus, or can be a pneumococcal capsular polysaccharide, e.g., Type 1 (CP1) capsular polysaccharide from S. pneumoniae.

In some embodiments, the SA-MAPS composition useful in the methods and compositions as disclosed herein is an immunogenic composition comprising, (i) a biotinylated immunogenic polysaccharide and (ii) at least one fusion protein, the fusion protein comprising a biotin-binding protein fused to at least one SA-antigen as disclosed herein, e.g., at least one Group A SA-antigen as disclosed herein, and where the biotinylated polysaccharide antigen is non-covalently associated with the biotin-binding moiety of the fusion protein to form an immunogenic complex. In some embodiments, the immunogenic polysaccharide is selected from any of: type 1 capsular polysaccharide of Streptococcus pneumoniae, a type 5 capsular polysaccharide of S. aureus, and/or a type 8 capsular polysaccharide of S. aureus.

Herein, the inventors have used a SA-specific MAPS immunogenic composition which comprises different SA peptide antigens to demonstrate that B- and T-cell mediated immune mechanisms contribute differentially to host defense against SA in models of skin necrosis, skin abscess, invasive disease or mucosal colonization.

In some embodiments, the SA-MAPS comprises at least one or more SA antigens, where the SA antigen is an antigenic protein or polypeptide selected from any of the group of: SA1739 (B2), SA1720 (B1), SA1890 (B3), SA0103 (TI), SA0377 (T2), SA0693 (T3) and SA2105 (T4). In some embodiments, the SA-MAPS immunogenic composition as disclosed herein comprises one or more peptide or polypeptide fragments of these proteins, as long as the fragment is antigenic, and/or comprises one or more epitopes to induce an immune response. In some embodiments, a SA-MAPS immunogenic composition as disclosed herein comprises at least 1 or at least 2, or at least 3, or at least 4, or at least 5, or all 6 peptide or polypeptide SA-antigens of SA1739 (B2), SA1720 (B1), SA1890 (B3), SA0103 (T1), SA0377 (T2), SA0693 (T3) and SA2105 (T4), or proteins or peptides of at least 85% sequence identity thereto. In some embodiments, any of the above listed SA antigens can be substituted for a different SA peptide or polypeptide antigen known to one of ordinary skill in the art. Exemplary SA antigens can be any peptide or polypeptide comprising at least part of the serine-aspirate repeat protein E (SdrE) protein, Leukotoxin D (LukD) protein, or Leukotoxin E (LukE) protein, provided that the any peptide or polypeptide is immunogenic, or is antigenic. Other SA antigens can be used, and are disclosed herein.

Accordingly, the embodiments herein provide for an immunogenic composition and methods useful for raising an immune response to Staphylococcus aureus in a subject, which can be used on its own or in conjunction or admixture with essentially any existing vaccine approaches.

Staphylococcus aureus Multiple-Antigen Presenting System (SA-MAPS)

While it is envisioned that the SA-MAPS immunogenic composition as disclosed herein comprises immunogenic polysaccharides from Staphylococcus aureus, the SA-MAPS can use immunogenic polysaccharides from a variety of different bacterial cells. In some embodiments, the immunogenic polysaccharide is for example, but not limited to, Type 5 (CP5) or Type 8 (CP8), or a combination of Type 5 or Type 8 capsular polysaccharide from Staphylococcus aureus, or can be a pneumococcal capsular polysaccharide, e.g., Type 1 (CP1) capsular polysaccharide from S. pneumoniae, or other capsular or noncaspular PS. In some embodiments, the polysaccharide is a capsular polysaccharide. In some embodiments, the polysaccharide is not a capsular polysaccharide (i.e., a noncapsular PS). With the different combinations of immunogenic polysaccharides and different combinations of SA peptide or polypeptide antigens, the SA-MAPS composition is a flexible and versatile composition that can be designed and manufactured to elicit a particular, broad spectrum immune response to Staphylococcus aureus. Table 2 provides a simple example guide for envisioning the flexibility of SA-MAPS embodiments.

Table 2 shows the versatility of the SA-MAPS platform: SA-MAPS comprises an antigenic polysaccharide backbone and at least one SA-antigen, and optionally one or more non-SA antigens. The antigenic or immunogenic polysaccharide backbone can be a synthetic or antigenic polysaccharide from Staphylococcus aureus or alternatively a different a pathogen (exemplary antigenic polysaccharides are listed in the last column). A SA-MAPS composition can comprise at least one SA-antigen (exemplary SA antigens are listed), and can optionally comprise non-SA antigens.

A. S. aureus Antigens

It is well recognized that any single animal model of SA infection is unlikely to adequately represent the pathophysiology of disease in humans; therefore, evaluation of any potential candidate in several models would appear prudent. At the same time, the large number of virulence factors (including polysaccharides, surface proteins, and secreted toxins produced by SA, may provide credence to the idea that multiple, genetically conserved antigens should be included in a candidate vaccine. Finally, a closer examination of mechanisms of immunity to SA in humans may also provide clues for an effective vaccine strategy. Indeed, while humoral immunity plays a leading role in host defense against many bacterial or viral pathogens, it is unlikely that antibodies are the only or even the primary factor for resistance to SA. Patients with B-cell deficiencies do not appear to be at significantly increased risk of SA infections, and individuals with high levels of pre-existing SA-specific antibodies can still get infected by SA. On the other hand, a growing body of literature now suggests that T-cell immunity, the other arm of acquired host defense, plays a critical role in SA defense. Indeed, individuals with suppressed or impaired cellular immunity, caused by high dose prednisone therapy, HIV infection, defective interferon-γ (IFN-γ) production, defective interleukin-17 (IL-17) production, are at very high risk for SA infection and recurrence. Moreover, in murine models, IFN-γ or IL-17A/F deficiency has been shown to induce hyper-susceptibility to SA skin infections, and IL-17A deficiency in mice is also associated with prolonged nasal carriage of SA. Therefore, the inventors have developed a SA-MAPS immunogenic composition that induces both B- and T-cell acquired immunes responses in the organism may provide optimal protection against this organism.

Herein, the inventors have generated a SA-MAPS immunogenic composition comprising containing several conserved SA antigens to elicit a broad range of immune responses. More specifically, the inventors demonstrate a vaccine platform, referred herein as the Staphylococcus aureus Multiple-Antigen-Presenting-System (SA-MAPS), which comprises an immunogenic polysaccharide with affinity-coupled complexes of SA antigens that can induce broad B- and T-cell responses. The immune response generated with the SA-MAPS vaccine was compared to a multi-component SA subunit vaccine using a conventional approach (i.e., immunization with purified proteins alone, and not attached to an immunogenic polysaccharide). The inventors demonstrated the immunogenicity of these two vaccines (the antigens alone, or antigens as part of the SA-MAPS complex) in mice, compared their protective efficacy in SA sepsis infection, dermonecrosis infection, skin abscess infection and gastrointestinal (GI) colonization models, and finally, studied the role of antigen-specific antibodies and T-cell immunity against different types of SA infection or colonization

An immunogenic SA antigen for use in the immunogenic compositions and methods described herein can be any SA antigen, including, but not limited to pathogenic peptides, toxins, toxoids, subunits thereof, or combinations thereof. In some embodiments, a SA polypeptide antigen is fused to a complementary affinity molecule, e.g., a biotin-binding protein or biotin-binding moeity such as rhizavidin as disclosed herein, can be any SA. antigen, peptide, polypeptide, polysaccharide, expressed by Staphylococcus aureus bacterium.

1. Group A S. aureus Antigens

In some embodiments, the SA-MAPS comprises at least one antigen from Group A, where the SA antigen is an antigenic protein or polypeptide, and can be selected from any of Group A SA polypeptide antigens of: SA1739 (B2), SA1720 (B1), SA1890 (B3), SA0103 (TI), SA0377 (T2), SA0693 (T3) and SA2105 (T4), or an antigenic fragment or portion thereof. In some embodiments, the SA-MAPS immunogenic composition as disclosed herein comprises one or more peptide or polypeptide fragments of Group A SA polypeptide antigens, as long as the protein fragment is antigenic, and/or comprises one or more epitopes to induce an immune response.

TABLE 3
Table 3 lists the amino acid sequence identification numbers of the S. aureus
polypeptide antigens. The amino acid sequences and nucleotide sequences of the
S. aureus antigens are available on world-wide web site: “xbase.ac.uk/genome/streptococcus-
pneumoniae-tigr4/NC_003028/features?page=1”, which is incorporated
herein in its entirety by reference.

| | | | DNA | | | |
|---|---|---|---|---|---|---|
| S. aureus | | Protein | SEQ | | | Immunogenic |
| antigen | Predicted | SEQ ID | ID | | GenBank | fragments of the |
| name | functions | NO: | NO: | Gene ID NO. | Accession No | SA antigen |
| SA1720 (B1) | mannosyl- | 50 | 57 | | Residues 41- | Residues 30-285 |
| | glycoprotein | | | | 295 of | of SA1720 |
| | endo-beta-N- | | | | ADA15666.1 | SA1720 (a.a 30- |
| | acetylglucosaminidase | | | | (GenBank: | 285): |
| | [Staphylococcus | | | | EES92546.1) | |
| | aureus subsp. | | | | | |
| | aureus | | | | | |
| | USA300_TCH959] | | | | | |
| SA1739 (B2) | | 51 | 58 | | Residues 28- | Residues 60-217 |
| | | | | | 184 of | of SA1739 B2: |
| | | | | | AAQ52145.1 | SA1739 (a.a. 60- |
| | | | | | or residues 60- | 217): |
| | | | | | 216 of | |
| | | | | | ADA15640.1 | |
| SA1890 (B3) | Staphopain A | 52 | 59 | WP_000827748.1 | Residues 26- | (B3: SA1890 (aa |
| | | | | https:// | 388 of SEQ ID | 26-389) |
| | | | | www.ncbi.nlm.nih.gov/ | NO: 1402 | |
| | | | | ipg/ADA15958.1 | (ADA15958.1) | |
| SA0103 (T1) | lipoprotein | 53 | 60 | WP_000826311.1 | Residues 27- | SA0103 (27-256) |
| | | | | https:// | 255 of SEQ ID | |
| | | | | www.ncbi.nlm.nih.gov/ | NO: 4612 | |
| | | | | ipg/ADA17563.1 | from U.S. | |
| | | | | | Pat. No. | |
| | | | | | 760,827 | |
| | | | | | (ADA17563.1) | |
| SA0377 (T2) | lipoprotein | 54 | 61 | WP_000746687.1 | Residues 22- | T2: SA0377 (25- |
| | | | | https:// | 208 of SEQ ID | 208 of |
| | | | | www.ncbi.nlm.nih.gov/ | NO: 1844 | WP_000746687.1) |
| | | | | ipg/ADA16179.1 | from U.S. | |
| | | | | | Pat. No. | |
| | | | | | 7,608,276 | |
| | | | | | (ADA16179.1); | |
| | | | | | residues 22- | |
| | | | | | 208 of U.S. | |
| | | | | | Pat. No. | |
| | | | | | 6,593,114 | |
| SA0693 (T3) | lipoprotein | 55 | 62 | RefSeq Selected | Residues 27- | T3: SA0693 (aa. |
| | | | | Product: | 146 of SEQ ID | 27-147): |
| | | | | WP_001093553.1, | NO 3166 from | lipoprotein |
| | | | | 146 amino acids | U.S. Pat. No. | |
| | | | | https:// | 7,608,276 | |
| | | | | www.ncbi.nlm.nih.gov/ | (ADA16815.1) | |
| | | | | ipg/ADA16815.1 | | |
| SA2105 (T4) | ABC | 56 | 63 | | | SA2105 (aa 341- |
| | transporter | | | | | 513): |

In some embodiments, a SA-MAPS immunogenic composition as disclosed herein comprises at least 2, or at least 3, or at least 4, or at least 5, at least 6 or all 7 Group A SA polypeptide antigens of: SA1739 (B2), SA1720 (B1), SA1890 (B3), SA0103 (T1), SA0377 (T2), SA0693 (T3) and SA2105 (T4), or proteins or peptides of at least 85% sequence identity thereto. It is envisioned that any of the above listed Group A SA antigens can be substituted for a different SA peptide or polypeptide antigen known to one of ordinary skill in the art. Exemplary Group A SA antigens can be any peptide or polypeptide comprising at least part of the SA1739 (B2), SA1720 (B1), SA1890 (B3), SA0103 (T1), SA0377 (T2), SA0693 (T3) and SA2105 (T4) protein, provided that the any peptide or polypeptide is immunogenic, or is antigenic. Other SA antigens can be used, and are disclosed herein.

In some embodiments, the SA-MAPS comprises at least one antigen from Group A(i), where the SA antigen is an antigenic protein or polypeptide and elicits a B-cell immune response, and can be selected from any of Group A(i) SA polypeptide antigens of: SA1739 (B2), SA1720 (B1) or SA1890 (B3), or an antigenic fragment or portion thereof. In some embodiments, the SA-MAPS immunogenic composition as disclosed herein comprises one or more peptide or polypeptide fragments of Group A(i) SA polypeptide antigens, as long as the protein fragment is antigenic, and/or comprises one or more epitopes to induce an immune response.

In some embodiments, the SA-MAPS comprises at least one antigen from Group A(ii), where the SA antigen is an antigenic protein or polypeptide and elicits a T-cell immune response, and can be selected from any of Group A(ii) SA polypeptide antigens of: SA0103 (T1), SA0377 (T2), SA0693 (T3) and SA2105 (T4), or an antigenic fragment or portion thereof. In some embodiments, the SA-MAPS immunogenic composition as disclosed herein comprises one or more peptide or polypeptide fragments of Group A(ii) SA polypeptide antigens, as long as the protein fragment is antigenic, and/or comprises one or more epitopes to induce an immune response.

In some embodiments, a SA-MAPS immunogenic composition as disclosed herein comprises at least 1, or at least 2, or all 3 B-cell SA antigens selected from: SA1739 (B2), SA1720 (B1), SA1890 (B3), and least 1, or at least 2, or at least 3 or all 4 T-cell SA antigens selected from: SA0103 (T1), SA0377 (T2), SA0693 (T3) and SA2105 (T4), or proteins or peptides of at least 85% sequence identity thereto. It is envisioned that any of the above listed Group A SA antigens can be substituted for a different SA peptide or polypeptide antigen known to one of ordinary skill in the art. In some embodiments, any Group A SA antigen can be fused to one or more SA antigens in Group A, for example, a B-cell SA antigen from Group A(i) can be fused to one or more B-cell antigens of Group A(i), or one or more T-cell antigens of Group A(ii). Exemplary fusions include, e.g, B1-B2, B1-B3, B1-T1, B1-T2, B1-T3, B2-B3, B2-T1, B2-T2, B2-T3, B3-T1, B3-T2, B3-T3, T1-T2, T2-T3. In some embodiments, the fusion protein can further comprise Rhizavadin, as disclosed herein. In some embodiments, the fusion protein can comprise more than two SA antigens as disclosed herein, for example, B-B-T, or B-T-B, or B-T-T, T-B-T, T-T-B, where B and T represent a B-cell or T-cell SA antigen as disclosed herein.

In some cases, the other appropriate S. aureus antigen is at least at least 70%, 80%, 85%, 90%, 95%, 97%, 98%, or 99% identity to the corresponding wild-type S. aureus protein disclosed in Table 3. Sequences of the above-mentioned polypeptides, and nucleic acids that encode them, are known; see, for example, the S. aureus ATCC 700669 complete genome sequence under GenBank accession number FM211187.1 and linked polypeptide sequences therein.

In addition to those nucleic acids and polypeptides described in Table 3 above, this application also provides immunogenic compositions that include one or more of the polypeptides or genes listed in Table 3, or variants or fragments thereof as described herein. The DNA and protein sequence of each gene and protein may be found by searching for the Locus Tag in the publicly available database, Entrez Gene, as described above.

In one aspect of the present invention provides SA-MAPS immunogenic compositions (comprising at least one isolated SA-antigen selected from the proteins SA1739 (B2), SA1720 (B1), SA1890 (B3), SA0103 (T1), SA0377 (T2), SA0693 (T3) and SA2105 (T4), or fragments thereof. In some embodiments, the SA polypeptide antigen has an amino acid sequence selected from any or a combination from SEQ ID NO: 50-56, or functional fragments thereof. In some embodiments, the SA antigen corresponding to SEQ ID NO: 50-56 are encoded by nucleic acids of SEQ ID NO: 57-63. In some embodiments, fragments of the SA antigen are encompassed for use in the methods and compositions as disclosed herein, for example, SA antigens corresponding to SEQ ID NO: 50-56 as disclosed in Table 3. Other functional fragments of the SA antigens are encompassed for use in the methods and compositions as disclosed herein, and can be assessed by one of ordinary skill in the art to determine if they provide protection against S. aureus colonization, according to the methods as disclosed in Examples herein. A functional fragment of a SA antigen of SEQ ID NO: 50-56 can also be assessed by one of ordinary skill in the art for protection against an invasive disease, such as sepsis according to the methods as disclosed in Example 4 and 5, in particular when used in a SA-MAPS composition herein, alone or as part of a fusion protein with another SA antigen disclosed herein.

In some embodiments, an immunogenic composition comprises at least one SA antigen selected from any one or a combination of the SA antigens with the amino acid sequences SEQ ID NO: 50-56. In some embodiments, the SA polypeptide antigens are the full length S. aureus proteins of SA1739 (B2), SA1720 (B1), SA1890 (B3), SA0103 (TI), SA0377 (T2), SA0693 (T3) and SA2105 (T4). In some embodiments, a SA polypeptide antigen useful in the methods and compositions as disclosed herein comprises a SA protein that lacks a signal sequence and/or transmembrane domain. In some embodiments, a SA polypeptide antigen comprises a mixture of a full length S. aureus proteins and fragments resulting from processing, or partial processing of a signal sequence by an expression host, e.g., E. coli or an insect cell line (e.g., the baculovirus expression system), or a mammalian (e.g., human or Chinese hamster Ovary (CHO)) cell line. As used herein, the terms “portion” and “fragment” or grammatical equivalents are used interchangeably.

In some embodiments, the SA polypeptide antigens are a fragment of the full length SA polypeptides of SA1739 (B2), SA1720 (B1), SA1890 (B3), SA0103 (T1), SA0377 (T2), SA0693 (T3) and SA2105 (T4), for example, at least 7, 8, 9, 10, 11, 12, 13, 14, 15, 16, 17, 18, 19, 20, 25, 30, 35, 40, 45, 50, 55, 60, 65, 70, 75, 80, 85, 90, 100, 150, 200, 250, 300, 350 or 400 consecutive amino acids of such proteins. In some embodiments, fragments of the full-length SA proteins corresponding to SEQ ID NO: 50-56 can be used. In some embodiments, a fragment of a SA protein is a functional fragment of any of SEQ ID NO: 50-56, for example, at least 7, 8, 9, 10, 11, 12, 13, 14, 15, 16, 17, 18, 19, 20, 25, 30, 35, 40, 45, 50, 55, 60, 65, 70, 75, 80, 85, 90, 100, 150, 200, 250, 300, 350 or 400 consecutive amino acids of SA polypeptide antigens corresponding to SEQ ID NO: 50-56.

In some embodiments, a SA polypeptide antigen comprises an amino acid sequence which is at least 60% (e.g., at least 60%, at least 65%, at least 70%, at least 75%, at least 80%, at least 85%, at least 90%, at least 95%, at least 98% or at least 99%) identical to at 7, 8, 9, 10, 11, 12, 13, 14, 15, 16, 17, 18, 19, 20, 25, 30, 35, 40, 45, 50, 55, 60, 65, 70, 75, 80, 85, 90, 100, 150, 200, 250, 300, 350 or 400 consecutive amino acids of the SA proteins of SA1739 (B2), SA1720 (B1), SA1890 (B3), SA0103 (T1), SA0377 (T2), SA0693 (T3) and SA2105 (T4), or fragments thereof, e.g., fragments of SA polypeptide antigens corresponding to SEQ ID NO: 50-56.

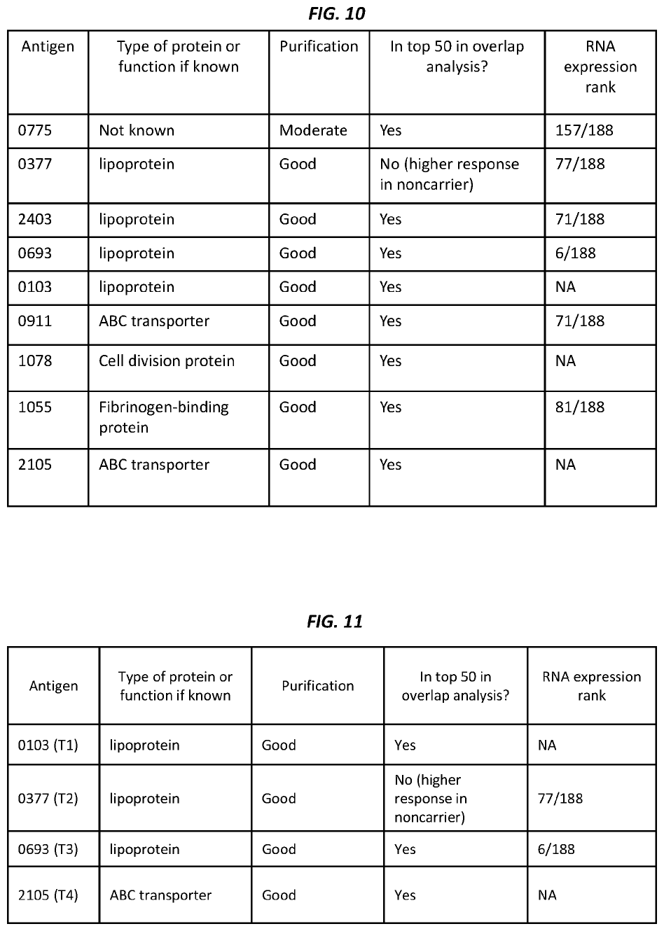

The inventors demonstrate herein that four T-cell SA polypeptide antigens SA0103 (T1), SA0377 (T2), SA0693 (T3) and SA2105 (T4) showed IL-17 response to protein stimulation and protection against S. aureus colonization and protection against abscess, either alone or as fusion proteins with respect to each other (See FIG. 12). The inventors also demonstrated that three B-cell SA polypeptide antigens SA1739 (B2), SA1720 (B1), SA1890 (B3), each individually protected 80% of mice from sepsis infection and protected against S. aureus infection. The inventors also demonstrated that the combination of T-cell SA polypeptide antigens and B-cell SA polypeptide antigens elicited an IL-17 response to protein stimulation and protected against abscess formation after S. aureus infection. The inventors also demonstrated that a MAPS composition comprising a B-cell antigen, e.g., SA1739 (B2), in combination with one or more T-cell antigens (including a fusion of one or more T cell SA antigens as disclosed herein, e.g., SA0103-SA0377 (T1-T2) or SA00103-SA0693 (T1-T3) fusion protein) resulted in a significant protection of mice from sepsis infection after S. aureus infection and the protection was significantly better than the MAPS4 composition alone. Accordingly, these SA polypeptide antigens disclosed in Table 3 provide novel compositions for eliciting immune responses with the aim of eliciting beneficial immune responses, e.g., to protect against S. aureus infections and associated pathogens. These antigens provide novel targets for characterizing S. aureus infections and immune responses to S. aureus infections.

Accordingly, in one aspect of the invention provides an immunogenic composition (e.g., vaccine) comprising an isolated SA polypeptide antigen selected from a SA1739 polypeptide SA antigen, a SA1720 polypeptide SA antigen, a SA1890 polypeptide SA antigen, a SA0103 polypeptide SA antigen, a SA0377 polypeptide SA antigen, a SA0693 polypeptide SA antigen, a SA2105 polypeptide SA antigen, and combinations thereof.

B2: In some embodiments, an immunogenic composition comprises a SA1739 polypeptide SA antigen. In some embodiments, a SA1739 polypeptide antigen comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 150 consecutive amino acids of a SA1739 polypeptide sequence. In some embodiments, a SA1739 polypeptide antigen comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 150 consecutive amino acids of the sequence shown in SEQ ID NO: 51. In some embodiments, a SA1739 polypeptide antigen comprises an amino acid sequence that is at least 60% (e.g., at least 65%, 70%, 75%, 80%, 85%, 90%, 95%, or 98%) identical to at least 7, 8, 9, 10, 11, 12, 13, 14, 15, 16, 17, 18, 19, 20, 25, 30, 35, 45, 50, 60, 65, 70, 75, 80, 85, 90, 95, 100 consecutive amino acids of the sequence shown in SEQ ID NO: 51. In some embodiments, a SP0785 S. aureus polypeptide antigen is encoded by SEQ ID NO: 58 or a fragment thereof. In some embodiments, functional fragment of a SA1739 S. aureus polypeptide antigen comprises amino acids of SEQ ID NO: 51 or comprises an amino acid sequence that is at least 60% (e.g., at least 65%, 70%, 75%, 80%, 85%, 90%, 95%, or 98%) identical to at least 7, 8, 9, 10, 11, 12, 13, 14, 15, 16, 17, 18, 19, 20, 25, 30, 35, 45, 50, 60, 65, 70, 75, 80, 85, 90, 95, 100, 150 consecutive amino acids of the sequence shown in SEQ ID NO: 58.

B1: In some embodiments, an immunogenic composition comprises a SA1720 SA polypeptide antigen. In some embodiments, a SA1720 polypeptide antigen comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250 consecutive amino acids of a SA1720 polypeptide sequence. In some embodiments, a SA1720 polypeptide antigen comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250 consecutive amino acids of the sequence shown in SEQ ID NO: 50. In some embodiments, a SA1720 polypeptide antigen comprises an amino acid sequence that is at least 60% (e.g., at least 65%, 70%, 75%, 80%, 85%, 90%, 95%, or 98%) identical to at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250 consecutive amino acids of the sequence shown in SEQ ID NO: 50. In some embodiments, a SA 1720 S. aureus polypeptide antigen is encoded by SEQ ID NO: 57 or a fragment thereof. In some embodiments, functional fragment of a SA1720 S. aureus polypeptide antigen comprises amino acids of SEQ ID NO: 50 or comprises an amino acid sequence that is at least 60% (e.g., at least 65%, 70%, 75%, 80%, 85%, 90%, 95%, or 98%) identical to at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, consecutive amino acids of the sequence shown in SEQ ID NO: 50.

B3: In some embodiments, an immunogenic composition comprises a SA1890 SA polypeptide antigen. In some embodiments, a SA1890 polypeptide antigen comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350 consecutive amino acids of a SA1890 polypeptide sequence. In some embodiments, a SA1890 polypeptide antigen comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350 consecutive amino acids of the sequence shown in SEQ ID NO: 52. In some embodiments, a SA1890 polypeptide antigen comprises an amino acid sequence that is at least 60% (e.g., at least 65%, 70%, 75%, 80%, 85%, 90%, 95%, or 98%) identical to at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350 consecutive amino acids of the sequence shown in SEQ ID NO: 52. In some embodiments, a SA1890 S. aureus polypeptide antigen is encoded by SEQ ID NO: 59 or a fragment thereof. In some embodiments, functional fragment of a SA1890 S. aureus polypeptide antigen comprises amino acids of SEQ ID NO: 52 or comprises an amino acid sequence that is at least 60% (e.g., at least 65%, 70%, 75%, 80%, 85%, 90%, 95%, or 98%) identical to at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350 consecutive amino acids of the sequence shown in SEQ ID NO: 50.

T1: In some embodiments, an immunogenic composition comprises a SA0103 polypeptide SA antigen. In some embodiments, a SA0103 polypeptide antigen comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 220 consecutive amino acids of a SA0103 polypeptide sequence. In some embodiments, a SA0103 polypeptide antigen comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 220 consecutive amino acids of the sequence shown in SEQ ID NO: 53. In some embodiments, a SA0103 polypeptide antigen comprises an amino acid sequence that is at least 60% (e.g., at least 65%, 70%, 75%, 80%, 85%, 90%, 95%, or 98%) identical to at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 220 consecutive amino acids of the sequence shown in SEQ ID NO: 53. In some embodiments, a SP0785 S. aureus polypeptide antigen is encoded by SEQ ID NO: 60 or a fragment thereof. In some embodiments, functional fragment of a SA0103 S. aureus polypeptide antigen comprises amino acids of SEQ ID NO: 53 or comprises an amino acid sequence that is at least 60% (e.g., at least 65%, 70%, 75%, 80%, 85%, 90%, 95%, or 98%) identical to at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 220 consecutive amino acids of the sequence shown in SEQ ID NO: 53.

T2: In some embodiments, an immunogenic composition comprises a SA0377 polypeptide SA antigen. In some embodiments, a SA0377 polypeptide antigen comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 170 consecutive amino acids of a SA0377 polypeptide sequence. In some embodiments, a SA0377 polypeptide antigen comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 170 consecutive amino acids of the sequence shown in SEQ ID NO: 54. In some embodiments, a SA0377 polypeptide antigen comprises an amino acid sequence that is at least 60% (e.g., at least 65%, 70%, 75%, 80%, 85%, 90%, 95%, or 98%) identical to at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 170 consecutive amino acids of the sequence shown in SEQ ID NO: 54. In some embodiments, a SP0785 S. aureus polypeptide antigen is encoded by SEQ ID NO: 61 or a fragment thereof. In some embodiments, functional fragment of a SA0377 S. aureus polypeptide antigen comprises amino acids of SEQ ID NO: 54 or comprises an amino acid sequence that is at least 60% (e.g., at least 65%, 70%, 75%, 80%, 85%, 90%, 95%, or 98%) identical to at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 170 consecutive amino acids of the sequence shown in SEQ ID NO: 54.

T3: In some embodiments, an immunogenic composition comprises a SA0693 polypeptide SA antigen. In some embodiments, a SA0693 polypeptide antigen comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 110 consecutive amino acids of a SA0693 polypeptide sequence. In some embodiments, a SA0693 polypeptide antigen comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 110 consecutive amino acids of the sequence shown in SEQ ID NO: 55. In some embodiments, a SA0693 polypeptide antigen comprises an amino acid sequence that is at least 60% (e.g., at least 65%, 70%, 75%, 80%, 85%, 90%, 95%, or 98%) identical to at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 110 consecutive amino acids of the sequence shown in SEQ ID NO: 55. In some embodiments, a SP0785 S. aureus polypeptide antigen is encoded by SEQ ID NO: 62 or a fragment thereof. In some embodiments, functional fragment of a SA0693 S. aureus polypeptide antigen comprises amino acids of SEQ ID NO: 55 or comprises an amino acid sequence that is at least 60% (e.g., at least 65%, 70%, 75%, 80%, 85%, 90%, 95%, or 98%) identical to at least 7, 8, 9, 10, 11, 12, 13, 14, 15, 16, 17, 18, 19, 20, 25, 30, 35, 45, 50, 60, 65, 70, 75, 80, 85, 90, 95, 100, 110 consecutive amino acids of the sequence shown in SEQ ID NO: 55.

T4: In some embodiments, an immunogenic composition comprises a SA2105 polypeptide SA antigen. In some embodiments, a SA2105 polypeptide antigen comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 170 consecutive amino acids of a SA2105 polypeptide sequence. In some embodiments, a SA2105 polypeptide antigen comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 170 consecutive amino acids of the sequence shown in SEQ ID NO: 56. In some embodiments, a SA2105 polypeptide antigen comprises an amino acid sequence that is at least 60% (e.g., at least 65%, 70%, 75%, 80%, 85%, 90%, 95%, or 98%) identical to at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 170 consecutive amino acids of the sequence shown in SEQ ID NO: 56. In some embodiments, a SP0785 S. aureus polypeptide antigen is encoded by SEQ ID NO: 63 or a fragment thereof. In some embodiments, functional fragment of a SA2105 S. aureus polypeptide antigen comprises amino acids of SEQ ID NO: 56 or comprises an amino acid sequence that is at least 60% (e.g., at least 65%, 70%, 75%, 80%, 85%, 90%, 95%, or 98%) identical to at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 170 consecutive amino acids of the sequence shown in SEQ ID NO: 56.

B1: SA1720 S. aureus Antigen

SA1720 (also referred to herein as B1) is a hypothetical S. aureus protein. The inventors have demonstrated herein that SA1720 is a reasonable candidate for a component of a vaccine against S. aureus. SA1720 is amino acids residues 33-285 of a larger S. aureus protein. That is, SA1720 antigen as disclosed herein does not include a signal sequence of amino acids 1-32 of a larger S. aureus protein.

In some embodiments, the SA1720 antigen for use in the SA-MAPS immunogenic composition as disclosed herein comprises a polypeptide comprising at least part of the 255 amino acid sequence of SEQ ID NO: 50, which corresponds to the full length SA1720 protein from S. aureus (USA300 TCH959 strain) (without the signal sequence).

(SEQ ID NO: 50; 255 aa)
ISDQIFFKHVKSDIKIEKLNVTLNDAAKKQINNYTSQQVSNKKND
AWRDASATEIKSAMDSGTFIDNEKQKYQFLDLSKYQGIDKNRIKR
MLVDRPTLLKHTDDFLKAAKDKHVNEVYLISHALLETGAVKSELA
NGVEIDGKKYYNFYGVGALDKDPIKTGAEYAKKHGWDTPEKAISG
GADFIHKHFLSSTDQNTLYSMRWNPKNPGEHQYATDIKWAESNAT
IIADFYKNMKTEGKYFKYFVYKDDSKHLNK

SA1720 as disclosed herein is a 255 amino acid fragment of a larger polypeptide of 295 amino acids which was previously disclosed as SEQ ID NO: 818 in U.S. Pat. Nos. 7,608,276, 8,287,884, 8,398,996, 8,465,750; and 8,747,864, which are incorporated herein by reference in their entirety. The SA polypeptide antigen of SA1720 as disclosed herein corresponds to amino acids 41-295 of SEQ ID NO: 818 from U.S. Pat. No. 7,608,276 (GenBank No: ADA15666.1), where U.S. Pat. No. 7,608,276 discloses more than 5642 polypeptides for use in a composition.

SA1720 Sequence: The nucleic acid sequence encoding the SA1720 polypeptide of SEQ ID NO: 50 is as follows:

(SEQ ID NO: 57)
ATATCCGATCAGATATTTTTCAAACATGTTAAATCCGACATTAAG
ATTGAAAAGTTAAATGTTACATTAAACGATGCAGCAAAGAAACAA
ATAAATAATTATACGAGTCAACAGGTATCAAATAAAAAGAATGAT
GCATGGAGAGATGCATCTGCAACTGAAATTAAAAGTGCAATGGAT
AGCGGTACTTTTATCGATAATGAAAAGCAAAAATATCAATTTTTA
GATTTATCAAAGTATCAAGGGATTGATAAAAATAGAATTAAACGT
ATGTTAGTAGATAGACCAACGTTATTGAAACATACGGATGATTTC
TTAAAAGCTGCTAAAGATAAGCACGTTAACGAAGTTTATTTAATT
TCACATGCATTATTAGAAACTGGCGCAGTTAAAAGTGAATTAGCT
AATGGAGTCGAAATTGATGGCAAAAAGTACTACAATTTCTATGGA
GTAGGAGCCCTTGATAAAGACCCAATTAAAACAGGTGCAGAATAT
GCTAAAAAGCATGGTTGGGATACACCTGAAAAAGCTATTTCAGGC
GGTGCTGATTTCATTCATAAGCACTTCTTATCAAGCACAGATCAA
AATACATTGTATAGTATGAGATGGAATCCAAAAAATCCAGGAGAA
CATCAATATGCTACAGATATTAAGTGGGCAGAAAGTAATGCAACA
ATTATCGCTGACTTTTATAAGAACATGAAGACTGAAGGAAAATAC
TTCAAATACTTTGTGTATAAAGATGACAGTAAACATTTGAATAAG
TAA

SEQ ID NO: 57 comprises a portion of nucleic acid sequence SEQ ID NO: 817 disclosed in U.S. Pat. Nos. 9,762,020, 9,296,796 and 10,226,524, which are incorporated herein in their entirety by reference.

In some embodiments, the SA1720 antigen for use in the SA-MAPS immunogenic composition as disclosed herein is SA1720 (33-285) (SEQ ID NO: 50), or a fragment or protein of at least 85% amino acid sequence identity thereto.

In one embodiment, a SA-MAPS composition may include a polypeptide, peptide, or protein that is or is at least 70%, 75%, 80%, 85%, 90%, 95%, 96%, 97%, 98%, or 99% identical or similar to a SA1720 of SEQ ID NO: 50. In certain aspects a SA1720 antigen peptide or polypeptide will have all, or part of the amino acid sequence of SEQ ID NO: 50, e.g., will comprise at least 50, or at least 60, or at least 70, or at least 80, or at least 90, or at least 100, or at least 120, or at least 140, or at least 160, or at least 180, or at least 200, or at least 220 or at least 240 amino acids of SEQ ID NO: 50. In one embodiment, a SA1720 antigen peptide or polypeptide present in the SA-MAPS immunogenic composition is a polypeptide, peptide, or protein that is or is at least 70%, 75%, 80%, 85%, 90%, 95%, 96%, 97%, 98%, or 99% identical or similar to SEQ ID NO: 50.

In alternative embodiments, a SA1720 antigen for use in the SA-MAPS immunogenic composition as disclosed herein is a protein or peptide having an amino acid sequence of SEQ ID NO: 818 from U.S. Pat. Nos. 7,608,276, 8,287,884; 8,398,996; 8,465,750 and 8,747,864. In some embodiments, a SA1720 antigen for use in the SA-MAPS immunogenic composition as disclosed herein is a SA1720 variant of SEQ ID NO: 50, or a mutant SA1720 from S. aureus strain PFESA0237 of SEQ ID NO: 50 or SEQ ID NO: 818 as disclosed in U.S. Pat. Nos. 7,608,276, 8,287,884; 8,398,996; 8,465,750 and 8,747,864 (SEQ ID NO: ADA15666.1) and are encompassed for use in the SA-MAPS immunogenic composition as disclosed herein.

The amino acid sequence of the full length (including the signal sequence) wild type SA1720 from S. aureus strain USA300 TCH959 SA strain is disclosed as SEQ ID NO: 181 in US patent 7,6008,276.

The term “SA1720 protein” refers to a protein that includes isolated wild-type SA1720 polypeptides without the signal sequence, from staphylococcus bacteria and segments thereof, as well as variants that stimulate an immune response against staphylococcus bacteria SA1720 proteins.

B2: SA1739 S. aureus Antigen

SA1739 (herein also referred to as “B2”) is a hypothetical S. aureus protein. The inventors have demonstrated herein that SA1739 is a reasonable candidate for a component of a vaccine against S. aureus. SA1739 is amino acids residues 60-217 of a larger S. aureus protein.

In some embodiments, the SA1739 antigen for use in the SA-MAPS immunogenic composition as disclosed herein comprises a polypeptide comprising at least part of the 157 amino acid sequence of SEQ ID NO: 51, which corresponds to the full length SA1739 protein from S. aureus strain USA300 (without the signal sequence).

(SEQ ID NO: 51; 157 aa)
NNDNSTNNQSSKTQLTQKDENKNEDKPEEKSETATDEDLQSTEEV
PANENTENNQHEIDEITTKDQSDDDINTPNVAEDKSQDDLKDDLK
EKQQSSNHHQSTQPKTSPSTETNTQQSFANCKQLRQVYPNGVTAD
HPAYRPHLDRDKDKRACEPDKY

SA1739 as disclosed herein is a 157 amino acid fragment of a larger polypeptide of 184 amino acids which was previously disclosed as SEQ ID NO: 5201 in U.S. Pat. Nos. 6,593,114 or 6,737,248, or a fragment of a 216 amino acid polypeptide disclosed as SEQ ID NO: 766 in U.S. Pat. Nos. 7,608,276; 8,287,884; 8,398,996; 8,465,750; 8,679,505, the references of which are incorporated herein by reference in their entirety. The SA polypeptide antigen of SA1739 as disclosed herein corresponds to amino acids 28-184 of SEQ ID NO: 184 from U.S. Pat. No. 6,593,114. The SA polypeptide antigen of SA1739 as disclosed herein lacks the signal sequence MLKGCGGCLISFILLIILLSACSMMFS (SEQ ID NO: 65) of SEQ ID NO: 5201 from U.S. Pat. No. 6,593,114 (AAQ52145.1), or SEQ ID NO: 766 from U.S. Pat. No. 7,608,276 (ADA 15640.1), or SEQ ID NO: 2 from U.S. Pat. No. 9,085,631 (Accession No: AMF69721.1), or SEQ ID NO: 38372 from patent U.S. Ser. No. 10/463,711 (Accession No: QHY66991.1).

SA1739 Sequence: The nucleic acid sequence encoding the SA1739 polypeptide of SEQ ID NO: 51 is as follows:

(SEQ ID NO: 58)
AATAATGACAATTCCACTAATAATCAATCATCAAAAACGCAATTA
ACTCAAAAAGATGAAAATAAAAATGAAGATAAGCCTGAGGAAAAA
TCAGAAACAGCAACAGATGAGGATTTACAATCAACCGAAGAAGTA
CCTGCAAATGAAAATACTGAAAATAATCAACATGAAATTGATGAA
ATAACAACAAAAGATCAATCAGACGATGATATTAACACACCAAAC
GTTGCAGAAGATAAATCACAAGACGACTTGAAAGATGATTTAAAA
GAAAAGCAACAATCAAGTAACCATCATCAATCCACGCAACCTAAG
ACCTCACCATCAACTGAAACAAACACGCAACAATCATTTGCTAAT
TGTAAGCAACTTAGACAAGTATATCCGAATGGTGTCACTGCCGAT
CATCCAGCATATCGACCACATTTAGATAGAGATAAAGATAAACGT
GCATGTGAACCTGATAAATATTAA

SEQ ID NO: 58 comprises a portion (e.g. bp243-715) of nucleic acid sequence SEQ ID NO: 228 (GenBank AR535666.1) disclosed in U.S. Pat. No. 6,737,248, which is incorporated herein in their entirety by reference.

In some embodiments, the SA1739 antigen for use in the SA-MAPS immunogenic composition as disclosed herein is SA1739 (aa 60-217) (SEQ ID NO: 51), or a fragment or protein of at least 85% amino acid sequence identity thereto.

In one embodiment, a SA-MAPS composition may include a polypeptide, peptide, or protein that is or is at least 70%, 75%, 80%, 85%, 90%, 95%, 96%, 97%, 98%, or 99% identical or similar to a SA1739 of SEQ ID NO: 51. In certain aspects a SA1739 antigen peptide or polypeptide will have all, or part of the amino acid sequence of SEQ ID NO: 51, e.g., will comprise at least 50, or at least 60, or at least 70, or at least 80, or at least 90, or at least 100, or at least 120, or at least 140, or at least 160, or at least 180, or at least 200, or at least 220 or at least 240 amino acids of SEQ ID NO: 51. In one embodiment, a SA1739 antigen peptide or polypeptide present in the SA-MAPS immunogenic composition is a polypeptide, peptide, or protein that is or is at least 70%, 75%, 80%, 85%, 90%, 95%, 96%, 97%, 98%, or 99% identical or similar to SEQ ID NO: 51.

In alternative embodiments, a SA1739 antigen for use in the SA-MAPS immunogenic composition as disclosed herein is a protein or peptide having an amino acid sequence of SEQ ID NO: 818 from U.S. Pat. No. 7,608,276. In some embodiments, a SA1720 antigen for use in the SA-MAPS immunogenic composition as disclosed herein is a SA1720 variant of SEQ ID NO: 50, or a mutant SA1720 from S. aureus strain PFESA0237 of SEQ ID NO: 51 or SEQ ID NO: 818 as disclosed in U.S. Pat. No. 7,608,276, and are encompassed for use in the SA-MAPS immunogenic composition as disclosed herein.

The term “SA1739 protein” refers to a protein that includes isolated wild-type SA1739 polypeptides from staphylococcus bacteria and segments thereof, as well as variants that stimulate an immune response against staphylococcus bacteria SA1739 proteins.

B3: SA1890 S. aureus Antigen (B3)

SA1890 (herein also referred to as “B3”) is a cysteine protease staphopain A S. aureus protein. The inventors have demonstrated herein that SA1890 is a reasonable candidate for a component of a vaccine against S. aureus. SA1890 is amino acids residues 26-388 of a larger 388 amino acid S. aureus protein corresponding to the protein with accession number WP_064305855.1. That is, SA1890 antigen as disclosed herein does not include a signal sequence of amino acids 1-25 of a larger S. aureus protein corresponding to Accession number WP_064305855.1.

In some embodiments, the SA1890 antigen for use in the SA-MAPS immunogenic composition as disclosed herein comprises a polypeptide comprising at least part of the 363 amino acid sequence of SEQ ID NO: 52, which corresponds to the full length SA1890 protein from S. aureus (USA300_TCH959 strain) (without the amino acids 1-26 signal sequence comprising MKRNFPKLIALSLIFSLSVTPIANA (SEQ ID NO: 66). The amino acid sequence of SA1890 antigen is as follows:

(SEQ ID NO: 52; 363 aa)
ESNSNIKAKDKKHVQVNVEDKSVPTDVRNLAQKDYLSYVTSLDKIYNKEK
ASYTLGEPFKIYKFNKKSDGNYYFPVLNTEGNIDYIVTISPKITKYSSSS
SKYTINVSPFLSKVLNQYKDQQITILTNSKGYYVVTQNHKAKLVLKTPRL
EDKKLKKTESIPTGNNVTQLKQKASVTMPTSQFKSNNYTYNEQYINKLEN
FKIRETQGNNGWCAGYTMSELLNATYNTNKYHAEAVMRFLHPNLQGQRFQ
FTGLTPREMIYFGQTQGRSPQLLNRMTTYNEVDNLTKNNKGIAVLGSRVE
SRNGMHAGHAMAVVGNAKLDNGQEVIIIWNPWDNGFMTQDAKNNVIPVSN
GDHYRWYSSIYGY

SA1890 as disclosed herein is a 363 amino acid fragment of a larger polypeptide of 388 amino acids which was previously disclosed as SEQ ID NO: 1402 in U.S. Pat. Nos. 7,608,276, 8,287,884, 8,398,996, 8,465,750; and 8,679,505, which are incorporated herein by reference in their entirety. The SA polypeptide antigen of SA1890 as disclosed herein corresponds to amino acids 26-388 of SEQ ID NO: 1402 in U.S. Pat. Nos. 7,608,276, (GenBank No: ADA15958.1), where U.S. Pat. No. 7,608,276 discloses more than 5642 polypeptides for use in a composition.

SA1890 Sequence: The nucleic acid sequence encoding the SA1890 polypeptide of SEQ ID NO: 52 is SEQ ID NO: 59 as follows:

(SEQ ID NO: 59)
GAGAGCAATTCAAATATCAAAGCAAAAGATAAGAAGCATGTGCAAGTTAA
TGTCGAGGACAAGAGTGTTCCTACTGACGTACGTAATTTAGCGCAAAAAG
ACTATCTTTCTTATGTTACTTCACTTGATAAAATCTATAATAAAGAGAAA
GCCTCGTATACACTTGGTGAACCTTTTAAAATTTATAAATTCAACAAAAA
GAGTGATGGTAATTATTATTTTCCAGTTTTGAACACTGAAGGTAATATCG
ATTATATAGTTACAATTTCACCAAAAATCACTAAGTATTCTAGTTCATCT
TCTAAATACACAATCAATGTTTCTCCATTTTTATCTAAAGTCTTAAATCA
ATATAAAGATCAACAAATTACTATTTTGACAAACTCAAAAGGTTACTATG
TGGTTACTCAAAATCATAAAGCTAAACTAGTACTAAAAACACCACGTCTA
GAAGATAAAAAGTTAAAAAAGACTGAGAGTATACCTACTGGCAATAATGT
AACGCAACTAAAGCAAAAAGCTTCTGTCACTATGCCAACTAGTCAATTTA
AAAGTAATAATTATACTTACAACGAGCAATATATAAATAAACTTGAGAAT
TTCAAGATAAGAGAAACTCAAGGTAACAATGGTTGGTGTGCAGGCTATAC
GATGTCTGAATTACTAAATGCGACTTATAATACTAACAAATATCATGCAG
AAGCTGTGATGAGATTTTTACACCCAAACTTACAAGGACAACGATTCCAA
TTTACTGGTTTAACGCCAAGAGAGATGATTTATTTTGGACAAACTCAGGG
AAGAAGTCCACAACTTCTTAATAGAATGACTACATATAATGAAGTTGACA
ATCTTACTAAAAATAATAAAGGAATTGCTGTTTTAGGTAGTAGAGTAGAA
TCAAGAAATGGTATGCACGCAGGGCATGCCATGGCAGTTGTTGGGAACGC
TAAATTAGATAATGGACAAGAGGTTATAATTATATGGAATCCATGGGATA
ATGGTTTTATGACTCAAGATGCTAAAAACAATGTTATTCCAGTATCCAAT
GGCGACCATTATCGATGGTATTCATCTATTTATGGTTATTAA

SEQ ID NO: 59 comprises a portion of nucleic acid sequence SEQ ID NO: 1401 disclosed in U.S. Pat. Nos. 9,762,020, 9,296,796 and 10,226,524, which are incorporated herein in their entirety by reference.

In some embodiments, the SA1890 antigen for use in the SA-MAPS immunogenic composition as disclosed herein is SA1720 (SEQ ID NO: 52), (i.e., aa 27-388 of the full length 388 protein) or a fragment or protein of at least 85% amino acid sequence identity thereto.

In one embodiment, a SA-MAPS composition may include a polypeptide, peptide, or protein that is or is at least 70%, 75%, 80%, 85%, 90%, 95%, 96%, 97%, 98%, or 99% identical or similar to a SA1890 of SEQ ID NO: 52. In certain aspects a SA1890 antigen peptide or polypeptide will have all, or part of the amino acid sequence of SEQ ID NO: 52, e.g., will comprise at least 50, or at least 60, or at least 70, or at least 80, or at least 90, or at least 100, or at least 120, or at least 140, or at least 160, or at least 180, or at least 200, or at least 220 or at least 240 amino acids of SEQ ID NO: 52. In one embodiment, a SA1890 antigen peptide or polypeptide present in the SA-MAPS immunogenic composition is a polypeptide, peptide, or protein that is or is at least 70%, 75%, 80%, 85%, 90%, 95%, 96%, 97%, 98%, or 99% identical or similar to SEQ ID NO: 52.

In alternative embodiments, a SA1890 antigen for use in the SA-MAPS immunogenic composition as disclosed herein is a protein or peptide having an amino acid sequences corresponding to residues 26-388 of SEQ ID NO: 1402 in U.S. Pat. Nos. 7,608,276, (GenBank No: ADA15958.1). In some embodiments, a SA1890 antigen for use in the SA-MAPS immunogenic composition as disclosed herein is a SA1890 variant of SEQ ID NO: 52, or a mutant SA1890 from S. aureus strain PFESA0237 of SEQ ID NO: 52 or residues 26-388 of SEQ ID NO: 1402 in U.S. Pat. Nos. 7,608,276, (GenBank No: ADA15958.1) and are encompassed for use in the SA-MAPS immunogenic composition as disclosed herein.

The term “SA1890 protein” refers to a protein that includes isolated wild-type SA1890 polypeptides without the signal sequence, from staphylococcus bacteria and segments thereof, as well as variants that stimulate an immune response against staphylococcus bacteria SA1890 proteins.

T1: SA0103 S. aureus Antigen

SA0103 (herein also referred to as “T1”) is a lipoprotein A S. aureus protein. The inventors have demonstrated herein that SA0103 is a reasonable candidate for a component of a vaccine against S. aureus. SA0103 is amino acids residues 27-255 of a larger 255 amino acid S. aureus protein corresponding to the protein with accession number WP_000826311.1. That is, SA0103 antigen as disclosed herein does not include a signal sequence of amino acids 1-26 of a larger S. aureus protein corresponding to Accession number WP_064305855.1.

In some embodiments, the SA0103 antigen for use in the SA-MAPS immunogenic composition as disclosed herein comprises a polypeptide comprising at least part of the 255 amino acid sequence of SEQ ID NO: 53, which corresponds to the full length SA0103 protein (WP_000826311.1) from S. aureus (USA300_TCH959 strain) (without the amino acids 1-26 signal sequence comprising MKRLNKLVLY ISFLILVISF TAGCGI (SEQ ID NO: 67). The amino acid sequence of SA0103 is as follows:

(SEQ ID NO: 53; 229 aa)
GKEAEVKKSFEKTLSMYPIKNLEDLYDKEGYRDDEFDKNDKGTWIIGSEM
VVQPKGERMKSKGMVLYMNRNTKTTTGKYIVSETLHDEDGRPKSKDKEYP
VKMVDNKIIPTKGIKDENIKKEIENFKFFAQYGSFKDLSKYKDGDISYNP
EVPSYSAKYQLTNDDYNVKQLRKRYKIPTNKAPKLLLKGSGDLKGSSVGY
KDIEFTFVEKKGENTFFTDSLHLEPSEDK

SA0103 as disclosed herein is a 229 amino acid fragment of a larger polypeptide of 255 amino acids which was previously disclosed as SEQ ID NO: 4612 in U.S. Pat. Nos. 7,608,276, 8,287,884, 8,398,996, 8,465,750; and 8,747,864, which are incorporated herein by reference in their entirety. The SA polypeptide antigen of SA0103 as disclosed herein corresponds to amino acids 27-255 of SEQ ID NO: 4612 in U.S. Pat. Nos. 7,608,276, (GenBank No: ADA17563.1), where U.S. Pat. No. 7,608,276 discloses more than 5642 polypeptides for use in a composition.

SA0103 Sequence: The nucleic acid sequence encoding the SA0103 polypeptide of SEQ ID NO: 53 is SEQ ID NO: 60 as follows:

(SEQ ID NO: 60)
GGTAAAGAAGCGGAAGTTAAGAAAAGCTTTGAAAAAACATTGAGTATGTA
TCCGATTAAAAATCTAGAGGATTTATACGATAAAGAAGGTTATCGTGATG
ACGAATTTGATAAAAATGATAAAGGTACATGGATTATTGGTTCTGAAATG
GTTGTTCAACCTAAAGGGGAACGTATGAAATCAAAAGGTATGGTTCTATA
TATGAATAGAAACACTAAGACTACAACCGGGAAGTATATAGTGAGTGAAA
CACTTCATGATGAAGATGGTAGACCTAAGAGTAAAGATAAAGAATATCCC
GTTAAAATGGTAGACAATAAAATCATTCCAACTAAAGGTATCAAAGACGA
AAATATAAAAAAAGAAATCGAGAATTTTAAGTTCTTCGCGCAATATGGCA
GCTTTAAAGATTTGTCGAAGTACAAAGACGGCGATATATCATATAATCCA
GAGGTACCGAGTTATTCAGCAAAATATCAATTAACTAATGATGACTATAA
TGTAAAACAATTACGAAAAAGATATAAAATACCAACGAATAAAGCACCAA
AGTTATTGTTGAAAGGTTCGGGAGACTTAAAAGGTTCATCAGTTGGATAT
AAAGACATTGAATTTACTTTTGTGGAGAAAAAAGGTGAAAATACTTTTTT
TACTGATAGTCTACATCTTGAACCGAGTGAGGATAAATAA

SEQ ID NO: 53 comprises a portion of nucleic acid sequence SEQ ID NO: 4611 disclosed in U.S. Pat. No. 7,608,276, which is incorporated herein in their entirety by reference.

In some embodiments, the SA0103 antigen for use in the SA-MAPS immunogenic composition as disclosed herein is SA0103 (SEQ ID NO: 53), (i.e., residues 27-255 of the full length 255 protein) or a fragment or protein of at least 85% amino acid sequence identity thereto.

In one embodiment, a SA-MAPS composition may include a polypeptide, peptide, or protein that is or is at least 70%, 75%, 80%, 85%, 90%, 95%, 96%, 97%, 98%, or 99% identical or similar to a SA0103 of SEQ ID NO: 53. In certain aspects a SA0103 antigen peptide or polypeptide will have all, or part of the amino acid sequence of SEQ ID NO: 53, e.g., will comprise at least 50, or at least 60, or at least 70, or at least 80, or at least 90, or at least 100, or at least 120, or at least 140, or at least 160, or at least 180, or at least 200, or at least 220 amino acids of SEQ ID NO: 53. In one embodiment, a SA0103 antigen peptide or polypeptide present in the SA-MAPS immunogenic composition is a polypeptide, peptide, or protein that is or is at least 70%, 75%, 80%, 85%, 90%, 95%, 96%, 97%, 98%, or 99% identical or similar to SEQ ID NO: 53.

In alternative embodiments, a SA0103 antigen for use in the SA-MAPS immunogenic composition as disclosed herein is a protein or peptide having an amino acid sequences corresponding to residues 27-255 of SEQ ID NO: 4612 in U.S. Pat. Nos. 7,608,276, (GenBank No: ADA17563.1). In some embodiments, a SA0103 antigen for use in the SA-MAPS immunogenic composition as disclosed herein is a SA0103 variant of SEQ ID NO: 52, or a mutant SA0103 from S. aureus strain PFESA0237 of SEQ ID NO: 52 or residues 27-255 of SEQ ID NO: 4612 in U.S. Pat. Nos. 7,608,276, (GenBank No: ADA17563.1), and are encompassed for use in the SA-MAPS immunogenic composition as disclosed herein.

The term “SA0103 protein” refers to a protein that includes isolated wild-type SA0103 polypeptides without the signal sequence, from staphylococcus bacteria and segments thereof, as well as variants that stimulate an immune response against staphylococcus bacteria SA0103 proteins.

T2: SA0377 S. aureus Antigen

SA0377 (also referred to herein as “T2”) is a NDxxF motif lipoprotein S. aureus protein. The inventors have demonstrated herein that SA0377 is a reasonable candidate for a component of a vaccine against S. aureus. SA0377 is amino acids residues 25-208 of a larger 208 amino acid S. aureus protein corresponding to the protein with accession number WP_000746687.1. That is, SA0377 antigen as disclosed herein does not include a signal sequence of amino acids 1-24 of a larger S. aureus protein corresponding to Accession number WP_000746687.1.

In some embodiments, the SA0377 antigen for use in the SA-MAPS immunogenic composition as disclosed herein comprises a polypeptide comprising at least part of the 187 amino acid sequence of SEQ ID NO: 54, which corresponds to the full length SA0377 protein (WP_000746687.1) from S. aureus (USA300_TCH959 strain) (without the amino acids 1-24 signal sequence comprising MKKRLLLSTFLASTLILTGCASDQ (SEQ ID NO: 68). The amino acid sequence of the SA0377 antigen is as follows:

(SEQ ID NO: 54; 187 aa)
SDQSDNEDHHTSTGIHAPKSAKKLETKDIFNSDKKNSDISDAEMKQAIEK
YLSVNSDILDNKYIMQHKLDKQIDSQTKVTEKQAETLSHLSNLAVKNDLH
FKKFVTENNIPKEYKKPVELMMNYFKALNSTIANVDEDIEKLSYQPQNKI
NVVDVPTKYAGDVNKKQQDKIKDFLKSKGIKSDVIDK

SA0377 as disclosed herein is a 187 amino acid fragment of a larger polypeptide of 208 amino acids which was previously disclosed as SEQ ID NO: 1844 in U.S. Pat. Nos. 7,608,276, 8,287,884, 8,398,996, 8,465,750; and 8,679,505, which are incorporated herein by reference in their entirety. The SA polypeptide antigen of SA0377 as disclosed herein corresponds to amino acids 22-208 of SEQ ID NO: 1844 in U.S. Pat. Nos. 7,608,276, (GenBank No: ADA16179.1), where U.S. Pat. No. 7,608,276 discloses more than 5642 polypeptides for use in a composition.

SA0377 Sequence: The nucleic acid sequence encoding the SA0377 polypeptide of SEQ ID NO: 54 is SEQ ID NO: 61 as follows:

(SEQ ID NO: 61)
TCCGATCAATCTGATAACGAAGATCATCATACAAGTACTGGCATCCATGC
ACCTAAAAGCGCTAAAAAATTAGAAACAAAAGATATCTTTAACTCAGATA
AGAAAAATAGTGATATTAGTGATGCAGAGATGAAGCAAGCTATAGAAAAG
TATTTGTCTGTAAATAGCGATATACTTGATAACAAATATATTATGCAACA
TAAACTAGATAAACAAATTGACAGTCAAACAAAAGTGACTGAAAAACAAG
CAGAAACACTTAGCCACTTATCTAACTTAGCTGTTAAAAACGATTTGCAT
TTCAAGAAATTTGTCACTGAAAACAATATTCCTAAAGAATATAAGAAACC
CGTTGAATTAATGATGAATTACTTCAAAGCTTTAAATAGTACAATCGCCA
ATGTTGATGAAGATATTGAAAAGCTTAGCTATCAACCGCAAAATAAAATC
AATGTTGTCGACGTGCCAACAAAATATGCCGGTGATGTTAATAAAAAGCA
ACAAGATAAAATTAAAGATTTCTTAAAATCTAAAGGTATCAAGAGTGATG
TTATCGATAAATAA

SEQ ID NO: 61 comprises a portion of nucleic acid sequence SEQ ID NO: 1843 disclosed in U.S. Pat. No. 7,608,276, which is incorporated herein in their entirety by reference.

In some embodiments, the SA0377 antigen for use in the SA-MAPS immunogenic composition as disclosed herein is SA0377 (SEQ ID NO: 54), (i.e., residues 25-208 of the full length 208 protein) or a fragment or protein of at least 85% amino acid sequence identity thereto.

In one embodiment, a SA-MAPS composition may include a polypeptide, peptide, or protein that is or is at least 70%, 75%, 80%, 85%, 90%, 95%, 96%, 97%, 98%, or 99% identical or similar to a SA0377 of SEQ ID NO: 54. In certain aspects a SA0377 antigen peptide or polypeptide will have all, or part of the amino acid sequence of SEQ ID NO: 54, e.g., will comprise at least 50, or at least 60, or at least 70, or at least 80, or at least 90, or at least 100, or at least 120, or at least 140, or at least 160, or at least 180 amino acids of SEQ ID NO: 54. In one embodiment, a SA0377 antigen peptide or polypeptide present in the SA-MAPS immunogenic composition is a polypeptide, peptide, or protein that is or is at least 70%, 75%, 80%, 85%, 90%, 95%, 96%, 97%, 98%, or 99% identical or similar to SEQ ID NO: 54.

In alternative embodiments, a SA0377 antigen for use in the SA-MAPS immunogenic composition as disclosed herein is a protein or peptide having an amino acid sequences corresponding to residues 22-208 of SEQ ID NO: 1844 in U.S. Pat. Nos. 7,608,276, (GenBank No: ADA16179.1). In some embodiments, a SA0377 antigen for use in the SA-MAPS immunogenic composition as disclosed herein is a SA0377 variant of SEQ ID NO: 54, or a mutant SA0377 from S. aureus strain PFESA0237 of SEQ ID NO: 52 or residues 22-208 of SEQ ID NO: 1844 in U.S. Pat. Nos. 7,608,276, (GenBank No: ADA16179.1, and are encompassed for use in the SA-MAPS immunogenic composition as disclosed herein.

The term “SA0377 protein” refers to a protein that includes isolated wild-type SA0377 polypeptides without the signal sequence, from staphylococcus bacteria and segments thereof, as well as variants that stimulate an immune response against staphylococcus bacteria SA0377 proteins.

T3: SA0693 S. aureus Antigen

SA0693 (referred to herein as “T3’) is a lipoprotein, or a DM13 domain-containing S. aureus protein. The inventors have demonstrated herein that SA0693 is a reasonable candidate for a component of a vaccine against S. aureus. SA0693 is amino acids 27-146 of a larger 146aa S. aureus protein corresponding to the protein with accession number WP_031790730.1, or is amino acids residues 4-123 of a larger 132 amino acid S. aureus protein corresponding to the protein with accession number KAB2217044.1. That is, SA0693 antigen as disclosed herein does not include a signal sequence of amino acids 1-26 of a larger S. aureus protein corresponding to Accession number WP_031790730.1.

In some embodiments, the SA0693 antigen for use in the SA-MAPS immunogenic composition as disclosed herein comprises a polypeptide comprising at least part of the 120 amino acid sequence of SEQ ID NO: 55, which corresponds to the full length SA0693 protein (WP_031790730.1) from S. aureus (USA300_TCH959 strain) but without the amino acids 1-26 signal sequence comprising MNTKYFLAVGAVASVLTLGACSNSNS (SEQ ID NO: 69). The amino acid sequence of the SA0693 antigen is as follows:

(SEQ ID NO: 55; 120 aa)
QDQGNKTEQKTKSEDSNVKTDKTKHLTGTFSSKNGETVEGKAEIKNGKLM
LTNYKSSKGPDLYVYLTKNGDIKNGKEIAMVDYDKEKQTFDLKNVDLSKY
DEVTIYCKKAHVIFGGAKLK

SA0693 as disclosed herein is a 120 amino acid fragment of a larger polypeptide of 146 amino acids which was previously disclosed as SEQ ID NO: 3116 in U.S. Pat. Nos. 7,608,276, 8,287,884, 8,398,996, 8,465,750; and 8,679,505, which are incorporated herein by reference in their entirety. The SA polypeptide antigen of SA0693 as disclosed herein corresponds to amino acids 27-146 of SEQ ID NO: 3116 in U.S. Pat. Nos. 7,608,276, (GenBank No: ADA16815.1), where U.S. Pat. No. 7,608,276 discloses more than 5642 polypeptides for use in a composition.

SA0693 Sequence: The nucleic acid sequence encoding the SA0693 polypeptide of SEQ ID NO: 55 is SEQ ID NO: 62 as follows:

(SEQ ID NO: 62)
CAAGATCAAGGTAACAAAACTGAACAAAAAACAAAGTCAGAAGATAGCAA
TGTTAAAACTGATAAAACAAAACACCTAACAGGTACATTCAGTTCTAAAA
ACGGTGAAACTGTTGAAGGTAAAGCTGAGATTAAAAATGGTAAATTAATG
CTTACTAACTACAAATCATCAAAAGGTCCAGATTTATACGTCTACCTAAC
AAAAAATGGCGACATTAAAAACGGTAAAGAAATCGCAATGGTTGACTACG
ATAAAGAAAAACAAACATTTGATCTTAAAAATGTAGATTTAAGCAAATAT
GATGAAGTAACAATCTATTGTAAAAAAGCTCACGTCATCTTCGGCGGCGC
TAAATTAAAATAA

SEQ ID NO: 62 comprises a portion of nucleic acid sequence SEQ ID NO: 9055 disclosed in International Application WO2005014857, which is incorporated herein in their entirety by reference.

In some embodiments, the SA0693 antigen for use in the SA-MAPS immunogenic composition as disclosed herein is SA0693 (SEQ ID NO: 55), (i.e., residues 27-146 of the full length 146 aa protein) or a fragment or protein of at least 85% amino acid sequence identity thereto.

In one embodiment, a SA-MAPS composition may include a polypeptide, peptide, or protein that is or is at least 70%, 75%, 80%, 85%, 90%, 95%, 96%, 97%, 98%, or 99% identical or similar to a SA0693 of SEQ ID NO: 55. In certain aspects a SA0693 antigen peptide or polypeptide will have all, or part of the amino acid sequence of SEQ ID NO: 55, e.g., will comprise at least 50, or at least 60, or at least 70, or at least 80, or at least 90, or at least 100, or at least 120, or at least 140, or at least 160, or at least 180 amino acids of SEQ ID NO: 55. In one embodiment, a SA0693 antigen peptide or polypeptide present in the SA-MAPS immunogenic composition is a polypeptide, peptide, or protein that is or is at least 70%, 75%, 80%, 85%, 90%, 95%, 96%, 97%, 98%, or 99% identical or similar to SEQ ID NO: 55.

In alternative embodiments, a SA0693 antigen for use in the SA-MAPS immunogenic composition as disclosed herein is a protein or peptide having an amino acid sequences corresponding to residues 27-146 of SEQ ID NO: 3116 in U.S. Pat. Nos. 7,608,276, (GenBank No: ADA16815.1). In some embodiments, a SA0693 antigen for use in the SA-MAPS immunogenic composition as disclosed herein is a SA0693 variant of SEQ ID NO: 55, or a mutant SA0693 from S. aureus strain PFESA0237 of SEQ ID NO: 55 or residues of SEQ ID NO: 3116 in U.S. Pat. Nos. 7,608,276, (GenBank No: ADA16815.1), and are encompassed for use in the SA-MAPS immunogenic composition as disclosed herein.

The term “SA0693 protein” refers to a protein that includes isolated wild-type SA0693 polypeptides without the signal sequence, from staphylococcus bacteria and segments thereof, as well as variants that stimulate an immune response against staphylococcus bacteria SA0693 proteins.

T4: SA2105 S. aureus Antigen

SA2105 (also referred to herein as “T4”) is a mannitol or ABC transporter S. aureus protein. The inventors have demonstrated herein that SA2105 is a reasonable candidate for a component of a vaccine against S. aureus. SA2105 is amino acids 139-310 of a larger 30laa S. aureus protein corresponding to the protein with accession number WP_042741942.1. That is, SA2105 antigen as disclosed herein does not include a signal sequence and/or N-terminal sequence of amino acids 1-138 of a larger S. aureus protein corresponding to Accession number WP_042741942.1.

In some embodiments, the SA2105 antigen for use in the SA-MAPS immunogenic composition as disclosed herein comprises a polypeptide comprising at least part of the 172 amino acid sequence of SEQ ID NO: 56, which corresponds to the full length SA2105 protein (WP_042741942.1) from S. aureus but without the N-terminal amino acid residues 1-138, the sequence of which is

(SEQ ID NO: 70)
VFTPLGADQAAKAGQSILYTIESNPGPGLGILLAYMIFGKGTAKATSYGA
GIIHFLGGIHEIYFPYVLMRPLLFIAVILGGMTGVATYQATGFGFKSPAS
PGSFIVYCLNAPRGEFLHMLLGVFLAALVSFVVATLIM

The amino acid sequence of the SA2105 antigen is as follows:

(SEQ ID NO: 56; 172 aa)
KFTREPKQDLEAATAQMENTKGKKSSVASKLVSSDKNVNTEENASGNVSE
TSSSDDDPEALLDNYNTEDVDAHNYNNINHVIFACDAGMGSSAMGASMLR
NKFKKAGINDITVTNTAINQLPKDAQLVITQKKLTDRAIKQTPNAIHISV
DNFLNSPRYEELLNNLKKDDQA

SA2105 as disclosed herein is a 172 amino acid fragment of a larger polypeptide of 512 amino acids which was previously disclosed as SEQ ID NO: 2270 in U.S. Pat. Nos. 7,608,276, 8,287,884, 8,398,996, 8,465,750 and 8,747,864, each of which are incorporated herein by reference in their entirety. The SA polypeptide antigen of SA2105 as disclosed herein corresponds to amino acids 341-512 of SEQ ID NO: 2270 in U.S. Pat. No. 7,608,276 (GenBank No: ADA16392.1), where U.S. Pat. No. 7,608,276 discloses more than 5642 polypeptides for use in a composition.

SA2105 Sequence: The nucleic acid sequence encoding the SA2105 polypeptide of SEQ ID NO: 56 is SEQ ID NO: 63 as follows:

(SEQ ID NO: 63)
AAGTTCACTAGAGAACCAAAGCAGGATTTAGAAGCTGCGACAGCTCAAAT
GGAAAATACTAAAGGGAAAAAATCAAGCGTTGCTTCTAAGTTAGTATCTT
CTGATAAAAATGTTAATACAGAAGAAAATGCTAGTGGTAATGTTAGTGAA
ACATCTTCATCAGATGATGATCCTGAAGCGCTATTGGATAATTACAACAC
TGAAGATGTTGATGCACACAATTACAATAATATAAATCATGTTATTTTTG
CCTGCGATGCGGGTATGGGTTCTAGTGCAATGGGTGCAAGCATGTTACGT
AATAAATTTAAAAAGGCGGGCATTAATGATATTACAGTTACAAATACTGC
GATTAATCAATTGCCAAAAGATGCTCAATTAGTTATCACTCAGAAAAAAC
TAACTGATCGTGCTATTAAACAAACACCAAATGCCATCCATATTTCAGTG
GATAATTTCCTTAATTCACCAAGATATGAAGAACTTTTAAATAATCTAAA
AAAAGATGATCAAGCATAA

SEQ ID NO: 63 comprises a portion of nucleic acid sequence SEQ ID NO: 226+9 disclosed in U.S. Pat. No. 10,226,524, which is incorporated herein in their entirety by reference.

In some embodiments, the SA2105 antigen for use in the SA-MAPS immunogenic composition as disclosed herein is SA2105 (SEQ ID NO: 56), (i.e., residues 139-310 of the full length 301 aa protein) or a fragment or protein of at least 85% amino acid sequence identity thereto.

In one embodiment, a SA-MAPS composition may include a polypeptide, peptide, or protein that is or is at least 70%, 75%, 80%, 85%, 90%, 95%, 96%, 97%, 98%, or 99% identical or similar to a SA2105 of SEQ ID NO: 56. In certain aspects a SA2105 antigen peptide or polypeptide will have all, or part of the amino acid sequence of SEQ ID NO: 56, e.g., will comprise at least 50, or at least 60, or at least 70, or at least 80, or at least 90, or at least 100, or at least 120, or at least 140, or at least 160, or at least 180 amino acids of SEQ ID NO: 56. In one embodiment, a SA2105 antigen peptide or polypeptide present in the SA-MAPS immunogenic composition is a polypeptide, peptide, or protein that is or is at least 70%, 75%, 80%, 85%, 90%, 95%, 96%, 97%, 98%, or 99% identical or similar to SEQ ID NO: 56.

In alternative embodiments, a SA2105 antigen for use in the SA-MAPS immunogenic composition as disclosed herein is a protein or peptide having an amino acid sequences corresponding to amino acid residues 341-512 of SEQ ID NO: 2270 in U.S. Pat. No. 7,608,276 (GenBank No: ADA16392.1). In some embodiments, a SA2105 antigen for use in the SA-MAPS immunogenic composition as disclosed herein is a SA2105 variant of SEQ ID NO: 56, or a mutant SA2105 from S. aureus strain PFESA0237 of SEQ ID NO: 56 or amino acid residues 341-512 of SEQ ID NO: 2270 in U.S. Pat. No. 7,608,276 (GenBank No: ADA16392.1), and are encompassed for use in the SA-MAPS immunogenic composition as disclosed herein.

The term “SA2105 protein” refers to a protein that includes isolated wild-type SA2105 polypeptides without the signal sequence, from staphylococcus bacteria and segments thereof, as well as variants that stimulate an immune response against staphylococcus bacteria SA2105 proteins.

2. Group B S. aureus Antigens:

In some embodiments, the SA-MAPS comprises at least one antigen from Group A and at least one or more SA antigens, where the SA antigen is an antigenic protein or polypeptide, and can be selected from any of Group B SA polypeptide antigens of: hemolysin (Hl) (e.g., hemolysin α or Hla), Clumping factor A (ClfA), Clumping factor B (ClfB), serine-aspirate repeat protein D (SdrD), Iron regulator surface protein A (IsdA) and Iron regulator surface protein B (IsdB), or an antigenic fragment or portion thereof. In some embodiments, the SA-MAPS immunogenic composition as disclosed herein comprises one or more peptide or polypeptide fragments of Group B SA polypeptide antigens, as long as the protein fragment is antigenic, and/or comprises one or more epitopes to induce an immune response.

Exemplary Group B SA polypeptide antigens for use in the SA-MAPS composition as disclosed herein can be, for example, but are not limited to: Hla209(27-319), ClfA(221-559), ClfB (203-542), SdrD (246-682), IsdA (47-324), IsdB (48-447).

In some embodiments, a SA-MAPS immunogenic composition as disclosed herein comprises at least 2, or at least 3, or at least 4, or at least 5, or all 6 Group B SA polypeptide antigens of: Hla209(27-319), ClfA(221-559), ClfB (203-542), SdrD (246-682), IsdA (47-324), IsdB (48-447), or proteins or peptides of at least 85% sequence identity thereto. It is envisioned that any of the above listed Group B SA antigens can be substituted for a different SA peptide or polypeptide antigen known to one of ordinary skill in the art. Exemplary Group B SA antigens can be any peptide or polypeptide comprising at least part of the serine-aspirate repeat protein E (SdrE) protein, Leukotoxin D (LukD) protein, or Leukotoxin E (LukE) protein, provided that the any peptide or polypeptide is immunogenic, or is antigenic. Other SA antigens can be used, and are disclosed herein.

a. Non-Hemolytic Hemolysin α (Hla)

Hemolysin α (Hla) is a secreted pore-forming toxin and an essential virulence factor of MRSA in a mouse model of S. aureus pneumonia. The level of Hla expression by independent S. aureus strains directly correlates with their virulence. In some embodiments, the SA antigen is a non-hemolytic Hla, e.g., Hla(209) as disclosed herein.

Hemolysins are exotoxins produced by bacteria that cause lysis of red blood cells. While highly immunogenic, their use in vaccines is limited because they cause lysis of red blood cells. Accordingly, in another aspect, provided herein are variants of Staphylococcus aureus alpha-hemolysin (Hla) as the SA antigen for use in the SA-MAPS composition as disclosed herein, as well as it in a fusion construct with biotin-binding protein and its uses. These variants, designated herein as “mHla,” have substantially non-hemolytic, i.e., have substantially low hemolytic activity. As used herein, the phrase “substantially non-hemolytic” means an inability to lyse red blood cells at equivalent titers of wild-type Hla. The term “wild-type Hla” is accorded the usual definition associated with such phrase, i.e., Hla that is naturally secreted by a capable bacterial source. “Wild-type Hla,” by definition, does not include, e.g., Hla fusion products derived via recombinant DNA techniques. In some embodiments, hemolytic activity of mHla is at least 5%, at least 10%, at least 15%, at least 20%, at least 20%, at least 30%, at least 30%, at least 35%, least 40%, at least 45%, at least 50%, at least 55%, at least 60%, at least 65%, at least 70%, at least 75%, at least 80%, at least 85%, at least 90%, at least 95% lower than an equivalent titers of wild-type Hla. In some embodiments, the mHla has no detectable hemolytic activity. The inventors have also discovered that hemolytic activity of mHla can be further reduced by linking the mHla with a biotin-binding protein, e.g., a rhizavidin biotin-binding protein as disclosed herein. Accordingly, the present disclosure also describes fusion proteins comprising an mHla protein and a biotin-binding protein.

In some embodiments, a mHla is where the tripeptide DRD209-211 is substituted with a tri-alanine peptide (AAA) in the wild-type Hla, and is referred to herein as Hla209 and comprises the following amino acid sequence:

(SEQ ID NO: 16)
ADSDINIKTGTTDIGSNTTVKTGDLVTYDKENGMHKKVFYSFIDDKNHNK
KLLVIRTKGTIAGQYRVYSEEGANKSGLAWPSAFKVQLQLPDNEVAQISD
YYPRNSIDTKEYMSTLTYGFNGNVTGDDTGKIGGLIGANVSIGHTLKYVQ
PDFKTILESPTDKKVGWKVIFNNMVNQNWGPYAAASWNPVYGNQLFMKTR
NGSMKAADNFLDPNKASSLLSSGFSPDFATVITMDRKASKQQTNIDVIYE
RVRDDYQLHWTSTNWKGTNTKDKWIDRSSERYKIDWEKEEMTN;

In some embodiments, a SA antigen for use in a SA-MAPS immunogenic composition as disclosed herein comprises SEQ ID NO: 16, or a protein or peptide fragment of at least 50 amino acids of SEQ ID NO: 16, or a protein or peptide having at least 85% amino acid identity to SEQ ID NO: 16, where Asp-Arg-Asp (DRD) is mutated to Ala-Ala-Ala (AAA).

In another embodiment, a non-hemolytic Hla can be created where residue W205 or W213 is substituted with alanine (A), and comprise the following sequences, respectively:

Hla W205A:
(SEQ ID NO: 17)
ADSDINIKTGTTDIGSNTTVKTGDLVTYDKENGMHKKVFYSFIDDKNHNK
KLLVIRTKGTIAGQYRVYSEEGANKSGLAWPSAFKVQLQLPDNEVAQISD
YYPRNSIDTKEYMSTLTYGFNGNVTGDDTGKIGGLIGANVSIGHTLKYVQ
PDFKTILESPTDKKVGWKVIFNNMVNQNAGPYDRDSWNPVYGNQLFMKTR
NGSMKAADNFLDPNKASSLLSSGFSPDFATVITMDRKASKQQTNIDVIYE
RVRDDYQLHWTSTNWKGTNTKDKWIDRSSERYKIDWEKEEMTN;

In some embodiments, a SA antigen for use in a SA-MAPS immunogenic composition as disclosed herein comprises SEQ ID NO: 17, or a protein or peptide fragment of at least 50 amino acids of SEQ ID NO: 17, or a protein or peptide having at least 85% amino acid identity to SEQ ID NO: 17, where amino acid W205 is mutated to Ala (W205A).

Hla W213A:
(SEQ ID NO: 18)
ADSDINIKTGTTDIGSNTTVKTGDLVTYDKENGMHKKVFYSFIDDKNHNK
KLLVIRTKGTIAGQYRVYSEEGANKSGLAWPSAFKVQLQLPDNEVAQISD
YYPRNSIDTKEYMSTLTYGFNGNVTGDDTGKIGGLIGANVSIGHTLKYVQ
PDFKTILESPTDKKVGWKVIFNNMVNQNWGPYDRDSANPVYGNQLFMKTR
NGSMKAADNFLDPNKASSLLSSGFSPDFATVITMDRKASKQQTNIDVIYE
RVRDDYQLHWTSTNWKGTNTKDKWIDRSSERYKIDWEKEEMTN;

In some embodiments, a SA antigen for use in a SA-MAPS immunogenic composition as disclosed herein comprises SEQ ID NO: 18, or a protein or peptide fragment of at least 50 amino acids of SEQ ID NO: 18, or a protein or peptide having at least 85% amino acid identity to SEQ ID NO: 18, where amino acid W205 is mutated to Ala (W213A).

A non-toxic non-hemolytic mHla protein can be expressed and purified in an E. coli expression system, and the mutants can be made by point mutation using quick change mutagenesis by a person of ordinary skill in the art. For example, the nucleotide sequence of a nucleic acid encoding the wild-type Hla can be changed to replace a given amino acid in the wild-type Hla to another amino acid.

In some embodiments, the Hla variants described herein, e.g., mHla, such as, Hla209 are ligands for Toll Like Receptors (TLRs), and as such can be used as TLR ligands. For example, the mHla variants can be used in a SA-MAPS immunogenic composition as disclosed herein can induce TLR2 stimulation, e.g., for inducing immunogenicity to other antigens/pathogens.

In some embodiments, a SA-MAPS immunogenic composition as disclosed herein comprising a mHla SA antigen can elicit an immunological response-local or systemic. The response can, but need not, be protective. Accordingly, a non-hemolytic mutant of Hla described herein can be as an antigen, adjuvant, or a co-stimulator in an immunological, immunogenic, or vaccine composition.

In some embodiments, the antigenic protein is a non-hemolytic Hla described herein.

In some embodiments, the non-hemolytic Hla protein is a fusion protein comprising a biotin-binding protein and a non-hemolytic Hla described herein.

In alternative embodiments, the Hla antigen is a mutant mHla of H35L (referred to as SEQ ID NO: 5 in US patent application 2011/0274720 which is incorporated herein in its entirety by reference), which cannot form pores (Menzies, B. E., et al., 1996. Passive immunization with antiserum to a nontoxic alpha-toxin mutant from Staphylococcus aureus is protective in a murine model. Infect Immun 64:1839-41; Jursch, R., et al., 1994. Histidine residues near the N terminus of staphylococcal alpha-toxin as reporters of regions that are critical for oligomerization and pore formation. Infect Immun 62(6): 2249-56), was shown to generate antigen-specific immunoglobulin G responses and to afford protection against staphylococcal pneumonia. Transfer of Hla-specific antibodies protects naive animals against S. aureus challenge and prevents the injury of human lung epithelial cells during infection (Bubeck Wardenburg, J., A. M. Palazzolo-Ballance, M. Otto, O, Schneewind, and F. R. DeLeo. 2008. Panton-Valentine leukocidin is not a virulence determinant in murine models of community-associated methicillin-resistant Staphylococcus aureus disease. J Infect Dis 198:1166-70). To be used as a vaccine, the H35L mutation in Hla is required to eliminate toxicity of the protein (Menzies, B. E., and D. S. Kernodle. 1994. Site-directed mutagenesis of the alpha-toxin gene of Staphylococcus aureus: role of histidines in toxin activity in vitro and in a murine model. Infect Immun 62:1843-7).

In one embodiment, a SA-MAPS composition may include a polypeptide, peptide, or protein that is or is at least 70%, 75%, 80%, 85%, 90%, 95%, 96%, 97%, 98%, or 99% identical or similar to a mHla protein. In certain aspects the mHla protein will have all, or part of the amino acid sequence of SEQ ID NO: 16, e.g., will comprise at least 50, or at least 60, or at least 70, or at least 80, or at least 90, or at least 100, or at least 120, or at least 140, or at least 160, or at least 180, or at least 200, or at least 220 or at least 240, or at least 260, or at least 280 amino acids of SEQ ID NO: 16. In one embodiment, a SA antigen of the SA-MAPS immunogenic composition is a polypeptide, peptide, or protein that is or is at least 70%, 75%, 80%, 85%, 90%, 95%, 96%, 97%, 98%, or 99% identical or similar to SEQ ID NO: 16.

The term “Hla protein” refers to a protein that includes isolated wild-type Hla polypeptides from staphylococcus bacteria and segments thereof, as well as variants that stimulate an immune response against staphylococcus bacteria Hla proteins.

b. Clumping Factor a (ClfA)

Clumping factor A (ClfA) is a S. aureus surface protein associated with binding to host matrix proteins via a fibrinogen binding site, and is functions as a cell wall-associated adhesin protein that mediates staphylococcal binding to fibrinogen and platelets. It is expressed on the cell surface of the bacterium, where it is thought to promote pathogenesis by binding to the fibrinogen and fibrin that is deposited at the site of tissue damage. ClfA is well conserved, and even the most diverse form (−85% identity) exhibits extensive cross-reactivity to both monoclonal and polyclonal antibodies.

ClfA is a member of a family of proteins containing the carboxyl terminal LPXTG (SEQ ID NO: 19) motif that enables the protein to become covalently linked to the cell surface. ClfA also belongs to another family of proteins (Microbial Surface Components Recognizing Adhesive Matrix Molecule, or MSCRAMMs) that are associated with binding host proteins such as fibrinogen (bound by CifA), the fibronectin binding proteins (FnbA and FnbB), the collagen binding protein (Cna) and others. These proteins all share the amino terminal signal sequence that mediates transport to the cell surface. The MSCRAMMs also include an A-domain that is the functional region containing the active site for ligand binding (e.g., fibrinogen, fibronectin, elastin, keratin). The A-domain is followed by a region composed of serine aspartate repeats (SD repeat), which is thought to span the peptidoglycan layer. The SD repeat is followed by a membrane-spanning region that includes the LPXTG (SEQ ID NO: 19) motif for covalent linkage of the protein to peptidoglycan. ClfA is described in U.S. Pat. No. 6,008,341.

Thus, ClfA is a reasonable candidate for a component of a vaccine against S. aureus. However, given the structural instability of ClfA, a formulation of ClfA is problematic since it can readily degrade over time in storage.

Full-length ClfA comprises several regions and domains: an N-terminal secretory domain (“S” domain); followed by a ligand-binding A region, which contains three domains (N1, N2, which contains an EF-hand motif, and N3); followed by an R region, which contains serine-aspartate dipeptide repeats; followed by a cell wall-binding region (“W” region) containing an LPXTG motif SEQ ID NO: 19); a hydrophobic membrane-spanning domain (“M” region); and a charged C-terminus (“C” region) containing positively charged amino acids. The N1 region contains a protease-sensitive site. Much of the instability of ClfA is attributed to the clipping of ClfA at N1, which results in fragments containing N1 and N2N3.

The structure and function of ClfA is disclosed in U.S. Patent Application Publication No. 2007/0087014A1 (Pavliak et al, Apr. 19, 2007), and U.S. Pat. No. 6,008,341 which are incorporated herein by reference in their entirety.

ClfA contains a protease resistant domain which is used for immunization. Passive immunization of mice with anti-ClfA and anti CP5 antibodies effectively sterilized mammary glands in mammary gland infection model (Tuchscherr, L. P., F. R. Buzzola, L. P. Alvarez, J. C. Lee, and D. O. Sordelli. 2008. Antibodies to capsular polysaccharide and clumping factor A prevent mastitis and the emergence of unencapsulated and small-colony variants of Staphylococcus aureus in mice. Infect Immun 76: 5738-44).

The ligand binding region of ClfA comprising N1N2N3 of the A domain spans amino acids 40-559. The N domains of ClfA have been assigned as follows: N1 encompasses residues 45-220; N2 encompasses residues 229-369; and N3 encompasses residues 370-559. See Deivanayagam et al. EMBO J. 21:6660-6672 (2002). For ease of reference the N1N2N3 domains may be referred to as N123, likewise N2N3 may be referred to as N23. In preparations of recombinant N1N2N3, the N1 domain has been found to be protease sensitive and is easily cleaved or hydrolyzed to leave the N2N3 as a stable ligand binding recombinant fragment. See Deivanayagam et al. EMBO J. 21:6660-6672 (2002). The crystal structure of the fibrinogen binding N2N3 fragment of ClfA A domain, revealed that both N2 and N3 are dominated by anti-parallel beta strands. In addition to the anti-parallel beta strands, the N2 domain contains a single turn alpha helix and two 3₁₀ helices and the N3 domain contains three 3₁₀ helices. See Deivanayagam et al. EMBO J. 21:6660-6672 (2002). Sequence alignment of N2 and N3 reveals only 13% sequence identity and 36% sequence similarity over their lengths. See Deivanayagam et al. EMBO J. 21:6660-6672 (2002). The topology of the N2 and N3 domains are similar to the classic IgG fold and have been proposed to be novel variants of the IgG fold. See Deivanayagam et al. EMBO J. 21:6660-6672 (2002).

ClfA Sequence: The gene for clumping factor protein A, designated ClfA, has been cloned, sequenced and analyzed in detail at the molecular level (McDevitt et al., Mol. Microbiol. 11: 237-248 (1994); McDevitt et al., Mol. Microbiol. 16:895-907 (1995)).

In some embodiments, the ClfA antigen for use in the SA-MAPS immunogenic composition as disclosed herein comprises a polypeptide or peptide comprising at least part of SEQ ID NO: 2, which corresponds to the full length ClfA mature protein from S. aureus strain USA300 (without the signal sequence).

(SEQ ID NO: 2)
SENSVTQSDSASNESKSNDSSSVSAAPKTDDTNVSDTKTSSNTNNGETSV
AQNPAQQETTQSSSTNATTEETPVTGEATTTTTNQANTPATTQSSNTNAE
ELVNQTSNETTFNDTNTVSSVNSPQNSTNAENVSTTQDTSTEATPSNNES
APQSTDASNKDVVNQAVNTSAPRMRAFSLAAVAADAPAAGTDITNQLTNV
TVGIDSGTTVYPHQAGYVKLNYGFSVPNSAVKGDTFKITVPKELNLNGVT
STAKVPPIMAGDQVLANGVIDSDGNVIYTFTDYVNTKDDVKATLTMPAYI
DPENVKKTGNVTLATGIGSTTANKTVLVDYEKYGKFYNLSIKGTIDQIDK
TNNTYRQTIYVNPSGDNVIAPVLTGNLKPNTDSNALIDQQNTSIKVYKVD
NAADLSESYFVNPENFEDVTNSVNITFPNPNQYKVEFNTPDDQITTPYIV
VVNGHIDPNSKGDLALRSTLYGYNSNIIWRSMSWDNEVAFNNGSGSGDGI
DKPVVPEQPDEPGEIEPIPEDSDSDPGSDSGSDSNSDSGSDSGSDSTSDS
GSDSASDSDSASDSDSASDSDSASDSDSASDSDSDNDSDSDSDSDSDSDS
DSDSDSDSDSDSDSDSDSDSDSDSDSDSDSDSDSDSDSDSDSDSDSDSDS
DSDSDSDSDSDSDSDSDSDSDSDSDSDSDSDSDSDSDSDSDSDSDSDSDS
DSDSDSDSDSDSDSDSDSDSDSDSDSDSASDSDSDSDSDSDSDSDSDSDS
DSDSDSDSDSDSDSDSDSESDSDSESDSDSDSDSDSDSDSDSDSDSDSAS
DSDSGSDSDSSSDSDSESDSNSDSESGSNNNVVPPNSPKNGTNASNKNEA
KDSKEPLPDTGSEDEANTSLIWGLLASIGSLLLFRRKKENKDKK

In some embodiments, the ClfA antigen for use in the SA-MAPS immunogenic composition as disclosed herein is ClfA (221-559) (SEQ ID NO: 3), or a fragment or protein of at least 85% amino acid sequence identity thereto. SEQ ID NO: 3 has the following amino acid sequence:

(SEQ ID NO: 3)
VAADAPAAGT DITNQLTNVT VGIDSGTTVY
PHQAGYVKLN YGFSVPNSAV KGDTFKITVP KELNLNGVTS
TAKVPPIMAG DQVLANGVID SDGNVIYTFT DYVNTKDDVK
ATLTMPAYID PENVKKTGNV TLATGIGSTT ANKTVLVDYE
KYGKFYNLSI KGTIDQIDKT NNTYRQTIYV NPENFEDVTN
SVNITFPNPN QYKVEFNTPD DQITTPYIVV VNGHIDPNSK
GDLALRSTLY GYNSNIIWRS MSWDNEVAFN NGSGSGDGID
KPVVPEQPDE PGEIEPIPE

In one embodiment, a SA-MAPS composition may include a polypeptide, peptide, or protein that is or is at least 70%, 75%, 80%, 85%, 90%, 95%, 96%, 97%, 98%, or 99% identical or similar to a ClfA of SEQ ID NO: 2 or SEQ ID NO: 3. In certain aspects a ClfA antigen peptide or polypeptide will have all, or part of the amino acid sequence of SEQ ID NO: 3, e.g., will comprise at least 50, or at least 60, or at least 70, or at least 80, or at least 90, or at least 100, or at least 120, or at least 140, or at least 160, or at least 180, or at least 200, or at least 220 or at least 240 amino acids of SEQ ID NO: 2 or SEQ ID NO: 3. In one embodiment, a ClfA antigen peptide or polypeptide present in the SA-MAPS immunogenic composition is a polypeptide, peptide, or protein that is or is at least 70%, 75%, 80%, 85%, 90%, 95%, 96%, 97%, 98%, or 99% identical or similar to SEQ ID NO: 2.

In alternative embodiments, a ClfA antigen for use in the SA-MAPS immunogenic composition as disclosed herein is a protein or peptide having an amino acid sequence of ClfA from 111 S. aureus disease-causing isolates disclosed in Table 10 of U.S. Pat. No. 8,568,735, which is incorporated herein in its entirety by reference. In some embodiments, a ClfA antigen for use in the SA-MAPS immunogenic composition as disclosed herein is a ClfA variant of SEQ ID NO: 61-108, or a mutant ClfA from S. aureus strain PFESA0237 of SEQ ID NO: 130, 131 and 123 as disclosed in U.S. Pat. No. 8,568,735, and are encompassed for use in the SA-MAPS immunogenic composition as disclosed herein.

The amino acid sequence of the full length (including the signal sequence) wild type ClfA from S. aureus strain PFESA0237 is disclosed as SEQ ID NO: 130 in U.S. Pat. No. 8,568,735. SEQ ID NO: 130 has a tyrosine at position 338, which is changed to an alanine in the mutated form of ClfA (mClfA). The full length gene encoding the wild type ClfA from S. aureus strain PFESA0237, comprising the N123 region, the repeat region and the anchor region is disclosed as SEQ ID NO: 131 in U.S. Pat. No. 8,568,735, and the amino acid sequence of the mClfA(Y338A) id disclosed as SEQ ID NO: 123 in U.S. Pat. No. 8,568,735. However, it should be noted that the change from a tyrosine to an alanine, which occurs in the wild type ClfA at position 338 of SEQ ID NO: 130, and which is designated as Y338A, is shown in the mutated form of ClfA, in SEQ ID NO: 123 at position 310. Furthermore, the mutated form of ClfA shown in the amino acid sequence of SEQ ID NO: 123 is the mature form of ClfA without the signal sequence, thus accounting for the difference in position of this mutation between SEQ ID NO: 130 and SEQ ID NO: 123.

The term “ClfA protein” refers to a protein that includes isolated wild-type ClfA polypeptides from staphylococcus bacteria and segments thereof, as well as variants that stimulate an immune response against staphylococcus bacteria ClfA proteins.

c. Clumping Factor B (ClfB)

Clumping factor B (ClfB) is a S. aureus protein having fibrinogen binding activity and triggers S. aureus to form clumps in the presence of plasma. ClfB is an MSCRAMM protein and displays the characteristic MSCRAMM domain organization including an A-domain that is the functional region containing the active site for ligand binding (e.g., fibrinogen, fibronectin, elastin, keratin). The A-domain is followed by a region composed of serine aspartate repeats (SD repeat), which is thought to span the peptidoglycan layer. The SD repeat is followed by a membrane-spanning region that includes the LPXTG (SEQ ID NO: 19) motif for covalent linkage of the protein to peptidoglycan. ClfB is described in WO 99/27109 and in U.S. Pat. Nos. 6,680,195 and 8,568,735, which are incorporated herein in their entirety by reference.

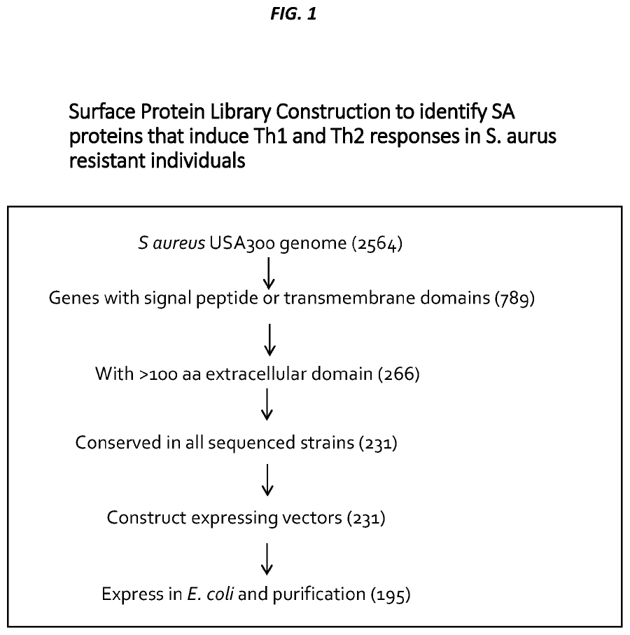

The internal organization of ClfB N-terminal A domain is very similar organization as found in ClfB. The A domain is composed of three subdomains N1, N2, and N3. The ligand binding region of ClfB comprising N1N2N3 of the A domain (FIG. 1) spans amino acids 44-585. For ease of reference the N1N2N3 domains may be referred to as N123, likewise N2N3 may be referred to as N23. The N domains of ClfB have been assigned as follows: N1 encompasses residues 44-197; N2 encompasses residues 198-375; and N3 encompasses residues 375-585. In ClfA, the crystal structure of the A domain was found to have a unique version of the immunoglobulin fold and by analogy the same may be speculated to be the case for ClfB. See Deivanayagam et al., EMBO J. 21:6660-6672 (2002). Even though organization of the A domains of ClfB and ClfA are similar, sequence identity is only 26%, See Ni Eidhin et al., Mol. Microbiol. 30:245-257 (2002).

ClfB Sequence: The gene encoding ClfB is classified as a core adhesion gene. In some embodiments, the ClfB antigen for use in the SA-MAPS immunogenic composition as disclosed herein comprises a polypeptide or peptide comprising at least part of SEQ ID NO: 4, which corresponds to the full length ClfB mature protein from S. aureus strain USA300 (without the signal sequence).

(SEQ ID NO: 4)
SEQSNDTTQSSKNNASADSEKNNMIETPQLNTTANDTSDISANTNSANVDSTTKPMSTQTSNTTT
TEPASTNETPQPTAIKNQATAAKMQDQTVPQEANSQVDNKTTNDANSIATNSELKNSQTLDLPQ
SSPQTISNAQGTSKPSVRTRAVRSLAVAEPVVNAADAKGTNVNDKVTASNFKLEKTTFDPNQSG
NTFMAANFTVTDKVKSGDYFTAKLPDSLTGNGDVDYSNSNNTMPIADIKSTNGDVVAKATYDI
LTKTYTFVFTDYVNNKENINGQFSLPLFTDRAKAPKSGTYDANINIADEMFNNKITYNYSSPIAGI
DKPNGANISSQIIGVDTASGQNTYKQTVFVNPKQRVLGNTWVYIKGYQDKIEESSGKVSATDTK
LRIFEVNDISKLSDSYYADPNDSNLKEVTDQFKNRIYYEHPNVASIKFGDITKTYVVLVEGHYDN
TGKNLKTQVIQENVDPVTNRDYSIFGWNNENVVRYGGGSADGDSAVNPKDPTPGPPVDPEPSP
DPEPEPSPDPDPDSDSDSDSGSDSDSGSDSDSESDSDSDSDSDSDSDSDSESDSDSESDSDSDSDSD
SDSDSDSDSDSDSDSDSDSDSDSDSESDSDSESDSESDSDSDSDSDSDSDSDSDSDSDSDSDSDSD
SDSDSDSDSDSDSDSDSDSDSDSDSDSDSDSDSDSDSDSDSDSDSDSDSDSDSDSDSDSDSDSDSD
SDSDSDSDSDSDSDSDSDSDSDSDSDSDSDSDSDSDSDSDSDSDSDSDSDSDSDSDSDSDSDSDSD
SDSRVTPPNNEQKAPSNPKGEVNHSNKVSKQHKTDALPETGDKSENTNATLFGAMMALLGSLL
LFRKRKQDHKEKA

In some embodiments, the ClfB antigen for use in the SA-MAPS immunogenic composition as disclosed herein is ClfB (203-542) (SEQ ID NO: 5), or a fragment or protein of at least 85% amino acid sequence identity thereto. SEQ ID NO: 5 has the following amino acid sequence:

(SEQ ID NO: 5)
PVVNAADAKGTNVNDKVTASNFKLEKTTFDPNQSGNTFMAANFTVTDKVKSGDYFTAKLPDSL
TGNGDVDYSNSNNTMPIADIKSTNGDVVAKATYDILTKTYTFVFTDYVNNKENINGQFSLPLFT
DRAKAPKSGTYDANINIADEMFNNKITYNYSSPIAGIDKPNGANISSQIIGVDTASGQNTYKQTVF
VNPKQRVLGNTWVYIKGYQDKIEESSGKVSATDTKLRIFEVNDISKLSDSYYADPNDSNLKEVT
DQFKNRIYYEHPNVASIKFGDITKTYVVLVEGHYDNTGKNLKTQVIQENVDPVTNRDYSIFGWN
NENVVRYGGGSADGDSA
VN

In one embodiment, a SA-MAPS composition may include a polypeptide, peptide, or protein that is or is at least 70%, 75%, 80%, 85%, 90%, 95%, 96%, 97%, 98%, or 99% identical or similar to a ClfB of SEQ ID NO: 4 or SEQ ID NO: 5. In certain aspects a ClfB antigen peptide or polypeptide will have all, or part of the amino acid sequence of SEQ ID NO: 5, e.g., will comprise at least 50, or at least 60, or at least 70, or at least 80, or at least 90, or at least 100, or at least 120, or at least 140, or at least 160, or at least 180, or at least 200, or at least 220 or at least 240 amino acids of SEQ ID NO: 4 or SEQ ID NO: 5. In one embodiment, a ClfB antigen peptide or polypeptide present in the SA-MAPS immunogenic composition is a polypeptide, peptide, or protein that is or is at least 70%, 75%, 80%, 85%, 90%, 95%, 96%, 97%, 98%, or 99% identical or similar to SEQ ID NO: 5.

In alternative embodiments, a ClfB antigen for use in the SA-MAPS immunogenic composition as disclosed herein is a protein or peptide having an amino acid sequence of one the ClfB proteins sequenced from 92 strains of S. aureus associated with multiple disease states, which are disclosed in Table 11 of U.S. Pat. No. 8,568,735. Other ClfB antigens not identified herein are disclosed encompassed for use in the SA-MAPS immunogenic composition, provided they are antigenic.

The term “ClfB protein” refers to a protein that includes isolated wild-type ClfB polypeptides from staphylococcus bacteria and segments thereof, as well as variants that stimulate an immune response against staphylococcus bacteria ClfB proteins.

d. Serine-Aspirate Repeat Protein D (SdrD)

SdrD Sequence: In some embodiments, the SdrD antigen for use in the SA-MAPS immunogenic composition as disclosed herein comprises a polypeptide or peptide comprising at least part of SEQ ID NO: 6, which corresponds to the full length SdrD mature protein (aa53-1831) from S. aureus strain USA300 (without the signal sequence).

(SEQ ID NO: 6)
AESTNKELNEATTSASDNQSSDKVDMQQLNQEDNTKNDNQKEMVSSQGNETTSNGNKLIEKESVQSTTGN
KVEVSTAKSDEQASPKSTNEDLNTKQTISNQEALQPDLQENKSVVNVQPTNEENKKVDAKTESTTLNVKS
DAIKSNDETLVDNNSNSNNENNADIILPKSTAPKRLNTRMRIAAVQPSSTEAKNVNDLITSNTTLTVVDA
DKNNKIVPAQDYLSLKSQITVDDKVKSGDYFTIKYSDTVQVYGLNPEDIKNIGDIKDPNNGETIATAKHD
TANNLITYTFTDYVDRFNSVQMGINYSIYMDADTIPVSKNDVEFNVTIGNTTTKTTANIQYPDYVVNEKN
SIGSAFTETVSHVGNKENPGYYKQTIYVNPSENSLTNAKLKVQAYHSSYPNNIGQINKDVTDIKIYQVPK
GYTLNKGYDVNTKELTDVTNQYLQKITYGDNNSAVIDFGNADSAYVVMVNTKFQYTNSESPTLVQMATLS
STGNKSVSTGNALGFTNNQSGGAGQEVYKIGNYVWEDTNKNGVQELGEKGVGNVTVTVFDNNTNTKVGEA
VTKEDGSYLIPNLPNGDYRVEFSNLPKGYEVTPSKQGNNEELDSNGLSSVITVNGKDNLSADLGIYKPKY
NLGDYVWEDTNKNGIQDQDEKGISGVTVTLKDENGNVLKTVTTDADGKYKFTDLDNGNYKVEFTTPEGYT
PTTVTSGSDIEKDSNGLTTTGVINGADNMTLDSGFYKTPKYNLGNYVWEDTNKDGKQDSTEKGISGVTVT
LKNENGEVLQTTKTDKDGKYQFTGLENGTYKVEFETPSGYTPTQVGSGTDEGIDSNGTSTTGVIKDKDND
TIDSGFYKPTYNLGDYVWEDTNKNGVQDKDEKGISGVTVTLKDENDKVLKTVTTDENGKYQFTDLNNGTY
KVEFETPSGYTPTSVTSGNDTEKDSNGLTTTGVIKDADNMTLDSGFYKTPKYSLGDYVWYDSNKDGKQDS
TEKGIKDVKVTLLNEKGEVIGTTKTDENGKYCFDNLDSGKYKVIFEKPAGLTQTGTNTTEDDKDADGGEV
DVTITDHDDFTLDNGYYEEETSDSDSDSDSDSDSDRDSDSDSDSDSDSDSDSDSDSDSDSDSDSDRDSDS
DSDSDSDSDSDSDSDSDSDSDSDSDSDSDSDSDSDSDSDSDSDSDSDSDSDSDSDSDSDSDSDSDSDSDS
DSDSDSDSDSDSDSDSDSDSDSDSDSDSDSDSDSDSDSDSDSDSDSDSDSDSDSDSDSDSDSDSDSDSDS
DSDSDSDSDAGKHTPVKPMSTTKDHHNKAKALPETGNENSGSNNATLFGGLFAALGSLLLFGRRKKQNK

In some embodiments, the SdrD antigen for use in the SA-MAPS immunogenic composition as disclosed herein is SdrD (246-682) (SEQ ID NO: 7), or a fragment or protein of at least 85% amino acid sequence identity thereto. SEQ ID NO: 7 has the following amino acid sequence:

(SEQ ID NO: 7)
NVNDLITSNTTLTVVDADKNNKIVPAQDYLSLKSQITVDDKVKSGDYFTIKYSDT
VQVYGLNPEDIKNIGDIKDPNNGETIATAKHDTANNLITYTFTDYVDRFNSVQMGINYSI
YMDADTIPVSKNDVEFNVTIGNTTTKTTANIQYPDYVVNEKNSIGSAFTETVSHVGNKEN
PGYYKQTIYVNPSENSLTNAKLKVQAYHSSYPNNIGQINKDVTDIKIYQVPKGYTLNKGY
DVNTKELTDVTNQYLQKITYGDNNSAVIDFGNADSAYVVMVNTKFQYTNSESPTLVQMAT
LSSTGNKSVSTGNALGFTNNQSGGAGQEVYKIGNYVWEDTNKNGVQELGEKGVGNVTVTV
FDNNTNTKVGEAVTKEDGSYLIPNLPNGDYRVEFSNLPKGYEVTPSKQGNNEELDSNGLS
SVITVNGKDNLSADLGIYKPKY

In one embodiment, a SA-MAPS composition may include a polypeptide, peptide, or protein that is or is at least 70%, 75%, 80%, 85%, 90%, 95%, 96%, 97%, 98%, or 99% identical or similar to a SdrD of SEQ ID NO: 6 or SEQ ID NO: 7. In certain aspects a SdrD antigen peptide or polypeptide will have all, or part of the amino acid sequence of SEQ ID NO: 7, e.g., will comprise at least 50, or at least 60, or at least 70, or at least 80, or at least 90, or at least 100, or at least 120, or at least 140, or at least 160, or at least 180, or at least 200, or at least 220 or at least 240 amino acids of SEQ ID NO: 6 or SEQ ID NO: 7. In one embodiment, a SdrD antigen peptide or polypeptide present in the SA-MAPS immunogenic composition is a polypeptide, peptide, or protein that is or is at least 70%, 75%, 80%, 85%, 90%, 95%, 96%, 97%, 98%, or 99% identical or similar to SEQ ID NO: 7.

The term “SdrD protein” refers to a protein that includes isolated wild-type SdrD polypeptides from staphylococcus bacteria and segments thereof, as well as variants that stimulate an immune response against staphylococcus bacteria SdrD proteins.

In some embodiments, other SdrD antigens can be used in the SA-MAPS composition as disclosed herein, e.g., SdrD antigenic proteins or peptides derived from various species of organisms, some of which include the following SdrD from S. aureus: strain USA300 FPR3757 (protein accession number SAUSA300 0547); strain NCTC8325 (protein accession number SAOUHSC 00545): strain MW2 (protein accession number MW0517); strain MSSA476 (protein accession number SAS0520; and strain Mu50 (protein accession number SAV0562).

e. Iron Regulator Surface Protein a (IsdA)

IsdA Sequence: In some embodiments, the IsdA antigen for use in the SA-MAPS immunogenic composition as disclosed herein comprises a polypeptide or peptide comprising at least part of SEQ ID NO: 10, which corresponds to the full length IsdA mature protein (aa 47-350) from S. aureus strain USA300 (without the signal sequence).

(SEQ ID NO: 10)
ATEATNATNNQSTQVSQATSQPINFQVQKDGSSEKSHMDDYMQHPGKVIKQNNKYYFQTVLNNASFWKEY
KFYNANNQELATTVVNDNKKADTRTINVAVEPGYKSLTTKVHIVVPQINYNHRYTTHLEFEKAIPTLADA
AKPNNVKPVQPKPAQPKTPTEQTKPVQPKVEKVKPTVTTTSKVEDNHSTKVVSTDTTKDQTKTQTAHTVK
TAQTAQEQNKVQTPVKDVATAKSESNNQAVSDNKSQQTNKVTKHNETPKQASKAKELPKTGLTSVDNFIS
TVAFATLALLGSLSLLLFKRKESK

In some embodiments, the IsdA antigen for use in the SA-MAPS immunogenic composition as disclosed herein is IsdA (47-324) (SEQ ID NO: 11), or a fragment or protein of at least 85% amino acid sequence identity thereto. SEQ ID NO: 11 has the following amino acid sequence:

(SEQ ID NO: 11)
ATEATNATNNQSTQVSQATSQPINFQVQKDGSSEKSHMDDYMQHPGKVIKQNNKYYFQTVLNNASFWKEY
KFYNANNQELATTVVNDNKKADTRTINVAVEPGYKSLTTKVHIVVPQINYNHRYTTHLEFEKAIPTLADA
AKPNNVKPVQPKPAQPKTPTEQTKPVQPKVEKVKPTVTTTSKVEDNHSTKVVSTDTTKDQTKTQTAHTVK
TAQTAQEQNKVQTPVKDVATAKSESNNQAVSDNKSQQTNKVTKHNETPKQASKAKELPKTGLTSVDNF

In one embodiment, a SA-MAPS composition may include a polypeptide, peptide, or protein that is or is at least 70%, 75%, 80%, 85%, 90%, 95%, 96%, 97%, 98%, or 99% identical or similar to a IsdA of SEQ ID NO: 10 or SEQ ID NO: 11. In certain aspects a IsdA antigen peptide or polypeptide will have all, or part of the amino acid sequence of SEQ ID NO: 11, e.g., will comprise at least 50, or at least 60, or at least 70, or at least 80, or at least 90, or at least 100, or at least 120, or at least 140, or at least 160, or at least 180, or at least 200, or at least 220 or at least 240 amino acids of SEQ ID NO: 10 or SEQ ID NO: 11. In one embodiment, a IsdA antigen peptide or polypeptide present in the SA-MAPS immunogenic composition is a polypeptide, peptide, or protein that is or is at least 70%, 75%, 80%, 85%, 90%, 95%, 96%, 97%, 98%, or 99% identical or similar to SEQ ID NO: 11.

The term “IsdA protein” refers to a protein that includes isolated wild-type IsdA polypeptides from staphylococcus bacteria and segments thereof, as well as variants that stimulate an immune response against staphylococcus bacteria IsdA proteins.

f. Iron Regulator Surface Protein B (IsdB)

In some embodiments, a SA antigen for use in the SA-MAPS composition as disclosed herein is the S. aureus surface protein iron surface determinant B (IsdB). This MSCRAMM was described by Mazmanian et al. (Mazmanian, S K et al. Proc. Natl. Acad. Sci., USA 99:2293-2298 (2002)) and it has subsequently been tested and shown to be effective as a vaccine candidate in a murine model of infection and a rhesus macaque immunogenicity study by Kuklin, et al. (Kuklin, N A, et al. Infection and Immunity, Vol. 74, No. 4, 2215-2223, (2006)).

IsdB Sequence: In some embodiments, the IsdB antigen for use in the SA-MAPS immunogenic composition as disclosed herein comprises a polypeptide or peptide comprising at least part of SEQ ID NO: 12, which corresponds to the full length IsdB mature protein (aa 41-652) from S. aureus strain USA300 (without the signal sequence).

(SEQ ID NO: 12)
AAEETGGTNTEAQPKTEAVASPTTTSEKAPETKPVANAVSVSNKEVEAPTSETKEAKEVKEVKAPKETKE
VKPAAKATNNTYPILNQELREAIKNPAIKDKDHSAPNSRPIDFEMKKKDGTQQFYHYASSVKPARVIFTD
SKPEIELGLQSGQFWRKFEVYEGDKKLPIKLVSYDTVKDYAYIRFSVSNGTKAVKIVSSTHFNNKEEKYD
YTLMEFAQPIYNSADKFKTEEDYKAEKLLAPYKKAKTLERQVYELNKIQDKLPEKLKAEYKKKLEDTKKA
LDEQVKSAITEFQNVQPTNEKMTDLQDTKYVVYESVENNESMMDTFVKHPIKTGMLNGKKYMVMETTNDD
YWKDFMVEGQRVRTISKDAKNNTRTIIFPYVEGKTLYDAIVKVHVKTIDYDGQYHVRIVDKEAFTKANTD
KSNKKEQQDNSAKKEATPATPSKPTPSPVEKESQKQDSQKDDNKQLPSVEKENDASSESGKDKTPATKPT
KGEVESSSTTPTKVVSTTQNVAKPTTASSKTTKDVVQTSAGSSEAKDSAPLQKANIKNTNDGHTQSQNNK
NTQENKAKSLPQTGEESNKDMTLPLMALLALSSIVAFVLPRKRKN

In some embodiments, the IsdB antigen for use in the SA-MAPS immunogenic composition as disclosed herein is IsdB (48-477) (SEQ ID NO: 13), or a fragment or protein of at least 85% amino acid sequence identity thereto. SEQ ID NO: 13 has the following amino acid sequence:

(SEQ ID NO: 13)
TNTEAQPKTEAVASPTTTSEKAPETKPVANAVSVSNKEVEAPTSETKEAKEVKEVKAPKETKEVKPAAKA
TNNTYPILNQELREAIKNPAIKDKDHSAPNSRPIDFEMKKKDGTQQFYHYASSVKPARVIFTDSKPEIEL
GLQSGQFWRKFEVYEGDKKLPIKLVSYDTVKDYAYIRFSVSNGTKAVKIVSSTHFNNKEEKYDYTLMEFA
QPIYNSADKFKTEEDYKAEKLLAPYKKAKTLERQVYELNKIQDKLPEKLKAEYKKKLEDTKKALDEQVKS
AITEFQNVQPTNEKMTDLQDTKYVVYESVENNESMMDTFVKHPIKTGMLNGKKYMVMETTNDDYWKDFMV
EGQRVRTISKDAKNNTRTIIFPYVEGKTLYDAIVKVHVKTIDYDGQYHVRIVDKEAFTKANTDKSNKKEQ
QDNSAKKEAT

In one embodiment, a SA-MAPS composition may include a polypeptide, peptide, or protein that is or is at least 70%, 75%, 80%, 85%, 90%, 95%, 96%, 97%, 98%, or 99% identical or similar to a IsdB of SEQ ID NO: 12 or SEQ ID NO: 13. In certain aspects a IsdA antigen peptide or polypeptide will have all, or part of the amino acid sequence of SEQ ID NO: 13, e.g., will comprise at least 50, or at least 60, or at least 70, or at least 80, or at least 90, or at least 100, or at least 120, or at least 140, or at least 160, or at least 180, or at least 200, or at least 220 or at least 240 amino acids of SEQ ID NO: 12 or SEQ ID NO: 31. In one embodiment, a IsdB antigen peptide or polypeptide present in the SA-MAPS immunogenic composition is a polypeptide, peptide, or protein that is or is at least 70%, 75%, 80%, 85%, 90%, 95%, 96%, 97%, 98%, or 99% identical or similar to SEQ ID NO: 13.

The term “IsdB protein” refers to a protein that includes isolated wild-type IsdB polypeptides from staphylococcus bacteria and segments thereof, as well as variants that stimulate an immune response against staphylococcus bacteria IsdB proteins.

In some embodiments, other IsdB antigens can be used in the SA-MAPS composition as disclosed herein, e.g., IsdB antigenic proteins or peptides derived from various species of organisms, some of which include the following IsdB from S. aureus strains, including strain MRSA252 (protein accession number CAG40104.1); strain Newman (protein accession number BAF67312.1); strain MSSA476 (protein accession number CAG42837.1); strain Mu3 (protein accession number BAF78003.1); strain RF122 (protein accession number CA180681.1).

g. Serine-Aspirate Repeat Protein E (SdrE)

The sdr genes are closely linked and tandemly arrayed, accordingly any one of the Sdr proteins (e.g., SdrC, SdrD, SdrE, ClfA, and ClfB) can be used in the SA-MAPS immunogenic composition as disclosed herein. The Sdr proteins characteristically comprise an A region where there is highly conserved amino acid sequence that can be used to derive a consensus TYTFTDYVD (SEQ ID NO: 20) motif. The motif exhibits slight variation between the different proteins. This variation, along with the consensus sequence of the motif is described in U.S. Pat. No. 6,680,195. In the Clf-Sdr proteins, this motif is highly conserved. The motif can be used in immunogenic compositions to impart broad spectrum immunity to bacterial infections, and also can be used as an antigen in the production of monoclonal or polyclonal antibodies. Such an antibody can be used to impart broad spectrum passive immunity.

The Sdr proteins differ from ClfA and ClfB by having two to five additional 110-113 residue repeated sequences (B-motifs) located between region A and the R-region. Each B-motif contains a consensus Ca2+-binding EF-hand loop normally found in eukaryotic proteins. The structural integrity of a recombinant protein comprising the five B-repeats of SdrD was shown by bisANS fluorescence analysis to be Ca2+-dependent, suggesting that the EF-hands are functional. When Ca2+ was removed the structure collapsed to an unfolded conformation. The original structure was restored by addition of Ca2+. The C-terminal R-domains of the Sdr proteins contain 132-170 SD residues. These are followed by conserved wall-anchoring regions characteristic of many surface proteins of Gram positive bacteria.

In the Sdr and Clf proteins this B motif is highly conserved while a degenerate version occurs in fibronectin binding MSCRAMMS, as well as the collagen binding protein Cna. The B motifs, in conjunction with the R regions, are necessary for displaying the ligand-binding domain at some distance from the cell surface. The repeated B motifs are one common denominator of the sub-group of SD repeat proteins described herein. These motifs are found in different numbers in the three Sdr proteins from strain PFESA0237. There are clear distinctions between the individual B motifs. The most conserved units are those located adjacent to the R regions (SdrC B2, SdrD B5 and SdrE B3). They differ from the rest at several sites, especially in the C-terminal half A noteworthy structural detail is that adjacent B repeats are always separated by a proline residue present in the C-terminal region, but a proline never occurs between the last B repeats and the R region. Instead this linker is characterized by a short acidic stretch. These differences are evidence that the end units have a different structural or functional role compared to the other B motifs. The N-terminal B motifs of SdrD and SdrE have drifted apart from the others, and there are numerous amino acid alterations, including small insertions and deletions whereas the remaining internal B motifs are more highly conserved. Note that each of the three Sdr proteins has at least one B motif of each kind.

The C-terminal R-domains of the Sdr proteins contain 132-170 SD residues. These are followed by conserved wall-anchoring regions characteristic of many surface proteins of Gram positive bacteria.

In some embodiments, a SdrE antigen can be used in the SA-MAPS immunogenic composition as disclosed herein, and can comprises a polypeptide or peptide comprising at least part of SEQ ID NO: 8, which corresponds to the full length SdrE mature protein from S. aureus strain USA300 (without the signal sequence).

(SEQ ID NO: 8)
AENTSTENAKQDDATTSDNKEVVSETENNSTTENNSTNPIKKETNTDSQPEAKKESTSSS
TQKQQNNVTATTETKPQNIEKENVKPSTDKTATEDTSVILEEKKAPNNTNNDVTTKPSTS
EPSTSEIQTKPTTPQESTNIENSQPQPTPSKVDNQVTDATNPKEPVNVSKEELKNNPEKL
KELVRNDSNTDHSTKPVATAPTSVAPKRVNAKMRFAVAQPAAVASNNVNDLIKVTKQTIK
VGDGKDNVAAAHDGKDIEYDTEFTIDNKVKKGDTMTINYDKNVIPSDLTDKNDPIDITDP
SGEVIAKGTEDKATKQITYTFTDYVDKYEDIKSRLTLYSYIDKKTVPNETSLNLTFATAG
KETSQNVTVDYQDPMVHGDSNIQSIFTKLDEDKQTIEQQIYVNPLKKSATNTKVDIAGSQ
VDDYGNIKLGNGSTIIDQNTEIKVYKVNSDQQLPQSNRIYDFSQYEDVTSQFDNKKSFSN
NVATLDFGDINSAYIIKVVSKYTPTSDGELDIAQGTSMRTTDKYGYYNYAGYSNFIVTSN
DTGGGDGTVKPEEKLYKIGDYVWEDVDKDGVQGTDSKEKPMANVLVTLTYPDGTTKSVRT
DANGHYEFGGLKDGETYTVKFETPTGYLPTKVNGTTDGEKDSNGSSVTVKINGKDDMSLD
TGFYKEPKYNLGDYVWEDTNKDGIQDANEPGIKDVKVTLKDSTGKVIGTTTTDASGKYKF
TDLDNGNYTVEFETPAGYTPTVKNTTADDKDSNGLTTTGVIKDADNMTLDSGFYKTPKYS
LGDYVWYDSNKDGKQDSTEKGIKDVTVTLQNEKGEVIGTTKTDENGKYRFDNLDSGKYKV
IFEKPAGLTQTVTNTTEDDKDADGGEVDVTITDHDDFTLDNGYFEEDTSDSDSDSDSDSD
SDSDSDSDSDSDSDSDSDSDSDSDSDSDSDSDSDSDSDSDSDSDSDSDSDSDSDSDSDSD
SDSDSDSDSDSDSDSDSDSDSDSDSDSDSDSDSDSDSDSDSDSDSDSDSDSDSDSDSDSD
SDSDSDSDSDSDSDSDSDSDSDAGKHTPVKPMSTTKDHHNKAKALPET

In some embodiments, a SA-MAPS composition can include a polypeptide, peptide, or protein that is or is at least 70%, 75%, 80%, 85%, 90%, 95%, 96%, 97%, 98%, or 99% identical or similar to a SdrE of SEQ ID NO: 8. In certain aspects a SdrE antigen peptide or polypeptide will have all, or part of the amino acid sequence of SEQ ID NO: 8, e.g., will comprise at least 50, or at least 60, or at least 70, or at least 80, or at least 90, or at least 100, or at least 120, or at least 140, or at least 160, or at least 180, or at least 200, or at least 220 or at least 240 amino acids of SEQ ID NO: 8. In one embodiment, a SdrD antigen peptide or polypeptide present in the SA-MAPS immunogenic composition is a polypeptide, peptide, or protein that is or is at least 70%, 75%, 80%, 85%, 90%, 95%, 96%, 97%, 98%, or 99% identical or similar to SEQ ID NO: 8.

The term “SdrE protein” refers to a protein that includes isolated wild-type SdrE polypeptides from staphylococcus bacteria and segments thereof, as well as variants that stimulate an immune response against staphylococcus bacteria SdrE proteins.

The term “SdrC protein” refers to a protein that includes isolated wild-type SdrC polypeptides from staphylococcus bacteria and segments thereof, as well as variants that stimulate an immune response against staphylococcus bacteria SdrC proteins.

h. LukD, LukE, LukF

In one embodiment, a SA-MAPS composition may include a polypeptide, peptide, or protein that is or is at least 70%, 75%, 80%, 85%, 90%, 95%, 96%, 97%, 98%, or 99% identical or similar to an LukD protein. In certain aspects the LukD protein will have all or part of the amino acid sequence of accession number CAA73668/GI:2765304. The term “LukD protein” refers to a protein that includes isolated wild-type LukD polypeptides from staphylococcus bacteria and segments thereof, as well as variants that stimulate an immune response against staphylococcus bacteria LukD proteins.

In one embodiment, a SA-MAPS composition may include a polypeptide, peptide, or protein that is or is at least 70%, 75%, 80%, 85%, 90%, 95%, 96%, 97%, 98%, or 99% identical or similar to an LukE protein. In certain aspects the LukE protein will have all or part of the amino acid sequence of accession number CAA73667.1/GI:2765303. The term “LukE protein” refers to a protein that includes isolated wild-type LukE polypeptides from staphylococcus bacteria and segments thereof, as well as variants that stimulate an immune response against staphylococcus bacteria LukE proteins.

In one embodiment, a SA-MAPS composition may include a polypeptide, peptide, or protein that is or is at least 70%, 75%, 80%, 85%, 90%, 95%, 96%, 97%, 98%, or 99% identical or similar to an LukF protein. In certain aspects the LukF protein will have all or part of the amino acid sequence of accession number AAC60446.1/GI:410007. The term “LukF protein” refers to a protein that includes isolated wild-type LukF polypeptides from staphylococcus bacteria and segments thereof, as well as variants that stimulate an immune response against staphylococcus bacteria LukF proteins.

i. Other SA Antigens

While exemplary SA antigens used in the SA-MAPS composition as disclosed herein can be one or more of, or all 6 of hemolysin (Hl) (e.g., hemolysin α or Hla209), Clumping factor A (ClfA), Clumping factor B (ClfB), serine-aspirate repeat protein D (SdrD), Iron regulator surface protein A (IsdA) and Iron regulator surface protein B (IsdB), or fragments thereof, e.g., Hla209(27-319), ClfA(221-559), ClfB (203-542), SdrD (246-682), IsdA (47-324), IsdB (48-447) or proteins or peptides having at least 85% sequence identity thereto, it is envisioned that any of the above listed SA antigens can be substituted for a different SA peptide or polypeptide antigen known to one of ordinary skill in the art.

For example, in some embodiments, any one or more SA antigens useful in the SA-MAPS composition as disclosed herein include, but are not limited to, a peptide or polypeptide comprising at least part of the serine-aspirate repeat protein E (SdrE) protein, SdrC, Leukotoxin D (LukD) protein, or Leukotoxin E (LukE) protein, provided that the any peptide or polypeptide is immunogenic, or is antigenic. Other SA antigens can be used, and are disclosed herein.

In some embodiments, other SA antigens can be used in the SA-MAPS composition as disclosed herein. For example, the S. aureus MntC protein (also known as Protein 305, P305, P305A, and ORF305) is a component of a manganese ABC transporter. This protein is expressed in vivo. S. aureus uses manganese as a cofactor for an enzyme that enhances the survival of S. aureus in neutraphils. MntC is, therefore, important for the in vivo survival of S. aureus during infection. Like ClfA, this protein is also unstable in solution. However, unlike ClfA, which can aggregate, or clip via hydrolysis, the primary mechanism of MntC degradation is deamidation when subject to basic pH and/or temperature around room temperature (about 25° C.) or higher.

In some embodiments SA antigens can be used in the SA-MAPS composition as disclosed herein can be selected from any one or, or a combination of: SdrC, SdrE, MntC/SitC/Saliva Binding Protein, Opp3a, DltA, HtsA, LtaS, SdrH, SrtA, SpA, SBI, beta-hemolysin, fibronectin-binding protein A (fnbA), coagulase, map, Panton-Valentine leukocidin (pvl), gamma-toxin (hlg), ica, immunodominant ABC transporter, RAP, autolysin, laminin receptors, SPOIIIE, SsaA, EbpS, Sasf, SasH, EFB (FIB), FnbB, Npase, EBP, bone sialo binding protein II; aureolysin precursor (AUR)/Sepp1, Cna, TSST-1, mecA, dPNAG, GehD, EbhA, EbhB, SSP-1, SSP-2 HBP, vitronectin binding protein, HarA, Enterotoxin A, Enterotoxin B, Enterotoxin C1, and novel autolysin.

In some embodiments SA antigens can be used in the SA-MAPS composition as disclosed herein can be selected from any one or, or a combination of Opp3a, DltD, HtsA, LtaS, IsdA, IsdC, SdrF, SdrG, SdrH, SrtA, SpA, Shi alpha-hemolysin (hla), beta-hemolysin, fibronectin-binding protein A (fnbA), fibronectin-binding protein B (fnbB), coagulase, Fig, map, Panton-Valentine leukocidin (pvl), alpha-toxin and its variants, gamma-toxin (hlg) and variants, ica, immunodominant ABC transporter, Mg2+ transporter, Ni ABC transporter, RAP, autolysin, laminin receptors, IsaA/PisA, IsaB/PisB, SPOIIIE, SsaA, EbpS, SasA, SasF, SasH, EFB (FIB), SBI, Npase, EBP, bone sialo binding protein II, aureolysin precursor (AUR)/Sepp1, Cna, and fragments thereof such as M55, TSST-1, mecA, poly-N-acetylglucosamine (PNAG/dPNAG) exopolysaccharide, GehD, EbhA, EbhB, SSP-1, SSP-2, HBP, vitronectin binding protein, HarA, EsxA, EsxB, Enterotoxin A, Enterotoxin B. Enterotoxin C1, and novel autolysin.

Bacterial antigens include, but are not limited to (i) a secreted virulence factor, and/or a cell surface protein or peptide, or (ii) a recombinant nucleic acid molecule encoding a secreted virulence factor, and/or a cell surface protein or peptide. The bacterial antigen can include one or more of at least or at most 1, 2, 3, 4, 5, 6, 7, 8, 9, 10, 11, 12, 13, 14, 15, 16, 17, 18, or 19 additional staphylococcal antigen or immunogenic fragment thereof, including, but not limited to FnBpA, FnBpB, LukD (GI:2765304), LukE (GI:2765303), LukF (GI: 12231006), SasA, SasD, SasG, SasI, SasK, SpA (and variants thereof), Eap, Ebh, Emp, EsaB, EsaC, EsxA, EsxB, SdrC, SdrE, Coa, Hla (e.g., H35 mutants), IsdC, SasF, vWbp, vWh, 52 kDa vitronectin binding protein (WO 01/60852), Aaa (GenBank CAC80837), Aap (GenBank accession AJ249487), Ant (GenBank accession NP-372518), autolysin glucosaminidase, autolysin amidase, Cna, collagen binding protein (U.S. Pat. No. 6,288,214), EFB (FIB), Elastin binding protein (EbpS), EPB, FbpA, fibrinogen binding protein (U.S. Pat. No. 6,008,341), Fibronectin binding protein (U.S. Pat. No. 5,840,846), FnbA, FnbB, GehD (US 2002/0169288), HarA, HBP, Immunodominant ABC transporter, IsaA/P isA, laminin receptor, Lipase GehD, MAP, Mg2+ transporter, MHC II analogue (U.S. Pat. No. 5,648,240), MRPII, Npase, RNA III activating protein (RAP), SasA, SasB, SasC, SasD, SasK, SBI, SdrF (WO 00/12689), SdrG/Fig (WO 00/12689), SdrH (WO 00/12689), SEA exotoxins (WO 00/02523), SEB exotoxins (WO 00/02523), SitC and Ni ABC transporter, SitC/MntC/saliva binding protein (U.S. Pat. No. 5,801,234), SsaA, SSP-1, SSP-2, and/or Vitronectin binding protein (see PCT publications WO2007/113222, WO2007/113223, WO2006/032472, WO2006/032475, WO2006/032500, each of which is incorporated herein by reference in their entirety).

In some embodiments, a SA-antigen for use in the SA-MAPS composition as disclosed herein is a Microbial Surface Components Recognizing Adhesive Matrix Molecule, or MSCRAMMs, which include, but are not limited to: EkeS, DsqA, KesK, KrkN, KrkN2, RkaS, RrkN, and KnkA. These MSCRAMMS are described in WO 02/102829, which is hereby incorporated by reference. Additional MSCRAMMS, identified by GenBank Accession No., include NP_373261.1, NP_373371.1, NP_374246.1, NP_374248.1, NP_374841.1, NP_374866.1, NP_375140.1, NP_375614.1, NP_375615.1, NP_375707.1, NP_375765.1, and NP_375773.1.

In certain aspects, a SA-MAPs composition can comprise a staphylococcal antigen selected from the group consisting of: FnBpA, FnBpB, LukD, LukE, LukF, SasA, SasD, SasG, SasI, SasK, SpA (and variants thereof), Eap, Ebh, Emp, EsaB, EsaC, EsxA, EsxB, SdrC, SdrD, SdrE, IsdA, IsdB, ClfA, ClfB, Coa, Hla (e.g., H35 mutants), IsdC, SasF, vWbp, vWh and immunogenic fragments thereof.

Some exemplary alternative SA antigens for use in the SA-MAPS immunogenic composition as disclosed herein are discussed below.

(i) S. aureus MntC SitC Saliva Binding Protein

In one embodiment, a SA-MAPS composition may include a polypeptide, peptide, or protein that is or is at least 70%, 75%, 80%, 85%, 90%, 95%, 96%, 97%, 98%, or 99% identical or similar to a MntC/SitC/Saliva Binding Protein. MntC/SitC/Saliva Binding Protein is an ABC transporter protein and has homologues in S. epidermidis and S. aureus. It is referred to herein as MntC. This protein is a 32 kDa lipoprotein and is located in the bacterial cell wall. See Sellman et al., and Cockayne et al., Infect. Immun. 66: 3767 (1998). In S. epidermidis, it is a component of an iron-regulated operon. It shows considerable homology to both adhesins including FimA of S. parasanguis, and with lipoproteins of a family of ABC transporters with proven or putative metal iron transport functions. The S. aureus homologue of MntC is known as saliva binding protein and was disclosed in U.S. Pat. No. 5,801,234, which is incorporated herein in its entirety by reference. The protein sequence for the S. aureus homologue of MntC/SitC/Saliva Binding Protein is found in GenBank accession number NP_371155 for strain Mu50, (also known as SAV0631), where the accession number for the nucleotide sequence for the complete genome of strain Mu50 is NC_002758.2 (coordinates 704988-705917).

In alternative embodiments, a MntC antigen for use in the SA-MAPS immunogenic composition as disclosed herein is a protein or peptide having an amino acid sequence of one the MntC proteins disclosed in Table 12 of U.S. Pat. No. 8,568,735, which is incorporated herein in its entirety by reference. Other MntC antigens not identified herein are disclosed encompassed for use in the SA-MAPS immunogenic composition, provided they are antigenic.

(ii) S. epidermidis SitC Protein

In one embodiment, a SA-MAPS composition may include a polypeptide, peptide, or protein that is or is at least 70%, 75%, 80%, 85%, 90%, 95%, 96%, 97%, 98%, or 99% identical or similar to a SitC protein. SitC is the S. epidermidis homologue of MntC/SitC/Saliva Binding Protein and was disclosed in Sellman et al. (Sellman et al., Infect. Immun. 2005 October; 73(10): 6591-6600). The protein sequence for SitC is found in GenBank accession number YP_1187886.1 (also known as SERP0290) and is disclosed as SEQ ID NO: 121 in U.S. Pat. No. 8,568,735, which is incorporated herein in its entirety by reference. The accession number for the nucleotide sequence for the complete genome of strain RP62A, is NC_002976 (coordinates 293030-293959). Other candidate SitC molecules may be derived from various species of organisms for use in an immunogenic composition of the invention, some of which include, but are not limited to: all or part of the amino acid sequence of accession number BAE03450.1 (S. haemolyticus, JCSC1435 strain), AA004002.1 (S. epidermidis, strain ATCC 12228); BAE19233.1 (S. saprophyticus, strain ATCC 15305); ABR57162.1 (S. xylosus, strain DSM20267); CAL27186.1 (S. carnosus, strain TM300).

The term “FnBpA protein” refers to a protein that includes isolated wild-type FnBpA polypeptides from staphylococcus bacteria and segments thereof, as well as variants that stimulate an immune response against staphylococcus bacteria FnBpA proteins.

The term “FnBpB protein” refers to a protein that includes isolated wild-type FnBpB polypeptides from staphylococcus bacteria and segments thereof, as well as variants that stimulate an immune response against staphylococcus bacteria FnBpB proteins.

The term “SasA protein” refers to a protein that includes isolated wild-type SasA polypeptides from staphylococcus bacteria and segments thereof, as well as variants that stimulate an immune response against staphylococcus bacteria SasA proteins.

The term “SasD protein” refers to a protein that includes isolated wild-type SasD polypeptides from staphylococcus bacteria and segments thereof, as well as variants that stimulate an immune response against staphylococcus bacteria SasD proteins.

The term “SasG protein” refers to a protein that includes isolated wild-type SasG polypeptides from staphylococcus bacteria and segments thereof, as well as variants that stimulate an immune response against staphylococcus bacteria SasG proteins.

The term “SasI protein” refers to a protein that includes isolated wild-type SasI polypeptides from staphylococcus bacteria and segments thereof, as well as variants that stimulate an immune response against staphylococcus bacteria SasI proteins.

The term “SasK protein” refers to a protein that includes isolated wild-type SasK polypeptides from staphylococcus bacteria and segments thereof, as well as variants that stimulate an immune response against staphylococcus bacteria SasK proteins.

The term “EsxA protein” refers to a protein that includes isolated wild-type EsxA polypeptides from staphylococcus bacteria and segments thereof, as well as variants that stimulate an immune response against staphylococcus bacteria EsxA proteins.

The term “EsxB protein” refers to a protein that includes isolated wild-type EsxB polypeptides from staphylococcus bacteria and segments thereof, as well as variants that stimulate an immune response against staphylococcus bacteria EsxB proteins.

The term “Eap protein” refers to a protein that includes isolated wild-type Eap polypeptides from staphylococcus bacteria and segments thereof, as well as variants that stimulate an immune response against staphylococcus bacteria Eap proteins.

The term “Ebh protein” refers to a protein that includes isolated wild-type Ebh polypeptides from staphylococcus bacteria and segments thereof, as well as variants that stimulate an immune response against staphylococcus bacteria Ebh proteins.

The term “Emp protein” refers to a protein that includes isolated wild-type Emp polypeptides from staphylococcus bacteria and segments thereof, as well as variants that stimulate an immune response against staphylococcus bacteria Emp proteins.

The term “EsaB protein” refers to a protein that includes isolated wild-type EsaB polypeptides from staphylococcus bacteria and segments thereof, as well as variants that stimulate an immune response against staphylococcus bacteria EsaB proteins.

The term “EsaC protein” refers to a protein that includes isolated wild-type EsaC polypeptides from staphylococcus bacteria and segments thereof, as well as variants that stimulate an immune response against staphylococcus bacteria EsaC proteins.

The term “Coa protein” refers to a protein that includes isolated wild-type Coa polypeptides from staphylococcus bacteria and segments thereof, as well as variants that stimulate an immune response against staphylococcus bacteria Coa proteins.

The term “SasF protein” refers to a protein that includes isolated wild-type SasF polypeptides from staphylococcus bacteria and segments thereof, as well as variants that stimulate an immune response against staphylococcus bacteria SasF proteins.

The term “vWbp protein” refers to a protein that includes isolated wild-type vWbp (von Willebrand factor binding protein) polypeptides from staphylococcus bacteria and segments thereof, as well as variants that stimulate an immune response against staphylococcus bacteria vWbp proteins.

The term “vWh protein” refers to a protein that includes isolated wild-type vWh (von Willebrand factor binding protein homolog) polypeptides from staphylococcus bacteria and segments thereof, as well as variants that stimulate an immune response against staphylococcus bacteria vWh proteins.

In certain embodiments, the claimed invention specifically excludes 1, 2, 3, 4, 5, 6, 7, 8, 9, 10 or more of FnBpA, FnBpB, LukD (GI:2765304), LukE (GI:2765303), LukF (GI: 12231006), SasA, SasD, SasG, SasI, SasK, SpA (and variants thereof), Eap, Ebh, Emp, EsaB, EsaC, EsxA, EsxB, SdrC, SdrD, SdrE, IsdA, IsdB, ClfA, ClfB, Coa, Hla (e.g., H35 mutants), IsdC, SasF, vWbp, vWh, 52 kDa vitronectin binding protein (WO 01/60852), Aaa (GenBank CAC80837), Aap (GenBank accession AJ249487), Ant (GenBank accession NP-372518), autolysin glucosaminidase, autolysin amidase, Cna, collagen binding protein (U.S. Pat. No. 6,288,214), EFB (FIB), Elastin binding protein (EbpS), EPB, FbpA, fibrinogen binding protein (U.S. Pat. No. 6,008,341), Fibronectin binding protein (U.S. Pat. No. 5,840,846), FnbA, FnbB, GehD (US 2002/0169288), HarA, HBP, Immunodominant ABC transporter, IsaA/P isA, laminin receptor, Lipase GehD, MAP, Mg2+ transporter, MIHC II analogue (U.S. Pat. No. 5,648,240), MRPII, Npase, RNA III activating protein (RAP), SasA, SasB, SasC, SasD, SasK, SBI, SdrF (WO 00/12689), SdrG/Fig (WO 00/12689), SdrH (WO 00/12689), SEA exotoxins (WO 00/02523), SEB exotoxins (WO 00/02523), SitC and Ni ABC transporter, SitC/MntC/saliva binding protein (U.S. Pat. No. 5,801,234), SsaA, SSP-1, SSP-2, and/or Vitronectin binding protein (see PCT publications WO2007/113222, WO2007/113223, WO2006/032472, WO2006/032475, WO2006/032500, each of which is incorporated herein by reference in their entirety). In certain aspects, the bacterial antigen is a staphylococcal antigen. The staphylococcal antigen can be selected from the group consisting of FnBpA, FnBpB, LukD, LukE, LukF, SasA, SasD, SasG, SasI, SasK, SpA (and variants thereof), Eap, Ebh, Emp, EsaB, EsaC, EsxA, EsxB, SdrC, SdrD, SdrE, IsdA, IsdB, ClfA, ClfB, Coa, Hla (e.g., H35 mutants), IsdC, SasF, vWbp, vWh and immunogenic fragments thereof. Certain embodiments are directed to an immunogenic composition comprising an isolated Protein A (SpA) specific antibody and a bacterial antigen, wherein the Protein A specific antibody enhances an immune response to the bacterial antigen. In certain aspects, the antibody is a polyclonal antibody, a monoclonal antibody, or an antibody fragment. In still further aspects, the bacterial antigen is comprised in or on a bacterium. The bacteria can be attenuated bacteria, in particular attenuated staphylococcal bacteria.

In certain embodiments a subject is administered a SA-MAPS composition comprising a SA antigen, wherein the SA antigen is Hla209 or any SA antigen selected from any of FnBpA antigen or immunogenic fragment thereof, FnBpB antigen or immunogenic fragment thereof, LukD antigen or immunogenic fragment thereof, LukE antigen or immunogenic fragment thereof, LukF antigen or immunogenic fragment thereof, SasA antigen or immunogenic fragment thereof, SasD antigen or immunogenic fragment thereof, SasG antigen or immunogenic fragment thereof, SasI antigen or immunogenic fragment thereof, SasK antigen or immunogenic fragment thereof, SpA (and variants thereof) antigen or immunogenic fragment thereof, Eap antigen or immunogenic fragment thereof, Ebh antigen or immunogenic fragment thereof, Emp antigen or immunogenic fragment thereof, EsaB antigen or immunogenic fragment thereof, EsaC antigen or immunogenic fragment thereof, EsxA antigen or immunogenic fragment thereof, EsxB antigen or immunogenic fragment thereof, SdrC antigen or immunogenic fragment thereof, SdrD antigen or immunogenic fragment thereof, SdrE antigen or immunogenic fragment thereof, IsdA antigen or immunogenic fragment thereof, IsdB antigen or immunogenic fragment thereof, ClfA antigen or immunogenic fragment thereof, ClfB antigen or immunogenic fragment thereof, Coa antigen or immunogenic fragment thereof, Hla (e.g., H35 mutants) antigen or immunogenic fragment thereof, IsdC antigen or immunogenic fragment thereof, SasF antigen or immunogenic fragment thereof, vWbp antigen or immunogenic fragment thereof, vWh antigen or immunogenic fragment thereof.

### B. Combinations of SA Antigens Present on the SA-MAPS Immunogenic Composition

In some embodiments, a SA-MAPS complex comprises at least 2 SA antigens, e.g., any one SA-antigen selected from any of: SA1720 (B1), SA1739 (B2), SA1890 (B3), SA0103 (T1), SA0377 (T2), SA0693 (T3) and SA2105 (T4), and any one SA antigen selected from any of: Hla, such as but not limited to Hla(209) as disclosed herein, and one or more SA antigens selected from a Clumping factor A (ClfA), Clumping factor B (ClfB), serine-aspirate repeat protein D (SdrD), serine-aspirate repeat protein E (SdrE), Iron regulator surface protein A (IsdA), Iron regulator surface protein B (IsdB), Leukotoxin D (LukD), or Leukotoxin E (LukE), or fragments thereof.

In some embodiments, a SA-MAPS complex comprises at least one SA antigens, e.g., any one SA-antigen selected from any of: SA1720 (B1), SA1739 (B2), SA1890 (B3), SA0103 (T1), SA0377 (T2), SA0693 (T3) and SA2105 (T4), and at least one antigen selected from hemolysin (Hl) (e.g., hemolysin α or Hla209), Clumping factor A (ClfA), Clumping factor B (ClfB), serine-aspirate repeat protein D (SdrD), Iron regulator surface protein A (IsdA) and Iron regulator surface protein B (IsdB), or fragments thereof, for example, but not limited to: Hla209(27-319), ClfA(221-559), ClfB (203-542), SdrD (246-682), IsdA (47-324), IsdB (48-447) or proteins or peptides having at least 85% sequence identity thereto. It is envisioned that any of the above listed SA antigens can be substituted for a different SA peptide or polypeptide antigen known to one of ordinary skill in the art.

In some embodiments, a SA-MAPS immunogenic composition as disclosed herein can comprise at least one SA-antigen selected from any of: SA1720 (B1), SA1739 (B2), SA1890 (B3), SA0103 (T1), SA0377 (T2), SA0693 (T3) and SA2105 (T4), and all 4 SA antigens selected from: hemolysin (Hl) (e.g., hemolysin α or Hla209), Clumping factor A (ClfA), Clumping factor B (ClfB), serine-aspirate repeat protein D (SdrD), or fragments thereof, for example, but not limited to: Hla209(27-319), ClfA(221-559), ClfB (203-542), SdrD (246-682), or proteins or peptides having at least 85% sequence identity thereto. It is envisioned that any of the above listed SA antigens can be substituted for a different SA peptide or polypeptide antigen known to one of ordinary skill in the art.

In alternative embodiments, the SA-MAPS immunogenic compositions as disclosed herein can comprise any SA antigen that elicits an immune response in a subject. In some embodiments, the SA-MAPS composition comprises at least one, or at least 2 SA antigens. In some embodiments, the SA-MAPS immunogenic composition comprises at least 2, or at least 3, or at least 4, or between 2-4, or between 3-5, or between 6-8, or between 8-10 or between 10-12, or between 10-15, or between 15-20 or more than 20 SA protein or polypeptide antigens. In some embodiments, the antigens can be the same, e.g., all SA1739 antigens, or a combination of different antigens, e.g., SA1739 (B2), SA1720 (B1), SA1890 (B3), SA0103 (T1), SA0377 (T2), and SA0693 (T3) etc.

In some embodiments, the SA-MAPS composition comprises at least SA1739 (B2) antigen and at least 1 more, or at least 2 more, or at least 3 more or at least 4 more, or at least 5 more SA antigens as disclosed herein. In some embodiments, the SA-MAPS composition comprises at least SA1720 (B1) antigen and at least 1 more, or at least 2 more, or at least 3 more or at least 4 more, or at least 5 more SA antigens as disclosed herein. In some embodiments, the SA-MAPS composition comprises at least SA 1890 (B3) antigen and at least 1 more, or at least 2 more, or at least 3 more or at least 4 more, or at least 5 more SA antigens as disclosed herein. In some embodiments, the SA-MAPS composition comprises at least SA0103 (T1) antigen and at least 1 more, or at least 2 more, or at least 3 more or at least 4 more, or at least 5 more SA antigens as disclosed herein. In some embodiments, the SA-MAPS composition comprises at least SA0377 (T2) antigen and at least 1 more, or at least 2 more, or at least 3 more or at least 4 more, or at least 5 more SA antigens as disclosed herein. In some embodiments, the SA-MAPS composition comprises at least SA0693 (T3) antigen and at least 1 more, or at least 2 more, or at least 3 more or at least 4 more, or at least 5 more SA antigens as disclosed herein.

Exemplary combinations of different SA antigen present on a SA-MAPS immunogenic composition as disclosed herein are shown in Tables 4A-4E.

TABLE 4A
SA-MAPS with at least 1 SA-antigens from Group
A and at least 1 SA antigen from Group B:

| Group A | At least one SA antigen selected |
|---|---|
| antigen | from the Group B antigens: |
| SA1739 (B2) | Hla, ClfA, ClfB, SdrD, IsdA or IsdB. |
| SA1720 (B1) | Hla, ClfA, ClfB, SdrD, IsdA or IsdB. |
| SA1890 (B3) | Hla, ClfA, ClfB, SdrD, IsdA or IsdB. |
| SA0103 (T1) | Hla, ClfA, ClfB, SdrD, IsdA or IsdB. |
| SA0377 (T2) | Hla, ClfA, ClfB, SdrD, IsdA or IsdB. |
| SA0693 (T3) | Hla, ClfA, ClfB, SdrD, IsdA or IsdB. |
| SA2105 (T4) | Hla, ClfA, ClfB, SdrD, IsdA or IsdB. |

TABLE 4B
SA-MAPS with at least two SA-antigens from Group
A and at least 1 SA antigen from Group B:

| Group A | | At least one SA antigen selected |
|---|---|---|
| antigen | At least one antigen selected from: | from the Group B antigens: |
| SA1739 (B2) | SA1720 (B1), SA1890 (B3), SA0103 (T1), | Hla, ClfA, ClfB, SdrD, IsdA or IsdB. |
| | SA0377 (T2), SA0693 (T3) and SA2105 | |
| | (T4). | |
| SA1720 (B1) | SA1739 (B2), SA1890 (B3), SA0103 (T1), | Hla, ClfA, ClfB, SdrD, IsdA or IsdB. |
| | SA0377 (T2), SA0693 (T3) and SA2105 | |
| | (T4). | |
| SA1890 (B3) | SA1739 (B2), SA1720 (B1), SA0103 (T1), | Hla, ClfA, ClfB, SdrD, IsdA or IsdB. |
| | SA0377 (T2), SA0693 (T3) and SA2105 | |
| | (T4). | |
| SA0103 (T1) | SA1739 (B2), SA1720 (B1), SA1890 (B3), | Hla, ClfA, ClfB, SdrD, IsdA or IsdB. |
| | SA0377 (T2), SA0693 (T3) and SA2105 | |
| | (T4). | |
| SA0377 (T2) | SA1739 (B2), SA1720 (B1), SA1890 (B3), | Hla, ClfA, ClfB, SdrD, IsdA or IsdB. |
| | SA0103 (T1), SA0693 (T3) and SA2105 | |
| | (T4). | |
| SA0693 (T3) | SA1739 (B2), SA1720 (B1), SA1890 (B3), | Hla, ClfA, ClfB, SdrD, IsdA or IsdB. |
| | SA0103 (T1), SA0377 (T2), and SA2105 | |
| | (T4). | |
| SA2105 (T4) | SA1739 (B2), SA1720 (B1), SA1890 (B3), | Hla, ClfA, ClfB, SdrD, IsdA or IsdB. |
| | SA0103 (T1), SA0377 (T2), and SA0693 | |
| | (T3) | |

TABLE 4C
SA-MAPS with at least 1 SA-antigens from Group
A and at four SA antigens from Group B(iii):

| Group A antigen | And four SA antigens from Group B(iii) antigens: |
|---|---|
| SA1739 (B2) | Hla209(27-319), ClfA(221-559), ClfB (203-542), SdrD (246-682) |
| SA1720 (B1) | Hla209(27-319), ClfA(221-559), ClfB (203-542), SdrD (246-682) |
| SA1890 (B3) | Hla209(27-319), ClfA(221-559), ClfB (203-542), SdrD (246-682) |
| SA0103 (T1) | Hla209(27-319), ClfA(221-559), ClfB (203-542), SdrD (246-682) |
| SA0377 (T2) | Hla209(27-319), ClfA(221-559), ClfB (203-542), SdrD (246-682) |
| SA0693 (T3) | Hla209(27-319), ClfA(221-559), ClfB (203-542), SdrD (246-682) |
| SA2105 (T4) | Hla209(27-319), ClfA(221-559), ClfB (203-542), SdrD (246-682) |

TABLE 4C
SA-MAPS with at least two SA-antigens from Group
A and at least four SA antigen from B(iii):

| Group A | | And And four SA antigens |
|---|---|---|
| antigen | And at least one antigen selected from: | from Group B(iii) antigens: |
| SA1739 (B2) | SA1720 (B1), SA1890 (B3), SA0103 | Hla209(27-319), ClfA(221-559), |
| | (T1), SA0377 (T2), SA0693 (T3) and | ClfB (203-542), SdrD (246-682) |
| | SA2105 (T4). | |
| SA1720 (B1) | SA1739 (B2), SA1890 (B3), SA0103 | Hla209(27-319), ClfA(221-559), |
| | (T1), SA0377 (T2), SA0693 (T3) and | ClfB (203-542), SdrD (246-682) |
| | SA2105 (T4). | |
| SA1890 (B3) | SA1739 (B2), SA1720 (B1), SA0103 | Hla209(27-319), ClfA(221-559), |
| | (T1), SA0377 (T2), SA0693 (T3) and | ClfB (203-542), SdrD (246-682) |
| | SA2105 (T4). | |
| SA0103 (T1) | SA1739 (B2), SA1720 (B1), SA1890 | Hla209(27-319), ClfA(221-559), |
| | (B3), SA0377 (T2), SA0693 (T3) and | ClfB (203-542), SdrD (246-682) |
| | SA2105 (T4). | |
| SA0377 (T2) | SA1739 (B2), SA1720 (B1), SA1890 | Hla209(27-319), ClfA(221-559), |
| | (B3), SA0103 (T1), SA0693 (T3) and | ClfB (203-542), SdrD (246-682) |
| | SA2105 (T4). | |
| SA0693 (T3) | SA1739 (B2), SA1720 (B1), SA1890 | Hla209(27-319), ClfA(221-559), |
| | (B3), SA0103 (T1), SA0377 (T2), and | ClfB (203-542), SdrD (246-682) |
| | SA2105 (T4). | |
| SA2105 (T4) | SA1739 (B2), SA1720 (B1), SA1890 | Hla209(27-319), ClfA(221-559), |
| | (B3), SA0103 (T1), SA0377 (T2), and | ClfB (203-542), SdrD (246-682) |
| | SA0693 (T3) | |

In particular, Tables 4A-4E show exemplary SA antigens present on SA-MAPS complexes which are useful in the compositions and methods as disclosed herein. Tables 4A-4C have used an exemplary set of SA antigens, with Table 4A showing at least a first SA antigen selected from SA1739 (B2), SA1720 (B1), SA1890 (B3), SA0103 (T1), SA0377 (T2), SA0693 (T3) and SA2105 (T4), and Table 2 showing at least a first and at least a second SA antigen selected from: SA1739 (B2), SA1720 (B1), SA1890 (B3), SA0103 (T1), SA0377 (T2), SA0693 (T3) and SA2105 (T4), and it is envisioned that any of the SA antigens can be substituted for a different SA peptide or polypeptide antigen known to one of ordinary skill in the art. In some embodiments, a SA-MAPS immunogenic composition comprises a combination of 2, 3, 4, 5 or 6 of the exemplary SA antigens selected from SA1739 (B2), SA1720 (B1), SA1890 (B3), SA0103 (T1), SA0377 (T2), SA0693 (T3) and SA2105 (T4). or proteins or peptides having at least 85% sequence identity thereto. Table 4D show exemplary combinations of 2, 3, 4, 5, 6, 7, 8 and 9 antigens present in the SA-MAPS complex.

In some embodiments, the SA-MAPS composition can comprise a variety of different combinations of SA-antigens as disclosed herein. These can be in the following combinations, for exemplary purposes only, the SA-MAPS composition can be a SA-MAPS which comprise a SA-antigen from Group 1, and at least one SA-antigen from the subgroup of SA antigens subgrouped in Groups 3, 4, 5, 6, 7 or 8, which are shown in Table 4E. For exemplary purposes, a SA-MAPS can comprise one or more SA-antigens in Group 1 and a combination of SA-antigens in Group 2.

In some embodiments, the SA-MAPS composition as disclosed herein can comprise a combinations of SA-antigens from Group 1, and a combination of SA-antigen from the subgroup of SA antigens subgrouped in Groups 3, 4, 5, 6, 7 or 8. In some embodiments, the SA-MAPS composition as disclosed herein can comprise a combinations of SA-antigens from Group 2, and a combination of SA-antigen from the subgroup of SA antigens subgrouped in Groups 3, 4, 5, 6, 7 or 8. In some embodiments, the SA-MAPS composition as disclosed herein can comprise a combinations of SA-antigens from Group 3, and a combination of SA-antigen from the subgroup of SA antigens subgrouped in Groups 3, 4, 5, 6, 7 or 8. In some embodiments, the SA-MAPS composition as disclosed herein can comprise a combinations of SA-antigens from Group 9, and a combination of SA-antigen from the subgroup of SA antigens subgrouped in Groups 3, 4, 5, 6, 7 or 8.

The SA antigens in subgroups 3-8 are shown in Table 4E.

For exemplary purposes, a SA-MAPS can comprise one or more SA-antigens in Group 1 and a combination of SA-antigens in Group 2.

The following combinations of SA antigens are envisioned in a SA-MAPs composition as disclosed in Table 4D.

TABLE 4D

| | |
|---|---|
| Specific | |
| Combination | |
| of Groups | Description of Group combinations |

Group 1 combinations: Group 1 (SA-antigen from Group A) plus a second Group (selected
from Group 2-8, which are different combinations of SA-antigens from Group B)

| | |
|---|---|
| Group 1 only | Group 1 only (1 SA antigens from Group A) |
| Group 1 and 3 | Group 1 (1 SA antigens from Group A only) + Group 3 (at least 1 SA |
| | antigen from Group B (2 valent; 1A + 1B)) |
| Group 1 and 4 | Group 1 (1 SA antigens from Group A only) + Group 4 (at least 2 SA |
| | antigen from Group B (3 valent; 1A + 2B)) |
| Group 1 and 5 | Group 1 (1 SA antigens from Group A only) + Group 5 (at least 3 SA |
| | antigen from Group B (4 valent; 1A + 3B) |
| Group 1 and 6 | Group 1 (1 SA antigens from Group A only) + a Group 6 (at least 4 SA |
| | antigen from Group B (5 valent; 1A + 4B)) |
| Group 1 and 7 | Group 1 (1 SA antigens from Group A only) + Group 7 (at least 5 SA |
| | antigen from Group B (6 valent; 1A + 5B)) |
| Group 1 and 8 | Group 1 (1 SA antigens from Group A only) + Group 8 (all four SA |
| | antigens Hla209(27-319), ClfA(221-559), ClfB (203-542), SdrD (246-682)) |
| | (5 valent) |

Group 2 combinations: Group 2 (2 SA-antigens from Group A) plus another Group
(selected from Group 3-8, which are different combinations of SA-antigens from Group B)

| | |
|---|---|
| Group 2 only | Group 2 only (2 SA antigens from Group A only) |
| Group 2 and Group 3 | Group 2 (2 SA antigens from Group A only) + Group 3 (at least 1 SA |
| | antigen from Group B (3 valent; 2A + 1B)) |
| Group 2 and Group 4 | Group 2 (2 SA antigens from Group A only) + Group 4 (at least 2 SA |
| | antigen from Group B (4 valent; 2A + 2B)) |
| Group 2 and Group 5 | Group 2 (2 SA antigens from Group A only) + Group 5 (at least 3 SA |
| | antigen from Group B (5 valent; 2A + 3B)) |
| Group 2 and Group 6 | Group 2 (2 SA antigens from Group A only) + Group 6 (at least 4 SA |
| | antigen from Group B (6 valent; 2A + 4B)) |
| Group 2 and Group 7 | Group 2 (2 SA antigens from Group A only) + Group 7 (at least 5 SA |
| | antigen from Group B (7 valent; 2A + 5B)) |
| Group 2 and Group 8 | Group 2 (2 SA antigens from Group A only) + Group 8 (four all SA |
| | antigens Hla209(27-319), ClfA(221-559), ClfB (203-542), SdrD (246-682)) |
| | (6 valent) |

Group 9 combinations: Group 9 (three SA-antigens from Group A) plus another Group
(selected from Group 3-8, which are different combinations of SA-antigens from Group B)

| | |
|---|---|
| Group 9 only | Group 9 only (3 SA antigens from Group A only) |
| Group 9 and Group 3 | Group 9 (3 SA antigens from Group A only) + Group 3 (at least 1 SA |
| | antigen from Group B (4 valent; 3A + 1B)) |
| Group 9 and Group 4 | Group 9 (3 SA antigens from Group A only) + Group 4 (at least 2 SA |
| | antigen from Group B (5 valent; 3A + 2B)) |
| Group 9 and Group 5 | Group 9 (3 SA antigens from Group A only) + Group 5 (at least 3 SA |
| | antigen from Group B (6 valent; 3A + 3B)) |
| Group 9 and Group 6 | Group 9 (3 SA antigens from Group A only) + Group 6 (at least 4 SA |
| | antigen from Group B (7 valent; 3A + 4B)) |
| Group 9 and Group 7 | Group 9 (3 SA antigens from Group A only) + Group 7 (at least 5 SA |
| | antigen from Group B (8 valent; 3A + 5B)) |
| Group 9 and Group 8 | Group 9 (3 SA antigens from Group A only) + Group 8 (four all SA |
| | antigens Hla209(27-319), ClfA(221-559), ClfB (203-542), SdrD (246-682) |
| | (7 valent) |

Group 10 combinations: Group 10 (four SA-antigens from Group A) plus another Group
(selected from Group 3-8, which are different combinations of SA-antigens from Group B)

| | |
|---|---|
| Group -10 only | Group 10 only (four SA antigens from Group A only) |
| Group 10 and Group | Group 10 (4 SA antigens from Group A only) + Group 3 (at least 1 SA |
| 3 | antigen from Group B (5 valent; 4A + 1B)) |
| Group 10 and Group | Group 10 (4 SA antigens from Group A only) + Group 4 (at least 2 SA |
| 4 | antigen from Group B (6 valent; 4A + 2B)) |
| Group 10 and Group | Group 10 (4 SA antigens from Group A only) + Group 5 (at least 3 SA |
| 5 | antigen from Group B (7 valent; 4A + 3B)) |
| Group 10 and Group | Group 10 (4 SA antigens from Group A only) + Group 6 (at least 4 SA |
| 6 | antigen from Group B (8 valent; 4A + 4B) )) |
| Group 10 and Group | Group 10 (4 SA antigens from Group A only) + Group 7 (at least 5 SA |
| 7 | antigen from Group B (9 valent; 4A + 5B)) |
| Group 10 and Group | Group 10 (4 SA antigens from Group A only) + Group 8 (four all SA |
| 8 | antigens Hla209(27-319), ClfA(221-559), ClfB (203-542), SdrD (246-682) |
| | (7 valent) |

It is envisioned that any of the above-identified antigens in Tables 4A4E can be switched out for a different SA antigen, including a different peptides or polypeptides of ClfA, ClfB, SdrD, SdrE, IsdA, IsdB, LukD, or LukE, or peptides or polypeptides at least 85% sequence identity thereto, or completely different SA antigens. In some embodiments, a SA antigen identified in tables 3A-3G can be substituted or switched out with a non-SA antigen, as disclosed herein.

Accordingly, in some embodiments, an ordinary skilled artisan can substitute any of the antigens listed in Tables 3A-3G with any other SA antigen not listed herein and known to an ordinary skilled artisan, or even substitute a SA antigen listed in Tables 3A-3G with a non-SA antigen.

In addition to one or more S. aureus antigens present in the MAPS complex, the MAPS complex may comprise non-S. aureus (non-SA) immunogenic antigens, including but not limited to pathogenic peptides, toxins, toxoids, subunits thereof, or combinations thereof (e.g., cholera toxin, tetanus toxoid).

In some embodiments, an antigen is derived (e.g., obtained) from a pathogenic organism. In some embodiments, the antigen is a cancer or tumor antigen, e.g., an antigen derived from a tumor or cancer cell.

In some embodiments, an antigen derived from a pathogenic organism is an antigen associated with an infectious disease; it can be derived from any of a variety of infectious agents, including virus, bacterium, fungus or parasite.

In some embodiments, a target antigen is any antigen associated with a pathology, for example an infectious disease or pathogen, or cancer or an immune disease such as an autoimmune disease. In some embodiments, an antigen can be expressed by any of a variety of infectious agents, including virus, bacterium, fungus or parasite. A target antigen for use in the methods and compositions as disclosed herein can also include, for example, pathogenic peptides, toxins, toxoids, subunits thereof, or combinations thereof (e.g., cholera toxin, tetanus toxoid).

Non-limiting examples of infectious viruses include: Retroviridae; Picornaviridae (for example, polio viruses, hepatitis A virus; enteroviruses, human coxsackie viruses, rhinoviruses, echoviruses); Calciviridae (such as strains that cause gastroenteritis); Togaviridae (for example, equine encephalitis viruses, rubella viruses); Flaviridae (for example, dengue viruses, encephalitis viruses, yellow fever viruses); Coronaviridae (for example, coronaviruses); Rhabdoviridae (for example, vesicular stomatitis viruses, rabies viruses); Filoviridae (for example, ebola viruses); Paramyxoviridae (for example, parainfluenza viruses, mumps virus, measles virus, respiratory syncytial virus); Orthomyxoviridae (for example, influenza viruses); Bungaviridae (for example, Hantaan viruses, bunga viruses, phleboviruses and Nairo viruses); Arena viridae (hemorrhagic fever viruses); Reoviridae (e.g., reoviruses, orbiviurses and rotaviruses); Birnaviridae; Hepadnaviridae (Hepatitis B virus); Parvoviridae (parvoviruses); Papovaviridae (papilloma viruses, polyoma viruses); Adenoviridae (most adenoviruses); Herpesviridae (herpes simplex virus (HSV) 1 and HSV-2, varicella zoster virus, cytomegalovirus (CMV), Marek's disease virus, herpes viruses); Poxviridae (variola viruses, vaccinia viruses, pox viruses); and Iridoviridae (such as African swine fever virus); and unclassified viruses (for example, the etiological agents of Spongiform encephalopathies, the agent of delta hepatitis (thought to be a defective satellite of hepatitis B virus), the agents of non-A, non-B hepatitis (class 1=internally transmitted; class 2=parenterally transmitted (i.e., Hepatitis C); Norwalk and related viruses, and astroviruses). The compositions and methods described herein are contemplated for use in treating infections with these viral agents.

Examples of fungal infections that may be addressed by inclusion of antigens in the present embodiments include aspergillosis; thrush (caused by Candida albicans); cryptococcosis (caused by Cryptococcus); and histoplasmosis. Thus, examples of infectious fungi include, but are not limited to, Cryptococcus neoformans, Histoplasma capsulatum, Coccidioides immitis, Blastomyces dermatitidis, Chlamydia trachomatis, Candida albicans. Components of these organisms can be included as antigens in the MAPS described herein.

In one aspect of the invention, an non-SA antigen to be used in combination with one or more SA antigens on the MAPS complex is derived from an infectious microbe such as Bordatella pertussis, Brucella, Enterococci sp., Neisseria meningitidis, Neisseria gonorrheae, Moraxella, typeable or nontypeable Haemophilus, Pseudomonas, Salmonella, Shigella, Enterobacter, Citrobacter, Klebsiella, E. coli, Helicobacter pylori, Clostridia, Bacteroides, Chlamydiaceae, Vibrio cholera, Mycoplasma, Treponemes, Borelia burgdorferi, Legionella pneumophilia, Mycobacteria sps (such as M. tuberculosis, M avium, M intracellulare, M kansaii, M gordonae, M leprae), Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus anthracis, Corynebacterium diphtheriae, Corynebacterium sp., Erysipelothrix rhusiopathiae, Clostridium perfringens, Clostridium tetani, Enterobacter aerogenes, Klebsiella pneumoniae, Leptospira sps., Pasteurella multocida, Bacteroides sp., Fusobacterium nucleatum, Streptobacillus monilformis, Treponema pallidium, Treponema pertenue, and Actinomyces israelli.

In some embodiments, a non-SA antigen useful in a SA-MAPS complex as disclosed herein is an antigen from an enteric bacterium, or non-enteric gram-negative bacteria. In some embodiments, a non-SA antigen useful in a SA-MAPS complex as disclosed herein can be selected from any of, or a combination of: a pneumococcal antigen, tuberculous antigen, HIV antigen, seasonal or epidemic influenza antigen, pertussis antigen, meningococcal antigen, haemophilus antigen, HPV antigen, E. coli antigens, salmonella antigens, enterobacter antigens, acinetobacter pathogen antigens, pseudomona antigens, klebsiella antigens, citrobacter antigens, serratia antigens, Clostridium difficile antigens from an enteric bacteria, antigens from non-enteric gram-negative bacteria, toxoids, toxins or toxin portions thereof.

In some embodiments, a non-SA antigen useful in a SA-MAPS complex as disclosed herein is a pneumococcal antigen, a tuberculosis antigen, an anthrax antigen, a HIV antigens, a seasonal or epidemic influenza antigen, a HPV antigen, an Acinetobacter antigens, a-Clostridium difficile antigen, an enteric Gram-negative bacterial antigen or nonenteric Gram-negative bacterial antigen, a Gram-positive bacterial antigens, a toxoid, toxin or toxin portion, a fungal antigen, a viral antigen, a cancer antigen or any combinations thereof.

In some embodiments, a non-SA antigen useful in a SA-MAPS complex as disclosed herein is an enteric Gram-negative bacterial antigen, selected from the group of: E. coli antigens, Salmonella antigens, Enterobacter antigens, Klebsiella antigens, Citrobacter antigens and Serratia antigens, or combinations thereof. In some embodiments, a non-SA antigen useful in a SA-MAPS complex as disclosed herein is a nonenteric Gram-negative bacterial antigens are selected from the group of: Pertussis antigens, Meningococcal antigens, Haemophilus antigens, and Pseudomonas antigens or combinations thereof.

Additional parasite pathogens from which antigens can be derived include, for example: Entamoeba histolytica, Plasmodium falciparum, Leishmania sp., Toxoplasma gondii, Rickettsia, and the Helminths.

In some embodiments, a non-SA antigen useful in a SA-MAPS complex as disclosed herein is a truncated pneumococcal PsaA protein, pneumolysin toxoid pneumococcal serine/threonine protein kinase (StkP), pneumococcal serine/threonine protein kinase repeating unit (StkPR), pneumococcal PcsB protein, staphylococcal alpha hemolysin, Mycobacterium tuberculosis mtb protein ESAT-6, M. tuberculosis cell wall core antigen, Chlamydia CT144, CT242 or CT812 polypeptides or fragments of these, Chlamydia DNA gyrase subunit B, Chlamydia sulfite synthesis/biphosphate phosphatase, Chlamydia cell division protein FtsY, Chlamydia methionyl-tRNA synthetase, Chlamydia DNA helicase (uvrD), Chlamydia ATP synthase subunit I (atpl), or Chlamydia metal dependent hydrolase.

In some embodiments, a non-SA antigen useful in a SA-MAPS complex as disclosed herein is an antigen from Mycobacterium tuberculosis (TB). One example of a TB antigen is TbH9 (also known as Mtb 39A). Other TB antigens include, but are not limited to, DPV (also known as Mtb8.4), 381, Mtb41, Mtb40, Mtb32A, Mtb64, Mtb83, Mtb9.9A, Mtb9.8, Mtb16, Mtb72f, Mtb59f, Mtb88f, Mtb71f, Mtb46f and Mtb31f, wherein “f” indicates that it is a fusion or two or more proteins.

In some embodiments, a non-SA antigen useful in a SA-MAPS complex as disclosed herein can be derived from a Chlamydia species for use in the immunogenic compositions of the present invention. Chlamydiaceae (consisting of Chlamydiae and Chlamydophila), are obligate intracellular gram-negative bacteria. Chlamydia trachomatis infections are among the most prevalent bacterial sexually transmitted infections, and perhaps 89 million new cases of genital chlamydial infection occur each year. The Chlamydia of the present invention include, for example, C. trachomatis, Chlamydophila pneumoniae, C. muridarum, C. suis, Chlamydophila abortus, Chlamydophila psittaci, Chlamydophila caviae, Chlamydophila feis, Chlamydophila pecorum, and C. pneumoniae. Animal models of chlamydial infection have established that T-cells play a critical role both in the clearance of the initial infection and in protection from re-infection of susceptible hosts. Hence, the immunogenic compositions as disclosed herein can be used to provide particular value by eliciting cellular immune responses against chlamydial infection.

More specifically, Chlamydial antigens useful as a non-SA antigen in a SA-MAPS complex as disclosed herein include DNA gyrase subunit B, sulfite synthesis/biphosphate phosphatase, cell division protein FtsY, methionyl-tRNA synthetase, DNA helicase (uvrD); ATP synthase subunit I (atpI) or a metal-dependent hydrolase (U.S. Patent Application Pub. No. 20090028891). Additional Chlamyidia trachomatis antigens include CT144 polypeptide, a peptide having amino acid residues 67-86 of CT144, a peptide having amino acid residues 77-96 of CT144, CT242 protein, a peptide having amino acids 109-117 of CT242, a peptide having amino acids 112-120 of CT242 polypeptide, CT812 protein (from the pmpD gene), a peptide having amino acid residues 103-111 of the CT812 protein; and several other antigenic peptides from C. trachomatis, which are disclosed in US Patent Application: 2014/0154287 and WO 2009/020553. Additionally, Chlamydia pneumoniae antigens including homologues of the foregoing polypeptides (see U.S. Pat. No. 6,919,187), can be used as antigens in the immunogenic compositions and methods as disclosed herein.

In some embodiments, an SA or non-SA antigen for use in the SA-MAPS composition can be an intact (i.e., an entire or whole) antigen, or a functional portion of an antigen that comprises more than one epitope. In some embodiments, an antigen is a peptide functional portion of an antigen. By “intact” in this context is meant that the antigen is the full length antigen as that antigen polypeptide occurs in nature. This is in direct contrast to delivery of only a small portion or peptide of the antigen. Delivering an intact antigen to a cell enables or facilitates eliciting an immune response to a full range of epitopes of the intact antigen, rather than just a single or selected few peptide epitopes. Accordingly, the methods and immunogenic compositions described herein encompass intact antigens associated with the polymer for a more sensitive and have higher specificity of immune response as compared to use of a single epitope peptide-based antigen.

Alternatively, in some embodiments, an intact SA antigen can be divided into many parts, depending on the size of the initial antigen. Typically, where a whole antigen is a multimer polypeptide, the whole protein can be divided into sub-units and/or domains where each individual sub-unit or domain of the antigen can be associated with the polymer according to the methods as disclosed herein. Alternatively, in some embodiments, an intact SA antigen can be divided into functional fragments, or parts, of the whole antigen, for example, at least two, or at least 3, or at least 4, or at least 5, or at least 6, or at least 7, or at least 8, or at least 9, or at least 10, or at least 11, or at least 12, or at least 13, or at least 15, or at least 20, or at least 25, or more than 25 portions (e.g., pieces or fragments), inclusive, and where each individual functional fragment of the antigen can be associated with the polymer according to the methods as disclosed herein.

The fragmentation or division of a full length SA antigen polypeptide can be an equal division of the full length antigen polypeptide, or alternatively, in some embodiments, the fragmentation is asymmetrical or unequal. As a non-limiting example, where an antigen is divided into two overlapping fragments, an antigen can be divided into fragments of approximately the same (equal) size, or alternatively one fragment can be about 45% of the whole antigen and the other fragment can be about 65%. As further non-limiting examples, a whole antigen can be divided into a combination of differently sized fragments, for example, where an antigen is divided into two fragments, fragments can be divided into about 40% and about 70%, or about 45% and about 65%; or about 35% and about 75%; or about 25% and about 85%, inclusive, of the whole antigen. Any combination of overlapping fragments of a full length whole antigen is encompassed for use in the generation of a panel of overlapping polypeptides of an antigen. As an illustrative example only, where an antigen is divided into 5 portions, the portions can divided equally (i.e., each overlapping fragment is about 21% to 25% of the entire full length if the antigen) or unequally (i.e., an antigen can be divided into the following five overlapping fragments; fragment 1 is about 25%, fragment 2 is about 5%, fragment 3 is about 35%, fragment 4 is about 10% and fragment 5 is about 25% of the size of the full length antigen, provided each fragment overlaps with at least one other fragment).

Typically, a panel of antigen portions can substantially cover the entire length of the whole (or intact) antigen polypeptide. Accordingly, in some embodiments, an immunogenic composition comprises a polymer with many different, and/or overlapping fragments of the same intact antigen. Overlapping protein fragments of an antigen can be produced much quicker and cheaper, and with increased stability as compared to the use of peptide antigens alone. Further in some embodiments, antigens which are polypeptides larger than simple peptides are preferred as conformation is important for epitope recognition, and the larger antigen polypeptides or fragments will provide a benefit over peptide fragments.

One of ordinary skill in the art can divide a whole antigen into overlapping proteins of an antigen to create a panel of polypeptides of the antigen. By way of an illustrative example only, a SA antigen ClfA can be divided into, for example at least 10 portions to generate a panel of 10 different polypeptides, each comprising a different but overlapping ClfA-specific antigens fragments.

A target antigen for use in the methods and compositions described herein can be expressed by recombinant means, and can optionally include an affinity or epitope tag to facilitate purification, which methods are well-known in the art. Chemical synthesis of an oligopeptide, either free or conjugated to carrier proteins, can be used to obtain antigen of the invention. Oligopeptides are considered a type of polypeptide. A SA-polypeptide antigen can be expressed as a fusion with a complementary affinity molecule, e.g., a biotin-binding moiety or biotin-binding protein, such as, but not limited to rhizavidin or a derivative or functional fragment thereof, such as an amino acid sequence having at least 85% sequence identity to SEQ ID NO: 1. Alternatively, it is also possible to prepare target antigen and then conjugate it to a complementary affinity molecule, e.g., but not limited to rhizavidin or a derivative or functional fragment thereof.

Polypeptides can also by synthesized as branched structures such as those disclosed in U.S. Pat. Nos. 5,229,490 and 5,390,111. Antigenic polypeptides include, for example, synthetic or recombinant B-cell and T-cell epitopes, universal T-cell epitopes, and mixed T-cell epitopes from one organism or disease and B-cell epitopes from another.

An antigen can be obtained through recombinant means or chemical polypeptide synthesis, as well as antigen obtained from natural sources or extracts, can be purified by means of the antigen's physical and chemical characteristics, such as by fractionation or chromatography. These techniques are well-known in the art.

In some embodiments, an antigen can be solubilized in water, a solvent such as methanol, or a buffer. Suitable buffers include, but are not limited to, phosphate buffered saline Ca²⁺/Mg²⁺ free (PBS), normal saline (150 mM NaCl in water), and Tris buffer. Antigen not soluble in neutral buffer can be solubilized in 10 mM acetic acid and then diluted to the desired volume with a neutral buffer such as PBS. In the case of antigen soluble only at acid pH, acetate-PBS at acid pH can be used as a diluent after solubilization in dilute acetic acid. Glycerol can be a suitable non-aqueous solvent for use the compositions, methods and kits described herein.

Typically, when designing a protein vaccine against a pathogen, an extracellular protein or one exposed to the environment on a virus is often the ideal candidate as the antigen component in the vaccine. Antibodies generated against that extracellular protein become the first line of defense against the pathogen during infection. The antibodies bind to the protein on the pathogen to facilitate antibody opsonization and mark the pathogen for ingestion and destruction by a phagocyte such as a macrophage. Antibody opsonization can also kill the pathogen by antibody-dependent cellular cytotoxicity. The antibody triggers a release of lysis products from cells such as monocytes, neutrophils, eosinophils, and natural killer cells.

In one embodiment of the invention described herein, antigens for use in the compositions as disclosed herein all wild type proteins, as in the amino acid residues have the sequences found in naturally occurring viruses and have not been altered by selective growth conditions or molecular biological methods.

In one embodiment, the immunogenic compositions described as herein can comprise antigens which are glycosylated proteins. In other words, an antigen of interest can each be a glycosylated protein. In one embodiment of the immunogenic compositions as described herein, antigens, or antigen-fusion polypeptides are O-linked glycosylated. In another embodiment of the immunogenic compositions as described herein, antigens, or antigen-fusion polypeptides are N-linked glycosylated. In yet another embodiment of the immunogenic compositions as described herein, antigens, or antigen-fusion are both O-linked and N-linked glycosylated. In other embodiments, other types of glycosylations are possible, e.g., C-mannosylation. Glycosylation of proteins occurs predominantly in eukaryotic cells. N-glycosylation is important for the folding of some eukaryotic proteins, providing a co-translational and post-translational modification mechanism that modulates the structure and function of membrane and secreted proteins. Glycosylation is the enzymatic process that links saccharides to produce glycans, and attaches them to proteins and lipids. In N-glycosylation, glycans are attached to the amide nitrogen of asparagine side chain during protein translation. The three major saccharides forming glycans are glucose, mannose, and N-acetylglucosamine molecules. The N-glycosylation consensus is Asn-Xaa-Ser/Thr, where Xaa can be any of the known amino acids. O-linked glycosylation occurs at a later stage during protein processing, probably in the Golgi apparatus. In O-linked glycosylation, N-acetyl-galactosamine, O-fucose, 0-glucose, and/or N-acetylglucosamine is added to serine or threonine residues. One skilled in the art can use bioinformatics software such as NetNGlyc 1.0 and NetOGlyc Prediction softwares from the Technical University of Denmark to find the N- and O-glycosylation sites in a polypeptide in the present invention. The NetNglyc server predicts N-Glycosylation sites in proteins using artificial neural networks that examine the sequence context of Asn-Xaa-Ser/Thr sequons. The NetNGlyc 1.0 and NetOGlyc 3.1 Prediction software can be accessed at the EXPASY website. In one embodiment, N-glycosylation occurs in the target antigen polypeptide of the fusion polypeptide described herein.

### C. SA-Antigen-Fusion Proteins

In some embodiments, the SA antigen as disclosed herein (e.g., SA1739 (B2), SA1720 (B1), SA1890 (B3), SA0103 (T1), SA0377 (T2), SA0693 (T3) and SA2105 (T4)) for use in the MAPS complex as disclosed herein is fused to a recombinant biotin-binding protein. In some embodiment, the recombinant biotin-binding protein is a rhizavidin protein. In some embodiments, the Rhizavidin (Rhavi) protein comprises SEQ ID NO: 1 or a protein or polypeptide of at least 85% amino acid sequence identity to SEQ ID NO: 1.

In some embodiments, the recombinant biotin-binding protein comprises an E. coli signal sequence fused to the N-terminus of an amino acid sequence comprising amino acids 45-179 of wild-type Rhizavidin (rhavi) which is as follows:

(SEQ ID NO: 1)
FDASNFKDFSSIASASSSWQNQSGSTMIIQVDSFGNVSGQYVNRAQGTG
CQNSPYPLTGRVNGTFIAFSVGWNNSTENCNSATGWTGYAQVNGNNTEI
VTSWNLAYEGGSGPAIEQGQDTFQYVPTTENKSLLKD.

In some embodiments, the recombinant biotin-binding protein consists of, or consists essentially of, the amino acid sequence corresponding to amino acids 45-179 of the wild-type Rhizavidin. Amino acid sequence of the wild-type Rhizavidin is:

(SEQ ID NO: 21)
MIITSLYATFGTIADGRRTSGGKTMIRTNAVAALVFAVATSALAFDASN
FKDFSSIASASSSWQNQSGSTMIIQVDSFGNVSGQYVNRAQGTGCQNSP
YPLTGRVNGTFIAFSVGWNNSTENCNSATGWTGYAQVNGNNTEIVTSWN
LAYEGGSGPAIEQGQDTFQYVPTTENKSLLKD.

In other words, the biotin-binding domain does not comprise (i.e., lacks) lacks amino acids 1-44

(MIITSLYATFGTIADGRRTSGGKTMIRTNAVAALVFAVATSALA, SEQ ID NO: 22).

In some embodiments, the recombinant biotin-binding protein useful in a fusion protein with at least one SA-antigen as disclosed herein comprises an amino acid sequence having at least 50% identity, at least 55% identity, at least 60% identity, at least 65% identity, at least 70% identity, at least 75% identity, at least 80% identity, preferably at least 85% identity, at least 90% identity, at least 95% identity, at least 96% identity, at least 97% identity, at least 98% identity, or at least 99% identity, and more preferably at least 99.3% identity to the amino acid sequence of SEQ ID NO: 1. In some embodiments, the recombinant biotin-binding protein comprises the amino acid sequence of SEQ ID NO: 1 that has at least 1, or at least 2, or at least 3 or at least 4 or at least 5 amino acid substitutions in SEQ ID NO: 1 to prevent physiologically specific N-linked glycosylation. In some embodiments, the recombinant biotin-binding protein comprises the amino acid sequence of SEQ ID NO: 1 that has one or more of the following modifications; N80A, T108A, N118A, S119A, N138A. In some embodiments, the recombinant biotin-binding protein comprises SEQ ID NO: 1 that has two, or 3, or 4 or 5 amino acid modifications selected from any of: N80A, T108A, Ni 18A, S119A, N138A.

A SA-antigen for use in the SA-MAPS composition as disclosed herein can be genetically fused to rhizavidin (rhavi), which is a dimeric biotin-binding protein from Rhizobium etli, according to the methods as disclosed in U.S. Pat. No. 9,499,593 which is incorporated herein in its entirety by reference.

In some embodiments, a biotin-binding protein useful in the SA-MAPS composition as disclosed herein comprises a sequence X¹-X²-X³, wherein X² is a peptide having the amino acid sequence corresponding to amino acids 45-179 of the wild-type Rhizavidin (i.e., SEQ ID NO: 1) and X¹ and X³ are independently absent, or a peptide of 1 to about 100 amino acids with the proviso that the N-terminus of X¹ does not comprise an amino acid sequence corresponding to N-terminus of amino acids 1-44 of the wild-type Rhizavidin.

In some embodiments, the biotin-binding proteins can comprise a signal peptide conjugated to the N-terminus of the biotin-binding protein, i.e. X¹ can comprise a signal peptide. The signal peptide is also called a leader peptide in the N-terminus, which may or may not be cleaved off after the translocation through the membrane. In some embodiments, the E. coli signal sequence is the Dsba signal sequence which comprises at least MKKIWLALAGLVLAFSASA (SEQ ID NO: 23) or MKKIWLALAGLVLAFSASAAQDP (SEQ ID NO: 24). In some embodiments, the signal sequence is MKKVAAFVALSLLMAGC (SEQ ID NO: 25). Secretion/signal peptides are described in more detail below. In some embodiments, the signal sequence is MKKIWLALAGLVLAFSASA (SEQ ID NO: 26), MAPFEPLASGILLLLWLIAPSRA (SEQ ID NO: 27), MKKVAAFVALSLLMAGC (SEQ ID NO: 28), or a derivative or functional portion thereof. The signal sequence can be fused with the sequence comprising amino acids 45-179 of wild-type rhavi by a flexible peptide linker.

In some embodiments, the biotin-binding protein is a fusion protein with one or more SA-antigens. For example, the amino acid sequence of SEQ ID NO: 1 (or a protein of at least 80% or 85% or more sequence identity thereto) is fused to at least 1, or at least 2 or at least 3, or at least 4 or more SA-antigens as disclosed herein. That is, in some embodiments, a fusion protein useful in the methods and compositions as disclosed herein comprises in the following order: an amino acid sequence comprising SEQ ID NO: 1 or a protein of at least 80% or 85% or more sequence identity thereto, fused to a at least one or more SA-antigens as disclosed herein (e.g., Rhavi-SA fusion polypeptide). In an alternative embodiment, a fusion protein useful in the methods and compositions as disclosed herein comprises in the following order: a fusion protein comprising at least one or more SA-antigens as disclosed herein fused to an amino acid sequence comprising SEQ ID NO: 1 or a protein of at least 80% or 85% or more sequence identity thereto (e.g., SA-Rhavi fusion polypeptide).

In some embodiments, a biotin-binding protein is a fusion protein comprising SEQ ID NO: 1 (or a polypeptide having at least 80% or 85% or more sequence identity thereto or a biotin-binding portion thereof) fused to a polypeptide comprising any of the amino acid sequences of: SA1739 (B2), SA1720 (B1), SA1890 (B3), SA0103 (T1), SA0377 (T2), SA0693 (T3) and SA2105 (T4), or fragments or polypeptides thereof comprising an amino acid sequence having at least 80%, 85%, 90% identity to any of: SEQ ID NO: 50 (SA1739; B1), SEQ ID NO: 51 (SA1720; B2), SEQ ID NO: 52 (SA1890; B3), SEQ ID NO: 53 (SA0103; T1), SEQ ID NO: 54 (SA0377; T2), SEQ ID NO: 55 (SA0693; T3), SEQ ID NO: 56 (SA2105; T4). In some embodiments, a fusion protein comprises a polypeptide comprising any of the amino acid sequences of: SA1739 (B2), SA1720 (B1), SA1890 (B3), SA0103 (T1), SA0377 (T2), SA0693 (T3) and SA2105 (T4), or fragments or polypeptides thereof comprising an amino acid sequence having at least 80%, 85%, 90% identity to any of: SEQ ID NO: 50 (SA1739; B1), SEQ ID NO: 51 (SA1720; B2), SEQ ID NO: 52 (SA1890; B3), SEQ ID NO: 53 (SA0103; T1), SEQ ID NO: 54 (SA0377; T2), SEQ ID NO: 55 (SA0693; T3), SEQ ID NO: 56 (SA2105; T4) fused to a biotin-binding protein comprising the amino acid sequence of SEQ ID NO: 1 (or a polypeptide having at least 80% or 85% or more sequence identity to SEQ ID NO: 1.

Aspects of the present invention are directed to an isolated recombinant rhizavidin fusion protein comprising, in any order, a biotin-binding moiety comprising an amino acid sequence at least 80%, or at least 85% or at least 90% identical to SEQ ID NO: 1, or a biotin binding portion thereof, and a SA1739 (B2) polypeptide comprising the amino acid sequence of at least 80%, or at least 85%, or at least 90% sequence identity to SEQ ID NO: 51 (Rhavi-SA1739). In some embodiments, the fusion protein comprises, in the following order: a biotin-binding moiety comprising an amino acid sequence at least 80%, or at least 85% or at least 90% identical to SEQ ID NO: 1, or a biotin binding portion thereof, and a SA1739 (B2) polypeptide comprising the amino acid sequence of at least 80%, or at least 85%, or at least 90% sequence identity to SEQ ID NO: 51 (Rhavi-SA 1739). In alternative embodiments, the fusion protein comprises, in the following order; a SA1739 (B2) polypeptide comprising the amino acid sequence of at least 80%, or at least 85%, or at least 90% sequence identity to SEQ ID NO: 51, and a biotin-binding moiety comprising an amino acid sequence at least 80%, or at least 85% or at least 90% identical to SEQ ID NO: 1, or a biotin binding portion (SA 1739-Rhavi).

Aspects of the present invention are directed to an isolated recombinant rhizavidin fusion protein comprising SEQ ID NO: 1 (or a protein of at least 80% or 85% or more sequence identity thereto) fused to SA1720(B1) (Rhavi-SA1720). Aspects of the present invention are directed to an isolated recombinant rhizavidin fusion protein comprising, in any order, a biotin-binding moiety comprising an amino acid sequence at least 80%, or at least 85% or at least 90% identical to SEQ ID NO: 1, or a biotin binding portion thereof, and a SA1720 (B1) polypeptide comprising the amino acid sequence of at least 80%, or at least 85%, or at least 90% sequence identity to SEQ ID NO: 50 (Rhavi-SA1720). In some embodiments, the fusion protein comprises, in the following order: a biotin-binding moiety comprising an amino acid sequence at least 80%, or at least 85% or at least 90% identical to SEQ ID NO: 1, or a biotin binding portion thereof, and a SA1720 (B1) polypeptide comprising the amino acid sequence of at least 80%, or at least 85%, or at least 90% sequence identity to SEQ ID NO: 50 (Rhavi-SA1720). In alternative embodiments, the fusion protein comprises, in the following order; a SA1720 (B1) polypeptide comprising the amino acid sequence of at least 80%, or at least 85%, or at least 90% sequence identity to SEQ ID NO: 50, and a biotin-binding moiety comprising an amino acid sequence at least 80%, or at least 85% or at least 90% identical to SEQ ID NO: 1, or a biotin binding portion (SA1720-Rhavi).

Aspects of the present invention are directed to an isolated recombinant rhizavidin fusion protein comprising SEQ ID NO: 1 (or a protein of at least 80% or 85% or more sequence identity thereto) fused to SA1890 (B3) (Rhavi-SA1890). In some embodiments, an isolated recombinant rhizavidin fusion protein comprising, in any order, a biotin-binding moiety comprising an amino acid sequence at least 80%, or at least 85% or at least 90% identical to SEQ ID NO: 1, or a biotin binding portion thereof, and a SA1890 (B3) polypeptide comprising the amino acid sequence of at least 80%, or at least 85%, or at least 90% sequence identity to SEQ ID NO: 52 (Rhavi-SA1890). In some embodiments, the fusion protein comprises, in the following order: a biotin-binding moiety comprising an amino acid sequence at least 80%, or at least 85% or at least 90% identical to SEQ ID NO: 1, or a biotin binding portion thereof, and a SA1890 (B3) polypeptide comprising the amino acid sequence of at least 80%, or at least 85%, or at least 90% sequence identity to SEQ ID NO: 52 (Rhavi-SA1890). In alternative embodiments, the fusion protein comprises, in the following order; a SA1890 (B3) polypeptide comprising the amino acid sequence of at least 80%, or at least 85%, or at least 90% sequence identity to SEQ ID NO: 52, and a biotin-binding moiety comprising an amino acid sequence at least 80%, or at least 85% or at least 90% identical to SEQ ID NO: 1, or a biotin binding portion (SA1890-Rhavi).

Aspects of the present invention are directed to an isolated recombinant rhizavidin fusion protein comprising SEQ ID NO: 1 (or a protein of at least 80% or 85% or more sequence identity thereto) fused to SA0103 (T1) (Rhavi-SA0103). In some embodiments, an isolated recombinant rhizavidin fusion protein comprising, in any order, a biotin-binding moiety comprising an amino acid sequence at least 80%, or at least 85% or at least 90% identical to SEQ ID NO: 1, or a biotin binding portion thereof, and a SA0103 (T1) polypeptide comprising the amino acid sequence of at least 80%, or at least 85%, or at least 90% sequence identity to SEQ ID NO: 53 (Rhavi-SA0103). In some embodiments, the fusion protein comprises, in the following order: a biotin-binding moiety comprising an amino acid sequence at least 80%, or at least 85% or at least 90% identical to SEQ ID NO: 1, or a biotin binding portion thereof, and a SA0103 (T1) polypeptide comprising the amino acid sequence of at least 80%, or at least 85%, or at least 90% sequence identity to SEQ ID NO: 53 (Rhavi-SA0103 (T1)). In alternative embodiments, the fusion protein comprises, in the following order; a SA0103 (T1) polypeptide comprising the amino acid sequence of at least 80%, or at least 85%, or at least 90% sequence identity to SEQ ID NO: 53, and a biotin-binding moiety comprising an amino acid sequence at least 80%, or at least 85% or at least 90% identical to SEQ ID NO: 1, or a biotin binding portion (SA0103-Rhavi).

Aspects of the present invention are directed to an isolated recombinant rhizavidin fusion protein comprising SEQ ID NO: 1 (or a protein of at least 80% or 85% or more sequence identity thereto) fused to SA0377 (T2) (Rhavi-SA0377). In some embodiments, an isolated recombinant rhizavidin fusion protein comprising, in any order, a biotin-binding moiety comprising an amino acid sequence at least 80%, or at least 85% or at least 90% identical to SEQ ID NO: 1, or a biotin binding portion thereof, and a SA0377 (T2) polypeptide comprising the amino acid sequence of at least 80%, or at least 85%, or at least 90% sequence identity to SEQ ID NO: 54 (Rhavi-SA0377). In some embodiments, the fusion protein comprises, in the following order: a biotin-binding moiety comprising an amino acid sequence at least 80%, or at least 85% or at least 90% identical to SEQ ID NO: 1, or a biotin binding portion thereof, and a SA0377 (T2) polypeptide comprising the amino acid sequence of at least 80%, or at least 85%, or at least 90% sequence identity to SEQ ID NO: 54 (Rhavi-SA0377). In alternative embodiments, the fusion protein comprises, in the following order; a SA0377 (T2) polypeptide comprising the amino acid sequence of at least 80%, or at least 85%, or at least 90% sequence identity to SEQ ID NO: 54, and a biotin-binding moiety comprising an amino acid sequence at least 80%, or at least 85% or at least 90% identical to SEQ ID NO: 1, or a biotin binding portion (SA0377-Rhavi).

Aspects of the present invention are directed to an isolated recombinant rhizavidin fusion protein comprising SEQ ID NO: 1 (or a protein of at least 80% or 85% or more sequence identity thereto) fused to SA0693 (T3) (Rhavi-SA0693). In some embodiments, an isolated recombinant rhizavidin fusion protein comprising, in any order, a biotin-binding moiety comprising an amino acid sequence at least 80%, or at least 85% or at least 90% identical to SEQ ID NO: 1, or a biotin binding portion thereof, and a SA0693 (T3) polypeptide comprising the amino acid sequence of at least 80%, or at least 85%, or at least 90% sequence identity to SEQ ID NO: 55 (Rhavi-SA0693). In some embodiments, the fusion protein comprises, in the following order: a biotin-binding moiety comprising an amino acid sequence at least 80%, or at least 85% or at least 90% identical to SEQ ID NO: 1, or a biotin binding portion thereof, and a SA0377 (T2) polypeptide comprising the amino acid sequence of at least 80%, or at least 85%, or at least 90% sequence identity to SEQ ID NO: 55 (Rhavi-SA0693). In alternative embodiments, the fusion protein comprises, in the following order; a SA0693 (T3) polypeptide comprising the amino acid sequence of at least 80%, or at least 85%, or at least 90% sequence identity to SEQ ID NO: 55, and a biotin-binding moiety comprising an amino acid sequence at least 80%, or at least 85% or at least 90% identical to SEQ ID NO: 1, or a biotin binding portion (SA0693-Rhavi).

Aspects of the present invention are directed to an isolated recombinant rhizavidin fusion protein comprising SEQ ID NO: 1 (or a protein of at least 80% or 85% or more sequence identity thereto) fused to SA2105 (T4) (Rhavi-SA2105). In some embodiments, an isolated recombinant rhizavidin fusion protein comprising, in any order, a biotin-binding moiety comprising an amino acid sequence at least 80%, or at least 85% or at least 90% identical to SEQ ID NO: 1, or a biotin binding portion thereof, and a SA2105 (T4) polypeptide comprising the amino acid sequence of at least 80%, or at least 85%, or at least 90% sequence identity to SEQ ID NO: 56 (Rhavi-SA2105). In some embodiments, the fusion protein comprises, in the following order: a biotin-binding moiety comprising an amino acid sequence at least 80%, or at least 85% or at least 90% identical to SEQ ID NO: 1, or a biotin binding portion thereof, and a SA0377 (T2) polypeptide comprising the amino acid sequence of at least 80%, or at least 85%, or at least 90% sequence identity to SEQ ID NO: 56 (Rhavi-SA2105). In alternative embodiments, the fusion protein comprises, in the following order; a SA2105 (T4) polypeptide comprising the amino acid sequence of at least 80%, or at least 85%, or at least 90% sequence identity to SEQ ID NO: 56, and a biotin-binding moiety comprising an amino acid sequence at least 80%, or at least 85% or at least 90% identical to SEQ ID NO: 1, or a biotin binding portion (SA2105 (T4)-Rhavi).

In some embodiments, there is a linker between the biotin binding protein, e.g., the rhizavidin polypeptide comprising SEQ ID NO: 1 and the SA-antigen. In some embodiments, there is no linker. Exemplary Rhizavidin fusion proteins comprising SA-antigens are shown in Table 5A.

TABLE 5A

| | |
|---|---|
| Rhavi-SA1739 | Rhavi-linker-SA 1739 |
| SA1739-Rhavi | SA1739-linker-Rhavi |
| Rhavi-SA1720 | Rhavi-linker-SA1720 |
| SA1720-Rhavi | SA1720-linker-Rhavi |
| Rhavi-SA1890 | Rhavi-linker-SA1890 |
| SA1890-Rhavi | SA1890-linker-Rhavi |
| Rhavi-SA0103 | Rhavi-linker-SA0103 |
| SA0103-Rhavi | SA0103-linker-Rhavi |
| Rhavi-SA0377 | Rhavi-linker-SA0377 |
| SA0377-Rhavi | SA0377-linker-Rhavi |
| Rhavi-SA0693 | Rhavi-linker-SA0693 |
| SA0693-Rhavi | SA0693-linker-Rhavi |
| Rhavi-SA2105 | Rhavi-linker-SA2105 |
| SA2105-Rhavi | SA2105-linker-Rhavi |

In some embodiments, an exemplary fusion protein disclosed in Table 5A can further comprise an additional SA-antigen. For example, in some embodiments, a fusion protein disclosed in Table 5A can comprise at least one SA-antigen selected from the group of polypeptides from the group of: SEQ ID NO: 50 (SA1739; B1), SEQ ID NO: 51 (SA1720; B2), SEQ ID NO: 52 (SA1890; B3), SEQ ID NO: 53 (SA0103; T1), SEQ ID NO: 54 (SA0377; T2), SEQ ID NO: 55 (SA0693; T3), or SEQ ID NO: 56 (SA2105; T4), or a polypeptide having at least 80%, or at least 85% sequence identity to SEQ ID NO: 50-56. In some embodiments, a fusion protein disclosed in Table 5A can comprise at least one SA-antigen selected from the group of polypeptides from the group of SEQ ID NO: 16 (Hla209), SEQ ID NO: 17 (HlaW205A); SEQ ID NO: 18 (HlaW213A); SEQ ID NO: 2 (ClfA); SEQ ID NO: 3 (ClfA 221-559); SEQ ID NO: 4 (ClfB) SEQ ID NO: 5 (ClfB 203-542), SEQ ID NO: 6 (SdrD); SEQ ID NO: 7 (SdrD 246-682); SEQ ID NO: 10 (IsdA); SEQ ID NO: 11 (IsdA 47-324), SEQ ID NO: 12 (IsdB); SEQ ID NO: 13 (IsdB 48-447) and SEQ ID NO: 8 (SrdE); or a polypeptide having at least 80%, or at least 85% sequence identity to SEQ ID NO: 16-18 or 3-13. It is envisioned that the biotin-binding protein of SEQ ID NO: 1, or a polypeptide having at least 80% or 85% sequence identity to SEQ ID NO: 1 is located at either the N-terminal, or the C-terminal of the fusion protein, or in the middle of the fusion protein (i.e., located between two SA-antigens). For example, where A1 is the first SA-antigen, and A2 is the second SA-antigen, and Rhavi is a polypeptide of SEQ ID NO: 1, the fusion protein can occur in the following order: Rhavi-A1-A2, Al-Rhavi-A2, or A1-A2-Rhavi.

In some embodiments, a biotin-binding protein fusion protein comprises, in any order: (i) a polypeptide having the amino acid sequence of SEQ ID NO: 1 (or a protein of at least 80% or 85% or more sequence identity thereto) and (ii) at least two antigens selected from any one of: SA1739 (B2), SA1720 (B1), SA1890 (B3), SA0103 (T1), SA0377 (T2), SA0693 (T3) and SA2105 (T4) or proteins or peptides having at least 85% sequence identity to any of: SEQ ID NO: 50-56.

In some embodiments, the SA-antigens may be the same antigens (e.g., SEQ ID NO: 1-A-A), or alternatively different SA antigens (e.g., SEQ ID NO: 1-A-B), where A and B are different SA-antigens selected from Group A as disclosed herein. Exemplary Rhizavidin fusion proteins comprising 2 SA-antigens are shown in Table 5B. The two SA-antigens fused to Rhavi can be both T-cell antigens (e.g., two selected from any of SA0103 (T1), SA0377 (T2), SA0693 (T3) and SA2105 (T4)), or can both be B-cell antigens, (e.g., two selected from any of SA1739 (B2), SA1720 (B1), SA1890 (B3)), or a T-cell antigen and a B-cell antigens.

TABLE 5B
Exemplary Rhizavidin fusion proteins comprising different combinations
of 2 SA-antigens from Group A. It is noted that the order of the 2
antigens fused to the Rhizavidin protein of SEQ ID NO: 1 (referred
to as “Rhavi”) or a homologue of at least 80% identity thereto
can be in any order, e.g., Rhavi-B1-T1, or alternatively, Rhavi-T1-
B1, or B1-Rhavi-T1 or T1-Rhavi-B1, or Rhavi-T1-T2, or Rhavi-B1-B2 etc.

| | | |
|---|---|---|
| Rhavi-SA0103-SA0377 | SA0103-SA0377-Rhavi | SA0103-Rhavi-SA0377 |
| Rhivi- SA0103-SA0693 | SA0103-SA0693-Rhavi | SA0103-Rhavi-SA0693 |
| Rhivi- SA0103-SA2105 | SA0103-SA2105-Rhavi | SA0103-Rhavi-SA2105 |
| Rhavi- SA0103-SA1739 | SA0103-SA1739-Rhavi | SA0103-Rhavi-SA1739 |
| Rhavi- SA0103-SA1720 | SA0103-SA1720-Rhavi | SA0103-Rhavi-SA1720 |
| Rhavi- SA0103-SA1890 | SA0103-SA1890-Rhavi | SA0103-Rhavi-SA1890 |
| Rhivi- SA0377-SA0693 | SA0377-SA0693-Rhavi | SA0377-Rhavi-SA0693 |
| Rhivi- SA0377-SA2105 | SA0377-SA2105-Rhavi | SA0377-Rhavi-SA2105 |
| Rhavi- SA0377-SA1739 | SA0377-SA1739-Rhavi | SA0377-Rhavi-SA1739 |
| Rhavi- SA0377-SA1720 | SA0377-SA1720-Rhavi | SA0377-Rhavi-SA1720 |
| Rhavi- SA0377-SA1890 | SA0377-SA1890-Rhavi | SA0377-Rhavi-SA1890 |
| Rhivi- SA0693-SA2105 | SA0693-SA2105-Rhavi | SA0693-Rhavi-SA2105 |
| Rhavi- SA0693-SA1739 | SA0693-SA1739-Rhavi | SA0693-Rhavi-SA1739 |
| Rhavi- SA0693-SA1720 | SA0693-SA1720-Rhavi | SA0693-Rhavi-SA1720 |
| Rhavi- SA0693-SA1890 | SA0693-SA1890-Rhavi | SA0693-Rhavi-SA1890 |
| Rhavi- SA2105-SA1739 | SA2105-SA1739-Rhavi | SA2105-Rhavi-SA1739 |
| Rhavi- SA2105-SA1720 | SA2105-SA1720-Rhavi | SA2105-Rhavi-SA1720 |
| Rhavi- SA2105-SA1890 | SA2105-SA1890-Rhavi | SA2105-Rhavi-SA1890 |
| Rhavi- SA1739-SA1720 | SA1739-SA1720-Rhavi | SA1739-Rhavi-SA1720 |
| Rhavi- SA1739-SA1890 | SA1739-SA1890-Rhavi | SA1739-Rhavi-SA1890 |
| Rhavi- SA1720-SA1890 | SA1720-SA1890-Rhavi | SA1720-Rhavi-SA1890 |

It is envisioned that any of the SA antigens in the Rhavi-antigen-antigen fusion proteins shown in Table 5A can be substituted or replaced with any other SA antigen as disclosed herein, or known to one of ordinary skill in the art.

In some embodiments, a rhizavidin fusion protein comprising a SA antigen can comprise a lipidation sequence at the N-terminus, e.g., MKKVAAFVALSLLMAGC (SEQ ID NO: 29) or an amino acid 85% identity thereto.

In some embodiments, a rhizavidin fusion protein comprising a SA antigen can comprise a signal peptide linked to the N-terminus of the biotin-binding domain either directly (e.g., via a bond) or indirectly (e.g., by a linker). In some embodiments, the signal peptide can be linked to the N-terminus of the biotin-binding domain by a peptide linker. The peptide linker sequence can be of any length. For example, the peptide linker sequence can be one, two, three, four, five, six, seven, eight, nine, ten, eleven, twelve, thirteen, fourteen, fifteen or more amino acids in length. In some embodiments, the peptide linker is four amino acids in length.

The peptide linker sequence can comprise any amino acid sequence. For example, the peptide linker can comprise an amino acid sequence which can be cleaved by a signal peptidase. In some embodiments, the peptide linker comprises the amino acid sequence AQDP (SEQ ID NO: 30) or VSDP (SEQ ID NO: 31). Other peptide linkers are known in the art, and include, GGGGSSS (SEQ ID NO: 71) or AAA (SEQ ID NO: 72) which can be located between the rhizavidin polypeptide comprising SEQ ID NO: 1 and a SA-antigen. If there are more than one SA-antigens in the fusion protein, a linker can also optionally be located between each of the SA-antigens.

In some embodiments, a rhizavidin fusion protein comprising a SA antigen can be conjugated at its C-terminus to a peptide of 1-100 amino acids. Such peptides at the C-terminus can be used for purification tags, linkers to other domains, and the like. In some embodiments, a rhizavidin fusion protein comprising a SA antigen comprises on its N- or C-terminus one or more (e.g., one, two, three, four, five, six, seven, eight, nine, ten or more) purification tags. Examples of purification tags include, but are not limited to a histidine tag, a c-my tag, a Halo tag, a Flag tag, and the like. In some embodiments, the biotin-binding protein comprises on its C-terminus a histidine tag, e.g. a (His)₆ (SEQ ID NO. 32). In some embodiments, a rhizavidin fusion protein comprising a SA antigen for use in the SA-MAPS immunogenic composition as disclosed herein comprises a peptide of amino acid sequence GGGGSSSVDKLAAALEHHHHHH (SEQ ID NO: 33). This peptide at the C-terminus provides a histidine tag for purification and a place for insertion of other domains, e.g. antigenic domains, in the biotin protein. Further, while Helppolainen et al. (Biochem J., 2007, 405: 397-405) describe expression of Rhizavidin in E. coli, there is no teaching or suggestion in Helppolainen et al. for conjugating an additional peptide to the C-terminus of the biotin-binding domain of Rhizavidin.

A purification tag can be conjugated to a rhizavidin fusion protein comprising a SA antigen as disclosed herein by a peptide linker to enhance the probability that the tag is exposed to the outside. The length of the linker can be at least one (e.g., one, two, three, four, five six, seven, eight, nine, ten, eleven, twelve, thirteen, fourteen, or fifteen) amino acid. The linker peptide can comprise any amino acid sequence without limitations. In some embodiments, the linker peptide comprises the amino acid sequence VDKLAAALE (SEQ ID NO: 34) or GGGGSSSVDKLAAALE (SEQ ID NO: 35). In some embodiments, a rhizavidin fusion protein comprising a SA antigen as disclosed herein can comprise at its C-terminus the amino acid sequence VDKLAAALEHIHHIHH (SEQ ID NO: 36) or GGGGSSSVDKLAAALEHHHHHH (SEQ ID NO: 37).

As discussed herein, a rhizavidin fusion protein comprising a SA antigen for use in the SA-MAPS immunogenic composition as disclosed herein consists of amino acids 45-179 of wild-type Rhizavidin.

In some embodiments, rhizavidin fusion protein comprising a SA antigen for use in the SA-MAPS immunogenic composition as disclosed herein can comprise an N-terminal signal sequence as disclosed herein. In some embodiments, the signal sequence is attached to the N-terminal of the complementary affinity molecule, e.g., biotin-binding protein such as, e.g., Rhavi, as disclosed herein.

In some embodiments, a rhizavidin fusion protein comprising a SA antigen for use in the SA-MAPS immunogenic composition as disclosed herein has a spacer peptide, e.g., a 14-residue spacer (GSPGISGGGGGILE) (SEQ ID NO: 38) separating the SA antigen from the rhizavidin protein. The coding sequence of such a short spacer can be constructed by annealing a complementary pair of primers. One of skill in the art can design and synthesize oligonucleotides that will code for the selected spacer. Spacer peptides should generally have non-polar amino acid residues, such as glycine and proline.

### Lipidated Rhizavidin Fusion Protein or Biotin-Binding Protein

In another aspect provided herein is a lipidated biotin-binding protein, e.g., a lipidated rhizavidin fusion protein comprising a SA antigen for use in the SA-MAPS immunogenic composition as disclosed herein. As used herein, the term “lipidated biotin-binding protein” refers to a biotin-binding protein that is covalently conjugated with a lipid. The lipid moieties could be a diacyl or triacyl lipid.

In some embodiments, a rhizavidin fusion protein comprising a SA antigen for use in the SA-MAPS immunogenic composition as disclosed herein comprises a lipidation sequence. As used herein, the term “lipidation sequence” refers to an amino acid sequence that facilitates lipidation in bacteria, e.g., E. coli, of a polypeptide carrying the lipidating sequence. The lipidation sequence can be present at the N-terminus or the C-terminus of the protein. The lipidation sequence can be linked to the recombinant biotin-binding protein to form a fusion protein, which is in lipidated form when expressed in E. coli by conventional recombinant technology. In some embodiments, a lipidation sequence is located at the N-terminus of the biotin-binding protein.

Any lipidation sequence known to one of ordinary skill in the art can be used. In some embodiments, the lipidating sequence is MKKVAAFVALSLLMAGC (SEQ ID NO: 39) or a derivative or functional portion thereof. Other exemplary lididationg sequences include, but are not limited to, MNSKKLCCICVLFSLLAGCAS (SEQ ID NO: 40), MRYSKLTMLIPCALLLSAC (SEQ ID NO: 41), MFVTSKKMTAAVLAITLAMSLSAC (SEQ ID NO: 42), MIKRVLVVSMVGLSLVGC (SEQ ID NO: 43), and derivatives or functional portions thereof.

In some embodiments, the lipidation sequence can be fused to a rhizavidin fusion protein comprising a SA antigen via a peptide linker, wherein the peptide linker attaches the lipidating sequence to the biotin-binding protein. In some embodiment, the peptide linker comprises the amino acid sequence VSDP (SEQ ID NO: 44) or AQDP (SEQ ID NO: 45).

In some embodiments, a rhizavidin fusion protein comprising a SA antigen for use in the SA-MAPS immunogenic composition as disclosed herein that is a lipoprotein as described herein have enhanced immunogenicity. Without wishing to be bound by a theory, lipid moieties at the N-terminals of the lipoproteins or lipopeptides contribute to the adjuvant activity. Accordingly, additional embodiments provide immunogenic or vaccine compositions for inducing an immunological response, comprising the isolated biotin-binding lipoprotein, or a suitable vector for in vivo expression thereof, or both, and a suitable carrier, as well as to methods for eliciting an immunological or protective response comprising administering to a host the isolated recombinant biotin-binding lipoprotein, the vector expressing the recombinant biotin-binding lipoprotein, or a composition containing the recombinant lipoprotein or vector, in an amount sufficient to elicit the response.

A SA-MAPS immunogenic composition comprising a rhizavidin fusion protein comprising a SA antigen that is a lipoprotein elicits an immunological response-local or systemic. The response can, but need not, be protective.

### D. Polysaccharides

One component of the SA-MAPS immunogenic composition as disclosed herein is a “backbone,” typically an antigenic or immunogenic polysaccharide (PS), and can comprise additional elements that do not negatively impact the antigenic polysaccharide's function of (i) inducing an immune response to the polysaccharide and (ii) presenting the associated SA-antigen(s) to the immune system in immunogenic fashion. In some embodiments, the immunogenic polysaccharide is a synthetic polysaccharide.

It is envisioned that the polysaccharide used in the SA-MAPS composition is immunogenic, that is, it helps induce a specific immune response, and herein is referred to as an “immunogenic polysaccharide” or “antigenic polysaccharide”. The specific immune response recognizes the particular immunogenic PS and provides a unique response to the immunogenic complex as opposed to a different immunogenic complex. As explained herein, the response includes both a humoral and cell-mediated response.

In some embodiments, the immunogenic polysaccharide is a naturally occurring polysaccharide, e.g., a polysaccharide derived or purified from bacterial cells, and can be, for example, a capsular or noncaspular PS. In some embodiments, the immunogenic polysaccharide is derived or purified from eukaryotic cells, e.g., fungi, insect or plant cells. In yet other embodiments, the immunogenic polysaccharide is derived from mammalian cells, such as virus-infected cells or cancer cells. In general, such immunogenic polysaccharides are well known in the art and are encompassed for use in the methods and compositions as disclosed herein.

Staphylococcal microorganisms capable of causing invasive disease generally also are capable of producing a capsule polysaccharide (CP) that encapsulates the bacterium and enhances its resistance to clearance by the host innate immune system. The CP serves to cloak the bacterial cell in a protective capsule that renders the bacteria resistant to phagocytosis and intracellular killing. Bacteria lacking a capsule are more susceptible to phagocytosis. Capsular polysaccharides are frequently an important virulence factor for many bacterial pathogens, including Haemophilus influenzae, Streptococcus pneumoniae and Group B streptococci. In some embodiments, an immunogenic polysaccharide for use in the SA-MAPS immunogenic composition as disclosed herein is a polysaccharide or oligosaccharide from Gram-positive bacteria, for example, a Staphylococcus aureus capsular polysaccharide.

Type 5 and Type 8 Polysaccharides from S. aureus

Most strains of S. aureus that cause infection in man contain either Type 5 or Type 8 polysaccharides. Approximately 60% of human strains are Type 8 and approximately 30% are Type 5. The structures of Type 5 and Type 8 capsular polysaccharide antigens are described in Moreau et al Carbohydrate Res. 201; 285 (1990) and Fournier et al Infect. Immun. 45; 87 (1984). Both have FucNAcp in their repeat unit as well as ManNAcA which can be used to introduce a sulfhydryl group.

Recently (Jones Carbohydrate Research 340, 1097-1106 (2005)) NMR spectroscopy revised the structures of the capsular polysaccharides to:
Type 5→4)-β-D-ManNAcA-(1→4)-α-L-FucNAc(3OAc)-(1→3)-β-D-FucNAc-(1→Type 8→3)-β-D-ManNAcA(4OAc)-(1→3)-α-L-FucNAc(1→3)-α-D-FucNAc(1→

Polysaccharides may be extracted from the appropriate strain of S. aureus using methods well known to the skilled man, for instance as described in U.S. Pat. No. 6,294,177 or Infection and Immunity (1990) 58(7); 2367, Fournier et al. (1984), supra; Fournier et al. (1987) Ann. Inst. Pasteur/Microbiol. 138:561-567; US Patent Application Publication No. 2007/0141077; and Int'l Patent Application Publication No. WO 00/56357; each of which is incorporated herein by reference as if set forth in its entirety). For example, ATCC 12902 is a Type 5 S. aureus strain and ATCC 12605 is a Type 8 S. aureus strain. In addition, they can be produced using synthetic protocols. Moreover, serotype 5 or 8 capsular polysaccharide can be recombinant produced using genetic engineering procedures also known to one of ordinary skill in the art (see, Sau et al. (1997) Microbiology 143:2395-2405; and U.S. Pat. No. 6,027,925; each of which is incorporated herein by reference as if set forth in its entirety).

One S. aureus strain that can be used to obtain isolated serotype 8 capsular polysaccharide (CP8) is S. aureus R2 PFESA0286. This strain was selected by flow cytometry with rabbit anti-serotype 8 polysaccharide antibodies after cultivation of S. aureus PFESA0286 (American Type Culture Collection; Manassas, Va.: ATCC Accession No. 495:25) in Modified Frantz Broth. Two populations, R1 and R2, were observed during flow cytometry. R1 and R2 were purified and re-cultured. R2 yielded a serotype 8 capsular polysaccharide. Flow cytometric analysis showed a homogenous fluorescence intensity. As such, R2 was selected for serotype 8 capsular polysaccharide production.

One S. aureus strain that can be used to obtain isolated serotype 5 capsular polysaccharide (CP5) is S. aureus PFESA0266. This strain produces serotype 5 capsular polysaccharide during growth, and production peaks when cells are in a stationary phase. Other S. aureus type 5 or type 8 strains can be used to make the respective polysaccharides that are obtained either from established culture collections or clinical specimens.

In some embodiments, a Becker or Newman S. aureus strain can be used to obtain isolated serotype 5 capsular polysaccharide (CP5). In some embodiments, the Newman S. aureus strain can be used to obtain isolated serotype 5 capsular polysaccharide (CP5).

In some embodiments, a Becker or Newman S. aureus strain can be used to obtain isolated serotype 8 capsular polysaccharide (CP8). In some embodiments, the Becker S. aureus strain can be used to obtain isolated serotype 8 capsular polysaccharide (CP8).

Polysaccharides are of native size or alternatively may be sized, for instance by microfluidisation, ultrasonic irradiation or by chemical treatment. The invention also covers oligosaccharides derived from the type 5 and 8 polysaccharides from S. aureus.

In some embodiments, an immunogenic polysaccharide for use in the SA-MAPS complex as disclosed herein can comprises a Type 5 (CP5), or Type 8 (CP8) capsular polysaccharides (CP), or any of the polysaccharides or oligosaccharides or lipopolysaccharides from Staphylococcus aureus. In some embodiments, an immunogenic polysaccharide for use in the SA-MAPS complex as disclosed herein can comprises a capsular polysaccharide from a non-typeable (NT) SA strain, e.g., a cell wall surface antigen 336 (Type 336) or a polyribitol phosphate N-acetylglucosamine, which resembles cell wall teichoic acid. Type 336 isolates do not express capsule but do express cell surface polysaccharide or the 336 polysaccharide (336PS), which resembles S. aureus cell wall teichoic acid (Ma, J., et al., 2004. Evaluation of serotypes of Staphylococcus aureus strains used in the production of a bovine mastitis bacterin. J. Dairy. Sci. 87:178-182 14, 17; O'Brien, et al., 2000. Production of antibodies to Staphylococcus aureus serotypes 5, 8, and 336 using poly(dl-lactide-co-glycolide) microspheres. J. Dairy Sci. 83:1758-1766).

In some embodiments, an immunogenic polysaccharide for use in the SA-MAPS complex as disclosed herein can comprises a capsular polysaccharide (CP) from a methicillin-resistant S. aureus (MRSA), including hospital-acquired MRSA (HA-MRSA), or community-acquired MRSA (CA-MRSA) or any polysaccharides or oligosaccharides or lipopolysaccharides from MRSA, e.g., e.g., any one or more of a CP1, CP2, CP5, or CP8 from HA-MSSA and/or CA-MRSA. In alternative embodiments, an immunogenic polysaccharide for use in the SA-MAPS complex as disclosed herein can comprises a capsular polysaccharide (CP) from a methicillin-sensitive S. aureus (MSSA), e.g., any one or more of a CP5, or CP8 from MSSA.

In some embodiments, an immunogenic polysaccharide for use in the SA-MAPS complex as disclosed herein can comprise more than one type of polysaccharide. For example, an immunogenic polysaccharide for use in the SA-MAPS complex as disclosed herein can comprise a portion of polysaccharide A (e.g., Type 5 from SA), and the remaining portion of polysaccharide B (Type 8 from SA). The antigenic polysaccharide does not need to be from the same organism, e.g., for example an immunogenic polysaccharide for use in the SA-MAPS complex as disclosed herein can comprise a portion of polysaccharide A (e.g., Type 5 or Type 8 from SA), and the remaining portion of polysaccharide B (e.g., a pneumococcus polysaccharide or other bacterial capsular PS or noncapsular PS). There is no limit to the amount of different types of immunogenic polysaccharides which can be used in a single MAPS backbone entity. In some embodiments, where the immunogenic polysaccharide for use in the SA-MAPS complex as disclosed herein is a branched polymer, the chain polysaccharide can be polysaccharide A, and the branches can be at least 1 or at least 2 or at least 3 or more different antigenic polysaccharides.

In some embodiments, the immunogenic polysaccharide for use in the SA-MAPS complex as disclosed herein is a branched polymer. In some embodiments, an immunogenic polysaccharide for use in the SA-MAPS complex as disclosed herein is a single chain polymer.

In some embodiments, the immunogenic polysaccharide for use in the SA-MAPS complex as disclosed herein comprises at least 10 carbohydrate repeating units, or at least 20, or at least 50, or at least 75, or at least 100, or at least 150, or at least 200, or at least 250, or at least 300, or at least 350, or at least 400, or at least 450, or at least 500, or more than 500 repeating units, inclusive.

In one aspect of the invention, the immunogenic polysaccharide (PS) for use in the SA-MAPS complex as disclosed herein can have a molecular mass of <500 kDa or >500 kDa. In another aspect of the invention, the PS has a molecular mass of <70 kDa. In some embodiments, an immunogenic polysaccharide for use in the SA-MAPS complex as disclosed herein is a large molecular weight polymer, e.g., a polymer can be of an average molecular weight of between about 425-500 kDa, inclusive, for example, at least 300 kDa, or at least 350 kDa, or at least 400 kDa, or at least 425 kDa, or at least 450 kDa, or at least 500 kDa or greater than 500 kDa, inclusive, but typically less than 500 kDa. In some embodiments, an immunogenic polysaccharide for use in the SA-MAPS complex as disclosed herein can be a small molecular weight polymer, e.g., a polymer can be of an average molecular weight of between about 60 kDA to about 90 kDa, for example, at least 50 kDa, or at least 60 kDa, or at least 70 kDa, or at least 80 kDa, or at least 90 kDa, or at least 100 kDa, or greater than 100 kDa, inclusive, but generally less than about 120 kDa.

In some embodiments, the immunogenic polysaccharide for use in the SA-MAPS complex as disclosed herein is harvested and purified from a natural source; and in other embodiments, the polysaccharide is synthetic. Methods to produce synthetic polymers, including synthetic polysaccharides, are known to persons of ordinary skill and are encompassed in the compositions and methods as disclosed herein.

In some embodiments, a type 5 and/or type 8 capsular polysaccharide or oligosaccharide included in a SA-MAPS immunogenic compositions as disclosed herein has a molecular weight of between 20 kDa and 1000 kDa. In some embodiments, the type 5 and/or type 8 and/or type 1 capsular polysaccharide or oligosaccharide of a SA-MAPS immunogenic compositions as disclosed herein has a molecular weight of between 200 kDa and 5000 kDa, or a molecular weight range of between 70 kDa and 300 kDa, or a molecular weight range of between 500 kDa and 2500 kDa.

High molecular weight capsular polysaccharides are able to induce certain antibody immune responses due to a higher valence of the epitopes present on the antigenic surface. The isolation of “high molecular weight capsular polysaccharides” is contemplated for use in the compositions and methods of the present invention. In some embodiments, high molecular weight serotype 5 or 8 capsular polysaccharide can be isolated and purified ranging from 20 kDa to 1000 kDa in molecular weight. In one embodiment, high molecular weight serotype 5 or 8 capsular polysaccharide can be isolated and purified ranging from 50 kDa to 700 kDa in molecular weight, or ranging from 50 kDa to 300 kDa in molecular weight, or ranging from 70 kDa to 300 kDa, or ranging from 90 kDa to 250 kDa, or ranging from 90 kDa to 150 kDa in molecular weight, or ranging from 90 kDa to 120 kDa in molecular weight, or ranging from 80 kDa to 120 kDa in molecular weight. In some embodiments, a type 5 and/or type 8 capsular polysaccharide or oligosaccharide included in a SA-MAPS immunogenic compositions as disclosed herein has a high molecular weight of any of 70 kDa to 100 kDa in molecular weight; 70 kDa to 110 kDa in molecular weight; 70 kDa to 120 kDa in molecular weight; 70 kDa to 130 kDa in molecular weight; 70 kDa to 140 kDa in molecular weight; 70 kDa to 150 kDa in molecular weight; 70 kDa to 160 kDa in molecular weight; 80 kDa to 110 kDa in molecular weight; 80 kDa to 120 kDa in molecular weight; 80 kDa to 130 kDa in molecular weight; 80 kDa to 140 kDa in molecular weight; 80 kDa to 150 kDa in molecular weight; 80 kDa to 160 kDa in molecular weight; 90 kDa to 110 kDa in molecular weight; 90 kDa to 120 kDa in molecular weight; 90 kDa to 130 kDa in molecular weight; 90 kDa to 140 kDa in molecular weight; 90 kDa to 150 kDa in molecular weight; 90 kDa to 160 kDa in molecular weight; 100 kDa to 120 kDa in molecular weight; 100 kDa to 130 kDa in molecular weight; 100 kDa to 140 kDa in molecular weight; 100 kDa to 150 kDa in molecular weight; 100 kDa to 160 kDa in molecular weight; and similar desired molecular weight ranges. Any whole number integer within any of the above ranges is contemplated as an embodiment of the invention.

In one embodiment, the conjugate has a molecular weight of between about 50 kDa and about 5000 kDa in molecular weight. In one embodiment, the conjugate has a molecular weight of between about 200 kDa and about 5000 kDa in molecular weight. In one embodiment, the immunogenic conjugate has a molecular weight of between about 500 kDa and about 2500 kDa. In one embodiment, the immunogenic conjugate has a molecular weight of between about 500 kDa and about 2500 kDa. In one embodiment, the immunogenic conjugate has a molecular weight of between about 600 kDa and about 2800 kDa. In one embodiment, the immunogenic conjugate has a molecular weight of between about 700 kDa and about 2700 kDa. In one embodiment, the immunogenic conjugate has a molecular weight of between about 1000 kDa and about 2000 kDa; between about 1800 kDa and about 2500 kDa; between about 1100 kDa and about 2200 kDa; between about 1900 kDa and about 2700 kDa; between about 1200 kDa and about 2400 kDa; between about 1700 kDa and about 2600 kDa; between about 1300 kDa and about 2600 kDa; between about 1600 kDa and about 3000 kDa. Any whole number integer within any of the above ranges is contemplated as an embodiment of the SA-MAPS immunogenic composition as disclosed herein.

In one embodiment, the serotype 5 or 8 capsular polysaccharide has a degree of O-acetylation between 10-100%. In one embodiment, the degree of O-acetylation is between 50-100%. In one embodiment, the degree of O-acetylation is between 75-100%. In one embodiment, the immunogenic conjugate generates an antibody that is functional as measured by killing bacteria in either an animal efficacy model or via an opsonophagocytic killing assay.

Most clinical isolates of S. aureus are encapsulated with either serotypes 5 or 8. Type 5 (CP5) and type 8 (CP8) capsular polysaccharides (CPs) have similar tri-saccharide repeating units comprised of N-acetyl mannosaminuronic acid, N-acetyl L-fucosamine, and N-acetyl D-fucosamine. See Fournier, J. M. et al., Infect. Immun. 45:97-93 (1984) and Moreau, M., et al, Carbohydrate Res. 201:285-297 (1990). The two CPs, which have the same sugars, but differ in the sugar linkages and in sites of O-acetylation, each produce serologically distinct patterns of immunoreactivity. CP5 and CP8 are serologically distinct, and this can be attributed to differences in the linkages between the sugars and in the sites of O-acetylation.

In some embodiments, a type 5 and/or 8 capsular polysaccharide or oligosaccharide included in a SA-MAPS immunogenic compositions as disclosed herein can be O-acetylated. In an embodiment, the degree of O-acetylation of type 5 capsular polysaccharide or oligosaccharide is 10-100%, 20-100%, 30-100%, 40-100%, 50-100%, 60-100%, 70-100%, 80-100%, 90-100%, 50-90%, 60-90%, 70-90% or 80-90%. In an embodiment, the degree of O-acetylation of type 8 capsular polysaccharide or oligosaccharide is 10-100%, 20-100%, 30-100%, 40-100%, 50-100%, 60-100%, 70-100%, 80-100%, 90-100%, 50-90%, 60-90%, 70-90% or 80-90%. In an embodiment, the degree of O-acetylation of type 5 and type 8 capsular polysaccharides or oligosaccharides is 10-100%, 20-100%, 30-100%, 40-100%, 50-100%, 60-100%, 70-100%, 80-100%, 90-100%, 50-90%, 60-90%, 70-90% or 80-90%.

The degree of O-acetylation of the polysaccharide or oligosaccharide can be determined by any method known in the art, for example, by proton NMR (Lemercinier and Jones 1996, Carbohydrate Research 296; 83-96, Jones and Lemercinier 2002, J Pharmaceutical and Biomedical analysis 30; 1233-1247, WO 05/033148 or WO 00/56357). A further commonly used method is that described by Hestrin (1949) J. Biol. Chem. 180; 249-261.

O-acetyl groups can be removed by hydrolysis, for example by treatment with a base such as anhydrous hydrazine (Konadu et al 1994; Infect. Immun. 62; 5048-5054) or treatment with 0.1N NaOH for 1-8 hours. In order to maintain high levels of O-acetylation on type 5 and/or 8 polysaccharide or oligosaccharide, treatments which would lead to hydrolysis of the O-acetyl groups are minimized. For example, treatment at extremes of pH are minimized.

The SA-MAPS immunogenic compositions as disclosed herein comprises, of consists essentially of either type 5 or type 8 polysaccharide or a conjugate of type 5 or type 8 polysaccharide. In some embodiments, the SA-MAPS immunogenic compositions as disclosed herein comprise PNAG, or type 5 or type 8 polysaccharides from S. aureus, where each or all can be between 30% and 100% O-acetylated.

In some embodiments, the serotype 5 and/or 8 capsular polysaccharides of the SA-MAPS immunogenic composition as disclosed herein are used to generate antibodies that are functional as measured by the killing of bacteria in an animal efficacy model or an opsonophagocytic killing assay that demonstrates that the antibodies kill the bacteria. Such functionality may not be observed using an assay that monitors the generation of antibodies alone, which is not indicative of the importance of O-acetylation in efficacy.

### 1. Capsule Epidemiology

The association of particular capsule serotypes with disease is possible through monitoring of clinical isolates. Of the eight different serotypes of S. aureus identified (Karakawa and Vann (1982) only serotypes 1 and 2 are heavily encapsulated, and these are rarely isolated. See Capsular Polysaccharides of Staphylococcus aureus, p. 285-293, In J. B. Robbins, J. C. Hill and J. C. Sadoff (ed.), Seminars in infectious disease, vol. 4, Bacterial Vaccines. Thieme Stratton, Inc. New York). Surveys have shown that approximately 85-90% of S. aureus clinical isolates express CP5 or CP8 (Arbeit R D, et al., Diagn. Microbiol. Infect. Dis. (1984) April; 2(2):85-91; Karakawa W W, et al., J. Clin. Microbiol. (1985) September; 22(3):445-7; Essawi T, et al., Trop. Med. Int. Health. (1998) July; 3(7):576-83; Na'was T, et al., J. Clin. Microbiol. (1998) 36(2):414-20. Most of CP5 and CP8 non-typeable strains are genetically type 5 or type 8 containing mutations in cap5/8 locus (Cocchiaro, Gomez et al., (2006), Mol. Microbiol. February 59(3):948-960). Capsulation for some strains is lost rapidly within few passages in vitro which is due to a repressive effect of high phosphate concentration in media used in clinical diagnosis on capsule production. It was also reported that non-capsulated isolates recover capsule expression after passing through cows. See Opdebeck, J. P. et al., J. Med. Microbiol. 19:275-278 (1985). Some non-typeable strains become capsule positive under appropriate growth conditions.

### 2. CP5 and CP8 Structure

The repeat unit of both CP5 and CP8 is comprised of 2-acetamido-2-deoxy-D-mannuronic acid, 2-acetamido-2-deoxy-L-fucose and 2-acetamido-2-deoxy-D-fucose. See C. Jones et al., Carbohydr. Res. 340:1097-1106 (2005). Although CP5 and CP8 have the same sugar composition, they have been demonstrated to be immunologically distinct. They differ in glycosidic linkages and site of O-acetylation of uronic acid. Strain dependent incomplete N-acetylation of one of the FucNAc residues was observed. See Tzianabos et al., PNAS V98: 9365 (2001).

It is important that the S. aureus Capsule Polysaccharide (CP) used in the SA-MAPS immunogenic composition as disclosed herein is immunogenic. The molecular weight of the S. aureus capsule polysaccharides is an important consideration, as a high molecular weight capsule polysaccharide can induce certain antibody immune responses due to a higher valency of the epitopes present on the antigenic surface. In some embodiments, a CP8 or CP5 used in a SA-MAPS immunogenic composition as disclosed herein is a high molecular weight capsule polysaccharide type 5 and type 8.

### Poly N-Acetylated Glucosamine (PNAG)

PNAG is a polysaccharide intercellular adhesion and is composed of a polymer of β-(1→6)-linked glucosamine, optionally substituted with N-acetyl and/or O-succinyl constituents. This polysaccharide is present in both S. aureus and S. epidermidis and can be isolated from either source (Joyce et al 2003, Carbohydrate Research 338; 903; Maira-Litran et al 2002, Infect. Imun. 70; 4433). For example, PNAG may be isolated from S. aureus strain MN8m (WO 04/43407). The preparation of dPNAG is described in WO 04/43405.

The polysaccharide previously known as poly-N-succinyl-β-(1→6)-glucosamine (PNSG) was recently shown not to have the expected structure since the identification of N-succinylation was incorrect (Maira-Litran et al 2002, Infect. Imun. 70; 4433). Therefore, the polysaccharide formally known as PNSG and now found to be PNAG is also encompassed by the term PNAG.

PNAG may be of different sizes varying from over 400 kDa to between 75 and 400 kDa to between 10 and 75 kDa to oligosaccharides composed of up to 30 repeat units (of β-(1→6)-linked glucosamine, optionally substituted with N-acetyl and O-succinyl constituents). Any size of PNAG polysaccharide or oligosaccharide may be used in an immunogenic composition of the invention, for example a size of over 40 kDa can be used. Sizing may be achieved by any method known in the art, for instance by microfluidisation, ultrasonic irradiation or by chemical cleavage (WO 03/53462, EP497524, EP497525).

Size ranges of PNAG are for example 40-400 kDa, 50-350 kDa, 40-300 kDa, 60-300 kDa, 50-250 kDa and 60-200 kDa.

PNAG can have different degree of acetylation due to substitution on the amino groups by acetate. PNAG produced in vitro is almost fully substituted on amino groups (95-100%). Alternatively, a deacetylated PNAG can be used having less than 50%, 40%, 30%, 20%, 10% or 5% N-acetylation. Use of a deacetylated PNAG allows opsonic killing of Gram positive bacteria, optionally S. aureus and/or S. epidermidis (WO 04/43405). In an embodiment, the PNAG has a size between 40 kDa and 300 kDa and is deacetylated so that less than 50%, 40%, 30%, 20%, 10% or 5% of amino groups are N acetylated.

In an embodiment, the PNAG is not O-succinylated or is O-succinylated on less than 25, 20, 15, 10, 5, 2, 1 or 0.1% of residues.

The term deacetylated PNAG (dPNAG) refers to a PNAG polysaccharide or oligosaccharide in which less than 50%, 40%, 30%, 20%, 10% or 5% of the amino groups are acetylated.

As used herein, the term PNAG encompasses both acetylated and deacetylated forms of the saccharide.

In an embodiment, PNAG is deacetylated to form dPNAG, by chemically treating the native polysaccharide. For example, the native PNAG is treated with a basic solution such that the pH rises to above 10. For instance, the PNAG is treated with 0.1-5M, 0.2-4M, 0.3-3M, 0.5-2M, 0.75-1.5M or 1M NaOH, KOH or NH4OH. Treatment is for at least 10 or 30 minutes, or 1, 2, 3, 4, 5, 10, 15 or 20 hours at a temperature of 20-100, 25-80, 30-60 or 30-50 or 3545° C. dPNAG may be prepared as described in WO 04/43405.

3. S. aureus 336 Antigen

In an embodiment, the SA-MAPS immunogenic composition as disclosed herein can comprise the S. aureus 336 antigen described in U.S. Pat. No. 6,294,177, which is incorporated herein in its entirety by reference. The 336 antigen comprises β-linked hexosamine, contains no O-acetyl groups and specifically binds to antibodies to S. aureus Type 336 deposited under ATCC 55804.

In an embodiment, the 336 antigen is a polysaccharide which is of native size or alternatively may be sized, for instance by microfluidisation, ultrasonic irradiation or by chemical treatment. The invention also covers oligosaccharides derived from the 336 antigen. The 336 antigen, where included in the immunogenic composition of the invention is optionally conjugated to a carrier protein as described below or are alternatively unconjugated.

### 4. Other Immunogenic Polysaccharides

In some embodiments, an immunogenic polysaccharide for use in the SA-MAPS complex as disclosed herein a polysaccharide or oligosaccharide that is not a S. aureus polysaccharide. For example, in some embodiments an immunogenic polysaccharide for use in the SA-MAPS complex as disclosed herein can be apneumococcal polysaccharide, e.g., a capsular polysaccharide from Streptococcus pneumoniae from any of the over 93 serotypes of pneumococcus that have been identified to date, for example, including but not limited to serotypes 1, 2, 3, 4, 5, 6A, 6B, 6C, 6D, 7F, 8, 9N, 9V, 10A, 11A, 12F, 14, 15B, 17F, 18C, 19A, 19F, 20, 22F, 23F, and 33F. Additional pneumococcal serotypes may be identified and included in the present SA-MAPS immunogenic composition as described herein. More than one pneumococcal polysaccharide can be included as the polymer backbone of the present immunogenic compositions or in a vaccine comprising the present SA-MAPS composition. In some embodiments, an immunogenic polysaccharide for use in the SA-MAPS complex as disclosed herein is Type 1 capsular polysaccharide (CP1) from Streptococcus pneumoniae.

In some embodiments, an immunogenic polysaccharide for use in the SA-MAPS complex as disclosed herein comprises a polysaccharide of Streptococcus pneumoniae having a serotype selected from one or more of 1, 2, 3, 4, 5, 6A, 6B, 6C, 6D, 6E, 6F, 6G, 6H, 7A, 7B, 7C, 7F, 8, 9A, 9L, 9N, 9V, 10A, 10B, 10C, 10F, 11A, 11B, 11C, 11D, 11E, 11F, 12A, 12B, 12F, 13, 14, 15A, 15B, 15C, 15F, 16A, 16F, 17A, 17F, 18A, 18B, 18C, 18F, 19A, 19B, 19C, 19F, 20A, 20B, 21, 22A, 22F, 23A, 23B, 23F, 24A, 24B, 24F, 25A, 25F, 27, 28A, 28F, 29, 31, 32A, 32F, 33A, 33B, 33C, 33D, 33E, 33F, 34, 35A, 35B, 35C, 35F, 36, 37, 38, 39, 40, 41A, 41F, 42, 43, 44, 45, 46, 47A, 47F, and 48, as disclosed in U.S. Pat. No. 11,013,793, which is incorporated herein in its entirety by reference. In some embodiments, the immunogenic polysaccharide is a polysaccharide antigen of Streptococcus pneumoniae which comprises a polysaccharide of Streptococcus pneumoniae having a serotype selected from one or more of 1, 2, 3, 4, 5, 6A, 6B, 7F, 8, 9N, 9V, 10A, 11A, 12F, 14, 15B, 17F, 18C, 19A, 19F, 20B, 22F, 23F, and 33F, as disclosed in U.S. Pat. No. 11,013,793, which is incorporated herein in its entirety by reference.

In some embodiments, a vaccine comprises a plurality of immunogenic complexes comprising: (a) a plurality of biotinylated polysaccharide antigens, wherein the plurality comprises polysaccharide antigens of one or more of Streptococcus pneumoniae serotypes 1, 2, 3, 4, 5, 6A, 6B, 6C, 6D, 6E, 6F, 6G, 6H, 7A, 7B, 7C, 7F, 8, 9A, 9L, 9N, 9V, 10A, 10B, 10C, 10F, 11A, 11B, 11C, 11D, 11E, 11F, 12A, 12B, 12F, 13, 14, 15A, 15B, 15C, 15F, 16A, 16F, 17A, 17F, 18A, 18B, 18C, 18F, 19A, 19B, 19C, 19F, 20A, 20B, 21, 22A, 22F, 23A, 23B, 23F, 24A, 24B, 24F, 25A, 25F, 27, 28A, 28F, 29, 31, 32A, 32F, 33A, 33B, 33C, 33D, 33E, 33F, 34, 35A, 35B, 35C, 35F, 36, 37, 38, 39, 40, 41A, 41F, 42, 43, 44, 45, 46, 47A, 47F, and 48; and (b) a plurality of fusion proteins, each fusion protein comprising (i) a biotin-binding moiety; (ii) a first polypeptide antigen comprising an amino acid sequence at least 90%, at least 91%, at least 92%, at least 93%, at least 94%, at least 95%, at least 96%, at least 97%, at least 98%, at least 99%, or at least 99.5% identical to any of SEQ ID NO: 50-56 as disclosed herein, or an antigenic fragment thereof, and (iii) a second polypeptide antigen which is different to the first polypeptide antigen comprising an amino acid sequence at least 90%, at least 91%, at least 92%, at least 93%, at least 94%, at least 95%, at least 96%, at least 97%, at least 98%, at least 99%, or at least 99.5% identical to SEQ ID NO:50-56, as disclosed herein, or an antigenic fragment thereof, wherein each of the first plurality of biotinylated polysaccharide antigens is non-covalently associated with the biotin-binding moiety of one or more of the plurality of fusion proteins to form an immunogenic complex.

In some embodiments, an immunogenic polysaccharide for use in the SA-MAPS complex as disclosed herein can comprises N. meningitidis capsular polysaccharides from at least one, two, three or four of the serogroups A, C, W, W135, or Y. In some embodiments, an immunogenic polysaccharide for use in the SA-MAPS complex as disclosed herein is selected from the group consisting of: Salmonella typhi Vi capsular polysaccharide, pneumococcal capsular polysaccharides, pneumococcal cell wall polysaccharide, Haemophilus influenzae Type b (Hibb) capsular polysaccharide, Haemophili polysaccharide, Meningococcal polysaccharide, polysaccharides or oligosaccharides from Gram-positive bacteria (e.g., Staphylococcus aureus capsular polysaccharide, Bacillus anthracis polysaccharide), Streptococcus polysaccharides (e.g., Gp A and Gp B), Pseudomonas polysaccharide, fungal polysaccharides (e.g., cryptococcys polysaccharides), viral polysaccharides (e.g., glycoprotein) and other bacterial capsular or cell wall polysaccharides. In some embodiments, an immunogenic polysaccharide is selected from any of the following, dextran, Vi polysaccharide of Salmonella typhi, pneumococcal capsular polysaccharide, pneumococcal cell wall polysaccharide (CWPS), meningococcal polysaccharide, Haemophilus influenzae type b polysaccharide, or any another polysaccharide of viral, prokaryotic, or eukaryotic origin.

In some embodiments, an immunogenic polysaccharide for use in the SA-MAPS complex as disclosed herein is selected from the group consisting of: Salmonella typhi Vi capsular polysaccharides, pneumococcal capsular polysaccharides, pneumococcal cell wall polysaccharides, Haemophilus influenzae Type b (Hib) polysaccharides, Haemophili polysaccharides, Meningococcal polysaccharides, polysaccharides or oligosaccharides or lipopolysaccharides from Gram-positive bacteria (e.g., Staphylococcus aureus capsular polysaccharides, Bacillus anthracis polysaccharides), Streptococcus polysaccharides (e.g., Gp A and Gp B), Pseudomonas polysaccharides, polysaccharides or oligosaccharides or lipopolysaccharides from Gram-negative bacteria, other bacterial capsular or cell wall polysaccharides, fungal polysaccharides (e.g., cryptococcus polysaccharides), viral polysaccharides (e.g., glycoprotein), or polysaccharides derived from cancer cells.

In some embodiments, an immunogenic polysaccharide for use in the SA-MAPS complex as disclosed herein consists of or comprises an antigenic sugar moiety. For example, in some embodiments, a polysaccharide for use in the methods and immunogenic compositions as disclosed herein is a Vi polysaccharide of Salmonella typhi. The Vi capsular polysaccharide has been developed against bacterial enteric infections, such as typhoid fever. Robbins et al., 150 J. Infect. Dis. 436 (1984); Levine et al., 7 Baillieres Clin. Gastroenterol. 501 (1993). Vi is a polymer of α-1→4-galacturonic acid with an N acetyl at position C-2 and variable O-acetylation at C-3. The virulence of S. typhi correlates with the expression of this molecule. Sharma et al., 101 PNAS 17492 (2004). The Vi polysaccharide vaccine of S. typhi has several advantages: Side effects are infrequent and mild, a single dose yields consistent immunogenicity and efficacy. Vi polysaccharide may be reliably standardized by physicochemical methods verified for other polysaccharide vaccines, Vi is stable at room temperature and it may be administered simultaneously with other vaccines without affecting immunogenicity and tolerability. Azze et al., 21 Vaccine 2758 (2003).

Thus, the Vi polysaccharide of S. typhi may be cross-linked to a first affinity molecule, such as biotin, as disclosed herein, for attaching at least one antigen to the polysaccharide. In some embodiments, the antigen can be from the same or from another organism, such that the resulting immunogenic composition confers at least some level of immunity against one pathogen, or two different pathogens: if the antigen confers protection against pneumococcus, an immunogenic composition where the polymer scaffold is a Vi polysaccharide can raise an immunogenic response against both S. typhi and pneumococci. Other examples include combining sugars from encapsulated bacteria (such as meningococcus, S. aureus, pneumococcus, Hib, etc.) and tuberculosis antigens, to provide an immunogenic composition that raises an immune response against two different pathogens.

In some embodiments, a polysaccharide for use in the SA-MAPS complex as disclosed herein is a capsular polysaccharide (CP) or oligosaccharide. In some embodiments, a polysaccharide for use in the SA-MAPS complex as disclosed herein is a noncapsular polysaccharide or oligosaccharide.

Other immunogenic polysaccharide (PS) for use in the SA-MAPS complex as disclosed herein can include bacterial cell wall polysaccharides (CWPS), or carbohydrate antigens of cancers.

In some embodiments, an immunogenic polysaccharide for use in the SA-MAPS complex as disclosed herein that can serve as a backbone for one or more SA-antigens or non-SA antigen types are exemplified in Table 6:

TABLE 6
Example immunogenic polysaccharides for the SA-
MAPS backbone and associated example antigens

| | | Protein Antigens | |
|---|---|---|---|
| Polysaccharide | | Number of antigens | Antigen origins |
| Dextran | D90 (60-90KD) | Two | Staphylococcus aureus |
| | D150 (150 KD) | Three | Staphylococcus aureus |
| | D270 (270 KD) | Three | Staphylococcus aureus |
| | D500 (425-575 KD) | two; three; six | Staphylococcus aureus |
| Pneumococcal | Serotype 1 | one, two, three, five | pneumococcus, tuberculosis, |
| capsular | | | Staphylococcus aureus |
| polysaccharide | Serotype 3 | Five | pneumococcus, tuberculosis, |
| | | | Staphylococcus aureus |
| | Serotype 5 | one; two; three; five | pneumococcus, tuberculosis, |
| | | | Staphylococcus aureus |
| | Serotype 6B | Two | Pneumococcus, |
| | | | Staphylococcus aureus |
| | Serotype 7 | Three | Pneumococcus, |
| | | | Staphylococcus aureus |
| | Serotype 14 | one; two; three; five | pneumococcus, tuberculosis |
| | Serotype 19 | Three | Pneumococcus, |
| | | | Staphylococcus aureus |
| Staphylococcus aureus | Serotype 5 | one; two; three; five, | Staphylococcus aureus |
| | | six | |
| | Serotype 8 | one; two; three; five, | Staphylococcus aureus |
| | | six | |
| | PNAG | one; two; three; five, | Staphylococcus aureus |
| | | six | |
| | S. aureus 336 | one; two; three; five, | Staphylococcus aureus |
| | antigen | six | |

| Pneumococcal cell wall polysaccharide | Five | Pneumococcus, |
|---|---|---|
| | | Staphylococcus aureus |
| S. typhi Vi polysaccharide | Five | Pneumococcus, |

Staphylococcus aureus

In some embodiments, an immunogenic polysaccharide for use in the SA-MAPS complex as disclosed herein can comprise additional polymers, for example, polyethylene glycol-based polymers, poly(ortho ester) polymers, polyacryl carriers, PLGA, polyethylenimine (PEI), polyamidoamine (PAMAM) dendrimers, β-amino ester polymers, polyphosphoester (PPE), liposomes, polymerosomes, nucleic acids, phosphorothioated oligonucleotides, chitosan, silk, polymeric micelles, protein polymers, virus particles, virus-like-particles (VLPs) or other micro-particles. See, e.g., El-Sayed et al., Smart Polymer Carriers for Enhanced Intracellular Delivery of Therapeutic Molecules, 5 Exp. Op. Biol. Therapy, 23 (2005). Biocompatible polymers developed for nucleic acid delivery may be adapted for use as a backbone herein. See, e.g., BIOCOMPATIBLE POL. NUCL. ACID. DELIV. (Domb et al., eds., John Wiley & Sons, Inc. Hoboken, NJ, 2011).

For example, VLPs resemble viruses, but are non-infectious because they do not contain any viral genetic material. The expression, including recombinant expression, of viral structural proteins, such as envelope or capsid components, can result in the self-assembly of VLPs. VLPs have been produced from components of a wide variety of virus families including Parvoviridae (e.g., adeno-associated virus), Retroviridae (e.g., HIV), and Flaviviridae (e.g., Hepatitis B or C viruses). VLPs can be produced in a variety of cell culture systems including mammalian cell lines, insect cell lines, yeast, and plant cells. Recombinant VLPs are particularly advantageous because the viral component can be fused to recombinant antigens as described herein.

### E. Affinity Molecule Pairs

As disclosed herein, a key aspect of the SA-MAPS composition is the attachment of the SA antigens to the immunogenic polysaccharide. As discussed herein, a SA antigen is connected to an immunogenic polysaccharide via a complementary affinity pair. This connecting of the SA antigen to the immunogenic polysaccharide is mediated by the immunogenic polysaccharide being connected to a first affinity molecule, which associates a second (e.g., complementary) affinity molecule, which is attached to the SA antigen. An example complementary affinity pair is biotin and a biotin-binding protein, e.g. biotin and rhizavidin protein or fragment thereof.

Exemplary examples of the affinity complementary affinity pairs for use in the SA-MAPS immunogenic composition include, but without limitation, biotin binding proteins or avidin-like proteins that bind to biotin. For example, where the first affinity binding molecule is biotin (which associates with the polymer), the complementary affinity molecule can be a biotin binding protein or an avidin-like protein or a derivative thereof, e.g., but not limited to, avidin, rhizavidin, or streptavidin or variants, derivatives or functional portions thereof.

In some embodiments, the first affinity binding molecule is biotin, a biotin derivative, or a biotin mimic, for example, but not limited to, amine-PEG3-biotin (((+)-biotinylation-3-6,9-trixaundecanediamine) or a derivative or functional fragment thereof. A specific biotin mimetic has a specific peptide motif containing sequence of DXₐAX_{b}PX_{c} (SEQ ID NO: 46), or CDXₐAX_{b}PX_{c}CG (SEQ ID NO: 47), where Xₐ is R or L, X_{b} is S or T, and X_{c} is Y or W. These motifs can bind avidin and Neutravidin, but streptavidin. See, e.g., Gaj et al., 56 Prot. Express. Purif. 54 (2006). In some embodiments the first affinity binding molecule is lipoic acid or a derivative thereof, or HABA (hydroxyazobenzene-benzoic acid, or dimethyl-HABA).

The linkage of the first affinity molecule to the immunogenic polysaccharide, and the complementary affinity molecule to the SA antigen can be a non-covalent linkage, or a chemical mechanism, for instance covalent binding, affinity binding, intercalation, coordinate binding and complexation. Covalent binding provides for very stable binding, and is particularly well-suited for the present embodiments. Covalent binding can be achieved either by direct condensation of existing side chains or by the incorporation of external bridging molecules.

For example, in some embodiments, a SA antigen can be non-covalently bonded to one of the pairs in a complementary affixing pair. In alternative embodiments, an antigen can be covalently bonded or fused to one of the pairs in a complementary affixing pair. Methods for generation of fusion proteins are well known in the art, and are discussed herein.

In other embodiments, a first affinity binding molecule, e.g., biotin, is linked to the immunogenic polysaccharide by a non-covalent bond, or by a covalent bond. In some embodiments, a cross-linking reagent is used to covalently bond the first affinity binding molecule to the immunogenic polysaccharide as disclosed herein.

In some embodiments, the first affinity binding molecule, e.g., biotin, associates with the complementary affinity molecule (e.g., biotin-binding protein) by non-covalent bond association as known in the art, including, but not limited to, electrostatic interaction, hydrogen bound, hydrophobic interaction (i.e., van der Waals forces), hydrophilic interactions, and other non-covalent interactions. Other higher order interactions with intermediate moieties are also contemplated.

In some embodiments, the complementary affinity molecule is an avidin-related polypeptide. In specific embodiments, the complementary affinity molecule is rhizavidin, such as recombinant rhizavidin of SEQ ID NO: 1 or a protein having an amino acid that has at least 85% sequence identity to SEQ ID NO: 1. In particular, the recombinant rhizavidin is a modified rhizavidin that can be expressed in E. coli with a high yield. The typical yield is >30 mg per liter of E. coli culture. Rhizavidin has a lower sequence homology to egg avidin (22.4% sequence identity and 35.0% similarity) compared with other avidin-like proteins. Use of the modified rhizavidin reduces the risk of the MAPS inducing an egg-related allergic reaction in a subject. Moreover, antibody to recombinant modified rhizavidin has no apparent cross-reactivity to egg avidin (and vice versa).

Additional affinity pairs that may be useful in the methods and compositions described herein include antigen-antibody, metal/ion-metal/ion-binding protein, lipid/lipid binding protein, saccharide/saccharide binding protein, amino acid/peptide/amino acid or peptide binding protein, enzyme-substrate or enzyme-inhibitor, ligand-agonist/receptor, or biotin mimetic. When using alternative affinity pairs, alternative means of attaching the respective polymer and antigen may also be employed, such as in vitro enzymatic reactions rather than genetic fusion. More specifically, antigen-antibody affinity pair provides for a very strong and specific interaction. The antigen can be any epitope including protein, peptide, nucleic acid, lipid, poly/oligosaccharide, ion, etc. The antibody can be any type of immunoglobulin, or the Ag-binding portion of an immunoglobulin, such as a Fab fragment. Regarding metal/ion-metal/ion binding protein, examples include Ni NTA vs. histidine-tagged protein, or Zn vs. Zn binding protein. Regarding lipid/lipid binding protein, examples include cholesterol vs. cholesterol binding protein. Regarding saccharide/saccharide binding protein, examples include maltose vs. maltose binding protein, mannose/glucose/oligosaccharide vs. lectin. Enzyme-substrate/inhibitors include substrates from a wide range of substances, including protein, peptide, amino acid, lipid, sugar, or ions. The inhibitor can be the analog of the real substrate which can generally bind to the enzymes more tightly and even irreversibly. For example, trypsin vs. soy trypsin inhibitor. The inhibitor can be natural or synthetic molecule. Regarding other ligand/agonist-receptor, ligand can be from a wide range of substance, including protein, peptide, amino acid, lipid, sugar, ion, agonist can be the analog of the real ligand. Examples include the LPS vs. TLR4 interaction.

### F. Cross-Linking Reagents

Many bivalent or polyvalent linking agents are useful in coupling at least one or more affinity molecules to the immunogenic polysaccharide of the SA-MAPS immunogenic composition as disclosed herein. For example, representative coupling agents can include organic compounds such as thioesters, carbodiimides, succinimide esters, disocyanates, glutaraldehydes, diazobenzenes and hexamethylene diamines. This listing is not intended to be exhaustive of the various classes of coupling agents known in the art but, rather, is exemplary of the more common coupling agents. See Killen & Lindstrom, 133 J. Immunol. 1335 (1984); Jansen et al., 62 Imm. Rev. 185 (1982); Vitetta et al.

In some embodiments, cross-linking reagents agents described in the literature are encompassed for use in the methods, immunogenic compositions and kits as disclosed herein. See, e.g., Ramakrishnan, et al., 44 Cancer Res. 201 (1984) (describing the use of MBS (M-maleimidobenzoyl-N-hydroxysuccinimide ester)); Umemoto et al., U.S. Pat. No. 5,030,719 (describing the use of a halogenated acetyl hydrazide derivative coupled to an antibody by way of an oligopeptide linker). Particular linkers include: (a) EDC (1-ethyl-3-(3-dimethylamino-propyl) carbodiimide hydrochloride; (b) SMPT (4-succinimidyloxycarbonyl-alpha-methyl-alpha-(2-pyridyl-dithio)-toluene (Pierce Chem. Co., Cat. (21558G); (c) SPDP (succinimidyl-6 [3-(2-pyridyldithio) propionamido]hexanoate (Pierce Chem. Co., Cat #21651G); (d) Sulfo-LC-SPDP (sulfosuccinimidyl 6 [3-(2-pyridyldithio)-propianamide]hexanoate (Pierce Chem. Co. Cat. #2165-G); and (f) sulfo-NHS (N-hydroxysulfo-succinimide: Pierce Chem. Co., Cat. #24510) conjugated to EDC.

The linkages or linking agents described above contain components that have different attributes, thus leading to conjugates with differing physio-chemical properties. For example, sulfo-NHS esters of alkyl carboxylates are more stable than sulfo-NHS esters of aromatic carboxylates. NHS-ester containing linkers are less soluble than sulfo-NHS esters. Further, the linker SMPT contains a sterically hindered disulfide bond, and can form conjugates with increased stability. Disulfide linkages, are in general, less stable than other linkages because the disulfide linkage can be cleaved in vitro, resulting in less conjugate available. Sulfo-NHS, in particular, can enhance the stability of carbodimide couplings. Carbodimide couplings (such as EDC) when used in conjunction with sulfo-NHS, forms esters that are more resistant to hydrolysis than the carbodimide coupling reaction alone.

Additional cross linkers for —SH (thiolated CP) to —NH₂ linkages include but are not limited to: sulfa-LC-SMPT; sulfo-LC-SMPT (4-sulfosuccinimidyl-6-methyl-a-(2-pyridyldithio)toluamido]hexanoate)); sulfo-KMUS (N-[k-maleimidoundecanoyloxy]sulfosuccinimide ester); sulfo-LC-SPDP (sulfosuccinimidyl 6-(3′-[2-pyridyldithio]-propionamido)hexanoate) which cleaves by thiols; sulfo-SMPB (sulfosuccinimidyl 4-[p-maleimidophenyl]butyrate); sulfo-SIAB (N-sulfosuccinimidyl[4-iodoacetyl]aminobenzoate); sulfa-EMCS ([N-e-maleimidocaproyloxy]sulfosuccinimide ester); EMCA (N-e-maleimidocaproic acid); sulfo-SMCC (sulfosuccinimidyl 4-[N-maleimidomethyl]cyclohexane-1-carboxylate); sulfo-MBS (m-maleimidobenzoyl-N-hydroxysulfosuccinimide ester); sulfo-GMBS (N-[g-maleimidobutyryloxy]sulfosuccinimide ester); BMPA (N-.beta.-maleimidopropionic acid); 2-immunothiolane hydrochloride; 3-(2-pyridyldithio)propionic acid N-succinimidyl ester; 3-malemidopropionic acid N-succinimidyl ester; 4-maleimidobutyric acid N-succinimidyl ester; SMPT (4-succinimidyloxycarbonyl-methyl-a-[2-pyridyldithio]toluene); LC-SMCC (succinimidyl-4-[N-maleimidomethyl]cyclohexane-1-carboxy-[6-amidocaproate-]); KMUA (N-k-maleimidoundecanoic acid); LC-SPDP (succinimidyl 6-(3-[2-pyridyldithio]-propionamido)hexanoate); SMPH (succinimidyl-6-[.beta.-maleimidopropionamido]hexanoate); SMPB (succinimidyl 4-[p-maleimidophenyl]butyrate); SIAB (N-succinimidyl[4-iodoacetyl]aminobenzoate); EMCS ([N-e-Maleimidocaproyloxy]succinimide ester); SMCC (succinimidyl 4-[N-maleimidomethyl]cyclohexane-1-carboxylate); MBS (m-Maleimidobenzoyl-N-hydroxysuccinimide ester); SBAP (succinimidyl 3-[bromoacetamido]propionate); BMPS (N-[.beta.-maleimidopropyloxylsuccinimide ester); AMAS N-(a-maleimidoacetoxy)succinimide ester); SIA (N-succinimidyl iodoacetate); and N-succinimidyl (4-iodoacetyl)-aminobenzoate.

The agents can also be crosslinked using crosslinkers for —SH to —OH groups. Such cross linkers include but are not limited to PMPI (N-[p-maleimidophenyl]isocyanate).

Exemplary cross-linking molecules for use in the methods and immunogenic compositions as disclosed herein include, but are not limited to those listed in Tables 7A and 7B.

TABLE 7A
Exemplary homobifunctional crosslinkers*

| | Crosslinker Reactive Groups, | |
|---|---|---|
| Crosslinking Target | Features | Example Products |
| Amine-to-Amine | NHS esters | DSG; DSS; BS3; TSAT (trifunctional); |
| | | Bioconjugate Toolkit Reagent Pairs |
| | NHS esters, PEG spacer | BS(PEG)5; BS(PEG)9 |
| | NHS esters, thiol-cleavable | DSP; DTSSP |
| | NHS esters, misc-cleavable | DST; BSOCOES; EGS; Sulfo-EGS |
| | Imidoesters | DMA; DMP; DMS |
| | Imidoesters, thiol-cleavable | DTBP |
| | Other | DFDNB; THPP (trifunctional); |
| | | Aldehyde-Activated Dextran Kit |
| Sulfhydryl-to-Sulfhydryl | Maleimides | BMOE; BMB; BMH; |
| | | TMEA (trifunctional) |
| | Maleimides, PEG spacer | BM(PEG)2; BM(PEG)3 |
| | Maleimides, cleavable | BMDB; DTME |
| | Pyridyldithiols (cleavable) | DPDPB |
| | Other | HBVS (vinylsulfone) |
| Nonselective | Aryl azides | BASED (thiol-cleavable) |

*crosslinking reagents that have the same type of reactive group at either end. Reagents are classified by what chemical groups they cross link (left column) and their chemical composition (middle column). Products are listed in order of increasing length within each cell.

TABLE 7B
Exemplary heterobifunctional crosslinkers*

| | Crosslinker Reactive | |
|---|---|---|
| Crosslinking Targets | Groups, Features | Example Products |
| Amine-to-Sulfhydryl | NHS ester / Maleimide | AMAS; BMPS; GMBS and Sulfo- |
| | | GMBS; MBS and Sulfo-MBS; |
| | | SMCC and Sulfo-SMCC; EMCS and |
| | | Sulfo-EMCS; SMPB and Sulfo- |
| | | SMPB; SMPH; LC-SMCC; Sulfo- |
| | | KMUS |
| | NHS ester / Maleimide, | SM(PEG)2; SM(PEG)4; SM(PEG)6; |
| | PEG spacer | SM(PEG)8; SM(PEG)12; |
| | | SM(PEG)24 |
| | NHS ester / Pyridyldithiol, | SPDP; LC-SPDP and Sulfo-LC- |
| | cleavable | SPDP; SMPT; Sulfo-LC-SMPT |
| | NHS esters / Haloacetyl | SIA; SBAP; SIAB; Sulfo-SIAB |
| Amine-to-Nonselective | NHS ester / Aryl Azide | NHS-ASA |
| | | ANB-NOS |
| | | Sulfo-HSAB |
| | | Sulfo-NHS-LC-ASA |
| | | SANPAH and Sulfo-SANPAH |
| | NHS ester / Aryl Azide, | Sulfo-SFAD; Sulfo-SAND; Sulfo- |
| | cleavable | SAED |
| | NHS ester / Diazirine | SDA and Sulfo-SDA; LC-SDA and |
| | | Sulfo-LC-SDA |
| | NHS ester / Diazirine, | SDAD and Sulfo-SDAD |
| | cleavable | |
| Amine-to-Carboxyl | Carbodiimide | DCC; EDC |
| Sulfhydryl-to-Nonselective | Pyridyldithiol / Aryl Azide | APDP |
| Sulfhydryl-to-Carbohydrate | Maleimide / Hydrazide | BMPH; EMCH; MPBH; KMUH |
| | Pyridyldithiol / Hydrazide | BMPH; EMCH; MPBH; KMUH |
| Carbohydrate-to-Nonselective | Hydrazide / Aryl Azide | ABH |
| Hydroxyl-to-Sulfhydryl | Isocyanate / Maleimide | PMPI |
| Amine-to-DNA | NHS ester / Psoralen | SPB |

*crosslinking reagents that have the different reactive groups at either end. Reagents are classified by what chemical groups they cross link (left column) and their chemical composition (middle column).
Products are listed in order of increasing length within each cell.

### G. Co-Stimulatory Factor

In some embodiments, an immunogenic composition comprising the SA-MAPS as disclosed herein comprises at least one co-stimulatory molecule. In some embodiments, the co-stimulatory factor is cross-linked to the immunogenic polysaccharide. In some embodiments, the co-stimulatory factor is associated to the immunogenic polysaccharide by a complementary affinity pair similar to how the SA antigen is associated with the immunogenic polysaccharide. In some embodiments, where the complementary affinity pair which links the co-stimulatory factor to the immunogenic polysaccharide is the same, or a different complementary affinity pair which links the SA antigen to the immunogenic polysaccharide.

In some embodiments, at least one, or at least 2, or at least 3, or at least 5, or at least 10, or at least 15, or at least 20, or at least 50, or at least 100, or more than about 100, inclusive, co-stimulatory factors can be associated with the immunogenic polysaccharide as disclosed herein. In some embodiments, the co-stimulatory factors can be the same co-stimulator factor, or they can be a variety of different co-stimulatory factors associated with the immunogenic polysaccharide.

In some embodiments, the co-stimulator factor is a ligand/agonist of Toll like receptors, e.g., but not limited to TLR1, TLR2, TLR3, TLR4, TLR5, TLR6, TLR7, TLR8, TLR9, TLR10, TLR11, etc. In some embodiments, a co-stimulator factor is a NOD ligand/agonist, or an activator/agonist of the inflammasome. Without wishing to be bound by theory, the inflammasome is a multiprotein oligomer consisting of caspase 1, PYCARD, NALP and sometimes caspase 5 or caspase 11 and promotes the maturation of inflammatory cytokines interleukin 1-β and interleukin 18.

In some embodiments, a co-stimulator factor is a cytokine. In some embodiments, a cytokine is selected from the group consisting of: GM-CSF; IL-1α; IL-1β; IL-2; IL-3; IL-4; IL-5; IL-6; IL-7; IL-8; IL-10; IL-12; IL-23; IFN-α; IFN-β; IFN-γ; MIP-1α; MIP-1β; TGF-β; TNFα, and TNFβ. In some embodiments, the co-stimulatory factor is an adjuvant, which may be associated with the polymer, as just discussed, or may be added to the MAPS composition prior to or concurrent with administration to a subject. Adjuvants are further described elsewhere herein.

### H. Production of SA Antigens and SA Antigens Fused to the Complementary Affinity Molecule

Recombinant proteins may be conveniently expressed and purified by a person skilled in the art, or by using commercially available kits, for example PROBOND™ Purification System (Invitrogen Corp., Carlsbad, CA). In some embodiments, recombinant antigens can be synthesized and purified by protein purification methods using bacterial expression systems, yeast expression systems, baculovirus/insect cell expression system, mammalian cell expression systems, or transgenic plant or animal systems as known to persons of ordinary skill in the art.

The fusion polypeptides as described herein, e.g., a SA antigen fused to a rhizavidin protein of SEQ ID NO: 1 (e.g., Rhavi-Hla209(27-319), Rhavi-ClfA(221-559), Rhavi-ClfB (203-542), Rhavi-SdrD (246-682), Rhavi-IsdA (47-324), Rhavi-IsdB (48-447)) can all be synthesized and purified by protein and molecular methods that are well known to one skilled in the art. Molecular biology methods and recombinant heterologous protein expression systems are used. For example, recombinant protein can be expressed in bacteria, mammalian, insect, yeast, or plant cells; or in transgenic plant or animal hosts.

In one embodiment, provided herein is an isolated polynucleotide encoding a fusion polypeptide or a non-fusion polypeptide described herein. Conventional polymerase chain reaction (PCR) cloning techniques can be used to construct a chimeric or fusion coding sequence encoding a fusion polypeptide as described herein. A coding sequence can be cloned into a general purpose cloning vector such as pUC19, pBR322, pBLUESCRIPT® vectors (Stratagene, Inc.) or pCR TOPO® (Invitrogen). The resultant recombinant vector carrying the nucleic acid encoding a polypeptide as described herein can then be used for further molecular biological manipulations such as site-directed mutagenesis to create a variant fusion polypeptide as described herein or can be subcloned into protein expression vectors or viral vectors for protein synthesis in a variety of protein expression systems using host cells selected from the group consisting of mammalian cell lines, insect cell lines, yeast, bacteria, and plant cells.

Each PCR primer should have at least 15 nucleotides overlapping with its corresponding templates at the region to be amplified. The polymerase used in the PCR amplification should have high fidelity such as PfuULTRA® polymerase (Stratagene) for reducing sequence mistakes during the PCR amplification process. For ease of ligating several separate PCR fragments together, for example in the construction of a fusion polypeptide, and subsequently inserting into a cloning vector, the PCR primers should also have distinct and unique restriction digestion sites on their flanking ends that do not anneal to the DNA template during PCR amplification. The choice of the restriction digestion sites for each pair of specific primers should be such that the fusion polypeptide coding DNA sequence is in-frame and will encode the fusion polypeptide from beginning to end with no stop codons. At the same time the chosen restriction digestion sites should not be found within the coding DNA sequence for the fusion polypeptide. The coding DNA sequence for the intended polypeptide can be ligated into cloning vector pBR322 or one of its derivatives, for amplification, verification of fidelity and authenticity of the chimeric coding sequence, substitutions/or specific site-directed mutagenesis for specific amino acid mutations and substitutions in the polypeptide.

Alternatively, the coding DNA sequence for the polypeptide can be PCR cloned into a vector using for example, the TOPO® cloning method comprising topoisomerase-assisted TA vectors such as pCR©-TOPO, pCR®-Blunt II-TOPO, pENTR/D-TOPO®, and pENTR/SD/D-TOPO® (Invitrogen, Inc., Carlsbad, CA). Both pENTR/D-TOPO®, and pENTR/SD/D-TOPO® are directional TOPO entry vectors which allow the cloning of the DNA sequence in the 5′→3′ orientation into a GATEWAY® expression vector. Directional cloning in the 5′→3′ orientation facilitates the unidirectional insertion of the DNA sequence into a protein expression vector such that the promoter is upstream of the 5′ ATG start codon of the fusion polypeptide coding DNA sequence, enabling promoter driven protein expression. The recombinant vector carrying the coding DNA sequence for the fusion polypeptide can be transfected into and propagated in general cloning E. coli such as XL1Blue, SURE® (STRATAGENE®) and TOP-10 cells (Invitrogen).

One skilled in the art would be able to clone and ligate the coding region of the SA polypeptide antigen of interest with the coding region of the complementary affinity molecule (e.g., biotin-binding protein, e.g., rhavi) to construct a chimeric coding sequence for a fusion polypeptide comprising the antigen or a fragment thereof and the complementary affinity molecule of a derivative thereof using specially designed oligonucleotide probes and polymerase chain reaction (PCR) methodologies that are well known in the art. One skilled in the art would also be able to clone and ligate the chimeric coding sequence for a fusion protein into a selected vector, e.g., bacterial expression vector, an insect expression vector or baculovirus expression vector. The coding sequences of antigen and the target antigen polypeptide or fragment thereof should be ligated in-frame and the chimeric coding sequence should be ligated downstream of the promoter, and between the promoter and the transcription terminator. Subsequent to that, the recombinant vector is transfected into regular cloning E. coli, such as XL1Blue. Recombinant E. coli harboring the transfer vector DNA is then selected by antibiotic resistance to remove any E. coli harboring non-recombinant plasmid DNA. The selected transformant E. coli are grown and the recombinant vector DNA can be subsequently purified for transfection into S. frugiperda cells.

In some embodiments, the SA antigens as disclosed herein can comprise a signal peptide for translocation into periplasmic space of bacteria. The signal peptide is also called a leader peptide in the N-terminus, which may or may not be cleaved off after the translocation through the membrane. One example of a signal peptide is MKKIWLALAGLVLAFSASA (SEQ ID NO: 23) as disclosed herein. Another signal sequence is MAPFEPLASGILLLLWLIAPSRA (SEQ ID NO: 48). Other examples of signal peptides can be found at SPdb, a Signal Peptide Database, which is found at the world wide web site of “proline.bic.nus.edu.sg/spdb/”.

In some embodiments, where the antigen is fused to the C-terminal of complementary affinity protein (e.g., biotin-binding protein, e.g., rhavi), the signal sequence can be located at the N-terminal of the complementary affinity protein. For example, if an antigen is fused to an avidin-like protein, the signal sequence can be located at the N-terminal of the complementary affinity protein. In some embodiments, where the antigen is fused to the N-terminal of complementary affinity protein (e.g., biotin-binding protein, e.g., rhavi), e.g., the N-terminal of the biotin-binding protein is attached the C-terminal of the antigen, the signal sequence can be located at the N-terminal of the SA polypeptide antigen. In some embodiments, the signal sequence is cleaved off from the complementary affinity protein before the complementary affinity protein associates with the first affinity molecule.

In some embodiments, a SA antigen and/or complementary affinity protein (e.g., biotin-binding protein, e.g., rhavi), as described herein lacks a signal sequence.

The fusion polypeptides as described herein can be expressed in a variety of expression host cells e.g., bacteria, yeasts, mammalian cells, insect cells, plant cells, algal cells such as Chlamadomonas, or in cell-free expression systems. In some embodiments the nucleic acid can be subcloned from the cloning vector into a recombinant expression vector that is appropriate for the expression of fusion polypeptide in bacteria, mammalian, insect, yeast, or plant cells or a cell-free expression system such as a rabbit reticulocyte expression system. Some vectors are designed to transfer coding nucleic acid for expression in mammalian cells, insect cells and year in one single recombination reaction. For example, some of the GATEWAY® (Invitrogen) destination vectors are designed for the construction of baculovirus, adenovirus, adeno-associated virus (AAV), retrovirus, and lentiviruses, which upon infecting their respective host cells, permit heterologous expression of fusion polypeptides in the appropriate host cells. Transferring a gene into a destination vector is accomplished in just two steps according to manufacturer's instructions. There are GATEWAY® expression vectors for protein expression in insect cells, mammalian cells, and yeast. Following transformation and selection in E. coli, the expression vector is ready to be used for expression in the appropriate host.

Examples of other expression vectors and host cells are the strong CMV promoter-based pcDNA3.1 (Invitrogen) and pCINEO vectors (Promega) for expression in mammalian cell lines such as CHO, COS, HEK-293, Jurkat, and MCF-7; replication incompetent adenoviral vector vectors pADENO-X™, pAd5F35, pLP-ADENO™-X-CMV (CLONTECH®), pAd/CMV/V5-DEST, pAd-DEST vector (Invitrogen) for adenovirus-mediated gene transfer and expression in mammalian cells; pLNCX2, pLXSN, and pLAPSN retrovirus vectors for use with the RETRO-X™ system from Clontech for retroviral-mediated gene transfer and expression in mammalian cells; pLenti4/V5-DEST™, pLenti6/V5-DEST™, and pLenti6.2/V5-GW/lacZ (Invitrogen) for lentivirus-mediated gene transfer and expression in mammalian cells; adenovirus-associated virus expression vectors such as pAAV-MCS, pAAV-IRES-hrGFP, and pAAV-RC vector (Stratagene) for adeno-associated virus-mediated gene transfer and expression in mammalian cells; BACpak6 baculovirus (Clontech) and pFASTBAC™ HT (Invitrogen) for the expression in S. frugiperda 9 (Sf9), Sf11, Tn-368 and BTI-TN-5B4-1 insect cell lines; pMT/BiP/V5-His (Invitrogen) for the expression in Drosophila schneider S2 cells; Pichia expression vectors pPICZa, pPICZ, pFLDa and pFLD (Invitrogen) for expression in P. pastoris and vectors pMETa and pMET for expression in P. methanolica; pYES2/GS and pYD1 (Invitrogen) vectors for expression in yeast S. cerevisiae.

Recent advances in the large scale expression heterologous proteins in Chlamydomonas reinhardtii are described. Griesbeck., 34 Mol. Biotechnol. 213 (2006); Fuhrmann, 94 Methods Mol Med. 191 (2006). Foreign heterologous coding sequences are inserted into the genome of the nucleus, chloroplast and mitochondria by homologous recombination. The chloroplast expression vector p64 carrying the most versatile chloroplast selectable marker aminoglycoside adenyl transferase (aadA), which confer resistance to spectinomycin or streptomycin, can be used to express foreign protein in the chloroplast. The biolistic gene gun method can be used to introduce the vector in the algae. Upon its entry into chloroplasts, the foreign DNA is released from the gene gun particles and integrates into the chloroplast genome through homologous recombination.

Other aspects of the invention are directed to fusion proteins comprising a complementary affinity molecule (e.g., biotin-binding protein, e.g., rhavi) fused to a SA-antigen as disclosed herein. In some embodiments, the fusion construct can also optionally comprise purification tags, and/or secretion signal peptides. These fusion proteins may be produced by any standard method. For example, for production of a stable cell line expressing an antigen-complementary affinity molecule fusion protein, PCR-amplified antigen nucleic acids may be cloned into the restriction site of a derivative of a mammalian expression vector. For example, KA, which is a derivative of pcDNA3 (Invitrogen) contains a DNA fragment encoding an influenza virus hemagglutinin tag (HA). Alternatively, vector derivatives encoding other tags, such as c-myc or poly Histidine tags, can be used. The antigen-complementary affinity molecule fusion expression construct may be co-transfected, with a marker plasmid, into an appropriate mammalian cell line (e.g., COS, HEK293T, or NIH 3T3 cells) using, for example, LIPOFECTAMINE™ (Gibco-BRL, Gaithersburg, MD) according to the manufacturer's instructions, or any other suitable transfection technique known in the art. Suitable transfection markers include, for example, β-galactosidase or green fluorescent protein (GFP) expression plasmids or any plasmid that does not contain the same detectable marker as the antigen-complementary affinity molecule fusion protein. The fusion protein expressing cells can be sorted and further cultured, or the tagged antigen-complementary affinity molecule fusion protein can be purified. In some embodiments, an antigen-complementary affinity molecule fusion protein is amplified with a signal peptide. In alternative embodiments, a cDNA encoding an antigen-complementary affinity molecule fusion protein can be amplified without the signal peptide and subcloned into a vector (pSecTagHis) having a strong secretion signal peptide. In another example, antigen-complementary affinity molecule fusion protein can have an alkaline phosphatase (AP) tag, or a histadine (His) tag for purification. Any method known to persons of ordinary skill in the art for protein purification of the antigen and/or antigen-complementary affinity molecule fusion protein is encompassed for use in the methods of the invention.

In some embodiments, any of the polypeptides described herein is produced by expression from a recombinant baculovirus vector. In another embodiment, any of the polypeptides described herein is expressed by an insect cell. In yet another embodiment, any of the polypeptides described herein is isolated from an insect cell. There are several benefits of protein expression with baculovirus in insect cells, including high expression levels, ease of scale-up, production of proteins with posttranslational modifications, and simplified cell growth. Insect cells do not require CO₂ for growth and can be readily adapted to high-density suspension culture for large-scale expression. Many of the post-translational modification pathways present in mammalian systems are also utilized in insect cells, allowing the production of recombinant protein that is antigenically, immunogenically, and functionally similar to the native mammalian protein.

Baculoviruses are DNA viruses in the family Baculoviridae. These viruses are known to have a narrow host-range that is limited primarily to Lepidopteran species of insects (butterflies and moths). The baculovirus Autographa californica Nuclear Polyhedrosis Virus (AcNPV), which has become the prototype baculovirus, replicates efficiently in susceptible cultured insect cells. AcNPV has a double-stranded closed circular DNA genome of about 130,000 base-pairs and is well characterized with regard to host range, molecular biology, and genetics. The Baculovirus Expression Vector System (BEVS) is a safe and rapid method for the abundant production of recombinant proteins in insect cells and insects. Baculovirus expression systems are powerful and versatile systems for high-level, recombinant protein expression in insect cells. Expression levels up to 500 mg/l have been reported using the baculovirus expression system, making it an ideal system for high-level expression. Recombinant baculoviruses that express foreign genes are constructed by way of homologous recombination between baculovirus DNA and chimeric plasmids containing the gene sequence of interest. Recombinant viruses can be detected by virtue of their distinct plaque morphology and plaque-purified to homogeneity.

Recombinant fusion proteins described herein can be produced in insect cells including, but not limited to, cells derived from the Lepidopteran species S. frugiperda. Other insect cells that can be infected by baculovirus, such as those from the species Bombyx mori, Galleria mellanoma, Trichplusia ni, or Lamanthria dispar, can also be used as a suitable substrate to produce recombinant proteins described herein. Baculovirus expression of recombinant proteins is well known in the art. See U.S. Pat. Nos. 4,745,051; 4,879,236; 5,179,007; 5,516,657; 5,571,709; 5,759,809. It will be understood by those skilled in the art that the expression system is not limited to a baculovirus expression system. What is important is that the expression system directs the N-glycosylation of expressed recombinant proteins. The recombinant proteins described herein can also be expressed in other expression systems such as Entomopox viruses (the poxviruses of insects), cytoplasmic polyhedrosis viruses (CPV), and transformation of insect cells with the recombinant gene or genes constitutive expression. A good number of baculovirus transfer vectors and the corresponding appropriately modified host cells are commercially available, for example, pAcGP67, pAcSECG2TA, pVL1392, pVL1393, pAcGHLT, and pAcAB4 from BD Biosciences; pBAC-3, pBAC-6, pBACgus-6, and pBACsurf-1 from NOVAGEN®, and pPolh-FLAG and pPolh-MAT from SIGMA ALDRICH®.

The region between the promoter and the transcriptional terminator can have multiple restriction enzyme digestion sites for facilitating cloning of the foreign coding sequence, in this instance, the coding DNA sequence for an antigen polypeptide, and a complementary affinity molecule (e.g., biotin-binding protein, e.g., rhavi). Additional sequences can be included, e.g., signal peptides and/or tag coding sequences, such as His-tag, MAT-Tag, FLAG tag, recognition sequence for enterokinase, honeybee melittin secretion signal, beta-galactosidase, glutathione S-transferase (GST) tag upstream of the MCS for facilitating the secretion, identification, proper insertion, positive selection of recombinant virus, and/or purification of the recombinant protein.

Standard techniques known to those of skill in the art can be used to introduce mutations (to create amino acid substitutions in an antigen polypeptide sequence of the fusion polypeptide described herein, e. g., in the antigen in the nucleotide sequence encoding the fusion polypeptide described herein, including, for example, site-directed mutagenesis and PCR-mediated mutagenesis. Preferably, the variant fusion polypeptide has less than 50 amino acid substitutions, less than 40 amino acid substitutions, less than 30 amino acid substitutions, less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions, inclusive, relative to the fusion polypeptides described herein.

Certain silent or neutral missense mutations can also be made in the DNA coding sequence that do not change the encoded amino acid sequence or the capability to promote transmembrane delivery. These types of mutations are useful to optimize codon usage, or to improve recombinant protein expression and production.

Specific site-directed mutagenesis of a coding sequence for the fusion polypeptide in a vector can be used to create specific amino acid mutations and substitutions. Site-directed mutagenesis can be carried out using, e. g., the QUICKCHANGE® site-directed mutagenesis kit from Stratagene according to the manufacturer's instructions.

In one embodiment, described herein are expression vectors comprising the coding DNA sequence for the polypeptides described herein for the expression and purification of the recombinant polypeptide produced from a protein expression system using host cells selected from, e.g., bacteria, mammalian, insect, yeast, or plant cells. The expression vector should have the necessary 5′ upstream and 3′ downstream regulatory elements such as promoter sequences, ribosome recognition and TATA box, and 3′ UTR AAUAAA transcription termination sequence for efficient gene transcription and translation in its respective host cell. The expression vector is, preferably, a vector having the transcription promoter selected from a group consisting of CMV (cytomegalovirus) promoter, RSV (Rous sarcoma virus) promoter, β-actin promoter, SV40 (simian virus 40) promoter and muscle creatine kinase promoter, and the transcription terminator selected from a group consisting of SV40 poly(A) and BGH terminator; more preferably, an expression vector having the early promoter/enhancer sequence of cytomegalovirus and the adenovirus tripartite leader/intron sequence and containing the replication orgin and poly(A) sequence of SV40. The expression vector can have additional coding regions, such as those encoding, for example, 6×-histidine SEQ ID NO: 32), V5, thioredoxin, glutathione-S-transferase, c-Myc, VSV-G, HSV, FLAG, maltose binding peptide, metal-binding peptide, HA and “secretion” signals (Honeybee melittin, D α-factor, PHO, Bip), which can be incorporated into the expressed fusion polypeptide. In addition, there can be enzyme digestion sites incorporated after these coding regions to facilitate their enzymatic removal if they are not needed. These additional nucleic acids are useful for the detection of fusion polypeptide expression, for protein purification by affinity chromatography, enhanced solubility of the recombinant protein in the host cytoplasm, and/or for secreting the expressed fusion polypeptide out into the culture media or the spheroplast of the yeast cells. The expression of the fusion polypeptide can be constitutive in the host cells or it can be induced, e.g., with copper sulfate, sugars such as galactose, methanol, methylamine, thiamine, tetracycline, infection with baculovirus, and (isopropyl-beta-D-thiogalactopyranoside) IPTG, a stable synthetic analog of lactose.

In another embodiment, the expression vector comprising a polynucleotide described herein is a viral vector, such as adenovirus, adeno-associated virus (AAV), retrovirus, and lentivirus vectors, among others. Recombinant viruses provide a versatile system for gene expression studies and therapeutic applications.

In some embodiments, the fusion polypeptides described herein are expressed from viral infection of mammalian cells. The viral vectors can be, for example, adenovirus, adeno-associated virus (AAV), retrovirus, and lentivirus. A simplified system for generating recombinant adenoviruses is presented by He et al., 95 PNAS 2509 (1998). The gene of interest is first cloned into a shuttle vector, e.g., pAdTrack-CMV. The resultant plasmid is linearized by digesting with restriction endonuclease PmeI, and subsequently cotransformed into E. coli. BJ5183 cells with an adenoviral backbone plasmid, e.g. pADEASY-1 of Stratagene's ADEASY™ Adenoviral Vector System. Recombinant adenovirus vectors are selected for kanamycin resistance, and recombination confirmed by restriction endonuclease analyses. Finally, the linearized recombinant plasmid is transfected into adenovirus packaging cell lines, for example HEK 293 cells (El-transformed human embryonic kidney cells) or 911 (El-transformed human embryonic retinal cells). Fallaux, et al. 7 Human Gene Ther. 215 (1996). Recombinant adenovirus are generated within the HEK 293 cells.

Recombinant lentivirus has the advantage of delivery and expression of fusion polypeptides in dividing and non-dividing mammalian cells. The HIV-1 based lentivirus can effectively transduce a broader host range than the Moloney Leukemia Virus (MoMLV)-based retroviral systems. Preparation of the recombinant lentivirus can be achieved using, for example, the pLenti4/V5-DEST™, pLenti6/V5-DEST™ or pLenti vectors together with VIRAPOWER™ Lentiviral Expression systems from Invitrogen, Inc.

Recombinant adeno-associated virus (rAAV) vectors are applicable to a wide range of host cells including many different human and non-human cell lines or tissues. rAAVs are capable of transducing a broad range of cell types and transduction is not dependent on active host cell division. High titers, >10⁸ viral particle/ml, are easily obtained in the supernatant and 10¹¹-10¹² viral particle/ml with further concentration. The transgene is integrated into the host genome so expression is long term and stable.

Large scale preparation of AAV vectors is made by a three-plasmid cotransfection of a packaging cell line: AAV vector carrying the coding nucleic acid, AAV RC vector containing AAV rep and cap genes, and adenovirus helper plasmid pDF6, into 50×150 mm plates of subconfluent 293 cells. Cells are harvested three days after transfection, and viruses are released by three freeze-thaw cycles or by sonication.

AAV vectors can be purified by two different methods depending on the serotype of the vector. AAV2 vector is purified by the single-step gravity-flow column purification method based on its affinity for heparin. Auricchio et. al., 12 Human Gene Ther. 71 (2001); Summerford & Samulski, 72 J. Virol. 1438 (1998); Summerford & Samulski, 5 Nat. Med. 587 (1999). AAV2/1 and AAV2/5 vectors are currently purified by three sequential CsCl gradients.

Without wishing to be bound to theory, when proteins are expressed by a cell, including a bacterial cell, the proteins are targeted to a particular part in the cell or secreted from the cell. Thus, protein targeting or protein sorting is the mechanism by which a cell transports proteins to the appropriate positions in the cell or outside of it. Sorting targets can be the inner space of an organelle, any of several interior membranes, the cell's outer membrane, or its exterior via secretion. This delivery process is camed out based on information contained in the protein itself. Correct sorting is crucial for the cell; errors can lead to diseases.

With some exceptions, bacteria lack membrane-bound organelles as found in eukaryotes, but they may assemble proteins onto various types of inclusions such as gas vesicles and storage granules. Also, depending on the species of bacteria, bacteria may have a single plasma membrane (Gram-positive bacteria), or both an inner (plasma) membrane and an outer cell wall membrane, with an aqueous space between the two called the periplasm (Gram-negative bacteria). Proteins can be secreted into the environment, according to whether or not there is an outer membrane. The basic mechanism at the plasma membrane is similar to the eukaryotic one. In addition, bacteria may target proteins into or across the outer membrane. Systems for secreting proteins across the bacterial outer membrane may be quite complex and play key roles in pathogenesis. These systems may be described as type I secretion, type II secretion, etc.

In most Gram-positive bacteria, certain proteins are targeted for export across the plasma membrane and subsequent covalent attachment to the bacterial cell wall. A specialized enzyme, sortase, cleaves the target protein at a characteristic recognition site near the protein C-terminus, such as an LPXTG motif (SEQ ID NO: 19) (where X can be any amino acid), then transfers the protein onto the cell wall. A system analogous to sortase/LPXTG (SEQ ID NO: 19), having the motif PEP-CTERM (SEQ ID NO: 49), termed exosortase/PEP-CTERM SEQ ID NO: 49), is proposed to exist in a broad range of Gram-negative bacteria.

Proteins with appropriate N-terminal targeting signals are synthesized in the cytoplasm and then directed to a specific protein transport pathway. During, or shortly after its translocation across the cytoplasmic membrane, the protein is processed and folded into its active form. Then the translocated protein is either retained at the periplasmic side of the cell or released into the environment. Since the signal peptides that target proteins to the membrane are key determinants for transport pathway specificity, these signal peptides are classified according to the transport pathway to which they direct proteins. Signal peptide classification is based on the type of signal peptidase (SPase) that is responsible for the removal of the signal peptide. The majority of exported proteins are exported from the cytoplasm via the general “Secretory (Sec) pathway”. Most well known virulence factors (e.g. exotoxins of Staphylococcus aureus, protective antigen of Bacillus anthracis, lysteriolysin 0 of Listeria monocytogenes) that are secreted by Gram-positive pathogens have a typical N-terminal signal peptide that would lead them to the Sec-pathway. Proteins that are secreted via this pathway are translocated across the cytoplasmic membrane in an unfolded state. Subsequent processing and folding of these proteins takes place in the cell wall environment on the trans-side of the membrane. In addition to the Sec system, some Gram-positive bacteria also contain the Tat-system that is able to translocate folded proteins across the membrane. Pathogenic bacteria may contain certain special purpose export systems that are specifically involved in the transport of only a few proteins. For example, several gene clusters have been identified in mycobacteria that encode proteins that are secreted into the environment via specific pathways (ESAT-6) and are important for mycobacterial pathogenesis. Specific ATP-binding cassette (ABC) transporters direct the export and processing of small antibacterial peptides called bacteriocins. Genes for endolysins that are responsible for the onset of bacterial lysis are often located near genes that encode for holin-like proteins, suggesting that these holins are responsible for endolysin export to the cell wall. Wooldridge, BACT. SECRETED PROTS: SECRETORY MECHS. & ROLE IN PATHOGEN. (Caister Academic Press, 2009)

In some embodiments, the signal sequence useful in the present invention is OmpA Signal sequence, however any signal sequence commonly known by persons of ordinary skill in the art which allows the transport and secretion of antimicrobial agents outside the bacteriophage infected cell are encompassed for use in the present invention.

Signal sequence that direct secretion of proteins from bacterial cells are well known in the art, for example as disclosed in International application WO 2005/071088. For example, one can use some of the non-limited examples of signal peptide shown in Table 8, which can be attached to the amino-terminus or carboxyl terminus of the antimicrobial peptide (Amp) or antimicrobial polypeptide to be expressed by the antimicrobial-agent engineered bacteriophage, e.g., AMP-engineered bacteriophage. Attachment can be via fusion or chimera composition with selected antigen or antigen-complementary affinity molecule fusion protein ((e.g., fusion protein comprising a biotin-binding protein, e.g., Rhavi and a SA-polypeptide antigen as disclosed herein), resulting in the secretion from the bacterium infected with the antimicrobial-agent engineered bacteriophage, e.g. AMP-engineered bacteriophage.

TABLE 8
Example signal peptides to direct secretion of a SA-polypeptide antigen or fusion
protein as disclosed herein from a bacterial cell

| Secretion | Signal Peptide Amino Acid | | |
|---|---|---|---|
| Pathway | sequence (NH₂-CO₂) | Gene | Genus/Species |
| secA1 | MKKIMLVITLILVSPIAQQTEAK | Hly (LLO) | Listeria monocytogenes |
| | D (SEQ ID NO: 50) | | |
| | MKKKIISAILMSTVILSAAAPLSG | Usp45 | Lactococcus lactis |
| | VYADT (SEQ ID NO: 73) | | |
| | MKKRKVLIPLMALSTILVSSTGN | Pag (protective | Bacillus anthracis |
| | LEVIQAEV (SEQ ID NO: 74) | antigen) | |
| secA2 | MNMKKATIAATAGIAVTAFAAP | Iap (invasion- | Listeria monocytogenes |
| | TIASAST (SEQ ID NO: 75) | associated protein | |
| | | p60) | |
| | MQKTRKERILEALQEEKKNKKS | NamA Imo2691 | Listeria monocytogenes |
| | KKFKTGATIAGVTAIATSITVPGI | (autolysin) | |
| | EVIVSADE (SEQ ID NO: 76) | | |
| | MKKLKMASCALVAGLMFSGLT | *BA_0281 | Bacillus anthracis |
| | PNAFAED (SEQ ID NO: 77) | (NLP/P60 family) | |
| | MAKKFNYKLPSMVALTLVGSA | *atl (autolysin) | Staphylococcus aureus |
| | VTAHQVQAAE (SEQ ID NO: 78) | | |
| Tat | MTDKKSENQTEKTETKENKGM | Imo0367 | Listeria monocytogenes |
| | TRREMLKLSAVAGTGIAVGATG | | |
| | LGTILNVVDQVDKALT (SEQ ID | | |
| | NO: 79) | | |
| | MAYDSRFDEWVQKLKEESFQN | PhoD (alkaline | Bacillus subtillis |
| | NTFDRRKFIQGAGKIAGLGLGLT | phosphatase) | |
| | IAQSVGAFG (SEQ ID NO: 80) | | |

The polypeptides as described herein, e.g., a SA-polypeptide antigen or a fusion protein as disclosed herein can be expressed and purified by a variety methods known to one skilled in the art, for example, the fusion polypeptides described herein can be purified from any suitable expression system. Fusion polypeptides can be purified to substantial purity by standard techniques, including selective precipitation with such substances as ammonium sulfate; column chromatography, immunopurification methods, and others; which are well-known in the art. See, e.g., Scopes, PROTEIN PURIFICATION: PRINCIPLES & PRACTICE (1982); U.S. Pat. No. 4,673,641.

A number of procedures can be employed when recombinant proteins are purified. For example, proteins having established molecular adhesion properties can be reversibly fused to the protein of choice. With the appropriate ligand, the protein can be selectively adsorbed to a purification column and then freed from the column in a relatively pure form. The fused protein is then removed by enzymatic activity. Finally, the protein of choice can be purified using affinity or immunoaffinity columns.

After the protein is expressed in the host cells, the host cells can be lysed to liberate the expressed protein for purification. Methods of lysing the various host cells are featured in “Sample Preparation-Tools for Protein Research” EMD Bioscience and in the Current Protocols in Protein Sciences (CPPS). An example purification method is affinity chromatography such as metal-ion affinity chromatograph using nickel, cobalt, or zinc affinity resins for histidine-tagged fusion polypeptides. Methods of purifying histidine-tagged recombinant proteins are described by Clontech using their TALON® cobalt resin and by NOVAGEN® in their pET system manual, 10th edition. Another preferred purification strategy is immuno-affinity chromatography, for example, anti-myc antibody conjugated resin can be used to affinity purify myc-tagged fusion polypeptides. When appropriate protease recognition sequences are present, fusion polypeptides can be cleaved from the histidine or myc tag, releasing the fusion polypeptide from the affinity resin while the histidine-tags and myc-tags are left attached to the affinity resin.

Standard protein separation techniques for purifying recombinant and naturally occurring proteins are well known in the art, e.g., solubility fractionation, size exclusion gel filtration, and various column chromatography.

Solubility fractionation: Often as an initial step, particularly if the protein mixture is complex, an initial salt fractionation can separate many of the unwanted host cell proteins (or proteins derived from the cell culture media) from the protein of interest. The preferred salt is ammonium sulfate. Ammonium sulfate precipitates proteins by effectively reducing the amount of water in the protein mixture. Proteins then precipitate on the basis of their solubility. The more hydrophobic a protein is, the more likely it is to precipitate at lower ammonium sulfate concentrations. A typical protocol includes adding saturated ammonium sulfate to a protein solution so that the resultant ammonium sulfate concentration is between 20-30%. This concentration will precipitate the most hydrophobic of proteins. The precipitate is then discarded (unless the protein of interest is hydrophobic) and ammonium sulfate is added to the supernatant to a concentration known to precipitate the protein of interest. The precipitate is then solubilized in buffer and the excess salt removed if necessary, either through dialysis or diafiltration. Other methods that rely on solubility of proteins, such as cold ethanol precipitation, are well known to those of skill in the art and can be used to fractionate complex protein mixtures.

Size exclusion filtration: The molecular weight of the protein of choice can be used to isolate it from proteins of greater and lesser size using ultrafiltration through membranes of different pore size (for example, AMICON® or MILLIPORE® membranes). As a first step, the protein mixture is ultrafiltered through a membrane with a pore size that has a lower molecular weight cut-off than the molecular weight of the protein of interest. The retentate of the ultrafiltration is then ultrafiltered against a membrane with a molecular cut off greater than the molecular weight of the protein of interest. The recombinant protein will pass through the membrane into the filtrate. The filtrate can then be chromatographed as described below.

Column chromatography: The protein of choice can also be separated from other proteins on the basis of its size, net surface charge, hydrophobicity, and affinity for ligands. In addition, antibodies raised against recombinant or naturally occurring proteins can be conjugated to column matrices and the proteins immunopurified. All of these methods are well known in the art. It will be apparent to one of skill that chromatographic techniques can be performed at any scale and using equipment from many different manufacturers (e.g., Pharmacia Biotech). For example, an antigen polypeptide can be purified using a PA63 heptamer affinity column. Singh et al., 269, J. Biol. Chem. 29039 (1994).

In some embodiments, a combination of purification steps comprising, for example: (a) ion exchange chromatography, (b) hydroxyapatite chromatography, (c) hydrophobic interaction chromatography, and (d) size exclusion chromatography can be used to purify the fusion polypeptides described herein.

Cell-free expression systems are also contemplated. Cell-free expression systems offer several advantages over traditional cell-based expression methods, including the easy modification of reaction conditions to favor protein folding, decreased sensitivity to product toxicity and suitability for high-throughput strategies such as rapid expression screening or large amount protein production because of reduced reaction volumes and process time. The cell-free expression system can use plasmid or linear DNA. Moreover, improvements in translation efficiency have resulted in yields that exceed a milligram of protein per milliliter of reaction mix. Commercially available cell-free expression systems include the TNT coupled reticulocyte lysate Systems (Promega) which uses rabbit reticulocyte-based in vitro system.

### I. Determining the Efficacy of a SA-MAPS Immunogenic Composition:

The effectiveness of a SA-MAPS immunogenic composition as disclosed herein can be measured either by proliferation assays, by cytolytic assays, such as chromium release assays to measure the ability of a T-cell to lyse its specific target cell, or by measuring the levels of B-cell activity by measuring the levels of circulating antibodies specific for the antigen in serum. An immune response may also be detected by measuring the serum levels of antigen specific antibody induced following administration of the antigen, and more specifically, by measuring the ability of the antibodies so induced to enhance the opsonophagocytic ability of particular white blood cells, as described herein. The level of protection of the immune response may be measured by challenging the immunized host with the antigen that has been administered. For example, if the antigen to which an immune response is desired is a bacterium, the level of protection induced by the immunogenic amount of the antigen is measured by detecting the percent survival or the percent mortality after challenge of the animals with the bacterial cells. In one embodiment, the amount of protection may be measured by measuring at least one symptom associated with the bacterial infection, e.g., a fever associated with the infection. The amount of each of the antigens in the multi-antigen or multi-component vaccine or immunogenic compositions will vary with respect to each of the other components and can be determined by methods known to the skilled artisan. Such methods would include procedures for measuring immunogenicity and/or in vivo efficacy. In certain embodiments, the term “about” leans within 20%, preferably within 10%, and more preferably within 5%.

In some embodiments, the invention further provides antibodies and antibody compositions which bind specifically and selectively to the SA-MAPS immunogenic composition as disclosed herein. In some embodiments, antibodies are generated upon administration of a SA-MAPS immunogenic composition as disclosed herein to a subject. In some embodiments, the antibodies of the present invention are functional as measured by killing bacteria in either an animal efficacy model or via an opsonophagocytic killing assay. In some embodiments, the antibodies of the invention confer passive immunity to a subject. The present invention further provides polynucleotide molecules encoding an antibody or antibody fragment of the invention, and a cell, cell line (such as hybridoma cells or other engineered cell lines for recombinant production of antibodies) or a transgenic animal that produces an antibody or antibody composition of the invention, using techniques well-known to those of skill in the art.

Antibodies or antibody compositions of the invention may be used in a method of treating or preventing a Staphylococcal infection, disease or condition associated with a Staphylococcus sp. in a subject, the method comprising generating a polyclonal or monoclonal antibody preparation, and using said antibody or antibody composition to confer passive immunity to the subject. Antibodies of the invention may also be useful for diagnostic methods, e.g., detecting the presence of or quantifying the levels of CP5, CP8 or a conjugate thereof.

Several animal models known in the art may be used to assess the efficacy of any one of the SA-MAPS immunogenic composition as disclosed herein. For example:

Passive Murine Sepsis Model: Mice are passively immunized intraperitoneally (i.p.) with SA-MAPS immunogenic composition as disclosed herein. The mice are challenged 24 hours later with a lethal dose of S. aureus. The bacterial challenge is administered intravenously (i.v. or i.p.) ensuring that any survival could be attributed to the specific in vivo interaction of the antibody with the bacteria. The bacterial challenge dose is determined to be the dose required to achieve lethal sepsis of approximately 20% of the un-immunized control mice. Statistical evaluation of survival studies can be carried out by Kaplan-Meier analysis.

Active Immunization and Challenge Model: In this model, mice are actively immunized subcutaneously (s.c.) with a SA-MAPS immunogenic composition as disclosed herein at 0, 2 and 4 weeks (or a similar schedule known to those skilled in the art) and challenged with S. aureus at week 8 (or other similar schedule known to those skilled in the art) by the intravenous or intraperitoneal route. The bacterial challenge dose is calibrated to achieve approximately 20% survival in the control group over a 14-day period. Statistical evaluation of survival studies can be carried out by Kaplan-Meier analysis.

Passive Infectious Endocarditis Model: A passive immunization model for infectious endocarditis (IE) caused by S. aureus has previously been used to show that ClfA can induce protective immunity. See, Vernachio et al. (2006) Antmicro. Agents & Chemo. 50:511-518. In this model of IE, rabbits or rats are used to simulate clinical infections that include a central venous catheter, bacteremia, and hematogenous seeding to distal organs. Catheterized rabbits or rats with sterile aortic valve vegetations are administered a SA-MAPS immunogenic composition as disclosed herein. After 24 hours, the animals are challenged i.v. with heterologous staphylococcal strains or a MRSA strain. Then 48 hours after challenge, cardiac vegetations, kidneys and blood are harvested and cultured. The frequency of staphylococcal infection in cardiac valve vegetations, kidneys, and blood is then measured. In one study, when animals were challenged with either MRSE ATCC 35984 or MRSA PFESA0003, significant reductions in infection rate were shown using either the polyclonal antibody preparation or the monoclonal antibody to ClfA. See, Vernachio et al., supra.

Passive Infectious Endocarditis Model: The infectious endocarditis model has also been adapted for active immunization studies. Rabbits or rats are immunized intramuscularly (i.m.) with a SA-MAPS immunogenic composition as disclosed herein and challenged with S. aureus two weeks later via the i.v. route.

Pyelonephritis Model: In the pyelonephritis model, mice are immunized on weeks 0, 3 and 6 (or a similar schedule known to those skilled in the art) with a SA-MAPS immunogenic composition as disclosed herein. On week 8, the animals are challenged by, e.g., i.p. injection of, e.g., 1.7×10⁸ cfu S. aureus PFESA0266. After 48 hours, the kidneys and/or other tissues are harvested and cultured. Finally, colony forming units of challenge bacteria are enumerated in the kidneys and/or other tissues. This model evaluates systemic dissemination in the animal.

Monitoring Functional Antibodies Using Opsonophagocytic Killing Assays: Differentiated effector cells from a cell line (e.g. HL60s) or polymorphonuclear cells (PMNs) isolated from donor human blood using LYMPHOLYTE™-poly solation (Cedarlane laboratories limited, Ontario, Canada) as per manufacturer's protocol can be used for this assay. Effector cells were resuspended in assay buffer (Modified Eagle's media containing 1% bovine serum albumin) at approximately 2×10⁷ cells/ml concentration and placed in 37° C. incubator until ready to use. S. aureus strain PFESA0266 was grown overnight on tryptic soy agar plates. Bacterial cells were scraped, washed twice and resuspended in assay buffer containing 5% glycerol to an OD⁶⁰⁰=, which equals to approximately 5.times.10.sup.8 cfu/ml concentration. One ml aliquots of the bacterial suspension were frozen and stored at −40° C. until ready to use. Frozen bacterial suspension were thawed and adjusted to a concentration of 10×10⁶ cfu/ml in assay buffer and placed on ice. The assay was performed using a sterile 96 deep well 1 ml polypropylene plates. Two fold serial dilutions of antibody samples (50 μl) were prepared and followed by addition of 300 μl of assay buffer to the antibody mix. Bacteria were added (50 μl) to the plates and placed on a rotary shaker at 4° C. for 30 minutes. The opsonization step was followed by addition of 50 μl of human complement (1% final concentration). Finally, 50 μl of effector cells (10×10⁷ cells/ml concentration) were added to the plate and the suspension mixed well by repeated pipetting. A 50 μl aliquot of the suspension was 10 fold serially diluted in sterile 1% saponin solution, vortexed to minimize bacterial clumping and plated on tryptic soy agar in duplicate. The assay plate was incubated at 37° C. for 1 hour with continuous mixing using rotisserie style shaker. At the end of the incubation a 50 μl aliquot of suspension was 10 fold serially diluted in sterile 1% saponin solution, mixed by vortexing to minimize bacterial clumping and plated on tryptic soy agar in duplicate. The percentage killing was calculated by determining the ratio of the number of cfu surviving at 60 minutes in wells with bacteria, antibodies, complement and effector cells to the number of cfu surviving in tubes lacking antibodies but containing bacteria, complement and effector cells. Controls containing bacteria, complement, and sera were included to adjust for any reduction in cfu due to clumping.

Complement Adsorption: Serum from human donors adsorbed against S. aureus strains PFESA0266, PFESA0286 and PFESA0270 can be used as a source of complement in the assay. S. aureus strains were grown overnight on TSA plates at 37° C. Cells were scraped from the plate and resuspended in sterile PBS. Bacterial cells were centrifuged at 10,000 rpm for 10 minutes at 4° C. and cell pellet was resuspended in human serum for adsorption. Serum was incubated with bacteria on a nutator at 4° C. for 30 minutes. Cells were centrifuged, serum transferred to another tube containing bacteria and the adsorption step repeated again for 30 minutes. Finally, the cells were centrifuged and the serum passed through a 0.2 micron filter before 0.5 ml aliquots were frozen down in liquid nitrogen.

Method II—OPA Using HL-60 Cells: HL-60 cells were differentiated according to S. Romero-Steiner, et al., Clin Diagn Lab Immunol 4 (4) (1997), pp. 415-422. Harvested HL-60 cells were resuspended in assay buffer (Modified Eagle's media containing 1% bovine serum albumin) at approximately 10.sup.8 cells/nil and placed in 37° C. incubator until ready to use. S. aureus was grown overnight on tryptic soy agar plates. Bacterial cells were scraped, washed twice and resuspended in assay buffer containing 5% glycerol to an OD⁶⁰⁰⁼¹, which equals to approximately 5×10⁸ cfu/ml. One ml aliquots of the bacterial suspension were frozen and stored at −40° C. until ready to use. Frozen bacterial suspension were thawed and adjusted to a concentration of 10×10⁶ cfu/ml in assay buffer and placed on ice. The assay was performed using a sterile 96 deep well 1 ml polypropylene plates. Two fold serial dilutions of monoclonal antibody samples (25 μl) were prepared and followed by addition of 150 μl of assay buffer to the antibody suspension. Bacteria were added (25 μl) to the plates and placed on a rotary shaker at 4° C. for 30 minutes followed by addition of 25 μl of human complement (1% final concentration). Finally, 25 μl of HL-60 cells (10×10⁷ cells/ml) were added to the plate and the suspension mixed well by repeated pipetting. A 25 μl aliquot of the suspension was 10 fold serially diluted in sterile 1% saponin solution, mixed by vortexing to minimize bacterial clumping and plated on tryptic soy agar in duplicates. The assay plate was incubated at 37° C. for 1 hour with continuous mixing using rotisserie style shaker. At the end of incubation a 25 μl aliquot of suspension was 10 fold serially diluted in sterile 1% saponin solution, mixed by vortexing to and plated on tryptic soy agar in duplicate. The percentage killing was calculated by determining the ratio of the number of cfu surviving at 60 minutes in wells with bacteria, antibodies, complement and HL-60 cells to the number of cfu surviving in tubes lacking antibodies but containing bacteria, complement and HL-60 cells. Controls containing bacteria complement and mAb was included to adjust for any reduction in cfu due to clumping.

### J. Formulations of an Immune Composition and Methods of Use

Specific embodiments of the present invention provide for use of the SA-MAPS immunogenic compositions as disclosed herein to elicit an immune response to S. aureus in an animal or human. More specifically, the compositions elicit both humoral and cellular immunity, and in many instances mucosal immunity. Embodiments of the present invention provide at least partial protection from or partial improvement after infection by, in particular, S. aureus.

In one embodiment, provided herein is a method of vaccinating a mammal, including a human, comprising administering the SA-MAPS immunogenic composition comprising at least one, or multiple SA antigens attached to an immunogenic polysaccharide for use in eliciting an immune response to the one or more antigens attached to the polymer when administered to a subject. In some embodiments, the immune response is a humoral and/or cellular immune response.

In one embodiment, the methods as disclosed herein are directed to using the SA-MAPS immunogenic composition as disclosed herein to administer to a subject for prevention of an infection with S. aureus. In some embodiments, the compostions and methods as disclosed herein can be used as a vaccine to prevent infection with any of: methicillin-resistant Staphylococcus aureus (MRSA), methicillin-susceptible Staphylococcus aureus (MSSA), vancomycin-intermediate Staphylococcus aureus (VISA), vancomycin-resistant Staphylococcus aureus (VRSA).

In some embodiments, the compostions and methods as disclosed herein can be used as a vaccine to prevent infection S. aureus and the prevention of any of S. aureus infection that lead to any of: Pneumonia (including ventilator-associated pneumonia), sinusitis, skin infections/abscess/cellulitis/furuncles/impetigo, bloodstream infection, meningitis, endocarditis (heart infection), osteomyelitis, septic arthritis (both could be summarized as bone/joint infections) myositis (muscle infection), mastitis, mastoiditis, toxic shock syndrome, necrotizing fasciitis catheter/central line infection, prosthetic material infection including joint prostheses, surgical site infections. In some embodiments, the SA-MAPS immunogenic composition as disclosed can be used to administer to a subject with a disease or disorder selected from: Pneumonia (including ventilator-associated pneumonia), sinusitis, skin infections/abscess/cellulitis/furuncles/impetigo, bloodstream infection, meningitis, endocarditis (heart infection), osteomyelitis, septic arthritis (both could be summarized as bone/joint infections) myositis (muscle infection), mastitis, mastoiditis, toxic shock syndrome, necrotizing fasciitis catheter/central line infection, prosthetic material infection including joint prostheses, surgical site infections as a result of infection with S. aureus.

Accordingly, one aspect of the present invention relates to methods to elicit an immune response in a subject, comprising administering to the subject a SA-MAPS immunogenic composition comprising at least one type of immunogenic polysaccharide (e.g., CP5, CP8, a CP5-CP8 conjugate, pneumococcal PS1(CP 1) etc., at least one SA antigen, and at least one complementary affinity-molecule pair comprising (i) a first affinity molecule which associates with the immunogenic polysaccharide, and (ii) a complementary affinity molecule which associates with the SA antigen, to attach the SA antigen to the immunogenic polysaccharide, (e.g., the first affinity molecule associates with the complementary affinity molecule to link the SA antigen to the immunogenic polysaccharide). That is, one aspect of the present invention relates to a method to elicit an immune response in a subject, comprising administering to the subject a SA-MAPS immunogenic composition comprising at least one type of immunogenic polysaccharide (e.g., s. aureus CP5, CP8, a CP5-CP8 conjugate, pneumococcal PS1(CP 1) etc., at least one SA antigen from Group A disclosed herein, where SA-MAPS immunogenic composition comprises (i) a biotinylated immunogenic polysaccharide and (ii) at least one fusion protein, the fusion protein comprising a biotin-binding protein fused to at least one SA-antigen as disclosed herein, e.g., at least one Group A SA-antigen as disclosed herein, and where the biotinylated polysaccharide antigen is non-covalently associated with the biotin-binding moiety of the fusion protein to form an immunogenic complex. In some embodiments, the immunogenic polysaccharide is selected from any of: type 1 capsular polysaccharide of Streptococcus pneumoniae, a type 5 capsular polysaccharide of S. aureus, and/or a type 8 capsular polysaccharide of S. aureus, or CP5-CP8 conjugate capsular polysaccharide of S. aureus.

Accordingly, one aspect of the present invention relates to methods to elicit a humoral and/or cellular immunity to multiple SA antigens at the same time, e.g., where the immunogenic composition administered to the subject comprises an immunogenic polysaccharide comprising at least 1, or at least 2, or a more, e.g., a plurality of the same or different SA antigens.

One aspect of the present invention relates to a method of immunization or vaccinating a subject, e.g., a bird or a mammal, e.g., a human against S. aureus comprising administering a SA-MAPS immune composition as disclosed herein comprising at least one SA antigen derived from one or more pathogens. In some embodiments, a subject can be immunized against at least 1, or at least 2, or at least 2, or at least 3, or at least 5, or at least 10, or at least 15, or at least about 20, or at least 50, or at least about 100, or more than 100 different SA antigens at the same time, where the immunogenic polysaccharide of the SA-MAPS immunogenic composition has different SA antigens attached.

In some embodiments, a subject can be administered several different SA-MAPS immunogenic compositions as disclosed herein, for example, a subject can be administered a SA-MAPS composition comprising an immunogenic polysaccharide with a SA antigen, or a plurality of SA antigens, e.g., antigens A, B, C, and D etc., and also administered a SA-MAPS composition comprising an immunogenic polysaccharide comprising a different SA antigen, or a different set of SA antigens, e.g., antigens W, X, Y, and Z etc. Alternatively, a subject can be administered a SA-MAPS composition comprising a immunogenic polysaccharide A (e.g., CP5) with an SA antigen, or a plurality of SA antigens, e.g., antigens A, B, C, and D, etc., and also administered a SA-MAPS composition comprising a immunogenic polysaccharide B (e.g. CP8) comprising the same e.g., antigens A, B, C, and D etc., or a different set of antigens. It is envisioned that the present invention provides a method for the immunization of a subject with as many SA antigens as desired, e.g., with a variety of different immunogenic complexes as described herein, to enable immunization with as many as 100 or more antigens.

Another aspect relates to a vaccine comprising an immunogenic complex, wherein the immunogenic complex comprises: (a) a biotinylated polysaccharide antigen, and (b) a fusion protein comprising (i) a biotin-binding moiety, such as, e.g., SEQ ID NO: 1 or a polypeptide having at least 85% sequence identity to SEQ ID NO: 1, and at least one SA-antigen selected from any of: SEQ ID NO: 50 (SA1739; B1), SEQ ID NO: 51 (SA1720; B2), SEQ ID NO: 52 (SA1890; B3), SEQ ID NO: 53 (SA0103; T1), SEQ ID NO: 54 (SA0377; T2), SEQ ID NO: 55 (SA0693; T3), SEQ ID NO: 56 (SA2105; T4), or a polypeptide having at least 80% or at least 85% sequence homology thereto, where the biotinylated polysaccharide antigen is non-covalently associated with the biotin-binding moiety of the fusion protein in the form of an immunogenic complex. In some embodiments, the biotinylated polysaccharide antigen comprises any polysaccharide disclosed in Table 6. In some embodiments, the biotinylated polysaccharide antigen comprises any polysaccharide from S. aureus, including, but not limited to CP1, CP2, CP5, CP8, PNAG or S. aureus 336 antigen. In some embodiments, the biotinylated polysaccharide antigen comprises a polysaccharide of Streptococcus pneumoniae having a serotype selected from 1, 2, 3, 4, 5, 6A, 6B, 6C, 6D, 6E, 6F, 6G, 6H, 7A, 7B, 7C, 7F, 8, 9A, 9L, 9N, 9V, 10A, 10B, 10C, 10F, 11A, 11B, 11C, 11D, 11E, 11F, 12A, 12B, 12F, 13, 14, 15A, 15B, 15C, 15F, 16A, 16F, 17A, 17F, 18A, 18B, 18C, 18F, 19A, 19B, 19C, 19F, 20A, 20B, 21, 22A, 22F, 23A, 23B, 23F, 24A, 24B, 24F, 25A, 25F, 27, 28A, 28F, 29, 31, 32A, 32F, 33A, 33B, 33C, 33D, 33E, 33F, 34, 35A, 35B, 35C, 35F, 36, 37, 38, 39, 40, 41A, 41F, 42, 43, 44, 45, 46, 47A, 47F, and 48.

Another aspect of the technology disclosed herein relates to a multivalent S. aureus vaccine comprising 4 our more species of immunogenic complexes, wherein each species of the immunogenic complex comprises: (a) a biotinylated polysaccharide antigen comprising biotin and a polysachharide antigen from either a distinct S. aureus serotype, or a Streptococcus pneumoniae serotype, and (b) a fusion protein comprising (i) a biotin-binding moiety, such as, e.g., SEQ ID NO: 1 or a polypeptide having at least 85% sequence identity to SEQ ID NO: 1, and at least one SA-antigen selected from any of: SEQ ID NO: 50 (SA1739; B1), SEQ ID NO: 51 (SA1720; B2), SEQ ID NO: 52 (SA1890; B3), SEQ ID NO: 53 (SA0103; T1), SEQ ID NO: 54 (SA0377; T2), SEQ ID NO: 55 (SA0693; T3), SEQ ID NO: 56 (SA2105; T4), or a polypeptide having at least 80% or at least 85% sequence homology thereto, wherein in each species of the immunogenic complex, the biotinylated polysaccharide antigen is non-covalently associated with the biotin-binding moiety of the fusion protein in the form of an immunogenic complex. In such a multivalent vaccine comprising 4- or more species of immunogenic complexes, the biotinylated S. aureus polysaccharide antigen is selected fromany of CP1, CP2, CP5, CP8, PNAG or S. aureus 336 antigen. In some embodiments, the Streptococcus pneumoniae biotinylated polysaccharide antigen is selected from a serotype selected from 1, 2, 3, 4, 5, 6A, 6B, 6C, 6D, 6E, 6F, 6G, 6H, 7A, 7B, 7C, 7F, 8, 9A, 9L, 9N, 9V, 10A, 10B, 10C, 10F, 11A, 11B, 11C, 11D, 11E, 11F, 12A, 12B, 12F, 13, 14, 15A, 15B, 15C, 15F, 16A, 16F, 17A, 17F, 18A, 18B, 18C, 18F, 19A, 19B, 19C, 19F, 20A, 20B, 21, 22A, 22F, 23A, 23B, 23F, 24A, 24B, 24F, 25A, 25F, 27, 28A, 28F, 29, 31, 32A, 32F, 33A, 33B, 33C, 33D, 33E, 33F, 34, 35A, 35B, 35C, 35F, 36, 37, 38, 39, 40, 41A, 41F, 42, 43, 44, 45, 46, 47A, 47F, and 48.

Another aspect of the technology disclosed herein relates to a S. aureus vaccine comprising:
(i) a first immunogenic complex comprising (a) a biotinylated polysaccharide antigen comprising biotin and a polysachharide antigen from S. aureus serotype 1 (CP1), and (b) a fusion protein;
(ii) a second immunogenic complex comprising (a) a biotinylated polysaccharide antigen comprising biotin and a polysachharide antigen from S. aureus serotype 2 (CP2), and (b) a fusion protein;
(iii) a third immunogenic complex comprising (a) a biotinylated polysaccharide antigen comprising biotin and a polysachharide antigen from S. aureus serotype 5 (CP5), and (b) a fusion protein;
(iv) a fourth immunogenic complex comprising (a) a biotinylated polysaccharide antigen comprising biotin and a polysachharide antigen from S. aureus serotype 8 (CP8), and (b) a fusion protein; wherein each fusion protein comprises (i) a biotin-binding moiety, such as, e.g., SEQ ID NO: 1 or a polypeptide having at least 85% sequence identity to SEQ ID NO: 1, and at least one SA-antigen selected from any of: SEQ ID NO: 50 (SA1739; B1), SEQ ID NO: 51 (SA1720; B2), SEQ ID NO: 52 (SA1890; B3), SEQ ID NO: 53 (SA0103; T1), SEQ ID NO: 54 (SA0377; T2), SEQ ID NO: 55 (SA0693; T3), SEQ ID NO: 56 (SA2105; T4), or a polypeptide having at least 80% or at least 85% sequence homology thereto, wherein in each species of the immunogenic complex, the biotinylated polysaccharide antigen is non-covalently associated with the biotin-binding moiety of the fusion protein in the form of an immunogenic complex.

Another aspect of the technology disclosed herein relates to a S. aureus vaccine comprising: (i) a first immunogenic complex comprising (a) a biotinylated polysaccharide antigen comprising biotin and a polysaccharide antigen from Streptococcus pneumoniae serotype 1, and (b) a fusion protein; (ii) a second immunogenic complex comprising (a) a biotinylated polysaccharide antigen comprising biotin and a polysaccharide antigen from Streptococcus pneumoniae serotype 2, and (b) a fusion protein; (iii) a third immunogenic complex comprising (a) a biotinylated polysaccharide antigen comprising biotin and a polysaccharide antigen from Streptococcus pneumoniae serotype 3, and (b) a fusion protein; (iv) a fourth immunogenic complex comprising (a) a biotinylated polysaccharide antigen comprising biotin and a polysaccharide antigen from Streptococcus pneumoniae serotype 4, and (b) a fusion protein; (v) a fifth immunogenic complex comprising (a) a biotinylated polysaccharide antigen comprising biotin and a polysaccharide antigen from Streptococcus pneumoniae serotype 5, and (b) a fusion protein; (vi) a sixth immunogenic complex comprising (a) a biotinylated polysaccharide antigen comprising biotin and a polysaccharide antigen from Streptococcus pneumoniae serotype 6A, and (b) a fusion protein; (vii) a seventh immunogenic complex comprising (a) a biotinylated polysaccharide antigen comprising biotin and a polysaccharide antigen from Streptococcus pneumoniae serotype 6B, and (b) a fusion protein; (viii) an eighth immunogenic complex comprising (a) a biotinylated polysaccharide antigen comprising biotin and a polysaccharide antigen from Streptococcus pneumoniae serotype 7F, and (b) a fusion protein; (ix) a ninth immunogenic complex comprising (a) a biotinylated polysaccharide antigen comprising biotin and a polysaccharide antigen from Streptococcus pneumoniae serotype 8, and (b) a fusion protein; a tenth immunogenic complex comprising (a) a biotinylated polysaccharide antigen comprising biotin and a polysaccharide antigen from Streptococcus pneumoniae serotype 9N, and (b) a fusion protein; an eleventh immunogenic complex comprising (a) a biotinylated polysaccharide antigen comprising biotin and a polysaccharide antigen from Streptococcus pneumoniae serotype 9V, and (b) a fusion protein; a twelfth immunogenic complex comprising (a) a biotinylated polysaccharide antigen comprising biotin and a polysaccharide antigen from Streptococcus pneumoniae serotype 10A, and (b) a fusion protein; a thirteenth immunogenic complex comprising (a) a biotinylated polysaccharide antigen comprising biotin and a polysaccharide antigen from Streptococcus pneumoniae serotype 11A, and (b) a fusion protein; a fourteenth immunogenic complex comprising (a) a biotinylated polysaccharide antigen comprising biotin and a polysaccharide antigen from Streptococcus pneumoniae serotype 12F, and (b) a fusion protein; a fifteenth immunogenic complex comprising (a) a biotinylated polysaccharide antigen comprising biotin and a polysaccharide antigen from Streptococcus pneumoniae serotype 14, and (b) a fusion protein; a sixteenth immunogenic complex comprising (a) a biotinylated polysaccharide antigen comprising biotin and a polysaccharide antigen from Streptococcus pneumoniae serotype 15B, and (b) a fusion protein; a seventeenth immunogenic complex comprising (a) a biotinylated polysaccharide antigen comprising biotin and a polysaccharide antigen from Streptococcus pneumoniae serotype 17F, and (b) a fusion protein; an eighteenth immunogenic complex comprising (a) a biotinylated polysaccharide antigen comprising biotin and a polysaccharide antigen from Streptococcus pneumoniae serotype 18C, and (b) a fusion protein; a nineteenth immunogenic complex comprising (a) a biotinylated polysaccharide antigen comprising biotin and a polysaccharide antigen from Streptococcus pneumoniae serotype 19A, and (b) a fusion protein; a twentieth immunogenic complex comprising (a) a biotinylated polysaccharide antigen, comprising biotin and a polysaccharide antigen from Streptococcus pneumoniae serotype 19F, and (b) a fusion protein; a twenty-first immunogenic complex comprising (a) a biotinylated polysaccharide antigen comprising biotin and a polysaccharide antigen from Streptococcus pneumoniae serotype 20B, and (b) a fusion protein; a twenty-second immunogenic complex comprising (a) a biotinylated polysaccharide antigen comprising biotin and a polysaccharide antigen from Streptococcus pneumoniae serotype 22F, and (b) a fusion protein; a twenty-third immunogenic complex comprising (a) a biotinylated polysaccharide antigen comprising biotin and a polysaccharide antigen from Streptococcus pneumoniae serotype 23F and (b) a fusion protein; and a twenty-fourth immunogenic complex comprising (a) a biotinylated polysaccharide antigen comprising biotin and a polysaccharide antigen from Streptococcus pneumoniae serotype 33F, and (b) a fusion protein; wherein each fusion protein comprises (i) a biotin-binding moiety, such as, e.g., SEQ ID NO: 1 or a polypeptide having at least 85% sequence identity to SEQ ID NO: 1, and at least one SA-antigen selected from any of: SEQ ID NO: 50 (SA1739; B1), SEQ ID NO: 51 (SA1720; B2), SEQ ID NO: 52 (SA1890; B3), SEQ ID NO: 53 (SA0103; T1), SEQ ID NO: 54 (SA0377; T2), SEQ ID NO: 55 (SA0693; T3), SEQ ID NO: 56 (SA2105; T4), or a polypeptide having at least 80% or at least 85% sequence homology thereto, wherein in each species of the immunogenic complex, the biotinylated polysaccharide antigen is non-covalently associated with the biotin-binding moiety of the fusion protein in the form of an immunogenic complex.

In some embodiments, a SA-MAPS immunogenic compositions as described herein can be administered with other vaccines, e.g., vaccines for other bacteria that cause disease in hospitalized settings such as Gram-negatives (Klebsiella, Pseudomonas, E. coli, Acinetobacter, Enterobacter, Citrobacter, Serratia, other enteric Gram-negative bacteria) and also Clostridium difficile.

In one embodiment, the SA-MAPS immunogenic compositions as described herein comprise a pharmaceutically acceptable carrier. In another embodiment, the SA-MAPS immunogenic composition described herein is formulated for administering to a bird, mammal, or human, or as a vaccine. Suitable formulations can be found in, for example, Remington's Pharmaceutical Sciences (2006), or Introduction to Pharmaceutical Dosage Forms (4th ed., Lea & Febiger, Philadelphia, 1985).

In one embodiment, the SA-MAPS immunogenic compositions as described herein comprise pharmaceutically acceptable carriers that are inherently nontoxic and nontherapeutic. Examples of such carriers include ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts, or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances, and polyethylene glycol. For all administrations, conventional depot forms are suitably used. Such forms include, for example, microcapsules, nano-capsules, liposomes, plasters, inhalation forms, nose sprays, sublingual tablets, and sustained release preparations. For examples of sustained release compositions, see U.S. Pat. No. 3,773,919, 3,887,699, EP 58,481A, EP 158,277A, Canadian Patent No. 1176565; Sidman et al., 22 Biopolymers 547 (1983); Langer et al., 12 Chem. Tech. 98 (1982). The proteins will usually be formulated at a concentration of about 0.1 mg/ml to 100 mg/ml per application per patient.

In one embodiment, other ingredients can be added to vaccine formulations, including antioxidants, e.g., ascorbic acid; low molecular weight (less than about ten residues) polypeptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, mannose, or dextrins; chelating agents such as EDTA; and sugar alcohols such as mannitol or sorbitol.

In some embodiments, the SA-MAPS immunogen composition as disclosed herein is administered with at least one adjuvant or an immune modulator, or both. Adjuvants are a heterogeneous group of substances that enhance the immunological response against an antigen that is administered simultaneously. In some instances, adjuvants improve the immune response so that less vaccine is needed. Adjuvants serve to bring the antigen—the substance that stimulates the specific protective immune response—into contact with the immune system and influence the type of immunity produced, as well as the quality of the immune response (magnitude or duration). Adjuvants can also decrease the toxicity of certain antigens; and provide solubility to some vaccine components. Almost all adjuvants used today for enhancement of the immune response against antigens are particles or form particles together with the antigen. In the book VACCINE DESIGN—SUBUNIT & ADJUVANT APPROACH (Powell & Newman, Eds., Plenum Press, 1995), many known adjuvants are described both regarding their immunological activity and regarding their chemical characteristics. The type of adjuvants that do not form particles are a group of substances that act as immunological signal substances and that under normal conditions consist of the substances that are formed by the immune system as a consequence of the immunological activation after administration of particulate adjuvant systems.

Adjuvants for immunogenic compositions and vaccines are well known in the art. Examples include, but not limited to, monoglycerides and fatty acids (e. g. a mixture of mono-olein, oleic acid, and soybean oil); mineral salts, e.g., aluminium hydroxide and aluminium or calcium phosphate gels; oil emulsions and surfactant based formulations, e.g., MF59 (microfluidised detergent stabilized oil-in-water emulsion), QS21 (purified saponin), AS02 [SBAS2](oil-in-water emulsion+MPL+QS-21), MPL-SE, Montanide ISA-51 and ISA-720 (stabilised water-in-oil emulsion); particulate adjuvants, e.g., virosomes (unilamellar liposomal vehicles incorporating influenza haemagglutinin), AS04 ([SBAS4] Al salt with MPL), ISCOMS (structured complex of saponins and lipids), polylactide co-glycolide (PLG); microbial derivatives (natural and synthetic), e.g., monophosphoryl lipid A (MPL), Detox (MPL+M. Phlei cell wall skeleton), AGP [RC-529](synthetic acylated monosaccharide), Detox-PC, DC_Chol (lipoidal immunostimulators able to self-organize into liposomes), OM-174 (lipid A derivative), CpG motifs (synthetic oligonucleotides containing immunostimulatory CpG motifs), or other DNA structures, modified LT and CT (genetically modified bacterial toxins to provide non-toxic adjuvant effects); endogenous human immunomodulators, e.g., hGM-CSF or hIL-12 (cytokines that can be administered either as protein or plasmid encoded), Immudaptin (C3d tandem array), MoGM-CSF, TiterMax-G, CRL-1005, GERBU, TERamide, PSC97B, Adjumer, PG-026, GSK-I, GcMAF, B-alethine, MPC-026, Adjuvax, CpG ODN, Betafectin, Alum, and MF59 and inert vehicles, such as gold particles. In some embodiments, the adjuvants is selected from the group of: aluminum phosphate, aluminum hydroxide, and phosphated aluminum hydroxide. Additional adjuvants are known in the art, see, e.g., U.S. Pat. No. 6,890,540; U. S. Patent Pub. No. 2005/0244420; PCT/SE97/01003.

Additional suitable adjuvants used to enhance an immune response of the SA-MAPS composition as disclosed herein further include, without limitation, MPL™ (3-O-deacylated monophosphoryi lipid A, Corixa; Hamilton, Mont.), which is described in U.S. Pat. No. 4,912,094. Also suitable for use as adjuvants are synthetic lipid A analogs or aminoalkyl glucosamine phosphate compounds (AGP), or derivatives or analogs thereof, which are available from Corixa, and those that are described in U.S. Pat. No. 6,113,918. One such AGP is 2-[(R)-3-Tetradecanoyloxytetradecanoylamino]ethyl 2-Deoxy-4-O-phosphono-3-O-[(R)-3-tetradecanoyoxytetradecanoyl]-2-[(R1-3-t-etradecanoyloxytetradecanoyl-amino]-b-D-glucopyranoside, which is also known as 529 (formerly known as RC529). This 529 adjuvant is formulated as an aqueous form (AF) or as a stable emulsion (SE). Still other adjuvants include muramyl peptides, such as N-acetyl-muramyl-L-threonyl-D-isoglutamine (thr-MDP), N-acetyl-normuramyl-L-alanine-2-(1′-2′ dipalmitoyl-sn-glycero-hydroxyphosphoryloxy)-ethylamine (MTP-PE); oil-in-water emulsions, such as MF59 (U.S. Pat. No. 6,299,884) (containing 5% Squalene, 0.5% polysorbate 80, and 0.5% Span 85 (optionally containing various amounts of MTP-PE) formulated into submicron particles using a microfluidizer such as Model 110Y microfluidizer (Microlluidics, Newton, Mass.)), and SAF (containing 10% Squalene, 0.4% polysorbate 80, 5% pluronic-blocked polymer L121, and thr-MDP, either microfluidized into a submicron emulsion or vortexed to generate a larger particle size emulsion); incomplete Freund's adjuvant (IFA); aluminum salts (alum), such as aluminum hydroxide, aluminum phosphate, aluminum sulfate; Amphigen; Avridine; L121/squalene; D-lactide-polylactide/glycoside; pluronic polyols; killed Bordetella; saponins, such as Stimulon™ QS-21 (Antigenics, Framingham, Mass.), described in U.S. Pat. No. 5,057,540, Iscomatrix®. (CSL Limited, Parkville, Australia), described in U.S. Pat. No. 5,254,339, and immunostimulating complexes (ISCOMS); Mycobacterium tuberculosis; bacterial lipopolysaccharides; synthetic polynucleotides such as oligonucleotides containing a CpG motif (e.g., U.S. Pat. No. 6,207,646); IC-31 (Intercell AG, Vienna, Austria), described in EP Patent Nos. 1,296,713 and 1,326,634; a pertussis toxin (PT) or mutant thereof a cholera toxin or mutant thereof (e.g., U.S. Pat. Nos. 7,285,281, 7,332,174, 7,361,355 and 7,384,640); or an E. coli heat-labile toxin (LT) or mutant thereof, particularly LT-K63, LT-R72 (e.g., U.S. Pat. Nos. 6,149,919, 7,115,730 and 7,291,588).

In some embodiments, the SA-MAPS immunogen composition as disclosed herein is administered with at least one immune modulator. An “immunomodulator” or “immune modulator” is an agent that perturb or alter the immune system, such that either up-regulation or down-regulation of humoral and/or cell-mediated immunity is observed. In one embodiment, up-regulation of the humoral and/or cell-mediated arms of the immune system is provided. Examples of certain immunomodulators include, e.g., an adjuvant or cytokine, or Iscomatrix™ (CSL Limited; Parkville, Australia), described in U.S. Pat. No. 5,254,339 among others. Non-limiting examples of adjuvants that can be used in the immunogenic composition of the present invention include the RIBI adjuvant system (Ribi Inc.; Hamilton, Mont.), alum, mineral gels such as aluminum hydroxide gel, oil-in-water emulsions, water-in-oil emulsions such as, e.g., Freund's complete and incomplete adjuvants, Block copolymer (CytRx; Atlanta, Ga.), QS-21 (Cambridge Biotech Inc.; Cambridge, Mass.), SAF-M (Chixon; Emeryville, Calif.), Amphigen™. adjuvant, saponin, Quil A or other saponin fraction, monophosphoryl lipid A, and Avridine lipid-amine adjuvant. Non-limiting examples of oil-in-water emulsions useful in the immunogenic composition of the invention include modified SEAM62 and SEAM 1/2 formulations. Modified SEAM62 is oil-in-water emulsion containing 5% (v/v) squalene (Sigma), 1% (v/v) Span®. 85 Detergent (ICI Surfactants), 0.7% (v/v) polysorbate 80 detergent (ICI Surfactants), 2.5% (v/v) ethanol, 200 mcg/ml Quil A, 100 mcg/ml cholesterol, and 0.5% (v/v) lecithin. Modified SEAM 1/2 is an oil-in-water emulsion comprising 5% (v/v) squalene, 1% (v/v) Span®. 85 Detergent, 0.7% v/v) polysorbate 80 detergent, 2.5% (v/v) ethanol, 100 mcg/ml Quil A, and 50 mcg/ml cholesterol. Other “immunomodulators” that can be included in the immunogenic composition include, e.g., one or more interleukins, interferons, or other known cytokines or chemokines. In one embodiment, the adjuvant may be a cyclodextrin derivative or a polyanionic polymer, such as those described in U.S. Pat. Nos. 6,165,995 and 6,610,310, respectively. It is to be understood that the immunomodulator and/or adjuvant to be used will depend on the subject to which the immunogenic composition will be administered, the route of injection and the number of injections to be given.

In some embodiments, the SA-MAPS immunogen composition as disclosed herein is administered with at least one immune modulator. A number of cytokines or lymphokines have been shown to have immune modulating activity, and thus may be useful in a manner the same or similar to adjuvants, including, but not limited to, the interleukins 1-.alpha., 1-.beta., 2, 4, 5, 6, 7, 8, 10, 12 (see, e.g., U.S. Pat. No. 5,723,127), 13, 14, 15, 16, 17 and 18 (and its mutant forms); the interferons-α, β and γ; granulocyte-macrophage colony stimulating factor (GM-CSF) (see, e.g., U.S. Pat. No. 5,078,996 and ATCC Accession Number 39900); macrophage colony stimulating factor (M-CSF); granulocyte colony stimulating factor (G-CSF); and the tumor necrosis factors α and β. Still other adjuvants that are useful with the immunogenic compositions described herein include chemokines, including without limitation, MCP-1, MfP-1.alpha., MIP-Lbeta., and RANTES; adhesion molecules, such as a selectin, e.g., L-selectin, P-selectin and E-selectin; mucin-like molecules, e.g., CD34, GlyCAM-1 and MadCAM-1; a member of the integrin family such as LFA-1, VLA-1, Mac-1 and p150.95; a member of the immunoglobulin superfamily such as PECAM, ICAMs, e.g., ICAM-1, ICAM-2 and ICAM-3, CD2 and LFA-3; co-stimulatory molecules such as B7-1, B7-2, CD40 and CD40L; growth factors including vascular growth factor, nerve growth factor, fibroblast growth factor, epidermal growth factor, PDGF, BL-1, and vascular endothelial growth factor; receptor molecules including Fas, TNF receptor, Flt, Apo-1, p55, WSL-1, DR3, TRAMP, Apo-3, AIR, LARD, NGRF, DR4, DR5, KILLER, TRAIL-R2, TRICK2, and DR6; and Caspases, including ICE.

In some embodiments an adjuvant is a particulate and can have a characteristic of being slowly biodegradable. Care must be taken to ensure that that the adjuvant do not form toxic metabolites. Preferably, in some embodiments, such adjuvants can be matrices used are mainly substances originating from a body. These include lactic acid polymers, poly-amino acids (proteins), carbohydrates, lipids and biocompatible polymers with low toxicity. Combinations of these groups of substances originating from a body or combinations of substances originating from a body and biocompatible polymers can also be used. Lipids are the preferred substances since they display structures that make them biodegradable as well as the fact that they are a critical element in all biological membranes.

In one embodiment, the immunogenic compositions as described herein for administration must be sterile for administration to a subject. Sterility is readily accomplished by filtration through sterile filtration membranes (e.g., 0.2 micron membranes), or by gamma irradiation.

In some embodiments, the immunogenic compositions described herein further comprise pharmaceutical excipients including, but not limited to biocompatible oils, physiological saline solutions, preservatives, carbohydrate, protein, amino acids, osmotic pressure controlling agents, carrier gases, pH-controlling agents, organic solvents, hydrophobic agents, enzyme inhibitors, water absorbing polymers, surfactants, absorption promoters and anti-oxidative agents. Representative examples of carbohydrates include soluble sugars such as hydropropyl cellulose, carboxymethyl cellulose, sodium carboxyl cellulose, hyaluronic acid, chitosan, alginate, glucose, xylose, galactose, fructose, maltose, saccharose, dextran, chondroitin sulfate, etc. Representative examples of proteins include albumin, gelatin, etc. Representative examples of amino acids include glycine, alanine, glutamic acid, arginine, lysine, and their salts. Such pharmaceutical excipients are well-known in the art.

In some embodiments, the immunogenic MAPS composition is administered in combination with other therapeutic ingredients including, e.g., y-interferon, cytokines, chemotherapeutic agents, or anti-inflammatory, or anti-viral agents. In some embodiments, the immunogenic composition as disclosed herein can be administered with one or more co-stimulatory molecules and/or adjuvants as disclosed herein.

In some embodiments, the immunogenic composition is administered in a pure or substantially pure form, but may be administered as a pharmaceutical composition, formulation or preparation. Such formulation comprises MAPS described herein together with one or more pharmaceutically acceptable carriers and optionally other therapeutic ingredients. Other therapeutic ingredients include compounds that enhance antigen presentation, e.g., gamma interferon, cytokines, chemotherapeutic agents, or anti-inflammatory agents. The formulations can conveniently be presented in unit dosage form and may be prepared by methods well known in the pharmaceutical art. For example, Plotkin and Mortimer, in VACCINES (2nd ed., W.B. Saunders Co., 1994) describes vaccination of animals or humans to induce an immune response specific for particular pathogens, as well as methods of preparing antigen, determining a suitable dose of antigen, and assaying for induction of an immune response.

Formulations of the SA-MAPS compositions as disclosed herein suitable for intravenous, intramuscular, intranasal, oral, sublingual, vaginal, rectal, subcutaneous, or intraperitoneal administration conveniently comprise sterile aqueous solutions of the active ingredient with solutions which are preferably isotonic with the blood of the recipient. Such formulations may be conveniently prepared by dissolving solid active ingredient in water containing physiologically compatible substances such as sodium chloride (e.g., 0.1M-2.0 M), glycine, and the like, and having a buffered pH compatible with physiological conditions to produce an aqueous solution, and rendering the solution sterile. These may be present in unit or multi-dose containers, for example, sealed ampoules or vials.

Liposomal suspensions can also be used as pharmaceutically acceptable carriers. These can be prepared according to methods known to those skilled in the art, for example, as described in U.S. Pat. No. 4,522,811.

Formulations for an intranasal delivery are described in U.S. Pat. Nos. 5,427,782; 5,843,451; 6,398,774.

The formulations of the SA-MAPS compositions as disclosed herein can incorporate a stabilizer. Illustrative stabilizers are polyethylene glycol, proteins, saccharide, amino acids, inorganic acids, and organic acids which may be used either on their own or as admixtures. Two or more stabilizers may be used in aqueous solutions at the appropriate concentration and/or pH. The specific osmotic pressure in such aqueous solution is generally in the range of 0.1-3.0 osmoses, preferably in the range of 0.80-1.2. The pH of the aqueous solution is adjusted to be within the range of pH 5.0-9.0, preferably within the range of pH 6-8.

In certain embodiments, a formulation of the invention which is compatible with parenteral administration comprises one or more non-ionic surfactants, including but not limited to polyoxyethylene sorbitan fatty acid esters, Polysorbate-80 (Tween 80), Polysorbate-60 (Tween 60), Polysorbate-40 (Tween 40) and Polysorbate-20 (Tween 20), polyoxyethylene alkyl ethers, including but not limited to Brij 58, Brij 35, as well as others such as Triton X-100; Triton X-114, NP40, Span 85 and the Pluronic series of non-ionic surfactants (e. g., Plutonic 121), with preferred components Polysorbate-80 at a concentration from about 0.001% to about 2% (with up to about 0.25% being preferred) or Polysorbate-40 at a concentration from about 0.001% to 1% (with up to about 0.5% being preferred).

In certain embodiments, a formulation of the SA-MAPS compositions as disclosed herein comprises one or more additional stabilizing agents suitable for parenteral administration, e.g., a reducing agent comprising at least one thiol (—SH) group (e.g., cysteine, N-acetyl cysteine, reduced glutathione, sodium thioglycolate, thiosulfate, monothioglycerol, or mixtures thereof). Alternatively, or optionally, preservative-containing immunogenic composition formulations of the invention may be further stabilized by removing oxygen from storage containers, protecting the formulation from light (e.g., by using amber glass containers).

Preservative-containing immunogenic composition formulations of the SA-MAPS composition may comprise one or more pharmaceutically acceptable carriers or excipients, which includes any excipient that does not itself induce an immune response. Suitable excipients include but are not limited to macromolecules such as proteins, saccharides, polylactic acids, polyglycolic acids, polymeric amino acids, amino acid copolymers, sucrose (Paoletti et al, 2001, Vaccine, 19:2118), trehalose, lactose and lipid aggregates (such as oil droplets or liposomes). Such carriers are well known to the skilled artisan. Pharmaceutically acceptable excipients are discussed, e.g., in Gennaro, 2000, Remington: The Science and Practice of Pharmacy, 20.sup.th edition, ISBN:0683306472.

In some embodiments, SA-MAPS compositions as disclosed herein may be lyophilized or in aqueous form, i.e. solutions or suspensions. Liquid formulations may advantageously be administered directly from their packaged form and are thus ideal for injection without the need for reconstitution in aqueous medium as otherwise required for lyophilized compositions of the invention.

When oral preparations are desired, the immunogenic compositions can be combined with typical carriers, such as lactose, sucrose, starch, talc magnesium stearate, crystalline cellulose, methyl cellulose, carboxymethyl cellulose, glycerin, sodium alginate or gum arabic among others.

In some embodiments, the SA-MAPS immunogenic compositions as described herein can be administered intravenously, intranasally, intramuscularly, subcutaneously, intraperitoneally, sublingually, vaginal, rectal or orally. In some embodiments, the route of administration is oral, intranasal, subcutaneous, or intramuscular. In some embodiments, the route of administration is intranasal administration.

Vaccination can be conducted by conventional methods. For example, a SA-MAPS immunogenic composition as disclosed herein can be used in a suitable diluent such as saline or water, or complete or incomplete adjuvants. The immunogenic composition can be administered by any route appropriate for eliciting an immune response. The SA-MAPS immunogenic composition can be administered once or at periodic intervals until an immune response is elicited. Immune responses can be detected by a variety of methods known to those skilled in the art, including but not limited to, antibody production, cytotoxicity assay, proliferation assay and cytokine release assays. For example, samples of blood can be drawn from the immunized mammal, and analyzed for the presence of antibodies against the antigens of the immunogenic composition by ELISA (see de Boer et. al., 115 Arch Virol. 147 (1990) and the titer of these antibodies can be determined by methods known in the art.

The precise dose of the SA-MAPS to be employed in the formulation will also depend on the route of administration and should be decided according to the judgment of the practitioner and each patient's circumstances. For example, a range of 25 g-900 g total protein can be administered monthly for three months.

### Packaging and Dosage Forms

In some embodiments, the SA-MAPS immunogenic compositions as disclosed herein may be packaged in unit dose or multi-dose form (e.g. 2 doses, 4 doses, or more). For multi-dose forms, vials are typically but not necessarily preferred over pre-filled syringes. Suitable multi-dose formats include but are not limited to: 2 to 10 doses per container at 0.1 to 2 mL per dose. In certain embodiments, the dose is a 0.5 mL dose. See, e.g., International Patent Application WO2007/127668, which is incorporated by reference herein.

In some embodiments, the SA-MAPS immunogenic compositions as disclosed herein can be presented in vials or other suitable storage containers, or may be presented in pre-filled delivery devices, e.g., single or multiple component syringes, which may be supplied with or without needles. A syringe typically but need not necessarily contains a single dose of the preservative-containing immunogenic composition of the invention, although multi-dose, pre-filled syringes are also envisioned. Likewise, a vial may include a single dose but may alternatively include multiple doses.

Effective dosage volumes can be routinely established, but a typical dose of the composition for injection has a volume of 0.5 mL. In certain embodiments, the dose is formulated for administration to a human subject. In certain embodiments, the dose is formulated for administration to an adult, teen, adolescent, toddler or infant (i.e., no more than one year old) human subject and may in preferred embodiments be administered by injection.

Ultimately, the attending physician will decide the amount of the SA-MAPS immunogenic composition or vaccine composition to administer to particular individuals. As with all immunogenic compositions or vaccines, the immunologically effective amounts of the immunogens (e.g., the immunogenic polysaccharide and the SA antigens) must be determined empirically. Factors to be considered include the immunogenicity of the composition as a whole (e.g., it is important to note that the SA antigens induce a greater immune response when present in a SA-MAPS complex as compared to the mixture of the SA antigens alone (not complexed), the presence of an adjuvant or co-stimulant as disclosed herein, routes of administrations and the number of immunizing dosages to be administered. Such factors are known in the vaccine art and it is well within the skill of immunologists to make such determinations without undue experimentation.

Liquid immunogenic compositions of the SA-MAPS immunogenic compositions as disclosed herein are also suitable for reconstituting other immunogenic compositions which are presented in lyophilized form. Where an immunogenic composition is to be used for such extemporaneous reconstitution, in some embodiment, the present invention provides a kit with two or more vials, two or more ready-filled syringes, or one or more of each, with the contents of the syringe being used to reconstitute the contents of the vial prior to injection, or vice versa.

Alternatively, in some embodiments, the SA-MAPS immunogenic compositions as disclosed herein may be lyophilized and reconstituted, e.g., using one of a multitude of methods for freeze drying well known in the art to form dry, regular shaped (e.g., spherical) particles, such as micropellets or microspheres, having particle characteristics such as mean diameter sizes that may be selected and controlled by varying the exact methods used to prepare them. In some embodiments, the SA-MAPS immunogenic compositions as disclosed herein may further comprise an adjuvant which may optionally be prepared with or contained in separate dry, regular shaped (e.g., spherical) particles such as micropellets or microspheres. In some embodiments, the SA-MAPS immunogenic compositions as disclosed herein are present in a kit comprising a first component that includes a stabilized, dry SA-MAPS immunogenic composition as disclosed herein, optionally further comprising one or more preservatives, and a second component comprising a sterile, aqueous solution for reconstitution of the first component. In certain embodiments, the aqueous solution comprises one or more preservatives, and may optionally comprise at least one adjuvant (see, e.g., WO2009/109550 (incorporated herein by reference).

In yet another embodiment, a container of the multi-dose format is selected from one or more of the group consisting of, but not limited to, general laboratory glassware, flasks, beakers, graduated cylinders, fermentors, bioreactors, tubings, pipes, bags, jars, vials, vial closures (e.g., a rubber stopper, a screw on cap), ampoules, syringes, dual or multi-chamber syringes, syringe stoppers, syringe plungers, rubber closures, plastic closures, glass closures, cartridges and disposable pens and the like. The container of the present invention is not limited by material of manufacture, and includes materials such as glass, metals (e.g., steel, stainless steel, aluminum, etc.) and polymers (e.g., thermoplastics, elastomers, thermoplastic-elastomers). In a particular embodiment, the container of the format is a 5 mL Schott Type I glass vial with a butyl stopper. The skilled artisan will appreciate that the format set forth above is by no means an exhaustive list, but merely serve as guidance to the artisan with respect to the variety of formats available for the present invention. Additional formats contemplated for use in the present invention may be found in published catalogues from laboratory equipment vendors and manufacturers such as United States Plastic Corp. (Lima, Ohio), VWR.

### K. Kits

The present invention also provides for kits for producing a SA-MAPS immunogenic composition as disclosed herein which is useful for an investigator to tailor an immunogenic composition with their preferred SA antigens, e.g., for research purposes to assess the effect of a SA antigen, or a combination of SA antigens on immune response. Such kits can be prepared from readily available materials and reagents. For example, such kits can comprise any one or more of the following materials: a container comprising an immunogenic polysaccharide as disclosed herein (e.g, selected from CP1 from Streptococcus pneumoniae, CP5 from s. aureus, CP8 from S. aureus), cross-linked with a plurality of first affinity molecules (e.g., biotin); and a container comprising a complementary affinity molecule (e.g., a biotin-binding protein, such, as Rhavi), or a vector comprising a nucleic acid construct, which comprises a nucleic acid sequence encoding a biotin-binding protein, such as Rhavi and cloning sites for insertion of a nucleic acid sequence for encoding a S.A polypeptide antigen, where the vector can be used to express a fusion protein comprising a biotin-binding protein, e.g., Rhavi, and a S.A polypeptide antigen.

In another embodiment, the kit can comprise a container comprising a immunogenic polysaccharide as disclosed herein, (e.g, selected from CP1 from Streptococcus pneumoniae, CP5 from s. aureus, CP8 from S. aureus), a container comprising a plurality of first affinity molecules (e.g., biotin), and a container comprising a cross-linking reagent for cross-linking the first affinity molecules to the immunogenic polysaccharide.

In some embodiments, the kit further comprises a means to attach the complementary affinity molecule (e.g., a biotin-binding protein, e.g., Rhavi) to the antigen, where the means can be by a cross-linking reagent or by some intermediary fusion protein. In some embodiments, the kit can comprise at least one co-stimulation factor which can be added to the polymer. In some embodiments, the kit comprises a cross-linking reagent, for example, but not limited to, CDAP (1-cyano-4-dimethylaminopyridinium tetrafluoroborate), EDC (1-Ethyl-3-[3-dimethylaminopropyl]carbodiimide hydrochloride), sodium cyanoborohydride; cyanogen bromide; ammonium bicarbonate/iodoacetic acid for linking the co-factor to the polymer.

A variety of kits and components can be prepared for use in the methods described herein, depending upon the intended use of the kit, the particular target antigen and the needs of the user.

In one embodiment, a SA-MAPS immunogenic composition or vaccine composition as described herein, when administered to mice, can provoke an immune response that prevents a disease symptom in at least 20% of animals challenged with 5 LD₅₀ of the immunogenic composition comprising antigens to which the disease symptom is prevented. Methods of vaccination and challenging an immunized animal are known to one skilled in the art. For example, a 10 μg aliquot of an immunogenic composition or vaccine composition as disclosed herein can be prepared in 100 μl PBS and/or with addition of alum adjuvants or incomplete Freund's adjuvant and injected subcutaneously per mouse per vaccination. Alternatively, parenteral, intraperitoneal and footpad injections can be used. Volumes of footpad injections are reduced to 50 μl. Mice can be immunized with an immunogenic composition or vaccine composition as disclosed herein on three separate occasions with several days, e.g., 14 days interval in between.

Efficacy of vaccination can be tested by challenge with the pathogen, e.g., S. aureus, or by the method disclosed herein. Seven days after the last dose of an immunogenic composition, the immunized mice are challenged intranasally with a pathogenic organism from which the antigen was derived. Ether anaesthetized mice (10 g to 12 g) can be infected intranasally with 50 μl of PBS-diluted allantoic fluid containing 5 LD₅₀ of the pathogenic organism. Protection can be measured by monitoring animal survival and body weight, which is assessed throughout an observation period of 21 days. Severely affected mice are euthanized. One LD₅₀ of A/Mallard/Pennsylvania/10218/84 is equal to 100-1000 the Tissue Culture Infectious Dose50 (TCID50) assay.

### Definitions

For convenience, certain terms employed in the entire application (including the specification, examples, and appended claims) are collected here. Unless defined otherwise, all technical and scientific terms used herein have the same meaning as commonly understood by one of ordinary skill in the art to which this invention belongs.

As used herein and in the claims, the singular forms include the plural reference and vice versa unless the context clearly indicates otherwise. The term “or” is inclusive unless modified, for example, by “either.” Other than in the operating examples, or where otherwise indicated, all numbers expressing quantities of ingredients or reaction conditions used herein should be understood as modified in all instances by the term “about.” It is further to be understood that all base sizes or amino acid sizes, and all molecular weight or molecular mass values, given for nucleic acids or polypeptides are approximate, and are provided for description.

Unless defined otherwise, all technical and scientific terms used herein have the same meaning as those commonly understood to one of ordinary skill in the art to which this invention pertains. Although any known methods, devices, and materials may be used in the practice or testing of the invention, the methods, devices, and materials in this regard are described herein.

The term “immunogenic” as used herein means an ability of an antigen (or an epitope of the antigen), such as a bacterial capsular polysaccharide or a conjugate immunogenic composition comprising the bacterial capsular polysaccharide and a polypeptide or peptide antigen, to elicit an immune response in a host such as a mammal, either humorally or cellularly mediated, or both.

The term “immunogenic composition” used herein is defined as a composition capable of eliciting an immune response, such as an antibody or cellular immune response, or both, when administered to a subject. The immunogenic compositions as disclosed herein may or may not be immunoprotective or therapeutic. When the immunogenic compositions as disclosed herein prevent, ameliorate, palliate or eliminate disease from the subject, then the immunogenic composition may optionally be referred to as a vaccine. As used herein, however, the term immunogenic composition is not intended to be limited to vaccines.

Accordingly, an “immunogenic composition” as used herein means any immunogenic polysaccharide conjugated to one or more first affinity molecules (e.g., biotin), where the first affinity molecule is bound to a complementary affinity molecule (e.g., biotin-binding protein) that is fused to, or otherwise attached to at least one S. aureus peptide or polypeptide antigen, whereby both the immunogenic polysaccharide and the S. aureus peptide or polypeptide antigen, each, serve as antigens or antigenic determinant (i.e., epitopes) of the immunogenic composition to elicit an immune response. That is, the immunogenic composition induces a more robust immune response than each of the components alone (i.e., the immunogenic polysaccharide alone, or one or more of the S. aureus peptide or polypeptide antigens alone (i.e., a mixture of one or more of the S. aureus peptide or polypeptide antigens that are not in a complex or conjugated to the polysaccharide). The immunogenic composition may serve to sensitize the host by the presentation of one or more of the S. aureus peptide or polypeptide antigens in association with MHC molecules at a cell surface. In addition, antigen-specific T-cells or antibodies can be generated to allow for the future protection of an immunized host. Immunogenic composition thus can protect the host from one or more symptoms associated with infection by the S. aureus, or may protect the host from death due to the infection with S. aureus. In some embodiments, the SA-MAPS immunogenic compositions as disclosed herein can also be used to generate polyclonal or monoclonal antibodies, which may be used to confer passive immunity to a subject. In some embodiments, the SA-MAPS immunogenic compositions as disclosed herein can also be used to generate antibodies that are functional as measured by the killing of bacteria in either an animal efficacy model or via an opsonophagocytic killing assay.

The term “antigen” generally refers to a biological molecule, usually a protein or polypeptide, peptide, polysaccharide or conjugate in an immunogenic composition, or immunogenic substance that can stimulate the production of antibodies or T-cell responses, or both, in an animal, including compositions that are injected or absorbed into an animal. The immune response may be generated to the whole molecule (i.e., such as the SA-MAPS immunogenic composition, or to the whole immunogenic polysaccharide, or the whole peptide or polypeptide antigen), or to a various portions of the molecule (e.g., an epitope or hapten within a part of the SA-MAPS immunogenic composition, or to the whole immunogenic polysaccharide, or the whole peptide or polypeptide antigen). The term may be used to refer to an individual molecule or to a homogeneous or heterogeneous population of antigenic molecules. An antigen is recognized by antibodies, T-cell receptors or other elements of specific humoral and/or cellular immunity. The term “antigen” also includes all related antigenic epitopes. Epitopes of a given antigen can be identified using any number of epitope mapping techniques, well known in the art. See, e.g., Epitope Mapping Protocols in Methods in Molecular Biology, Vol. 66 (Glenn E. Morris, Ed., 1996) Humana Press, Totowa, N.J. For example, linear epitopes may be determined by, e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al. (1984) Proc. Natl. Acad. Sci. USA 81:3998-4002; Geysen et al. (1986) Molec. Immunol. 23:709-715; each of which is incorporated herein by reference as if set forth in its entirety. Similarly, conformational epitopes may be identified by determining spatial conformation of amino acids such as by, e.g., x-ray crystallography and 2-dimensional nuclear magnetic resonance. See, e.g., Epitope Mapping Protocols, supra. Furthermore, for purposes of the present invention, “antigen” also can be used to refer to a protein that includes modifications, such as deletions, additions and substitutions (generally conservative in nature, but they may be non-conservative), to the native sequence, so long as the protein maintains the ability to elicit an immunological response. These modifications may be deliberate, as through site-directed mutagenesis, or through particular synthetic procedures, or through a genetic engineering approach, or may be accidental, such as through mutations of hosts, which produce the antigens. Furthermore, the antigen can be derived, obtained, or isolated from a microbe, e.g., a bacterium, or can be a whole organism. Similarly, an oligonucleotide polynucleotide, which expresses an antigen, such as in nucleic acid immunization applications, is also included in the definition. Synthetic antigens are also included, e.g., polyepitopes, flanking epitopes, and other recombinant or synthetically derived antigens (Bergmann et al. (1993) Eur. J. Immunol. 23:2777 2781; Bergmann et al. (1996) J. Immunol. 157:3242-3249; Suhrbier (1997) Immunol. Cell Biol. 75:402 408; Gardner et al. (1998) 12th World AIDS Conference, Geneva, Switzerland, June 28 to Jul. 3, 1998). In some embodiments, an antigen is a peptide or a polypeptide, e.g., a S. aureus peptide or a polypeptide, or immunogenic polysaccharide and in other embodiments, it can be any chemical or moiety, e.g., a carbohydrate, that elicits an immune response directed against the substance.

An “immune response” to an antigen or immunogenic composition is the development in a subject of a humoral and/or a cell-mediated immune response to molecules present in the antigen or vaccine composition of interest. For purposes of the present invention, a “humoral immune response” is an antibody-mediated immune response and involves the induction and generation of antibodies that recognize and bind with some affinity for the antigen in the immunogenic composition of the invention, while a “cell-mediated immune response” is one mediated by T-cells and/or other white blood cells. A “cell-mediated immune response” is elicited by the presentation of antigenic epitopes in association with Class I or Class II molecules of the major histocompatibility complex (MHC), CD1 or other non-classical MHC-like molecules. This activates antigen-specific CD4+ T helper cells or CD8+ cytotoxic lymphocyte cells (“CTLs”). CTLs have specificity for peptide antigens that are presented in association with proteins encoded by classical or non-classical MHCs and expressed on the surfaces of cells. CTLs help induce and promote the intracellular destruction of intracellular microbes, or the lysis of cells infected with such microbes. Another aspect of cellular immunity involves an antigen-specific response by helper T-cells. Helper T-cells act to help stimulate the function, and focus the activity of, nonspecific effector cells against cells displaying peptide or other antigens in association with classical or non-classical MHC molecules on their surface. A “cell-mediated immune response” also refers to the production of cytokines, chemokines and other such molecules produced by activated T-cells and/or other white blood cells, including those derived from CD4+ and CD8+ T-cells. The ability of a particular antigen or composition to stimulate a cell-mediated immunological response may be determined by a number of assays, such as by lymphoproliferation (lymphocyte activation) assays, CTL cytotoxic cell assays, by assaying for T-lymphocytes specific for the antigen in a sensitized subject, or by measurement of cytokine production by T cells in response to re-stimulation with antigen. Such assays are well known in the art. See, e.g., Erickson et al. (1993) J. Immunol. 151:4189-4199; and Doe et al. (1994) Eur. J. Immunol. 24:2369-2376.

The term “treatment” (including variations thereof, e.g., “treat” or “treated”) as used herein means any one or more of the following: (i) the prevention of infection or re-infection, as in a traditional vaccine, (ii) the reduction in the severity of, or, in the elimination of symptoms, and (iii) the substantial or complete elimination of the pathogen or disorder in question. Hence, treatment may be effected prophylactically (prior to infection) or therapeutically (following infection). In the present invention, prophylactic treatment is the preferred mode. According to a particular embodiment of the present invention, compositions and methods are provided that treat, including prophylactically and/or therapeutically immunize, a host animal against a microbial infection (e.g., a bacterium such as Staphylococcus). The methods of the present invention are useful for conferring prophylactic and/or therapeutic immunity to a subject. The methods of the present invention can also be practiced on subjects for biomedical research applications.

The term “mammal” as used herein means a human or non-human animal. More particularly, mammal refers to any animal classified as a mammal, including humans, domestic and farm animals, and zoo, sports and pet companion animals such as a household pet and other domesticated animal including, but not limited to, cattle, sheep, ferrets, swine, horses, rabbits, goats, dogs, cats, and the like. In some embodiments, a companion animal is a dog or cat. Preferably, the mammal is human.

The term an “immunogenic amount,” and “immunologically effective amount,” both of which are used interchangeably herein, refers to the amount of the antigen or immunogenic composition sufficient to elicit an immune response, either a cellular (T-cell) or humoral (B-cell or antibody) response, or both, as measured by standard assays known to one skilled in the art. The “immunogenic amount” of a particular immunogenic composition is generally dosed based on total immunogenic polysaccharide and attached or associated SA peptide or polypeptide antigens. For example, an SA-MAPS immunogenic composition as disclosed herein will have at least about 80% or more of, e.g., a serotype 5 or 8 capsular polysaccharide with attached SA-antigens via the affinity binding pair. Accordingly, in some embodiments, a SA-MAPS immunogenic composition as disclosed herein can have 20%, or less, free immunogenic (e.g. CP5 or CP8 or a CP5/CP8 conjugate) polysaccharide, and as such, a 100 mcg dose can have about 80 mcg of immunogenic polysaccharide-antigen SA-MAPS complex and about 20 mcg, or less, of a non-conjugated immunogenic polysaccharide. In some embodiments, the dose of the SA-antigens associated with the immunogenic polysaccharide is important and considered when calculating the dose of a SA-MAPS composition to administer to a subject. The amount of SA-MAPS complex can vary depending upon the number and types of the attached SA antigens, the immunogenic polysaccharide (e.g., the staphylococcal serotype) as well as any associated co-stimulants as disclosed herein, as well as route of administration, subject and disease to be treated. Generally, each SA-MAPS dose will comprise 0.1 to 100 mcg of an immunogenic polysaccharide and attached SA antigens, particularly 0.1 to 10 mcg, and more particularly 1 to 10 mcg.

The amount of a SA-MAPS immunogenic composition as disclosed herein can vary depending upon the staphylococcal serotype. Generally, each dose will comprise 0.1 to 100 mcg of immunogenic polysaccharide, particularly 0.1 to 10 mcg, and more particularly 1 to 10 mcg. The “immunogenic amount” of the different polysaccharide components in the immunogenic composition, may diverge and each may comprise 1 mcg, 2 mcg, 3 mcg, 4 mcg, 6 mcg, 6 mcg, 7 mcg, 8 mcg, 9 mcg, 10 mcg, 15 mcg, 20 mcg, 30 mcg, 40 mcg, 50 mcg, 60 mcg, 70 mcg, 80 mcg, 90 mcg, or about 100 mcg of any particular polysaccharide antigen.

S. aureus “invasive disease” is the isolation of bacteria from a normally sterile site, where there is associated clinical signs/symptoms of disease. Normally sterile body sites include blood, CSF, pleural fluid, pericardial fluid, peritoneal fluid, joint/synovial fluid, bone, internal body site (lymph node, brain, heart, liver, spleen, vitreous fluid, kidney, pancreas, ovary) or other normally sterile sites. Clinical conditions characterizing invasive diseases include bacteremia, pneumonia, cellulitis, osteomyelitis, endocarditis, septic shock and more.

S. aureus infections in a human subject lead to diseases and disorders selected from: Pneumonia (including ventilator-associated pneumonia), sinusitis, skin infections/abscess/cellulitis/furuncles/impetigo, bloodstream infection, meningitis, endocarditis (heart infection), osteomyelitis, septic arthritis (both could be summarized as bone/joint infections) myositis (muscle infection), mastitis, mastoiditis, toxic shock syndrome, necrotizing fasciitis catheter/central line infection, prosthetic material infection including joint prostheses, surgical site infections.

The term “associates” as used herein refers to the linkage of two or more molecules by non-covalent or covalent bonds. In some embodiments, where linking of two or more molecules occurs by a covalent bond, the two or more molecules can be fused together, or cross-linked together. In some embodiments, where linking of two or more molecules occurs by a non-covalent bond, the two or more molecules can form a complex. Two entities are “associated” with one another, as that term is used herein, if the presence, level and/or form of one is correlated with that of the other. In some embodiments, two or more entities are physically “associated” with one another if they interact, directly or indirectly, so that they are and/or remain in physical proximity with one another. In some embodiments, two or more entities that are physically associated with one another are covalently linked to one another. In some embodiments, two or more entities that are physically associated with one another are not covalently linked to one another but are non-covalently associated, for example by means of affinity interactions, electrostatic interactions, hydrogen bonds, van der Waals interaction, hydrophobic interactions, magnetism, and combinations thereof.

The term “antigen”, as used herein, refers to (i) an agent that induces an immune response; and/or (ii) an agent that binds to a T cell receptor (e.g., when presented by an MEW molecule) or to an antibody. In some embodiments, an antigen induces a humoral response (e.g., including production of antigen-specific antibodies); in some embodiments, an antigen induces a cellular response (e.g., involving T cells whose receptors specifically interact with the antigen). In some embodiments, an antigen induces a humoral response and a cellular response. In some embodiments, an antigen binds to an antibody and may or may not induce a particular physiological response in an organism. In general, an antigen may be or include any chemical entity such as, for example, a small molecule, a nucleic acid, a polypeptide, a carbohydrate, a lipid, a polymer (in some embodiments other than a biologic polymer (e.g., other than a nucleic acid or amino acid polymer)), etc. In some embodiments, an antigen is or comprises a polypeptide. In some embodiments, an antigen is or comprises a polysaccharide. Those of ordinary skill in the art will appreciate that, in general, an antigen may be provided in isolated or pure form, or alternatively may be provided in crude form (e.g., together with other materials, for example in an extract such as a cellular extract or other relatively crude preparation of an antigen-containing source). In some embodiments, antigens utilized in accordance with the present invention are provided in a crude form. In some embodiments, an antigen is a recombinant antigen. In some embodiments, an antigen is a polypeptide or a polysaccharide that, upon administration to a subject, induces a specific and/or clinically relevant immune response to such polypeptide or polysaccharide. In some embodiments, an antigen is selected to induce a specific and/or clinically relevant immune response to such polypeptide or polysaccharide.

The term “complex” as used herein refers to a collection of two or more molecules, connected spatially by means other than a covalent interaction; for example, they can be connected by electrostatic interactions, hydrogen bound or by hydrophobic interactions (i.e., van der Waals forces).

The term “cross-linked” as used herein refers to a covalent bond formed between a polymer chain and a second molecule. The term “cross-linking reagent” refers to an entity or agent which is an intermediate molecule to catalyze the covalent linkage of a polymer with an entity, e.g., first affinity molecule or co-stimulatory factor.

As used herein, the term “fused” means that at least one protein or peptide is physically associated with a second protein or peptide. In some embodiments, fusion is typically a covalent linkage, however, other types of linkages are encompassed in the term “fused” include, for example, linkage via an electrostatic interaction, or a hydrophobic interaction and the like. Covalent linkage can encompass linkage as a fusion protein or chemically coupled linkage, for example via a disulfide bound formed between two cysteine residues.

As used herein, the term “fusion polypeptide” or “fusion protein” means a protein created by joining two or more polypeptide sequences together. The fusion polypeptides encompassed in this invention include translation products of a chimeric gene construct that joins the DNA sequences encoding one or more antigens, or fragments or mutants thereof, with the DNA sequence encoding a second polypeptide to form a single open-reading frame. In other words, a “fusion polypeptide” or “fusion protein” is a recombinant protein of two or more proteins which are joined by a peptide bond or via several peptides.

The term “polypeptide”, as used herein, generally has its art-recognized meaning of a polymer of at least three amino acids, e.g, linked to each other by peptide bonds. Those of ordinary skill in the art will appreciate that the term “polypeptide” is intended to be sufficiently general as to encompass not only polypeptides having a complete sequence recited herein, but also to encompass polypeptides that represent functional fragments (i.e., fragments retaining at least one activity) of such complete polypeptides. Moreover, those of ordinary skill in the art understand that protein sequences generally tolerate some substitution without destroying activity. Thus, any polypeptide that retains activity and shares at least about 30-40% overall sequence identity, often greater than about 50%, 60%, 70%, or 80%, and further usually including at least one region of much higher identity, often greater than 90% or even 95%, 96%, 97%, 98%, or 99% in one or more highly conserved regions, usually encompassing at least 3-4 and often up to 20 or more amino acids, with another polypeptide of the same class, is encompassed within the relevant term “polypeptide” as used herein. Polypeptides may contain L-amino acids, D-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof.

As used herein, the term “protein” encompasses a polypeptide. Proteins may include moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence), or can be a characteristic portion thereof. Those of ordinary skill will appreciate that a protein can sometimes include more than one polypeptide chain, for example linked by one or more disulfide bonds or associated by other means. Polypeptides may contain 1-amino acids, d-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof. The term “peptide” is generally used to refer to a polypeptide having a length of less than about 100 amino acids, less than about 50 amino acids, less than 20 amino acids, or less than 10 amino acids. In some embodiments, proteins are antibodies, antibody fragments, biologically active portions thereof, and/or characteristic portions thereof. The terms “protein” can encompass a multimeric protein, e.g., a protein containing more than one domain or subunit. The term “peptide” as used herein refers to a sequence of peptide bond-linked amino acids containing less than 25 amino acids, e.g., between about 4 amino acids and 25 amino acids in length. Proteins and peptides can be composed of linearly arranged amino acids linked by peptide bonds, whether produced biologically, recombinantly, or synthetically and whether composed of naturally occurring or non-naturally occurring amino acids, are included within this definition. Both full-length proteins and fragments thereof greater than 25 amino acids are encompassed by the definition of protein. The terms also include polypeptides that have co-translational (e.g., signal peptide cleavage) and post-translational modifications of the polypeptide, such as, for example, disulfide-bond formation, glycosylation, acetylation, phosphorylation, lipidation, proteolytic cleavage (e.g., cleavage by metalloproteases), and the like. Furthermore, as used herein, a “polypeptide” refers to a protein that includes modifications, such as deletions, additions, and substitutions (generally conservative in nature as would be known to a person in the art) to the native sequence, as long as the protein maintains the desired activity. These modifications can be deliberate, as through site-directed mutagenesis, or can be accidental, such as through mutations of hosts that produce the proteins, or errors due to PCR amplification or other recombinant DNA methods.

The term “polysaccharide” as used herein refers to a polymeric carbohydrate molecule composed of long chains of monosaccharide units bound together by glycosidic, phosphodiester, or other linkages and on hydrolysis give the constituent monosaccharides or oligosaccharides. Polysaccharides range in structure from linear to highly branched. Examples include storage polysaccharides such as starch and glycogen, structural polysaccharides such as cellulose and chitin and microbial polysaccharides, and antigenic polysaccharides found in microorganisms including, but not limited to, capsular polysaccharides (CPS), O polysaccharides (OPS), core O polysaccharides (COPS), and lipopolysaccharides (LP S).

By “signal sequence” is meant a nucleic acid sequence which, when operably linked to a nucleic acid molecule, facilitates secretion of the product (e.g., protein or peptide) encoded by the nucleic acid molecule. In some embodiments, the signal sequence is preferably located 5′ to the nucleic acid molecule.

As used herein, the term “linker” is used to refer to an entity that connects two or more elements to form a multi-element agent. For example, those of ordinary skill in the art appreciate that a polypeptide whose structure includes two or more functional or organizational domains often includes a stretch of amino acids between such domains that links them to one another. In some embodiments, a polypeptide comprising a linker element has an overall structure of the general form S1-L-S2, wherein S1 and S2 may be the same or different and represent two domains associated with one another by the linker (L). In some embodiments, a polypeptide linker is at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100 or more amino acids in length. In some embodiments, a linker is characterized in that it tends not to adopt a rigid three-dimensional structure, but rather provides flexibility to the polypeptide. A variety of different linker elements that can appropriately be used when engineering polypeptides (e.g., fusion polypeptides) are known in the art (Holliger et al, 1993; Poljak, 1994).

As used herein, the term “N-glycosylated” or “N-glycosylation” refers to the covalent attachment of a sugar moiety to asparagine residues in a polypeptide. Sugar moieties can include but are not limited to glucose, mannose, and N-acetylglucosamine. Modifications of the glycans are also included, e.g., siaylation.

An “antigen presenting cell” or “APC” is a cell that expresses the Major Histocompatibility complex (MHC) molecules and can display foreign antigen complexed with MHC on its surface. Examples of antigen presenting cells are dendritic cells, macrophages, B-cells, fibroblasts (skin), thymic epithelial cells, thyroid epithelial cells, glial cells (brain), pancreatic beta cells, and vascular endothelial cells.

The term “functional portion” or “functional fragment” as used in the context of a “functional portion of an antigen” refers to a portion of the antigen or antigen polypeptide that mediates the same effect as the full antigen moiety, e.g., elicits an immune response in a subject, or mediates an association with other molecule, e.g., comprises at least on epitope.

A “portion” of a target antigen as that term is used herein will be at least 3 amino acids in length, and can be, for example, at least 6, at least 8, at least 10, at least 14, at least 16, at least 17, at least 18, at least 19, at least 20 or at least 25 amino acids or greater, inclusive.

A “fragment” of a material or entity as described herein has a structure that includes a discrete portion of the whole, but lacks one or more moieties found in the whole. In some embodiments, a fragment consists of such a discrete portion. In some embodiments, a fragment includes a discrete portion of the whole which discrete portion shares one or more functional characteristics found in the whole. In some embodiments, a fragment consists of such a discrete portion. In some embodiments, a fragment consists of or comprises a characteristic structural element or moiety found in the whole. In some embodiments, a fragment of a polymer, e.g., a polypeptide or a polysaccharide, comprises or consists of at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500 or more monomeric units (e.g., residues) as found in the whole polymer. In some embodiments, a polymer fragment comprises or consists of at least about 5%, 10%, 15%, 20%, 25%, 30%, 25%, 40%, 45%, 50%, 55%, 60%, 65%, 70%, 75%, 80%, 85%, 90%, 95%, 96%, 97%, 98%, 99% or more of the monomeric units (e.g., residues) found in the whole polymer. The whole material or entity may in some embodiments be referred to as the “parent” of the whole.

As used herein, the term “homology” refers to the overall relatedness between polymeric molecules, e.g., between nucleic acid molecules (e.g., DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, 96%, 97%, 98%, or 99% identical. In some embodiments, polymeric molecules are considered to be “homologous” to one another if their sequences are at least 25%, 30%, 35%, 40%, 45%, 50%, 55% 60%, 65%, 70%, 75%, 80%, 85%, 90%, 95%, 96%, 97%, 98%, or 99% similar (e.g., containing residues with related chemical properties at corresponding positions). For example, as is well known by those of ordinary skill in the art, certain amino acids are typically classified as similar to one another as “hydrophobic” or “hydrophilic” amino acids, and/or as having “polar” or “non-polar” side chains. Substitution of one amino acid for another of the same type may often be considered a “homologous” substitution.

As used herein, the term “identity” refers to the overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, 96%, 97%, 98%, or 99% identical. Calculation of the percent identity of two nucleic acid or polypeptide sequences, for example, can be performed by aligning the two sequences for optimal comparison purposes (e.g., gaps can be introduced in one or both of a first and a second sequence for optimal alignment and non-identical sequences can be disregarded for comparison purposes). In certain embodiments, the length of a sequence aligned for comparison purposes is at least 30%, at least 40%, at least 50%, at least 60%, at least 70%, at least 75%, at least 80%, at least 85%, at least 90%, at least 95%, at least 96%, at least 97%, at least 98%, at least 99%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller, 1989, which has been incorporated into the ALIGN program (version 2.0). In some exemplary embodiments, nucleic acid sequence comparisons made with the ALIGN program use a PAM120 weight residue table, a gap length penalty of 12 and a gap penalty of 4. The percent identity between two nucleotide sequences can, alternatively, be determined using the GAP program in the GCG software package using an NWSgapdna.CMP matrix.

The terms “Cytotoxic T Lymphocyte” or “CTL” refers to lymphocytes which induce death via apoptosis or other mechanisms in targeted cells. CTLs form antigen-specific conjugates with target cells via interaction of TCRs with processed antigen (Ag) on target cell surfaces, resulting in apoptosis of the targeted cell. Apoptotic bodies are eliminated by macrophages. The term “CTL response” is used to refer to the primary immune response mediated by CTL cells.

The term “cell mediated immunity” or “CMI” as used herein refers to an immune response that does not involve antibodies or complement but rather involves the activation of, for example, macrophages, natural killer cells (NK), antigen-specific cytotoxic T-lymphocytes (T-cells), T-helper cells, neutrophils, and the release of various cytokines in response to a target antigen. Stated another way, CMI refers to immune cells (such as T cells and other lymphocytes) which bind to the surface of other cells that display a target antigen (such as antigen presenting cells (APC)) and trigger a response. The response may involve either other lymphocytes and/or any of the other white blood cells (leukocytes) and the release of cytokines. Cellular immunity protects the body by: (i) activating antigen-specific cytotoxic T-lymphocytes (CTLs) that are able to destroy body cells displaying epitopes of foreign antigen on their surface, such as virus-infected cells and cells with intracellular bacteria; (2) activating macrophages and NK cells, enabling them to destroy intracellular pathogens; and (3) stimulating cells to secrete a variety of cytokines or chemokines that influence the function of other cells such as T cells, macrophages or neutrophils involved in adaptive immune responses and innate immune responses.

The term “immune cell” as used herein refers to any cell which can release a cytokine, chemokine or antibody in response to a direct or indirect antigenic stimulation. Included in the term “immune cells” herein are lymphocytes, including natural killer (NK) cells, T-cells (CD4+ and/or CD8+ cells), B-cells, macrophages; leukocytes; dendritic cells; mast cells; monocytes; and any other cell which is capable of producing a cytokine or chemokine molecule in response to direct or indirect antigen stimulation. Typically, an immune cell is a lymphocyte, for example a T-cell lymphocyte.

A “protective” immune response refers to the ability of an immunogenic composition as disclosed herein to elicit an immune response, either humoral or cell mediated, or both, which serves to protect a subject from an infection. The protection provided need not be absolute, i.e., the infection need not be totally prevented or eradicated, if there is a statistically significant improvement compared with a control population of subjects, e.g. infected animals not administered the vaccine or immunogenic composition. Protection may be limited to mitigating the severity or rapidity of onset of symptoms of the infection. In general, a “protective immune response” would include the induction of an increase in antibody levels specific for a particular antigen in at least 50% of subjects, including some level of measurable functional antibody responses to each antigen. In particular situations, a “protective immune response” could include the induction of a two-fold increase in antibody levels or a fourfold increase in antibody levels specific for a particular antigen in at least 50% of subjects, including some level of measurable functional antibody responses to each antigen. In certain embodiments, opsonising antibodies correlate with a protective immune response. Thus, protective immune response may be assayed by measuring the percent decrease in the bacterial count in an opsonophagocytosis assay, for instance those described below. Preferably, there is a decrease in bacterial count of at least 10%, 25%, 50%, 65%, 75%, 80%, 85%, 90%, 95% or more.

The term “cytokine” as used herein refers to a molecule released from an immune cell in response to stimulation with an antigen. Examples of such cytokines include, but are not limited to: GM-CSF; IL-1α; IL-1β; IL-2; IL-3; IL-4; IL-5; IL-6; IL-7; IL-8; IL-10; IL-12; IL-17A, IL-17F, or other members of the IL-17 family, IL-22, IL-23, IFN-α; IFN-β; IFN-γ; MIP-1α; MIP-1β; TGF-β; TNFα, or TNFβ. The term “cytokine” does not include antibodies.

The term “subject” as used herein refers an organism, typically a mammal (e.g., a human, in some embodiments including prenatal human forms) which it is useful to elicit an immune response. The subject can be a wild, domestic, commercial or companion animal such as a bird or mammal. The subject can be a human. Although in one embodiment of the invention it is contemplated that the immunogenic compositions as disclosed herein can also be suitable for the therapeutic or preventative treatment in humans, it is also applicable to warm-blooded vertebrates, e.g., mammals, such as non-human primates, (particularly higher primates), sheep, dog, rodent (e.g., mouse or rat), guinea pig, goat, pig, cat, rabbits, cows, and non-mammals such as chickens, ducks, or turkeys. In another embodiment, the subject is a wild animal, for example a bird such as for the diagnosis of avian flu. In some embodiments, the subject is an experimental animal or animal substitute as a disease model. The subject may be a subject in need of veterinary treatment, where eliciting an immune response to an antigen is useful to prevent a disease and/or to control the spread of a disease, for example from infection with methicillin-resistant Staphylococcus aureus (MRSA), methicillin-susceptible Staphylococcus aureus (MSSA), vancomycin-intermediate Staphylococcus aureus (VISA), vancomycin-resistant Staphylococcus aureus (VRSA), or in the case of live-stock, chickens, pigs, sheep, cows and heifers that can be carriers for S. aureus, and which can lead to mastitis and other skin-diseases (e.g., dermatitis) in livestock, such as dairy-producing livestock. In some embodiments, a subject is susceptible to a disease, disorder, or condition. S. aureus is the main causative agent of mastitis in cattle, sheep, goats, and horses, as well as may cause dermatitis in sheep and goats, botryomycosis in pigs and horses, and suppurative infections in cats and dogs. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is a subject to whom diagnosis and/or therapy is and/or has been administered.

As used herein, the term “pathogen” refers to an organism or molecule that causes a disease or disorder in a subject. For example, pathogens include but are not limited to viruses, fungi, bacteria, parasites, and other infectious organisms or molecules therefrom, as well as taxonomically related macroscopic organisms within the categories algae, fungi, yeast, protozoa, or the like.

The term “wild type” refers to the naturally-occurring, normal polynucleotide sequence encoding a protein, or a portion thereof, or protein sequence, or portion thereof, respectively, as it normally exists in vivo.

The term “mutant” refers to an organism or cell with any change in its genetic material, in particular a change (i.e., deletion, substitution, addition, or alteration) relative to a wild-type polynucleotide sequence or any change relative to a wild-type protein sequence. The term “variant” may be used interchangeably with “mutant”. Although it is often assumed that a change in the genetic material results in a change of the function of the protein, the terms “mutant” and “variant” refer to a change in the sequence of a wild-type protein regardless of whether that change alters the function of the protein (e.g., increases, decreases, imparts a new function), or whether that change has no effect on the function of the protein (e.g., the mutation or variation is silent).

As used herein, the term “pharmaceutical composition” refers to a composition in which an active agent is formulated together with one or more pharmaceutically acceptable carriers. In some embodiments, the active agent is present in unit dose amount appropriate for administration in a therapeutic regimen that shows a statistically significant probability of achieving a predetermined therapeutic effect when administered to a relevant population. In some embodiments, a pharmaceutical composition may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

The term “pharmaceutically acceptable” refers to compounds and compositions which may be administered to mammals without undue toxicity. The term “pharmaceutically acceptable carriers” excludes tissue culture medium. Exemplary pharmaceutically acceptable salts include but are not limited to mineral acid salts such as hydrochlorides, hydrobromides, phosphates, sulfates, and the like, and the salts of organic acids such as acetates, propionates, malonates, benzoates, and the like. Pharmaceutically acceptable carriers are well-known in the art.

Improve, increase, inhibit or reduce: As used herein, the terms “improve”, “increase”, “inhibit”, “reduce”, or grammatical equivalents thereof, indicate values that are relative to a baseline or other reference measurement. In some embodiments, an appropriate reference measurement may be or comprise a measurement in a particular system (e.g., in a single subject) under otherwise comparable conditions absent presence of (e.g., prior to and/or after) a particular agent or treatment, or in presence of an appropriate comparable reference agent. In some embodiments, an appropriate reference measurement may be or comprise a measurement in comparable system known or expected to respond in a particular way, in presence of the relevant agent or treatment.

Immunologically effective amount or immunologically effective dose: As used herein, “immunologically effective amount” or “immunologically effective dose” refers to an amount of an antigenic or immunogenic substance, e.g., an antigen, immunogen, immunogenic complex, immunogenic composition, vaccine, or pharmaceutical composition, which when administered to a subject, either in a single dose or as part of a series of doses, that is sufficient to enhance a subject's own immune response against a subsequent exposure to a pathogen. In some embodiments, the pathogen is S. aureus. In some embodiments, the immune response is against one or more different serotypes of S. aureus. In some embodiments, the immune response is against two or more different serotypes of S. aureus. In some embodiments, the immune response is against three or more different serotypes of S. aureus. In some embodiments, the immune response is against four or more different serotypes of S. aureus. An immunologically effective amount may vary based on the subject to be treated, the species of the subject, the degree of immune response desired to induce, etc. In some embodiments, an immunologically effective amount is sufficient for treatment or protection of a subject having or at risk of having disease. In some embodiments, an immunologically effective amount refers to a non-toxic but sufficient amount that can be an amount to treat, attenuate, or prevent infection and/or disease (e.g., bacterial infection, pneumococcal infection, bacterial colonization, pneumococcal colonization, complications associated with bacterial infection, complications associated with pneumococcal infection, etc.) in any subject. In some embodiments, an immunologically effective amount is sufficient to induce an immunoprotective response upon administration to a subject.

As used herein, “immunoprotective response” or “protective response” refers to an immune response that mediates antigen or immunogen-induced immunological memory. In some embodiments, an immunoprotective response is induced by the administration of a substance, e.g., an antigen, immunogen, immunogenic complex, immunogenic composition, vaccine, or pharmaceutical composition to a subject. In some embodiments, immunoprotection involves one or more of active immune surveillance, a more rapid and effective response upon immune activation as compared to a response observed in a naïve subject, efficient clearance of the activating agent or pathogen, followed by rapid resolution of inflammation. In some embodiments, an immunoprotective response is an adaptive immune response. In some embodiments, an immunoprotective response is sufficient to protect an immunized subject from productive infection by a particular pathogen or pathogens to which a vaccine is directed (e.g., S. pneumoniae infection).

As used herein, “immunization”, or grammatical equivalents thereof, refers to a process of inducing an immune response to an infectious organism or agent in a subject (“active immunization”), or alternatively, providing immune system components against an infectious organism or agent to a subject (“passive immunization”). In some embodiments, immunization involves the administration of one or more antigens, immunogens, immunogenic complexes, vaccines, immune molecules such as antibodies, immune sera, immune cells such as T cells or B cells, or pharmaceutical compositions to a subject. In some embodiments, immunization is performed by administering an immunologically effective amount of a substance, e.g., an antigen, immunogen, immunogenic complex, immunogenic composition, vaccine, immune molecule such as an antibody, immune serum, immune cell such as a T cell or B cell, or pharmaceutical composition to a subject. In some embodiments, immunization results in an immunoprotective response in the subject. In some embodiments, active immunization is performed by administering to a subject an antigenic or immunogenic substance, e.g., an antigen, immunogen, immunogenic complex, vaccine, or pharmaceutical composition. In some embodiments, passive immunization is performed by administering to a subject an immune system component, e.g., an immune molecule such as an antibody, immune serum, or immune cell such as a T cell or B cell.

As used herein, the term “colonization” generally refers to the ability of a microbe to grow at a target site or surface. For example, the terms “colonization” refers to the ability of a microbe (e.g., a bacterium) to grow at an anatomical site (e.g., a mucosal membrane, gastrointestinal tract, injury site, organ, etc.) of a host.

As used herein, the term “combination therapy” refers to those situations in which a subject is exposed to two or more therapeutic regimens (e.g., two or more therapeutic agents). In some embodiments, the two or more regimens may be administered simultaneously; in some embodiments, such regimens may be administered sequentially (e.g., all “doses” of a first regimen are administered prior to administration of any doses of a second regimen); in some embodiments, such agents are administered in overlapping dosing regimens. In some embodiments, “administration” of combination therapy may involve administration of one or more agent(s) or modality(ies) to a subject receiving the other agent(s) or modality(ies) in the combination. For clarity, combination therapy does not require that individual agents be administered together in a single composition (or even necessarily at the same time), although in some embodiments, two or more agents, or active moieties thereof, may be administered together in a combination composition, or even in a combination compound (e.g., as part of a single chemical complex or covalent entity).

As used herein, the term “treatment” (also “treat” or “treating”) refers to any administration of a therapy that partially or completely alleviates, ameliorates, relieves, inhibits, delays onset of, reduces severity of, and/or reduces incidence of one or more symptoms, features, and/or causes of a particular disease, disorder, and/or condition. In some embodiments, such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

As used herein, the term “vaccination” refers to the administration of a composition intended to generate an immune response, for example to a disease-causing agent. For the purposes of the present invention, vaccination can be administered before, during, and/or after exposure to a disease-causing agent, and in certain embodiments, before, during, and/or shortly after exposure to the agent. In some embodiments, vaccination includes multiple administrations, appropriately spaced in time, of a vaccinating composition. In some embodiments, vaccination initiates immunization.

The term “prevent” or “prevention”, as used herein in connection with a disease, disorder, and/or medical condition, refers to reducing the risk of developing the disease, disorder and/or condition, and/or a delay of onset, and/or reduction in frequency and/or severity of one or more characteristics or symptoms of a particular disease, disorder or condition. In some embodiments, prevention is assessed on a population basis such that an agent is considered to “prevent” a particular disease, disorder or condition if a statistically significant decrease in the development, frequency, and/or intensity of one or more symptoms of the disease, disorder or condition is observed in a population susceptible to the disease, disorder, or condition. In some embodiments, prevention may be considered complete when onset of a disease, disorder or condition has been delayed for a pre-defined period of time.

As used herein, the term “serotype”, also referred to as a serovar, refers to a distinct variation within a species of bacteria or virus or among immune cells of different subjects. These microorganisms, viruses, or cells are classified together based on their cell surface antigens, allowing the epidemiologic classification of organisms to the sub-species level. A group of serovars with common antigens may be referred to as a serogroup or sometimes serocomplex.

Dosage form or unit dosage form: Those skilled in the art will appreciate that the term “dosage form” may be used to refer to a physically discrete unit of an active agent (e.g., a therapeutic or diagnostic agent) for administration to a subject. Typically, each such unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or agent administered to a particular subject is determined by one or more attending physicians and may involve administration of multiple dosage forms.

Dosing regimen: Those skilled in the art will appreciate that the term “dosing regimen” may be used to refer to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which is separated in time from other doses. In some embodiments, individual doses are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

It will be appreciated that proteins or polypeptides often contain amino acids other than the 20 amino acids commonly referred to as the 20 naturally occurring amino acids, and that many amino acids, including the terminal amino acids, can be modified in a given polypeptide, either by natural processes such as glycosylation and other post-translational modifications, or by chemical modification techniques which are well known in the art. Known modifications which can be present in polypeptides of the present invention include, but are not limited to, acetylation, acylation, ADP-ribosylation, amidation, covalent attachment of flavin, covalent attachment of a heme moiety, covalent attachment of a polynucleotide or polynucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formulation, gamma-carboxylation, glycation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

As used herein, the terms “homologous” or “homologues” are used interchangeably, and when used to describe a polynucleotide or polypeptide, indicate that two polynucleotides or polypeptides, or designated sequences thereof, when optimally aligned and compared, for example using BLAST, version 2.2.14 with default parameters for an alignment are identical, with appropriate nucleotide insertions or deletions or amino-acid insertions or deletions, typically in at least 70% of the nucleotides of the nucleotides for high homology. For a polypeptide, there should be at least 30% of amino acid identity in the polypeptide, or at least 50% for higher homology. The term “homolog” or “homologous” as used herein also refers to homology with respect to structure. Determination of homologs of genes or polypeptides can be easily ascertained by the skilled artisan. When in the context with a defined percentage, the defined percentage homology means at least that percentage of amino acid similarity. For example, 85% homology refers to at least 85% of amino acid similarity.

As used herein, the term “heterologous” reference to nucleic acid sequences, proteins or polypeptides mean that these molecules are not naturally occurring in that cell. For example, the nucleic acid sequence coding for a fusion antigen polypeptide described herein that is inserted into a cell, e.g. in the context of a protein expression vector, is a heterologous nucleic acid sequence.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. Where necessary or desired, optimal alignment of sequences for comparison can be conducted by any variety of approaches, as these are well-known in the art.

The term “variant” as used herein may refer to a polypeptide or nucleic acid that differs from the naturally occurring polypeptide or nucleic acid by one or more amino acid or nucleic acid deletions, additions, substitutions or side-chain modifications, yet retains one or more specific functions or biological activities of the naturally occurring molecule. Amino acid substitutions include alterations in which an amino acid is replaced with a different naturally-occurring or a non-conventional amino acid residue. Such substitutions may be classified as “conservative,” in which case an amino acid residue contained in a polypeptide is replaced with another naturally occurring amino acid of similar character either in relation to polarity, side chain functionality or size. Substitutions encompassed by variants as described herein may also be “non conservative,” in which an amino acid residue which is present in a peptide is substituted with an amino acid having different properties (e.g., substituting a charged or hydrophobic amino acid with alanine), or alternatively, in which a naturally-occurring amino acid is substituted with a non-conventional amino acid. Also encompassed within the term “variant,” when used with reference to a polynucleotide or polypeptide, are variations in primary, secondary, or tertiary structure, as compared to a reference polynucleotide or polypeptide, respectively (e.g., as compared to a wild-type polynucleotide or polypeptide).

The term “substantially similar,” when used in reference to a variant of an antigen or a functional derivative of an antigen as compared to the original antigen means that a particular subject sequence vanes from the sequence of the antigen polypeptide by one or more substitutions, deletions, or additions, but retains at least 50%, or higher, e.g., at least 60%, 70%, 80%, 90% or more, inclusive, of the function of the antigen to elicit an immune response in a subject. In determining polynucleotide sequences, all subject polynucleotide sequences capable of encoding substantially similar amino acid sequences are considered to be substantially similar to a reference polynucleotide sequence, regardless of differences in codon sequence. A nucleotide sequence is “substantially similar” to a given antigen nucleic acid sequence if: (a) the nucleotide sequence hybridizes to the coding regions of the native antigen sequence, or (b) the nucleotide sequence is capable of hybridization to nucleotide sequence of the native antigen under moderately stringent conditions and has biological activity similar to the native antigen protein; or (c) the nucleotide sequences are degenerate as a result of the genetic code relative to the nucleotide sequences defined in (a) or (b). Substantially similar proteins will typically be greater than about 80% similar to the corresponding sequence of the native protein.

Variants can include conservative or non-conservative amino acid changes, as described below. Polynucleotide changes can result in amino acid substitutions, additions, deletions, fusions and truncations in the polypeptide encoded by the reference sequence. Variants can also include insertions, deletions or substitutions of amino acids, including insertions and substitutions of amino acids and other molecules) that do not normally occur in the peptide sequence that is the basis of the variant, for example but not limited to insertion of ornithine which do not normally occur in human proteins. “Conservative amino acid substitutions” result from replacing one amino acid with another that has similar structural and/or chemical properties. Conservative substitution tables providing functionally similar amino acids are well known in the art. For example, the following six groups each contain amino acids that are conservative substitutions for one another: (1) Alanine (A), Serine (S), Threonine (T); (2) Aspartic acid (D), Glutamic acid (E); (3) Asparagine (N), Glutamine (Q); (4) Arginine (R), Lysine (K); (5) Isoleucine (I), Leucine (L), Methionine (M), Valine (V); and (6) Phenylalanine (F), Tyrosine (Y), Tryptophan (W). See, e.g., Creighton, PROTEINS (W.H. Freeman & Co., 1984).

The choice of conservative amino acids may be selected based on the location of the amino acid to be substituted in the peptide, for example if the amino acid is on the exterior of the peptide and exposed to solvents, or on the interior and not exposed to solvents. Selection of such conservative amino acid substitutions is within the skill of one of ordinary skill in the art. Accordingly, one can select conservative amino acid substitutions suitable for amino acids on the exterior of a protein or peptide (i.e. amino acids exposed to a solvent). These substitutions include, but are not limited to the following: substitution of Y with F, T with S or K, P with A, E with D or Q, N with D or G, R with K, G with N or A, T with S or K, D with N or E, I with L or V, F with Y, S with T or A, R with K, G with N or A, K with R, A with S, K or P.

Alternatively, one can also select conservative amino acid substitutions suitable for amino acids on the interior of a protein or peptide (i.e., the amino acids are not exposed to a solvent). For example, one can use the following conservative substitutions: where Y is substituted with F, T with A or S, I with L or V, W with Y, M with L, N with D, G with A, T with A or S, D with N, I with L or V, F with Y or L, S with A or T and A with S, G, T or V. In some embodiments, LF polypeptides including non-conservative amino acid substitutions are also encompassed within the term “variants.” As used herein, the term “non-conservative” substitution refers to substituting an amino acid residue for a different amino acid residue that has different chemical properties. Non-limiting examples of non-conservative substitutions include aspartic acid (D) being replaced with glycine (G); asparagine (N) being replaced with lysine (K); and alanine (A) being replaced with arginine (R).

The term “derivative” as used herein refers to proteins or peptides which have been chemically modified, for example by ubiquitination, labeling, pegylation (derivatization with polyethylene glycol) or addition of other molecules. A molecule is also a “derivative” of another molecule when it contains additional chemical moieties not normally a part of the molecule. Such moieties can improve the molecule's solubility, absorption, biological half-life, etc. The moieties can alternatively decrease the toxicity of the molecule, or eliminate or attenuate an undesirable side effect of the molecule, etc. Moieties capable of mediating such effects are disclosed in REMINGTON'S PHARMACEUTICAL SCIENCES (21st ed., Tory, ed., Lippincott Williams & Wilkins, Baltimore, MD, 2006).

The term “functional” when used in conjunction with “derivative” or “variant” refers to a protein molecule which possesses a biological activity that is substantially similar to a biological activity of the entity or molecule of which it is a derivative or variant. “Substantially similar” in this context means that the biological activity, e.g., antigenicity of a polypeptide, is at least 50% as active as a reference, e.g., a corresponding wild-type polypeptide, e.g., at least 60% as active, 70% as active, 80% as active, 90% as active, 95% as active, 100% as active or even higher (i.e., the variant or derivative has greater activity than the wild-type), e.g., 110% as active, 120% as active, or more, inclusive.

The term “recombinant” when used to describe a nucleic acid molecule, means a polynucleotide of genomic, cDNA, viral, semisynthetic, and/or synthetic origin, which, by virtue of its origin or manipulation, is not associated with all or a portion of the polynucleotide sequences with which it is associated in nature. The term recombinant as used with respect to a peptide, polypeptide, protein, or recombinant fusion protein, means a polypeptide produced by expression from a recombinant polynucleotide. The term recombinant as used with respect to a host cell means a host cell into which a recombinant polynucleotide has been introduced. Recombinant is also used herein to refer to, with reference to material (e.g., a cell, a nucleic acid, a protein, or a vector) that the material has been modified by the introduction of a heterologous material (e.g., a cell, a nucleic acid, a protein, or a vector).

The term “vectors” refers to a nucleic acid molecule capable of transporting or mediating expression of a heterologous nucleic acid to which it has been linked to a host cell; a plasmid is a species of the genus encompassed by the term “vector.” The term “vector” typically refers to a nucleic acid sequence containing an origin of replication and other entities necessary for replication and/or maintenance in a host cell. Vectors capable of directing the expression of genes and/or nucleic acid sequence to which they are operatively linked are referred to herein as “expression vectors”. In general, expression vectors of utility are often in the form of “plasmids” which refer to circular double stranded DNA molecules which, in their vector form are not bound to the chromosome, and typically comprise entities for stable or transient expression or the encoded DNA. Other expression vectors that can be used in the methods as disclosed herein include, but are not limited to plasmids, episomes, bacterial artificial chromosomes, yeast artificial chromosomes, bacteriophages or viral vectors, and such vectors can integrate into the host's genome or replicate autonomously in the particular cell. A vector can be a DNA or RNA vector. Other forms of expression vectors known by those skilled in the art which serve the equivalent functions can also be used, for example self replicating extrachromosomal vectors or vectors which integrates into a host genome. Preferred vectors are those capable of autonomous replication and/or expression of nucleic acids to which they are linked.

The term “reduced” or “reduce” or “decrease” as used herein generally means a decrease by a statistically significant amount relative to a reference. For avoidance of doubt, “reduced” means statistically significant decrease of at least 10% as compared to a reference level, for example a decrease by at least 20%, at least 30%, at least 40%, at least t 50%, or least 60%, or least 70%, or least 80%, at least 90% or more, up to and including a 100% decrease (i.e., absent level as compared to a reference sample), or any decrease between 10-100% as compared to a reference level, as that term is defined herein.

The term “low” as used herein generally means lower by a statically significant amount; for the avoidance of doubt, “low” means a statistically significant value at least 10% lower than a reference level, for example a value at least 20% lower than a reference level, at least 30% lower than a reference level, at least 40% lower than a reference level, at least 50% lower than a reference level, at least 60% lower than a reference level, at least 70% lower than a reference level, at least 80% lower than a reference level, at least 90% lower than a reference level, up to and including 100% lower than a reference level (i.e., absent level as compared to a reference sample).

The terms “increased” or “increase” as used herein generally mean an increase by a statically significant amount; such as a statistically significant increase of at least 10% as compared to a reference level, including an increase of at least 20%, at least 30%, at least 40%, at least 50%, at least 60%, at least 70%, at least 80%, at least 90%, at least 100% or more, inclusive, including, for example at least 2-fold, at least 3-fold, at least 4-fold, at least 5-fold, at least 10-fold increase or greater as compared to a reference level, as that term is defined herein.

The term “high” as used herein generally means a higher by a statically significant amount relative to a reference; such as a statistically significant value at least 10% higher than a reference level, for example at least 20% higher, at least 30% higher, at least 40% higher, at least 50% higher, at least 60% higher, at least 70% higher, at least 80% higher, at least 90% higher, at least 100% higher, inclusive, such as at least 2-fold higher, at least 3-fold higher, at least 4-fold higher, at least 5-fold higher, at least 10-fold higher or more, as compared to a reference level.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of elements that do not materially affect the basic and novel or functional characteristic(s) of that embodiment of the invention.

Although the foregoing invention has been described in some detail by way of illustration and example for purposes of clarity of understanding it will be readily apparent to one of ordinary skill in the art in light of the teachings of this invention that certain changes and modifications may be made thereto without departing from the spirit or scope of the appended claims. The following is meant to be illustrative of the present invention; however, the practice of the invention is not limited or restricted in any way by the examples.

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

In some embodiments, the present invention may be defined in any of the following numbered paragraphs:

1. An immunogenic composition comprising at least one immunogenic complex, wherein the immunogenic complex comprises:
at least one biotinylated immunogenic polysaccharide antigen, and
at least one fusion protein, the fusion protein comprising:
   a biotin-binding protein or biotin-binding moiety, and at least one S. aureus polypeptide antigen,
wherein the biotinylated polysaccharide antigen is non-covalently associated with the biotin-binding moiety of the fusion protein to form an immunogenic complex,
wherein the at least one of the S. aureus polypeptide antigen is selected from any of: a SA1739 (B2) antigen comprising the amino acid sequence of SEQ ID NO: 51 or at least 85% sequence identity to SEQ ID NO: 51; a SA1720 (B1) antigen comprising the amino acid sequence of SEQ ID NO: 50 or at least 85% sequence identity to SEQ ID NO: 50; a SA1890 (B3) antigen comprising the amino acid sequence of SEQ ID NO: 52 or at least 85% sequence identity to SEQ ID NO: 52, a SA0103 (T1) antigen comprising the amino acid sequence of SEQ ID NO: 53 or at least 85% sequence identity to SEQ ID NO: 53; a SA0377 (T2) antigen comprising the amino acid sequence of SEQ ID NO: 54 or at least 85% sequence identity to SEQ ID NO: 54; a SA0693 (T3) antigen comprising the amino acid sequence of SEQ ID NO: 55 or at least 85% sequence identity to SEQ ID NO: 55, and a SA2105 (T4) antigen comprising the amino acid sequence of SEQ ID NO: 56 or at least 85% sequence identity to SEQ ID NO: 56, and optionally, wherein the at least one immunogenic polysaccharide is selected from: type 1 capsular polysaccharide of Streptococcus pneumoniae, a type 5 capsular polysaccharide of S. aureus, and/or a type 8 capsular polysaccharide of S. aureus. The at least one immunogenic polysaccharide can be readily substituted with an immunogenic polypeptide from other bacterial species.

### Paragraph 1 (of [00611]) in the Alternative:

1. An immunogenic composition comprising: (a) at least one immunogenic polysaccharide, (b) at least one S. aureus polypeptide antigen, and (c) at least one pair of affinity molecules, wherein the at least one pair of affinity molecules comprises:
(i) a first affinity molecule comprising a biotin, and
(ii) a second affinity molecule comprising a biotin-binding protein, wherein in each pair of affinity molecules:
   wherein the first affinity molecule is associated with the at least one immunogenic polysaccharide, and the second affinity molecule is associated with the at least S. aureus polypeptide antigens, and wherein the first affinity molecule non-covalently associates with the second affinity molecule to link the S. aureus polypeptide antigen(s) and the immunogenic polysaccharide;
   wherein the at least one immunogenic polysaccharide is selected from: type 1 capsular polysaccharide of Streptococcus pneumoniae, a type 5 capsular polysaccharide of S. aureus, and/or a type 8 capsular polysaccharide of S. aureus; and
   wherein the at least one of the S. aureus polypeptide antigen is selected from any of: a SA1739 (B2) antigen comprising the amino acid sequence of SEQ ID NO: 51 or at least 85% sequence identity to SEQ ID NO: 51; a SA1720 (B1) antigen comprising the amino acid sequence of SEQ ID NO: 50 or at least 85% sequence identity to SEQ ID NO: 50; a SA1890 (B3) antigen comprising the amino acid sequence of SEQ ID NO: 52 or at least 85% sequence identity to SEQ ID NO: 52, a SA0103 (T1) antigen comprising the amino acid sequence of SEQ ID NO: 53 or at least 85% sequence identity to SEQ ID NO: 53; a SA0377 (T2) antigen comprising the amino acid sequence of SEQ ID NO: 54 or at least 85% sequence identity to SEQ ID NO: 54; a SA0693 (T3) antigen comprising the amino acid sequence of SEQ ID NO: 55 or at least 85% sequence identity to SEQ ID NO: 55, and a SA2105 (T4) antigen comprising the amino acid sequence of SEQ ID NO: 56 or at least 85% sequence identity to SEQ ID NO: 56.

2. The immunogenic composition of paragraph 1, wherein the immunogenic complex comprises at least two S. aureus polypeptide antigens, comprising (i) a first S. aureus polypeptide antigens comprising a SA1739 (B2) antigen comprising the amino acid sequence of SEQ ID NO: 51 or a polypeptide that has at least 85% sequence identity to SEQ ID NO: 51, and (ii) a second polypeptide antigen selected from any of the group comprising: hemolysin (Hl), Clumping factor A (ClfA), Clumping factor B (ClfB), serine-aspirate repeat protein D (SdrD), serine-aspirate repeat protein E (SdrE), Iron regulator surface protein A (IsdA), Iron regulator surface protein B (IsdB), Leukotoxin D (LukD), Leukotoxin E (LukE), SA1720 (B1), SA1890 (B3), SA0103 (T1), SA0377 (T2), SA0693 (T3) or SA2105 (T4).

3. The immunogenic composition of paragraph 2, wherein the immunogenic complex comprises at least three S. aureus polypeptide antigens, comprising (i) a first S. aureus polypeptide antigens comprising a SA1739 (B2) antigen comprising the amino acid sequence of SEQ ID NO: 51 or a polypeptide that has at least 85% sequence identity to SEQ ID NO: 51, and (ii) a second and third S. aureus antigen selected from any of the group comprising: hemolysin (Hl), Clumping Clumping factor A (ClfA), Clumping factor B (ClfB), serine-aspirate repeat protein D (SdrD), serine-aspirate repeat protein E (SdrE), Iron regulator surface protein A (IsdA), Iron regulator surface protein B (IsdB), Leukotoxin D (LukD), Leukotoxin E (LukE), SA1720 (B1), SA1890 (B3), SA0103 (T1), SA0377 (T2), SA0693 (T3) or SA2105 (T4).

4. The immunogenic composition of paragraph 3, wherein the immunogenic complex comprises at least four S. aureus polypeptide antigens, comprising (i) a first S. aureus polypeptide antigens comprising a SA1739 (B2) antigen comprising the amino acid sequence of SEQ ID NO: 51 or a polypeptide that has at least 85% sequence identity to SEQ ID NO: 51, and (ii) at least three additional S. aureus antigen selected from any of the group comprising: hemolysin (Hl), Clumping Clumping factor A (ClfA), Clumping factor B (ClfB), serine-aspirate repeat protein D (SdrD), serine-aspirate repeat protein E (SdrE), Iron regulator surface protein A (IsdA), Iron regulator surface protein B (IsdB), Leukotoxin D (LukD), Leukotoxin E (LukE), SA1720 (B1), SA1890 (B3), SA0103 (T1), SA0377 (T2), SA0693 (T3) or SA2105 (T4).

5. The immunogenic composition of paragraph 4, wherein the immunogenic complex comprises at least five S. aureus polypeptide antigens, comprising (i) a first S. aureus polypeptide antigens comprising a SA1739 (B2) antigen comprising the amino acid sequence of SEQ ID NO: 51 or a polypeptide that has at least 85% sequence identity to SEQ ID NO: 51, and (ii) at least four additional S. aureus antigen selected from any of the group comprising: hemolysin (Hl), Clumping Clumping factor A (ClfA), Clumping factor B (ClfB), serine-aspirate repeat protein D (SdrD), serine-aspirate repeat protein E (SdrE), Iron regulator surface protein A (IsdA), Iron regulator surface protein B (IsdB), Leukotoxin D (LukD), Leukotoxin E (LukE), SA1720 (B1), SA1890 (B3), SA0103 (T1), SA0377 (T2), SA0693 (T3) or SA2105 (T4).

6. The immunogenic composition of paragraph 5, wherein the immunogenic complex comprises (i) a first S. aureus polypeptide antigens comprising a SA1739 (B2) antigen comprising the amino acid sequence of SEQ ID NO: 51 or a polypeptide that has at least 85% sequence identity to SEQ ID NO: 51, and (ii) at least S. aureus antigens hemolysin (Hl), Clumping Clumping factor A (ClfA), Clumping factor B (ClfB), serine-aspirate repeat protein D (SdrD).

7. The immunogenic composition of paragraph 5, wherein the immunogenic complex comprises (i) a first S. aureus polypeptide antigens comprising a SA1739 (B2) antigen comprising the amino acid sequence of SEQ ID NO: 51 or a polypeptide that has at least 85% sequence identity to SEQ ID NO: 51, and (ii) a second S. aureus polypeptide antigen selected from SA0103 (T1), SA0377 (T2) or SA0693 (T3), and (iii) a third S. aureus polypeptide antigen selected from: hemolysin (Hl), Clumping Clumping factor A (ClfA), Clumping factor B (ClfB), serine-aspirate repeat protein D (SdrD).

8. The immunogenic composition of paragraph 7, wherein the immunogenic complex comprises (i) a first S. aureus polypeptide antigens comprising a SA1739 (B2) antigen comprising the amino acid sequence of SEQ ID NO: 51 or a polypeptide that has at least 85% sequence identity to SEQ ID NO: 51 and (ii) a second S. aureus polypeptide antigen comprising SA0103 (T1), and (iii) a third S. aureus polypeptide antigen selected from: hemolysin (Hl), Clumping Clumping factor A (ClfA), Clumping factor B (ClfB), serine-aspirate repeat protein D (SdrD).

9. The immunogenic composition of paragraph 7, wherein the immunogenic complex comprises (i) a first S. aureus polypeptide antigen comprising a SA1739 (B2) antigen comprising the amino acid sequence of SEQ ID NO: 51 or a polypeptide that has at least 85% sequence identity to SEQ ID NO: 51 and (ii) a second S. aureus polypeptide antigen comprising SA0103 (T1) and SA0693 (T3), and (iii) a third S. aureus polypeptide antigen selected from; hemolysin (Hl), Clumping Clumping factor A (ClfA), Clumping factor B (ClfB), serine-aspirate repeat protein D (SdrD).

10. The immunogenic composition of paragraph 7, wherein the immunogenic complex comprises (i) a first S. aureus polypeptide antigens comprising a SA1739 (B2) antigen comprising the amino acid sequence of SEQ ID NO: 51 or a polypeptide that has at least 85% sequence identity to SEQ ID NO: 51 and (ii) a second S. aureus polypeptide antigen comprising SA0103 (T1) and SA0377 (T2), and (iii)) a third S. aureus polypeptide antigen selected from; hemolysin (Hl), Clumping Clumping factor A (ClfA), Clumping factor B (ClfB), serine-aspirate repeat protein D (SdrD).

11. The immunogenic composition of paragraph 9 or 10, wherein the second S. aureus polypeptide antigen is a S. aureus antigen fusion protein, comprising SA0103 (T1) fused to SA0377 (T2), or SA0103 (T1) fused to SA0693 (T3).

12. The immunogenic composition of paragraph 1, wherein the immunogenic complex comprises at least two S. aureus polypeptide antigens, which are (i) a S. aureus antigen selected from SA0103 (T1), SA0377 (T2) or SA0693 (T3), or SA2105 (T4) and (ii) a polypeptide antigen selected from any of the group comprising: SA1720 (B1), SA1739 (B2), SA1890 (B3), hemolysin (Hl), Clumping factor A (ClfA), Clumping factor B (ClfB), serine-aspirate repeat protein D (SdrD), serine-aspirate repeat protein E (SdrE), Iron regulator surface protein A (IsdA), Iron regulator surface protein B (IsdB), Leukotoxin D (LukD), or Leukotoxin E (LukE) 13. The immunogenic composition of paragraph 12, wherein the immunogenic complex comprises at least three S. aureus polypeptide antigens, which are (i) a S. aureus antigen selected from SA0103 (T1), SA0377 (T2) or SA0693 (T3), or SA2105 (T4) and (ii) at least two additional S. aureus antigen selected from any of the group comprising: SA1720 (B1), SA1739 (B2), SA1890 (B3), hemolysin (Hl), Clumping Clumping factor A (ClfA), Clumping factor B (ClfB), serine-aspirate repeat protein D (SdrD), serine-aspirate repeat protein E (SdrE), Iron regulator surface protein A (IsdA), Iron regulator surface protein B (IsdB), Leukotoxin D (LukD), or Leukotoxin E (LukE).

14. The immunogenic composition of paragraph 13, wherein the immunogenic complex comprises at least four S. aureus polypeptide antigens, which are (i) a S. aureus antigen selected from SA0103 (T1), SA0377 (T2) or SA0693 (T3) or SA2105 (T4) and, (ii) at least three additional S. aureus antigen selected from any of the group comprising: SA1720 (B1), SA1739 (B2), SA1890 (B3), hemolysin (Hl), Clumping Clumping factor A (ClfA), Clumping factor B (ClfB), serine-aspirate repeat protein D (SdrD), serine-aspirate repeat protein E (SdrE), Iron regulator surface protein A (IsdA), Iron regulator surface protein B (IsdB), Leukotoxin D (LukD), or Leukotoxin E (LukE).

15. The immunogenic composition of paragraph 14, wherein the immunogenic complex comprises at least five S. aureus polypeptide antigens, which are (i) a S. aureus antigen selected from SA0103 (T1), SA0377 (T2) or SA0693 (T3) or SA2105 (T4) and, (ii) at least four additional S. aureus antigen selected from any of the group comprising: SA1720 (B1), SA1739 (B2), SA1890 (B3), hemolysin (Hl), Clumping Clumping factor A (ClfA), Clumping factor B (ClfB), serine-aspirate repeat protein D (SdrD), serine-aspirate repeat protein E (SdrE), Iron regulator surface protein A (IsdA), Iron regulator surface protein B (IsdB), Leukotoxin D (LukD), or Leukotoxin E (LukE).

16. The immunogenic composition of paragraph 15, wherein the immunogenic complex comprises (i) a SA0103 (T1) S. aureus antigen and (ii) at least S. aureus antigens hemolysin (Hl), Clumping Clumping factor A (ClfA), Clumping factor B (ClfB), serine-aspirate repeat protein D (SdrD).

17. The immunogenic composition of paragraph 16, wherein the immunogenic composition comprises (i) S. aureus antigens SA0103 (T1) and SA0377 (T2), or SA0103 (T1) and SA0693 (T3), or SA0103 (T1) and SA2105 (T4) and (ii) at least S. aureus antigens hemolysin (Hl), Clumping Clumping factor A (ClfA), Clumping factor B (ClfB), serine-aspirate repeat protein D (SdrD).

18. The immunogenic composition of paragraph 17, wherein the immunogenic complex comprises (i) S. aureus antigens: SA0103 (T1) and SA0377 (T2), or SA0103 (T1) and SA0693 (T3) or SA0103 (T1) and SA2105 (T4) and (ii) a S. aureus antigen selected from: SA1720 (B1), SA1739 (B2), SA1890 (B3), and (iii) at least S. aureus antigens hemolysin (Hl), Clumping Clumping factor A (ClfA), Clumping factor B (ClfB), serine-aspirate repeat protein D (SdrD).

19. The immunogenic composition of paragraph 17 or 18, wherein the immunogenic complex comprises a S. aureus antigen fusion protein, comprising SA0103 (T1) fused to any one of: SA0377 (T2), SA0693 (T3) or SA2105 (T4).

20. The immunogenic composition of paragraph 12, wherein the immunogenic complex comprises (i) a SA0377 (T2) S. aureus antigen and (ii) at least S. aureus antigens hemolysin (Hl), Clumping Clumping factor A (ClfA), Clumping factor B (ClfB), serine-aspirate repeat protein D (SdrD).

21. The immunogenic composition of paragraph 12, wherein the immunogenic complex comprises (i) a SA0693 (T3) S. aureus antigen and (ii) at least S. aureus antigens hemolysin (Hl), Clumping Clumping factor A (ClfA), Clumping factor B (ClfB), serine-aspirate repeat protein D (SdrD).

22. The immunogenic composition of paragraph 12, wherein the immunogenic complex comprises (i) a SA2015 (T4) S. aureus antigen and (ii) at least S. aureus antigens hemolysin (Hl), Clumping Clumping factor A (ClfA), Clumping factor B (ClfB), serine-aspirate repeat protein D (SdrD).

23. The immunogenic composition of any of paragraphs 1-19, wherein the immunogenic complex comprises S. aureus antigens Hla209(27-319), ClfA(221-559), ClfB (203-542), SdrD (246-682), IsdA (47-324) and IsdB (48-447), or functional modifications thereof.

24. The immunogenic composition of any of paragraphs 1 to 20, wherein Hl antigen is a α-hemolysin (Hla), a β-hemolysin (Hlb) or a γ-hemolysin (Hl-gamma) from S. aureus.

25. The immunogenic composition of any of paragraphs 1 to 21, wherein Hl is wildtype Hla (WT Hla) or a Hla with a reduced hemolytic activity or is a non-hemolytic Hla protein.

26. The immunogenic composition of any of paragraphs 1 to 22, wherein the Hla antigen with a reduced hemolytic activity comprises amino acids of SEQ ID NO: 14, SEQ ID NO: 15, SEQ ID NO: 17 or SEQ ID NO: 18 or a polypeptide with at least 85% sequence identity thereto.

27. The immunogenic composition of any of paragraphs 1 to 24, wherein the Hla antigen with a reduced hemolytic activity is amino acids of SEQ ID NO: 16 or a polypeptide with at least 85% sequence identity thereto.

28. The immunogenic composition of any of paragraphs 1 to 20, wherein the ClfA antigen comprises at least SEQ ID NO: 3 or a polypeptide with at least 85% sequence identity to SEQ ID NO: 3.

29. The immunogenic composition of any of paragraphs 1 to 20, wherein the ClfA antigen comprises a fragment of at least 30 amino acids of SEQ ID NO: 2 or a polypeptide of at least 30 amino acids that has at least 85% sequence identity to a portion of SEQ ID NO: 2.

30. The immunogenic composition of any of paragraphs 1 to 20, wherein the ClfB antigen comprises at least SEQ ID NO: 5 or a polypeptide with at least 85% sequence identity to SEQ ID NO: 5.

31. The immunogenic composition of any of paragraphs 1 to 20, wherein the ClfB antigen comprises a fragment of at least 30 amino acids of SEQ ID NO: 4 or a polypeptide of at least 30 amino acids that has at least 85% sequence identity to a portion of SEQ ID NO: 4.

32. The immunogenic composition of any of paragraphs 1 to 20, wherein the SdrD antigen comprises at least SEQ ID NO: 7 or a polypeptide with at least 85% sequence identity to SEQ ID NO: 7.

33. The immunogenic composition of any of paragraphs 1 to 20, wherein the SdrD antigen comprises a fragment of at least 30 amino acids of SEQ ID NO:6 or a polypeptide of at least 30 amino acids that has at least 85% sequence identity to a portion of SEQ ID NO: 6.

34. The immunogenic composition of any of paragraphs 1-33, wherein the biotin-binding is selected from the group consisting of: rhizavidin, avidin, streptavidin, or a homologue or derivative thereof.

35. The immunogenic composition of paragraph 34, wherein the rhizavidin is amino acids of SEQ ID NO: 1, or 85% sequence identity to amino acids of SEQ ID NO: 1.

36. The immunogenic composition of any of paragraphs 1 to 35, wherein the fusion protein comprises at least one S. aureus polypeptide antigen fused to the biotin-binding molecule.

37. The immunogenic composition of any of paragraphs 1 to 36, wherein the fusion protein comprises at least two S. aureus polypeptide antigens, wherein a first S. aureus polypeptide antigen fused to the biotin-binding molecule and a second S. aureus polypeptide antigen is fused to the first S. aureus polypeptide antigen, or wherein the first S. aureus polypeptide antigen, and the second S. aureus polypeptide antigen is fused to the biotin-binding molecule.

38. The immunogenic composition of paragraph 37, wherein the first S. aureus polypeptide antigen is a SA0103 (T1) S. aureus polypeptide antigen, which is fused to a second S. aureus polypeptide antigen selected from any of: SA0377 (T2), SA0693 (T3), SA2104 (T4), wherein SA0103 (T1) is fused to the second affinity molecule.

39. The immunogenic composition of paragraph 38, wherein the the first S. aureus polypeptide is a SA0103 (T1) S. aureus polypeptide antigen which is fused to to a second S. aureus polypeptide antigen selected from any of: SA0377 (T2), SA0693 (T3) or SA2104 (T4), wherein SA0377 (T2) or SA0693 (T3) or SA2104 (T4) is fused to the second affinity molecule.

40. The immunogenic composition of paragraph 1, the fusion protein further comprising at least one linker peptide located between the at least one of the S. aureus polypeptide antigen and the biotin-binding protein or biotin-binding moiety.

41. The immunogenic composition of any of paragraphs 1 to 40, further comprising at least one adjuvant or co-stimulation factor.

42. The immunogenic composition of paragraph 1 for use in any one or more of: as a diagnostic for exposure to a S. aureus pathogen or immune threat, to prevent or treat an infection by S. aureus, to prevent colonization of a subject by S. aureus, to elicit an immune response to S. aureus in a subject, wherein the immune response is selected from any of: (i) an antibody or B-cell response, (ii) an antibody or B-cell response and T-cell response, (iii) an immune response to the at least one immunogenic polysaccharide and at least one of the S. aureus polypeptide antigens, (iv) an immune response that is a CD4+ T cell response, including Th1, Th2, or Th17 or Th22 response, or a CD8+ T cell response, or CD4+ and CD8+ T cell response, (v) an antibody or B cell response to the at least one immunogenic polysaccharide and a CD4+ T cell response, including Th1, Th2, or Th17 or Th22 response, or a CD8+ T cell response, or CD4+/CD8+ T cell response to at least one of the S. aureus polypeptide antigens, (vi) an antibody or B cell response to the at least one immunogenic polysaccharide, and an antibody or B cell response and a CD4+ T cell response, including Th1, Th2, Th17 or Th22 responses, or a CD8+ T cell response, or CD4+/CD8+ T cell response to at least one of the S. aureus polypeptide antigens, (vii) an immune response results in activation of INF-γ, IL-17A or IL-22 producing cells, or INF-γ, IL-17A and IL-22 producing cells, or (viii) an antibody or B-cell response against at least one of the S. aureus polypeptide antigens which associates with the at least one immunogenic polysaccharide.

43. A method for inducing an immune response in a subject to S. aureus, comprising administering to the subject an immunogenic composition of paragraph 1 to 42.

44. A method of vaccinating a mammal against S. aureus infection, the method comprising administering an immunogenic composition of paragraph 1 to 42.

45. The method of any of paragraphs 43 or 44, wherein the subject is a human.

46. The method of any of paragraphs 43 or 44, wherein the subject is an agricultural or non-domestic animal.

47. The method of any of paragraphs 43 or 44, wherein the subject is a domestic animal.

48. The method of any of paragraphs 43 or 44, wherein administration is via subcutaneous, intranasal, intradermal, or intra muscular injection, or via transdermal skin patch.

49. The method of paragraph 43, wherein the immune response is selected from any of: (i) an antibody or B-cell response, (ii) an antibody or B-cell response and T-cell response, (iii) an immune response to the at least one immunogenic polysaccharide and at least one of the S. aureus polypeptide antigens, (iv) an immune response that is a CD4+ T cell response, including Th1, Th2, or Th17 or Th22 response, or a CD8+ T cell response, or CD4+ and CD8+ T cell response, (v) an antibody or B cell response to the at least one immunogenic polysaccharide and a CD4+ T cell response, including Th1, Th2, or Th17 or Th22 response, or a CD8+ T cell response, or CD4+/CD8+ T cell response to at least one of the S. aureus polypeptide antigens, (vi) an antibody or B cell response to the at least one immunogenic polysaccharide, and an antibody or B cell response and a CD4+ T cell response, including Th1, Th2, Th17 or Th22 responses, or a CD8+ T cell response, or CD4+/CD8+ T cell response to at least one of the S. aureus polypeptide antigens, (vii) an immune response results in activation of INF-γ, IL-17A or IL-22 producing cells, or INF-γ, IL-17A and IL-22 producing cells, or (viii) an antibody or B-cell response against at least one of the S. aureus polypeptide antigens which associates with the at least one immunogenic polysaccharide.

50. A fusion protein comprising, in any order, a rhizavidin protein and at least one S. aureus polypeptide antigen, wherein the rhizavidin protein comprises amino acids of SEQ ID NO: 1, or 85% sequence identity to amino acids of SEQ ID NO: 1, and the S. aureus polypeptide antigen comprises a polypeptide comprising the amino acids selected from the group of: SEQ ID NO: 50 (SA1739; B1), SEQ ID NO: 51 (SA1720; B2), SEQ ID NO: 52 (SA1890; B3), SEQ ID NO: 53 (SA0103; T1), SEQ ID NO: 54 (SA0377; T2), SEQ ID NO: 55 (SA0693; T3), SEQ ID NO: 56 (SA2105; T4), or a fragment of at least 20 amino acids thereof, or a a polypeptide having at least 80%, or at least 85% or 90% sequence identity to any of SEQ ID NO: 51-56.

51. The fusion protein of paragraph 50, where the fusion protein comprises, in the following order:
(a) (i) a rhizavidin protein and at least one S. aureus polypeptide antigen, wherein the rhizavidin protein comprises amino acids of SEQ ID NO: 1, or 85% sequence identity to amino acids of SEQ ID NO: 1, and (ii) a S. aureus polypeptide antigen comprises a polypeptide comprising the amino acids selected from the group of: SEQ ID NO: 50 (SA1739; B1), SEQ ID NO: 51 (SA1720; B2), SEQ ID NO: 52 (SA1890; B3), SEQ ID NO: 53 (SA0103; T1), SEQ ID NO: 54 (SA0377; T2), SEQ ID NO: 55 (SA0693; T3), SEQ ID NO: 56 (SA2105; T4), or a fragment of at least 20 amino acids thereof, or a a polypeptide having at least 80%, or at least 85% or 90% sequence identity to any of SEQ ID NO: 51-56, or
(b) (i) a S. aureus polypeptide antigen comprises a polypeptide comprising the amino acids selected from the group of: SEQ ID NO: 50 (SA1739; B1), SEQ ID NO: 51 (SA1720; B2), SEQ ID NO: 52 (SA1890; B3), SEQ ID NO: 53 (SA0103; T1), SEQ ID NO: 54 (SA0377; T2), SEQ ID NO: 55 (SA0693; T3), SEQ ID NO: 56 (SA2105; T4), or a fragment of at least 20 amino acids thereof, or a a polypeptide having at least 80%, or at least 85% or 90% sequence identity to any of SEQ ID NO: 51-56, and (ii) a rhizavidin protein and at least one S. aureus polypeptide antigen, wherein the rhizavidin protein comprises amino acids of SEQ ID NO: 1, or 85% sequence identity to amino acids of SEQ ID NO: 1.

52. A fusion protein comprising, in any order, a rhizavidin protein and an S. aureus polypeptide antigen, wherein the rhizavidin protein comprises amino acids of SEQ ID NO: 1, or a polypeptide having at least 85% sequence identity to amino acids of SEQ ID NO: 1, and the S. aureus polypeptide comprises a SA1739 (B2) S. aureus polypeptide antigen comprising the amino acid sequence of at least 80%, or at least 85%, or at least 90% sequence identity to SEQ ID NO: 51.

53. The fusion protein of paragraph 52, wherein the SA1739 (B2) S. aureus polypeptide antigen comprises at least SEQ ID NO: 51 or a protein of at least 20 amino acids that has at least 85% sequence identity to SEQ ID NO: 51.

54. The fusion protein of paragraph 53, wherein the fusion protein further comprises, at least one additional S. aureus polypeptide antigen selected from any of: hemolysin (Hl), Clumping factor A (ClfA), Clumping factor B (ClfB), serine-aspirate repeat protein D (SdrD), serine-aspirate repeat protein E (SdrE), Iron regulator surface protein A (IsdA), Iron regulator surface protein B (IsdB), Leukotoxin D (LukD), Leukotoxin E (LukE), SA1720 (B1), SA1890 (B3), SA0103 (T1), SA0377 (T2), SA0693 (T3) or SA2105 (T4).

55. The fusion protein of paragraph 53, wherein the fusion protein further comprises (ii) at least one additional S. aureus polypeptide antigen selected from any of: SEQ ID NO: 50 (SA1739; B1), SEQ ID NO: 52 (SA1890; B3), SEQ ID NO: 53 (SA0103; T1), SEQ ID NO: 54 (SA0377; T2), SEQ ID NO: 55 (SA0693; T3) and SEQ ID NO: 56 (SA2105; T4), or a polypeptide having at least 80%, or at least 85% or 90% sequence identity to any of SEQ ID NO: 50, 52-56.

56. The fusion protein of any of paragraphs 52-55, wherein the fusion protein is selected from any of:
(a) a fusion protein comprising (i) a rhizavidin protein or biotin-binding protein comprising the amino acid sequence of SEQ ID NO: 1, or a polypeptide having at least 85% sequence identity to amino acids of SEQ ID NO: 1, and (ii) a SA1739 (B2) S. aureus polypeptide antigen comprising the amino acid sequence having at least 80%, or at least 85%, or at least 90% sequence identity to SEQ ID NO: 51,
(b) a fusion protein comprising (i) a SA1739 (B2) S. aureus polypeptide antigen comprising the amino acid sequence having at least 80%, or at least 85%, or at least 90% sequence identity to SEQ ID NO: 51, and (ii) a rhizavidin protein or biotin-binding protein comprising the amino acid sequence of SEQ ID NO: 1, or a polypeptide having at least 85% sequence identity to amino acids of SEQ ID NO: 1.

57. A fusion protein comprising, in any order, a rhizavidin protein and an S. aureus polypeptide antigen, wherein the rhizavidin protein comprises amino acids of SEQ ID NO: 1, or a polypeptide having at least 85% sequence identity to amino acids of SEQ ID NO: 1, and the S. aureus polypeptide selected from SA1720 (B1) or SA1890 (B3) S. aureus polypeptide antigen, wherein the SA1720 (B1) S. aureus polypeptide antigen comprises the amino acid sequence of at least 80%, or at least 85%, or at least 90% sequence identity to SEQ ID NO: 50, and wherein the SA1890 (B3) S. aureus polypeptide antigen comprises the amino acid sequence of at least 80%, or at least 85%, or at least 90% sequence identity to SEQ ID NO: 52.

58. The fusion protein of paragraph 57, wherein the SA1720 (B1) S. aureus polypeptide antigen comprises at least SEQ ID NO: 50 or a protein of at least 20 amino acids that has at least 85% sequence identity to SEQ ID NO: 50, and wherein the SA1890 (B3) S. aureus polypeptide antigen comprises at least SEQ ID NO: 52 or a protein of at least 20 amino acids that has at least 85% sequence identity to SEQ ID NO: 52.

59. The fusion protein of paragraph 57, wherein the fusion protein is selected from any of: (a) a fusion protein comprising, in the following order, (i) a rhizavidin protein or biotin-binding protein comprising the amino acids of SEQ ID NO: 1, or a polypeptide having at least 85% sequence identity to amino acids of SEQ ID NO: 1, and (ii) a SA1720 (B1) S. aureus polypeptide comprising the amino acid sequence of at least 80%, or at least 85%, or at least 90% sequence identity to SEQ ID NO: 50;
(b) a fusion protein comprising, in the following order, (i) a SA1720 (B1) S. aureus polypeptide comprising the amino acid sequence of at least 80%, or at least 85%, or at least 90% sequence identity to SEQ ID NO: 50, and (ii) a rhizavidin protein or biotin-binding protein comprising the amino acids of SEQ ID NO: 1, or a polypeptide having at least 85% sequence identity to amino acids of SEQ ID NO: 1,
(c) a fusion protein comprising, in the following order, (i) a rhizavidin protein or biotin-binding protein comprising the amino acids of SEQ ID NO: 1, or a polypeptide having at least 85% sequence identity to amino acids of SEQ ID NO: 1, and (ii) a or SA1890 (B3) S. aureus polypeptide comprising the amino acid sequence of at least 80%, or at least 85%, or at least 90% sequence identity to SEQ ID NO: 52; and
(d) a fusion protein comprising, in the following order, (i) a SA1890 (B3) S. aureus polypeptide comprising the amino acid sequence of at least 80%, or at least 85%, or at least 90% sequence identity to SEQ ID NO: 52, and (ii) a rhizavidin protein or biotin-binding protein comprising the amino acids of SEQ ID NO: 1, or a polypeptide having at least 85% sequence identity to amino acids of SEQ ID NO: 1.

60. The fusion protein of paragraph 57, wherein the fusion protein further comprises, fused to the SA1720 (B1) or SA1890 (B3) S. aureus polypeptide, at least one additional S. aureus polypeptide antigen selected from any of: hemolysin (Hl), Clumping factor A (ClfA), Clumping factor B (ClfB), serine-aspirate repeat protein D (SdrD), serine-aspirate repeat protein E (SdrE), Iron regulator surface protein A (IsdA), Iron regulator surface protein B (IsdB), Leukotoxin D (LukD), Leukotoxin E (LukE), SA1720 (B1), SA1739 (B2), SA1890 (B3), SA0103 (T1), SA0377 (T2), SA0693 (T3) or SA2105 (T4).

61. The fusion protein of any of paragraphs 57-60, wherein the fusion protein comprises (i) a SA1720 (B1) S. aureus antigen and further comprises (ii) at least one additional S. aureus polypeptide antigen selected from any of: SEQ ID NO: 50 (SA1739; B1), SEQ ID NO: 51 (SA1720; B2), SEQ ID NO: 52 (SA1890; B3), SEQ ID NO: 53 (SA0103; T1), SEQ ID NO: 54 (SA0377; T2), SEQ ID NO: 55 (SA0693; T3), SEQ ID NO: 56 (SA2105; T4), or a fragment of at least 20 amino acids thereof, or a a polypeptide having at least 80%, or at least 85% or 90% sequence identity to any of SEQ ID NO: 51-56.

62. The fusion protein ofany of paragraphs 57-60, wherein the fusion protein comprises (i) a SA1890 (B3) S. aureus antigen and further comprises (ii) at least one additional S. aureus polypeptide antigen selected from any of: SEQ ID NO: 50 (SA1739; B1), SEQ ID NO: 51 (SA1720; B2), SEQ ID NO: 52 (SA1890; B3), SEQ ID NO: 53 (SA0103; T1), SEQ ID NO: 54 (SA0377; T2), SEQ ID NO: 55 (SA0693; T3), SEQ ID NO: 56 (SA2105; T4), or a fragment of at least 20 amino acids thereof, or a polypeptide having at least 80%, or at least 85% or 90% sequence identity to any of SEQ ID NO: 51-56.

63. A fusion protein comprising, in any order, a rhizavidin protein and an S. aureus polypeptide antigen, wherein the rhizavidin protein comprises amino acids of SEQ ID NO: 1, or a polypeptide having at least 85% sequence identity to amino acids of SEQ ID NO: 1, and the S. aureus polypeptide comprises a SA0103 (T1) S. aureus polypeptide antigen comprising the amino acid sequence of at least 80%, or at least 85%, or at least 90% sequence identity to SEQ ID NO: 53.

64. The fusion protein of paragraph 63, wherein the fusion protein is selected from any of:
(a) a fusion protein comprising (i) a rhizavidin protein or biotin-binding protein comprising the amino acid sequence of SEQ ID NO: 1, or a polypeptide having at least 85% sequence identity to amino acids of SEQ ID NO: 1, and (ii) SA0103 (T1) S. aureus polypeptide antigen comprising the amino acid sequence having at least 80%, or at least 85%, or at least 90% sequence identity to SEQ ID NO: 53,
(b) a fusion protein comprising (i) a SA0103 (T1) S. aureus polypeptide antigen comprising the amino acid sequence having at least 80%, or at least 85%, or at least 90% sequence identity to SEQ ID NO: 53, and (ii) a rhizavidin protein or biotin-binding protein comprising the amino acid sequence of SEQ ID NO: 1, or a polypeptide having at least 85% sequence identity to amino acids of SEQ ID NO: 1.

65. The fusion protein of paragraph 63 or 64, wherein the SA0103 (T1) S. aureus polypeptide antigen comprises at least SEQ ID NO: 53 or a protein of at least 20 amino acids that has at least 85% sequence identity to SEQ ID NO: 53.

66. The fusion protein of any of paragraphs 63-65, wherein the fusion protein comprises (i) a SA0103 (T1) S. aureus antigen, and further comprises (ii) at least one additional S. aureus polypeptide antigen selected from any of: SA1720 (B1), SA1739 (B2), SA1890 (B3), hemolysin (Hl), Clumping factor A (ClfA), Clumping factor B (ClfB), serine-aspirate repeat protein D (SdrD), serine-aspirate repeat protein E (SdrE), Iron regulator surface protein A (IsdA), Iron regulator surface protein B (IsdB), Leukotoxin D (LukD) or Leukotoxin E (LukE).

67. The fusion protein of any of paragraphs 63-67, wherein the fusion protein further comprises (ii) at least one additional S. aureus polypeptide antigen selected from: SEQ ID NO: 54 (SA0377; T2), SEQ ID NO: 55 (SA0693; T3), SEQ ID NO: 56 (SA2105; T4), or T4) or a polypeptide having at least 80%, or at least 85% or 90% sequence identity to any of SEQ ID NO: 54-56.

68. The fusion protein of any of paragraphs 63-67, wherein the fusion protein comprises (i) a SA0103 (T1) S. aureus polypeptide antigen comprising the amino acid sequence of at least 80%, or at least 85%, or at least 90% sequence identity to SEQ ID NO: 53 and further comprises (ii) at least a SA0377 (T2) S. aureus polypeptide antigen comprising the amino acid sequence of at least 80%, or at least 85%, or at least 90% sequence identity to SEQ ID NO: 54.

69. The fusion protein of any of paragraphs 63-67, wherein the fusion protein comprises (i) SA0103 (T1) S. aureus polypeptide antigen comprising the amino acid sequence of at least 80%, or at least 85%, or at least 90% sequence identity to SEQ ID NO: 53 (T1) S. aureus antigen and further comprises (ii) at least a SA0693 (T3) S. aureus polypeptide antigen comprising the amino acid sequence of at least 80%, or at least 85%, or at least 90% sequence identity to SEQ ID NO: 55.

70. The fusion protein of any of paragraphs 63-67, wherein the fusion protein comprises (i) SA0103 (T1) S. aureus polypeptide antigen comprising the amino acid sequence of at least 80%, or at least 85%, or at least 90% sequence identity to SEQ ID NO: 53 (T1) S. aureus antigen and further comprises (ii) at least a SA2105 (T4) S. aureus polypeptide antigen comprising the amino acid sequence of at least 80%, or at least 85%, or at least 90% sequence identity to SEQ ID NO: 56.

71. A fusion protein comprising, in any order, (i) a rhizavidin protein or biotin-binding protein, (ii) a first S. aureus polypeptide antigen and (iii) a second S. aureus polypeptide antigen, wherein the fusion protein is selected from any of:
(a) a fusion protein comprising, in any order, a rhizavidin protein or biotin-binding protein comprises amino acids of SEQ ID NO: 1 or a polypeptide having at least 85% sequence identity to amino acids of SEQ ID NO: 1, a first S. aureus polypeptide antigen is selected from any of: SA1720 (B1), SA1739 (B2) or SA1890 (B3), and a second S. aureus polypeptide antigen is selected from any of: SA0103 (T1), SA0377 (T2), SA0693 (T3) or SA2105 (T4);
(b) a fusion protein comprising, in any order, a rhizavidin protein or biotin-binding protein comprises amino acids of SEQ ID NO: 1 or a polypeptide having at least 85% sequence identity to amino acids of SEQ ID NO: 1, a first S. aureus polypeptide antigen is selected from any of: SA0103 (T1), SA0377 (T2), SA0693 (T3) or SA2105 (T4); and a second S. aureus polypeptide antigen is selected from any of: SA0103 (T1), SA0377 (T2), SA0693 (T3) or SA2105 (T4); and
(c) a fusion protein comprising, in any order, a rhizavidin protein or biotin-binding protein comprises amino acids of SEQ ID NO: 1 or a polypeptide having at least 85% sequence identity to amino acids of SEQ ID NO: 1, a first S. aureus polypeptide antigen is selected from any of: SA1720 (B1), SA1739 (B2) or SA1890 (B3), and a second S. aureus polypeptide antigen is selected from any of: SA1720 (B1), SA1739 (B2) or SA1890 (B3),
and wherein, the S. aureus polypeptide antigens comprise the amino acid sequence selected from from the group of: SEQ ID NO: 50 (SA1739; B1), SEQ ID NO: 51 (SA1720; B2), SEQ ID NO: 52 (SA1890; B3), SEQ ID NO: 53 (SA0103; T1), SEQ ID NO: 54 (SA0377; T2), SEQ ID NO: 55 (SA0693; T3), SEQ ID NO: 56 (SA2105; T4), or a fragment of at least 20 amino acids thereof, or a polypeptide having at least 80%, or at least 85% or 90% sequence identity to any of SEQ ID NO: 51-56.

72. The fusion protein of paragraph 71, wherein the fusion protein is selected from any of the fusion proteins disclosed in Table 5B.

73. The fusion protein of paragraph 71 or 72, wherein the fusion protein comprises a rhizavidin protein or biotin-binding protein and a first S. aureus polypeptide antigen and second S. aureus polypeptide antigen, in any order, where the first and second S. aureus polypeptide antigens are selected from the groups of: SA1720 (B1) and SA0103 (T1), SA1720 (B1) and SA0377 (T2),SA1720 (B1) and SA0693 (T3), SA1720 (B1) and SA2105 (T4), SA1739 (B2) and SA0103 (T1), SA1739 (B2) and SA0377 (T2), SA1739 (B2) and SA0693 (T3), SA1739 (B2) and SA2105 (T4), SA1890 (B3) and SA0103 (T1), SA1890 (B3) and SA0377 (T2), SA1890 (B3) and SA0693 (T3), SA1890 (B3) and SA2105 (T4), SA0103 (T1) and SA0377 (T2), SA0103 (T1) and SA0693 (T3), SA0103 (T1) and SA2105 (T4), SA0377 (T2) and SA0693 (T3),SA0377 (T2) and SA2105 (T4), and SA0693 (T3) and SA2105 (T4). (i.e., each group of two antigens represent the first S. aureus polypeptide antigen and second S. aureus polypeptide antigens, respectively).

74. The fusion protein of any of paragraphs 71-73, wherein the SA1720 (B1) S. aureus polypeptide antigen comprises the amino acid sequence of SEQ ID NO: 50 or a polypeptide with at least 80%, or at least 85%, or at least 90% amino acid sequence identity to SEQ ID NO: 50, or a protein of at least 20 amino acids that has at least 85% sequence identity to SEQ ID NO: 50.

75. The fusion protein of any of paragraphs 71-73, wherein the SA1739 (B2) S. aureus polypeptide antigen comprises the amino acid sequence of SEQ ID NO: 51 or a polypeptide with at least 80%, or at least 85%, or at least 90% amino acid sequence identity to SEQ ID NO: 51, or a protein of at least 20 amino acids that has at least 85% sequence identity to SEQ ID NO: 51.

76. The fusion protein of any of paragraphs 71-73, wherein the SA1809(B3) S. aureus polypeptide antigen comprises the amino acid sequence of SEQ ID NO: 52 or a polypeptide with at least 80%, or at least 85%, or at least 90% amino acid sequence identity to SEQ ID NO: 52, or or a protein of at least 20 amino acids that has at least 85% sequence identity to SEQ ID NO: 52.

77. The fusion protein of any of paragraphs 71-73, wherein the SA0103 (T1) S. aureus polypeptide antigen comprises the amino acid sequence of SEQ ID NO: 53 or a polypeptide with at least 80%, or at least 85%, or at least 90% amino acid sequence identity to SEQ ID NO: 53, or a protein of at least 20 amino acids that has at least 85% sequence identity to SEQ ID NO: 53.

78. The fusion protein of any of paragraphs 71-73, wherein the SA0377 (T2) S. aureus polypeptide antigen comprises the amino acid sequence of SEQ ID NO: 54 or a polypeptide with at least 80%, or at least 85%, or at least 90% amino acid sequence identity to SEQ ID NO: 54, or a protein of at least 20 amino acids that has at least 85% sequence identity to SEQ ID NO: 54.

79. The fusion protein of any of paragraphs 71-73, wherein the SA0693 (T3) S. aureus polypeptide antigen comprises the amino acid sequence of SEQ ID NO: 55 or a polypeptide with at least 80%, or at least 85%, or at least 90% amino acid sequence identity to SEQ ID NO: 55, or a protein of at least 20 amino acids that has at least 85% sequence identity to SEQ ID NO: 55.

80. The fusion protein of any of paragraphs 71-73, wherein the SA2105 (T4) S. aureus polypeptide antigen comprises the amino acid sequence of SEQ ID NO: 56 or a polypeptide with at least 80%, or at least 85%, or at least 90% amino acid sequence identity to SEQ ID NO: 56, or a protein of at least 20 amino acids that has at least 85% sequence identity to SEQ ID NO: 56.

81. A vaccine composition comprising any of the fusion proteins of paragraphs 50-80.

82. A vaccine composition comprising the immunogenic composition of any of paragraphs 1-42.

83. A pharmaceutical composition comprising the immunogenic composition of any of paragraphs 1-42 and a pharmaceutically acceptable carrier.

84. The pharmaceutical composition of paragraph 83, comprising any of the fusion proteins of paragraphs 50-82.

85. The pharmaceutical composition of paragraph 83, further comprising one or more adjuvants.

86. The pharmaceutical composition of paragraph 85, wherein the one or more adjuvants is or comprises a co-stimulation factor.

87. The pharmaceutical composition of paragraph 86, wherein the one or more adjuvants are selected from the group consisting of aluminum phosphate, aluminum hydroxide, and phosphated aluminum hydroxide.

88. The pharmaceutical composition of any one of paragraphs 83-87, wherein upon administration to a subject, the pharmaceutical composition induces an opsonic/bactericidal response against one or more serotypes of S. aureus.

89. The pharmaceutical composition of any one of paragraphs 83-87, wherein upon administration to a subject, the pharmaceutical composition reduces or inhibits transmission of one or more serotypes of S. aureus. from the subject to another subject.

90. The pharmaceutical composition of any one of paragraphs 83-87, wherein upon administration to a subject, the pharmaceutical composition inhibits, or reduces the rate of occurrence of, or reduced the severity of any of the diseases or disorders selected from: sepsis, Pneumonia (including ventilator-associated pneumonia), sinusitis, skin infections/abscess/cellulitis/furuncles/impetigo, bloodstream infection, meningitis, endocarditis (heart infection), osteomyelitis, septic arthritis (or bone/joint infections), myositis (muscle infection), mastitis, mastoiditis, toxic shock syndrome, necrotizing fasciitis catheter/central line infection, prosthetic material infection including joint prostheses, surgical site infections associated with or induced by one or more serotypes of S. aureus.

91. The pharmaceutical composition of any one of paragraphs 83-87, wherein upon administration to a subject, the pharmaceutical composition inhibits, or reduces the rate of occurrence of, or reduced the severity of pneumonia associated with or induced by one or more serotypes of S. aureus.

92. The pharmaceutical composition of any one of paragraphs 83-87, wherein upon administration to a subject, the pharmaceutical composition inhibits, or reduces the rate of, colonization of mucosal surfaces by one or more serotypes of S. aureus.

93. The pharmaceutical composition of any one of paragraphs 83-87, wherein upon administration to a subject, the pharmaceutical composition inhibits, or reduces the rate of, colonization of the nasopharynx by one or more serotypes of S. aureus.

94. The pharmaceutical composition of any one of paragraphs 83-93, wherein the pharmaceutical composition is formulated for injection.

95. The pharmaceutical composition of any one of paragraphs 83-94, wherein upon administration to a subject, the pharmaceutical composition induces an immune response.

96. The pharmaceutical composition of paragraph 90, wherein the immune response comprises an antibody and/or B cell response.

97. The pharmaceutical composition of paragraph 95 or paragraph 96, wherein the immune response comprises a CD4+ T cell response (e.g., TH1, TH2, or TH17 response); a CD8+ T cell response; a CD4+ and CD8+ T cell response; or a CD4−/CD8− T cell response.

98. The pharmaceutical composition of any one of paragraphs 95-97, wherein the immune response comprises (i) an antibody or B cell response and (ii) a T cell response.

99. The pharmaceutical composition of any one of paragraphs 95-98, wherein the immune response is to (i) at least one polysaccharide antigen of the immunogenic complex, and/or (ii) at least one polypeptide antigen of the immunogenic complex.

100. The pharmaceutical composition of any one of paragraphs 95-99, wherein the immune response comprises (i) an antibody or B cell response to at least one polysaccharide antigen of the immunogenic complex, and (ii) a CD4+ T cell response (e.g., TH1, TH2, or TH17 response), a CD8+ T cell response, a CD4+ and CD8+ T cell response, or a CD4−/CD8− T cell response to at least one polypeptide antigen of the immunogenic complex.

101. The pharmaceutical composition of any one of paragraphs 95-100, wherein the immune response comprises (i) an antibody or B cell response to at least one polysaccharide antigen of the immunogenic complex, and (ii) an antibody or B cell response to at least one polypeptide antigen of the vaccine or immunogenic complex.

102. The pharmaceutical composition of any one of paragraphs 95-96, wherein the immune response comprises (i) an antibody or B cell response to at least one polysaccharide antigen of the immunogenic complex, and (ii) an antibody or B cell response; and a CD4+ T cell response (including TH1, TH2, or TH17 response), a CD8+ T cell response, a CD4+ and CD8+ T cell response, or a CD4−/CD8− T cell response to at least one polypeptide antigen of the immunogenic complex.

103. A heterologous nucleic acid sequence, comprising, in any order, (i) a nucleic acid sequence encoding a biotin-binding moiety having an amino acid sequence of at least 80%, or 90%, or 95% sequence identity to SEQ ID NO: 1, and (ii) a nucleic acid sequence encoding a S. aureus polypeptide antigen comprises a polypeptide comprising the amino acids selected from the group of: SEQ ID NO: 50 (SA1739; B1), SEQ ID NO: 51 (SA1720; B2), SEQ ID NO: 52 (SA1890; B3), SEQ ID NO: 53 (SA0103; T1), SEQ ID NO: 54 (SA0377; T2), SEQ ID NO: 55 (SA0693; T3), SEQ ID NO: 56 (SA2105; T4), or a fragment of at least 20 amino acids thereof, or a polypeptide having at least 80%, or at least 85% or 90% sequence identity to any of SEQ ID NO: 51-56.

104. The heterologous nucleic acid sequence of paragraph 103, wherein the nucleic acid sequence comprises, in the following order:
(a) a nucleic acid sequence encoding a biotin-binding moiety having an amino acid sequence of at least 80%, or 90%, or 95% sequence identity to SEQ ID NO: 1, and a nucleic acid sequence encoding a S. aureus polypeptide antigen comprises a polypeptide comprising the amino acids selected from the group of: SEQ ID NO: 50 (SA1739; B1), SEQ ID NO: 51 (SA1720; B2), SEQ ID NO: 52 (SA1890; B3), SEQ ID NO: 53 (SA0103; T1), SEQ ID NO: 54 (SA0377; T2), SEQ ID NO: 55 (SA0693; T3), SEQ ID NO: 56 (SA2105; T4), or a fragment of at least 20 amino acids thereof, or a polypeptide having at least 80%, or at least 85% or 90% sequence identity to any of SEQ ID NO: 51-56, or
(b) (i) a nucleic acid sequence encoding a S. aureus polypeptide antigen, the S. aureus polypeptide antigen comprising the amino acids selected from the group of: SEQ ID NO: 50 (SA1739; B1), SEQ ID NO: 51 (SA1720; B2), SEQ ID NO: 52 (SA1890; B3), SEQ ID NO: 53 (SA0103; T1), SEQ ID NO: 54 (SA0377; T2), SEQ ID NO: 55 (SA0693; T3), SEQ ID NO: 56 (SA2105; T4), or a fragment of at least 20 amino acids thereof, or a polypeptide having at least 80%, or at least 85% or 90% sequence identity to any of SEQ ID NO: 51-56, and (ii) a nucleic acid sequence encoding a biotin-binding moiety having an amino acid sequence of at least 80%, or 90%, or 95% sequence identity to SEQ ID NO: 1.

105. The heterologous nucleic acid sequence of paragraphs 104 or 105, wherein the nucleic acid sequence comprises, in the following order:
(a) a nucleic acid sequence comprising (i) a nucleic acid sequence comprising SEQ ID NO: 81, or a nucleic acid sequence having 80%, or 90%, or 95% sequence identity to SEQ ID NO: 81 that encodes a biotin-binding moiety having an amino acid sequence of at least 80%, or 90%, or 95% sequence identity to SEQ ID NO: 1, and (ii) a nucleic acid sequence selected from the group of: SEQ ID NO: 57 (SA1739; B1), SEQ ID NO: 58 (SA1720; B2), SEQ ID NO: 59 (SA1890; B3), SEQ ID NO: 60 (SA0103; T1), SEQ ID NO: 67 (SA0377; T2), SEQ ID NO: 62 (SA0693; T3), SEQ ID NO: 63 (SA2105; T4), or a nucleic acid sequence having at least 80%, or at least 85% or 90% sequence identity to any of SEQ ID NO: 57-63 that encodes a polypeptide selected from SEQ ID NO: 51-56 or a polypeptide having at least 85% amino acid sequence identity to SEQ ID NO: 51-56,
(b) a nucleic acid sequence comprising (i) a nucleic acid sequence selected from the group of SEQ ID NO: 57 (SA1739; B1), SEQ ID NO: 58 (SA1720; B2), SEQ ID NO: 59 (SA1890; B3), SEQ ID NO: 60 (SA0103; T1), SEQ ID NO: 67 (SA0377; T2), SEQ ID NO: 62 (SA0693; T3), SEQ ID NO: 63 (SA2105; T4), or a nucleic acid sequence having at least 80%, or at least 85% or 90% sequence identity to any of SEQ ID NO: 57-63 that encodes a polypeptide selected from SEQ ID NO: 51-56 or a polypeptide having at least 85% amino acid sequence identity to SEQ ID NO: 51-56, and (ii) a nucleic acid sequence comprising (i) a nucleic acid sequence comprising SEQ ID NO: 81, or a nucleic acid sequence having 80%, or 90%, or 95% sequence identity to SEQ ID NO: 81 that encodes a biotin-binding moiety having an amino acid sequence of at least 80%, or 90%, or 95% sequence identity to SEQ ID NO: 1.

106. The heterologous nucleic acid sequence of any of paragraphs 103-105, further comprising (iii) a third nucleic acid sequence selected from the group of: SEQ ID NO: 57 (SA1739; B1), SEQ ID NO: 58 (SA1720; B2), SEQ ID NO: 59 (SA1890; B3), SEQ ID NO: 60 (SA0103; T1), SEQ ID NO: 67 (SA0377; T2), SEQ ID NO: 62 (SA0693; T3), SEQ ID NO: 63 (SA2105; T4), or a nucleic acid sequence having at least 80%, or at least 85% or 90% sequence identity to any of SEQ ID NO: 57-63 that encodes a polypeptide selected from SEQ ID NO: 51-56 or a polypeptide having at least 85% amino acid sequence identity to SEQ ID NO: 51-56.

107. The heterologous nucleic acid sequence of any of paragraphs 103-106, wherein the nucleic acid encodes a fusion protein comprising a biotin-binding moiety having an amino acid sequence of at least 80%, or 90%, or 95% sequence identity to SEQ ID NO: 1 that has any one or more of the amino acid modifications: N80, T108, N118, S119A, N138A.

108. The heterologous nucleic acid sequence of any of paragraphs 103-106, operatively linked to a promoter.

109. The heterologous nucleic acid sequence of any of paragraphs 103-108, wherein the heterologous nucleic acid further comprises any one or more of: a nucleic acid that encodes at least one linker, or a nucleic acid that encodes an expression tag, or a nucleic acid that encodes a lipidation sequence.

110. The heterologous nucleic acid sequence of any of paragraphs 103-109, wherein the nucleic acid sequence encoding at least one linker is located between the nucleic acid sequence of SEQ ID NO: 81 and the nucleic acid sequence selected from the group of: SEQ ID NO: 57 (SA1739; B1), SEQ ID NO: 58 (SA1720; B2), SEQ ID NO: 59 (SA1890; B3), SEQ ID NO: 60 (SA0103; T1), SEQ ID NO: 67 (SA0377; T2), SEQ ID NO: 62 (SA0693; T3), SEQ ID NO: 63 (SA2105; T4).

111. The heterologous nucleic acid sequence of paragraph 109 or 110, wherein the nucleic acid sequence encodes at least one linker having an amino acid sequence selected from any of: GGGGSSS (SEQ ID NO: 71) or AAA (SEQ ID NO: 72) and any of: SEQ ID NOS: 30-37 or SEQ ID NO: 44 or 45.

112. An expression vector comprising a nucleic acid sequence of any of paragraphs 103-110.

113. The expression vector of paragraph 112, wherein the vector is an expression vector.

114. A cell comprising the heterologous nucleic acid of sequence of any of paragraphs 103-110, or an expression vector of any of paragraphs 112 or 113.

115. The cell of paragraph 114, where the cell is an expression host cell.

116. The cell of paragraph 115, wherein the expression host cell is selected from the group consisting of: E. coli, an insect cell line or a mammalian cell line.

117. The cell of paragraph 116, wherein the insect cell line is baculovirus expression system.

118. The cell of paragraph 116, wherein the mammalian cell line is a human cell line or Chinese Hamster ovary (CHO) cell line.

119. The cell of any of paragraphs 114-119, wherein the heterologous nucleic acid sequence is codon-optimized to improve expression in the host cell.

120. The cell of any of paragraphs 114-119, wherein the heterologous nucleic acid sequence further comprises a nucleic acid sequence encoding one or more of: polyadenylation sequence or termination sequence, a signal sequence.

121. The fusion protein of any of paragraphs 50-80, encoded by the heterologous nucleic acid sequence of paragraphs 103-110, or expressed by the expression vector of any of paragraphs 112 or 113, or produced in a cell of any of paragraphs 114-120.

### EXAMPLES

The examples presented herein relate to methods to generate an immunogenic complex as described herein and methods and compositions thereof. In particular, the examples relate to methods to produce a S. aureus multiple antigen presentation system (SA-MAPS) complex as disclosed herein, and methods of use to generate an immune response in a subject.

### Materials and METHODS

Bacterial strains: S. aureus strains USA 300/TCH959 and ATCC 29213 were originally purchased from ATCC. S. aureus strain USA 300 LAC was provided by BEI. Streptomycin-resistant USA 300 LAC strain (USA 300 LAC^{strep}) was obtained by streaking USA 300 LAC strain on blood agar plate containing 0.5 g/L streptomycin and selecting a spontaneous mutant the following morning.

Cloning and purification of S. aureus antigens. DNA sequences encoding SA1739 (B2), SA1720 (B1), SA1890 (B3), SA0103 (T1), SA0377 (T2), SA0693 (T3) and SA2105 (T4) were amplified from S. aureus genomic DNA (purified from USA 300 TCH959 strain) via PCR and then cloned into a pET-21b vector. For rhizavidin fusion proteins, DNA sequences encoding SA antigens were inserted at the 3′ end of the gene encoding rhizavidin moiety in a pET-21b vector. All constructs were transformed into E. coli BL21 (DE3) strain for expression. His-tagged recombinant proteins were purified using Ni-nitrilotriacetic acid (NTA) affinity chromatography (Quagen) followed by size-exclusion chromatography using a Superdex 200 column (GE lifescience). Purified proteins were stored at −80° C. until use.

OPA Killing Assay: Serum from Balb/c mice (n=5 per Group 1-8) after innocuation with SA proteins (20 proteins/group) was assessed using a OPA killing assay. In particular, the 159 proteins from the result of surface library screen were allocated into 8 groups of 20 and used for immunization of 5 Balb/c mice per group (each mouse group received a combination of 20 proteins at 5 μg per protein). Mice were bled two weeks after the second immunization for initial test and terminally bled at 5 weeks post-second immunization. Serum was used in OPA killing assays with neutrophils derived from HL-60 cells. Serum was heat-inactivated at 56C for 30 minutes, bacteria was washed and resuspend at 2×105 CFU/ml. 10 μl of bacteria suspension was added to each well with serum. 20 μl of serum was added per well of assay and incubated at room temperature for 30 min on shaker. After 30 mins, 10 μl baby rabbit complement was added to each well and followed by incubation at room temperature for 30 min on shaker. After 30 mins, the HL60 cells were washed and resuspend to 5×106 cells/ml. 40 μl HL60 suspension was added to each well (100:1 HL60 to bacteria ratio) and the plate incubated shaking at 37° C. with 5% CO2 for 1 hour. The plates were then incubated on ice for 20 minutes to stop phagocytosis. Different dilutions (neat, 1/5, and 1/25 dilutions (in water)) of each well was plated on 5% blood agar plates and incubate overnight at 37° C. with 5% CO2. After 24 hours, the number of colonies was counted and determined.

### Inhibition of Killing by Purified Proteins:

1 μl of serum plus 9 ul of buffer was added per well of assay. 10 ul of each protein was added and incubated at RT for 30 minutes. After 30 mins, the bacteria was washed and resuspend at 2×10⁵ CFU/ml. 10 μl of bacteria suspension was added to each well with serum and incubated at room temperature for 30 min on shaker. 10 μl baby rabbit complement was added to each well and incubated at room temperature for 30 min on shaker. After 30 mins, the HL60 cells were washed and resuspend to 5×10⁶ cells/ml. 40 μl of HL60 suspension was added to each well (100:1 HL60 to bacteria ratio) and the plate incubated with shaking at 37° C. with 5% CO₂ for 1 hour. After 1 hour, the plate was placed on ice for 20 minutes to stop phagocytosis. Serial dilutions (neat, ⅕, and 1/25 dilutions (in water)) of each well was performed on 5% blood agar plates and incubate overnight at 37° C. with 5% CO₂. After 12-18 hours, the colonies were counted.

Preparation of MAPS Complex. Type 1 pneumococcal capsular polysaccharide (referred to herein as CP1 or PS1 or SP PS1) was purchased from ATCC. Biotinylation of the polysaccharide was done as described previously. The SA-MAPS complex was assembled by incubation of biotinylated immunogenic polysaccharide with a mixture of rhizavidin fusions of S. aureus antigens at room temperature overnight. Typically, the rhizavidin fusion comprises 1:1 or 1:2 ratio of Rhizavidin: SA antigen. In some embodiments, the rhizavidin fusion comprises 1:2 ratio of Rhizavidin: SA antigen, in that the Rhizavidin protein can comprise 2 SA antigens, e.g., a Rhavi-A-A fusion protein, or a Rhavi-A-B fusion protein, where A is one SA antigen, and B is a different SA antigen. In some embodiments, the rhizavidin fusion comprises 1:3 ratio of Rhizavidin: SA antigen, in that the Rhizavidin protein can comprise 3 SA antigens, e.g., a Rhavi-A-A-A fusion protein, or a Rhavi-A-B-A fusion protein, or a Rhavi-A-B-C fusion protein, where A is one SA antigen, and B is a different SA antigen, and C is a different SA antigen. The order of SA antigens A, B and C attached to the rhizavidin (Rhavi) protein of SEQ ID NO: 1 can be in any order, and in some embodiments, any one of A, B or C can be a non-SA antigen as disclosed herein.

In some embodiments, the input ratio of Rhizavidin fusion proteins to polysaccharide was 3:1 (w/w). The assembled complex was isolated by size-exclusion chromatography. The fractions containing MAPS complex were pooled and concentrated by ultrafiltration. The protein concentration in a MAPS complex was measured using a bicinchoninic acid (BCA) protein assay kit (Pierce). The incorporation of target antigens was examined on a reduced SDS-PAGE gel.

Immunization and infection. Vaccines were prepared one day prior to immunization. The antigens were diluted to the appropriate concentration in saline, then mixed with aluminum hydroxide (Brenntag) and incubated at 4° C. overnight with rotation. 4-6-week-old C57BL/6 wild type or μMT^{-/-} female mice (Jackson Laboratories) received three subcutaneous immunizations two weeks apart. Animals were bled two week after the last immunization for analysis of antibody and T-cell responses. Mice were infected with S. aureus 7-10 days later.

Rabbit antisera against S. aureus antigens were generated at Cocalico Biologicals (Reamstown, PA). New Zealand White rabbits were given three intramuscular immunizations, two weeks apart, with SA-MAPS vaccine. Sera were collected before the first immunization and two weeks after the last immunization. For passive immunization, 8-week-old C57BL/6 female mice received 200 μl of heat-inactivated pre- or post-vaccination rabbit sera one day prior to infection via intraperitoneal injection.

For all infections, S. aureus strains were streaked on blood agar plates and grown at 37° C. overnight. An overnight culture was then started by inoculating colonies into Tryptic soy broth (TSB, Sigma), shaking at 37° C. Cells were re-inoculated into fresh TSB medium at 1:100 dilution in the morning and incubated at 37° C. with shaking for 3 hours. Bacterial cells were collected by centrifugation, washed twice with saline and adjusted to certain concentration in saline before infection.

Sepsis infection of S. aureus was performed using the ATCC 29213 strain. Mice were anesthetized with isoflurane and infected with 2-3×10⁷ CFU in 100 μl via retro-orbital injection. Mice were monitored for sign of illness for 14 days; any ill-appearing animal was immediately and humanely euthanized.

In the dermonecrosis infection model, mice were anesthetized and injected subcutaneously on the shaved lower back with 0.5-1×107 CFU of USA300 TCH959 strain in 100 μl volume. Mice were monitored for 14 days after infection. Pictures of infected area were taken and the sizes of dermonecrotic plaques/lesions were measured using ImageJ software.

In the skin abscess model, mice were shaved, anesthetized and infected subcutaneously with 2-5×10⁵ of USA300 TCH959 strain in 100 μl volume. Mice were then humanely euthanized on day 4 after infection. Abscesses were dissected and homogenized by bead beater. Serial dilutions of homogenate were plated on mannitol salt plates and colonies were counted after overnight culture at 37° C. Detection limit is 22.5 CFU. In animals that were abscess-free or for culture negative samples, their CFU was set as the detection limit.

In the GI colonization model, mice were gently restrained and inoculated intranasally with 5×10⁷ of USA 300 LAC^{strep} strain in 10 μl volume. Fecal pellets were collected on day 1 and day 7 after infection or as indicated. Samples were weighted, resuspended in sterile PBS at 0.1 g/ml, homogenized and then passed through CellTrics 30 μm filter. Serial dilutions of the flow-through samples were plated on mannitol salt plates containing 0.5 g/L streptomycin and colonies were counted after overnight culture at 37° C. Detection limit is 40 CFU. For culture-negative samples, CFU was set as the detection limit.

Antibody and T-cell response analysis. Antigen-specific IgG antibody was measured by ELISA using Immulon 2 HB 96-microwell plates (Thermo Scientific) coated with individual recombinant S. aureus protein (non-rhizavidin fusion). The plates were washed with PBS containing 0.05% Tween 20 (PBS-T) and then blocked with 1% BSA in PBS for 1 hour. After blocking, serial dilutions of mouse or rabbit sera were added and incubated for 2 hours, followed by 1 hour incubation with HRP-conjugated secondary antibody against mouse or rabbit IgG. The plates were then washed and developed with SureBlue TMB Microwell Peroxidase Substrate (KPL). 1 M HCl was used to terminate the reactions before the A₄₅₀ₙₘ was analyzed using an ELISA reader. Antibody titers were expressed in arbitrary units relative to a standard serum.

For T-cell response analysis, 25 μl of heparinized blood were added to 225 μl DMEM (BioWhittaker) containing 10% low-endotoxin defined FBS (Hyclone), 50 μM 2-mercaptoethanol (Sigma) and ciprofloxacin (10 μg/ml, Cellgro). The cultures were incubated at 37° C. for 6 days in the presence of 2.5 g/ml of the mixture of six S. aureus protein antigens (equal weight ratio, non-rhizavidin fusion). Supernatants were collected following centrifugation and analyzed by ELISA for INF-γ, IL-17A and IL-22 concentration (R&D Systems).

Hemolysis analysis. The hemolytic activity of wild type Hla, Hla209 and their rhizavidin fusions was measured as follows: 200 μl of heparinized rabbit blood was washed with cold PBS three times. Red blood cells were then resuspended in 10 ml of cold PBS (2% rabbit red blood cells) and 100 μl of 2-fold serial dilution of Hla samples in PBS with 0.1% BSA, starting from 100 μg/ml, was added into a V-bottom 96-well plate before the addition of 100 μl of red blood cells to each well. PBS containing 0.1% Triton X-100 was used as a positive control (100% hemolysis), and PBS with 0.1% BSA was used as a negative control (0% hemolysis). The plate was incubated at 37° C. for 30 min and then subjected to centrifugation at 800 g for 5 minutes. The supernatants were transferred into a flat-bottom 96-well plate and the A₅₄₅ₙₘ was measured by an ELISA reader. One hemolytic unit (HU) was defined as the activity that causes 50% lysis of 1% rabbit red blood cells after 30 min incubation at 37° C. The activity of each Hla construct was expressed as the HU of 1 mg/ml of purified protein.

Statistical analysis. All statistical analyses were done using PRISM (version 5.01 for Windows, GraphPad Software, Inc). Antibody titer, cytokine release, size of lesions and CFU counts in abscesses or in feces were compared between groups using the Mann-Whitney Utest. Differences in survival were analyzed by the Mantel-Cox test. Percentage of abscess formation was analyzed by the Fisher's exact test.

### Example 1

It is well recognized that any single animal model of SA infection is unlikely to adequately represent the pathophysiology of disease in humans; therefore, evaluation of any potential candidate in several models would appear prudent. At the same time, the large number of virulence factors (including polysaccharides, surface proteins, and secreted toxins produced by SA, may provide credence to the idea that multiple, genetically conserved antigens should be included in a candidate vaccine. Finally, a closer examination of mechanisms of immunity to SA in humans may also provide clues for an effective vaccine strategy. Indeed, while humoral immunity plays a leading role in host defense against many bacterial or viral pathogens, it is unlikely that antibodies are the only or even the primary factor for resistance to SA. Patients with B-cell deficiencies do not appear to be at significantly increased risk of SA infections, and individuals with high levels of pre-existing SA-specific antibodies can still get infected by SA. On the other hand, a growing body of literature now suggests that T-cell immunity, the other arm of acquired host defense, plays a critical role in SA defense. Indeed, individuals with suppressed or impaired cellular immunity, caused by high dose prednisone therapy, HIV infection, defective interferon-γ (IFN-γ) production, defective interleukin-17 (IL-17) production, are at very high risk for SA infection and recurrence. Moreover, in murine models, IFN-γ or IL-17A/F deficiency has been shown to induce hyper-susceptibility to SA skin infections, and IL-17A deficiency in mice is also associated with prolonged nasal carriage of SA. Therefore, an approach the combines both B- and T-cell acquired immunes responses to the organism may provide optimal protection against this organism.

For protective immunity against S. aureus infection, antibody against surface proteins have been demonstrated to be protective in animal models, however, antibody against polysaccharides (type 5 and 8) was not protective in clinical trial. Accordingly, a combination vaccine (i.e., a polyscaharride plus a few S. aureus polypeptide antigens) is the main focus for S. aureus vaccine development. Herein, the inventors have demonstrated that a SA-MAPS comprising a number of SA antigens that results in eliciting both a B-cell response and a T-cell response (e.g., a Th1 and Th17 immunity) are important for protection against staphylococcal colonization and abscess formation. Antibody is important for protection against skin lesion and invasive disease.

Herein, the inventors discovered, identified and developed additional SA antigens containing several conserved antigens, using a vaccine platform that can elicit a broad range of immune responses, e.g., both B- and T-cell acquired responses. The inventors have previously developed a subunit vaccine platform, referred to as the Multiple-Antigen-Presenting-System (MAPS), which is disclosed in US Application 2014/0154287, which is incorporated herein in its entirety by reference. The MAPS generates affinity-coupled complexes of antigens that can then induce broad B- and T-cell responses, and interestingly generated an immune response to both the immunogenic polypeptide as well as the antigens.

The inventors have also previously described in US application US2021/0008192 (″192 application), which is incorporated herein by reference in its entirety, an immunogenic MAPS composition comprising at least one Staphylococcus aureus (SA) peptide antigens (SA-MAPS). In the '192 application, the SA peptide antigens are selected from any of: hemolysin (Hl) (e.g., hemolysin α or Hla), Clumping factor A (ClfA), Clumping factor B (ClfB), serine-aspirate repeat protein D (SdrD), Iron regulator surface protein A (IsdA) and Iron regulator surface protein B (IsdB). The '192 application discloses a specific SA-MAPS immunogenic composition.

Herein, the inventors have built upon and improved on the SA-MAPS composition disclosed in the '192 application by discovering additional Staphylococcus aureus (SA) peptide antigens to those disclosed in the '192 application. In some embodiments, at least one of these newly discovered SA polypeptide antigens can used alone, or added to, a selection of at least 1, or at least 2, or at least 3 or at least 4 or more SA polypeptide antigens disclosed in the '192 application for in an improved SA-MAPS immunogenic composition. In some embodiments, these newly discovered SA peptide antigens that can be used in the improved SA-MAPS immunogenic composition as disclosed herein are selected at least one of SA peptide antigens selected from any of: SA1739 (B2), SA1720 (B1), SA1890 (B3), SA0103 (T1), SA0377 (T2), SA0693 (T3) and SA2105 (T4).

Herein, the inventors prepared a multi-component SA subunit vaccine using either a conventional approach (immunization with purified proteins alone) or proteins incorporated onto a scaffold using the MAPS technology, as disclosed in US application 2014/0154287. The inventors evaluated the immunogenicity of a number of different vaccine approaches in mice, compared their protective efficacy in SA sepsis infection, dermonecrosis infection, skin abscess infection and gastrointestinal (GI) colonization models, and finally, studied the role of antigen-specific antibodies and T-cell immunity against different types of SA infection or colonization.

### Example 2

Selection of S. aureus Polypeptide Antigens:

Seven S. aureus polypeptide antigens and virulence factors were identified, and studied and assessed as vaccine candidates.

### In Silica Selection of SA Proteins:

In particular, a surface protein library was constructed from S. aureus USA300 genome, and screened through a number of parameters as disclosed in FIG. 1, including (i) selection of genes with signal peptides or transmembrane domains (2564 genes), (ii) selection of genes with >100 amino acids in the extracellular domain (789 genes), (iii) selection of genes convered in all sequences strains (266 genes), (iv) construction of vectors and and screening of constructs expressing the genes (231 genes), and (v) expression and purification of SA antigens in E. coli (195 genes).From about 190 SA proteins in the surface protein library, the inventors excluded toxins and proteins that had been studied previously.

### OPA Killing Assay:

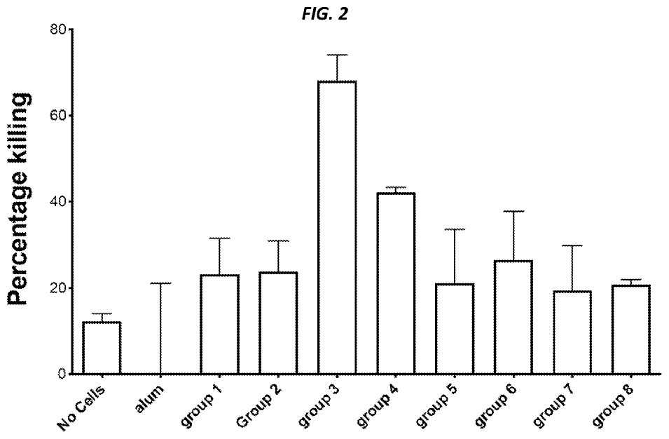

The remaining 159 proteins were allocated into 8 groups of 20 and used for immunization of 5 Balb/c mice per group (each mouse group received a combination of 20 proteins at 5 μg per protein). Mice were bled two weeks after the second immunization for initial test and terminally bled at 5 weeks post-second immunization. Serum was used in OPA killing assays with neutrophils derived from HL-60 cells. Serum that had active killing was mixed with each protein before killing assay to measure inhibition by each protein and demonstrate specificity. Mice were then immunized with proteins that showed promise, in order to confirm killing by specific antibody. The results from the OPA killing assay are shown in FIG. 2, which shows the highest percentage killing from Group 3.

### Inhibition of Killing by Purified Proteins:

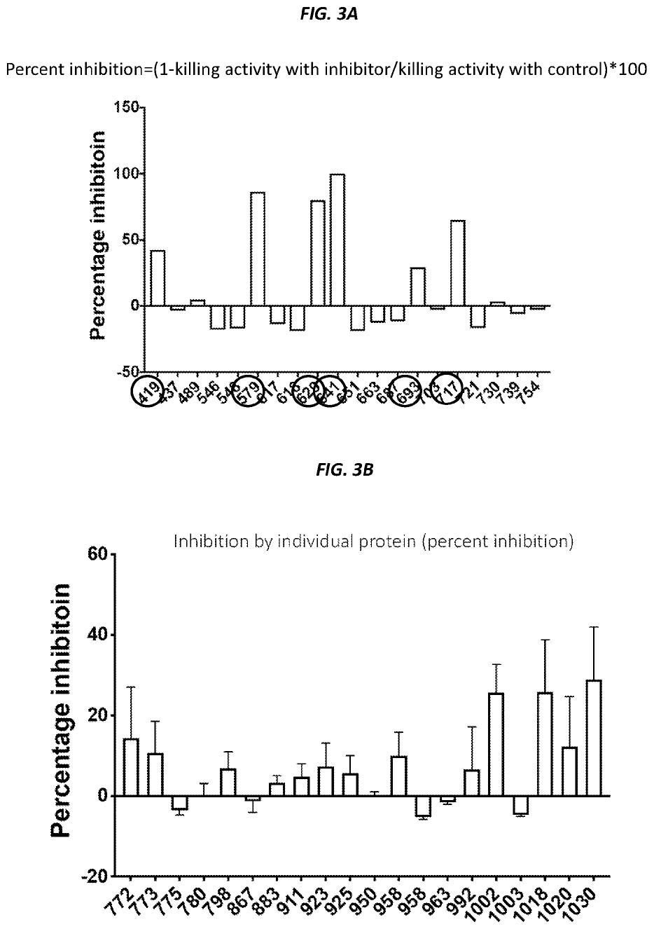

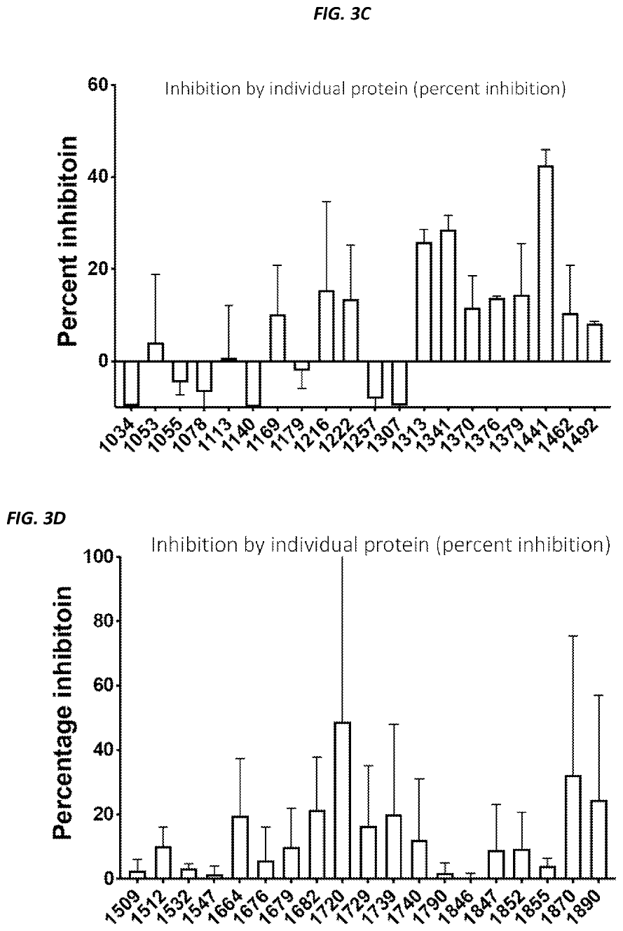

Next, the inventors assessed the inhibition of killing by purified proteins. FIG. 3A-3F shows results of the percent inhibition of selected SA proteins, showing specific SA proteins that shows significant percentage inhibition of killing HL-60 cells. For example SA proteins 419, 579, 629, 641, 693 and 717 show a significant percentage inhibition of killing HL-60 cells. A final list of the SA proteins selected for further analysis are shown in FIG. 4.

### Example 3

### In Vitro Assessment of Protection of in the Killing Assay SA Proteins in Balb C Mice:

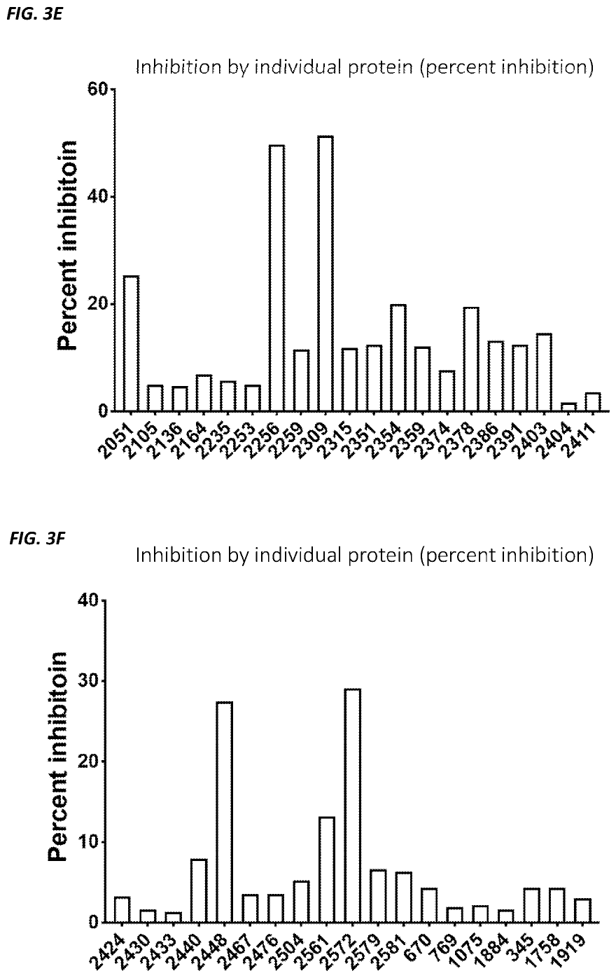

The inventors further assessed 14 candidate SA proteins shown in FIG. 4 in Balb/c mice. Balb/C were immunized with each of 14 selected proteins twice at two-week interval, and the mice were bled two weeks after the second immunization for evaluation of killing activity of sera and boosted again at 4 weeks after the second immunization. Sera were used in OPA killing assays with neutrophils derived from HL-60 cells. Mice were infected two weeks after last immunization. Results of the killing by serum from mice inoculated with individual proteins is shown in FIG. 5.

In Vivo Assessment of SA Antigens to Protect Balb C or B16 Mice from S. aureus Infection

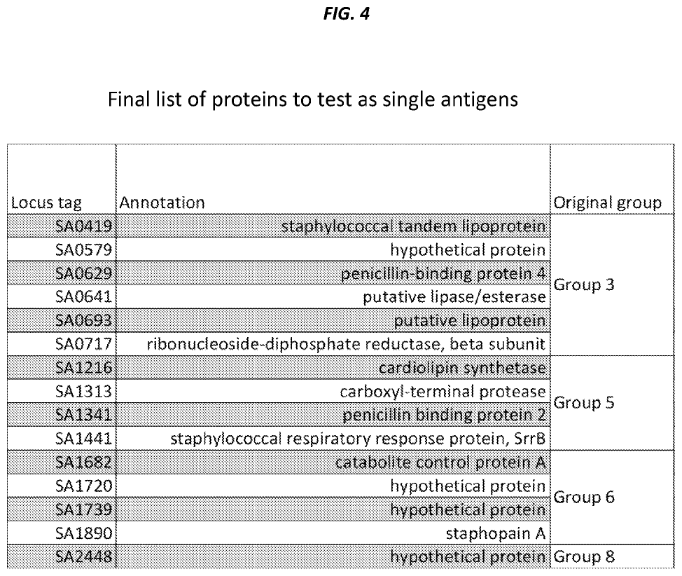

Next, the inventors assessed the protection of the mice inoculated with the SA proteins from i.v. infection with S. aureus. The results shown as percent survival of the mice is shown in Kaplien Mier plots of FIG. 6A-6D. In particular, FIG. 6A shows results of single SA protein protection of Balb/C mice infected with S. aureus, showing that 100% of Balb/C mice innoculated with SA1216 survived by day 14, and 60% of Balb/C mice innoculated with SA1890, SA1739 survived by 14 days after infection with S. aureus. Additionally, Balb/C mice innoculated with SA1720, SA0579 had a 40% survival rate after 14-days (FIG. 6A). B16 mice inoculated with one SA protein selected from: SA1890, or SA1739 or SA1720 showed a 50%, 30% and 20% survival rate, respectively, 14-days after infection with s. aureus (FIG. 6B).

In Vivo Assessment of Combination of SA Antigens to Protect Bl6 Mice from S. aureus Infection

Next, the inventors assessed the protection of mice by inoculation with a combination of SA proteins after i.v. infection with S. aureus. FIG. 6C shows B16 mice innoculated with three SA proteins (SA1720, 1739 and SA1890), or two SA proteins (SA1739 and SA1890) had a 60% survival 14 days after infection with s. aureus. Additionally, B16 mice innoculated with SA1720 and SA1739 together, or SA1739 alone had a 50% survival 14 days after infection with S. aureus (FIG. 6C).

The inventors also assessed the effect of selected candidate SA proteins in combination with known SA antigens previously disclosed in US2021/0008192 (″192 application), which is incorporated herein by reference in its entirety. In particular, the inventor assessed SA proteins SA1739 or SA1720 in combination with a MAPS4 complex, which is an immunogenic MAPS composition comprising a pneumococcal type 1 polysacharide, and comprising 4 SA proteins: hemolysin (Hl) (e.g., Hla209AA), Clumping factor A (ClfA), Clumping factor B (ClfB) and serine-aspirate repeat protein D (SdrD). FIG. 6D shows that B16 mice inoculated with MAPS4 complex alone had a 20% survival rate 14-days after infection with S. aureus, whereas when B16 mice were inoculated with MAPS4 and either SA1739 or the combination of SA1720 and SA1739, they had a 40% or 20% survival rate, respectively, 14-days after infection with S. aureus. This demonstrates that specific SA proteins disclosed herein, such as SA1739 can enhance the protective effect of the MAPS4 complex.

### Example 4

Identification and in Silica Screening of SA Proteins that are T-Cell Antigens for Protection Against S. aureus Skin Infection and Colonization

T-cell immunity is required for protection against staphylococcal infection, especially colonization and SSTI. Herein, using SA-MAPS, the inventors showed that Th17 and Th1 cells are important for protection against skin abscess and colonization.

Blocking colonization should greatly reduce the risk of S. aureus infection. Some individuals are virtually always colonized with S. aureus whereas in others, colonization is either short-lived and non-detectable. The inventors assessed if the existence of protective memory T cells, generated by exposure, may explain this dichotomy

The inventors performed screening of a surface protein library against human immune cells to identify antigens that specifically induce Th1 and Th17 responses in the resistant population, which could be candidates for inclusion in a vaccine.

In particular, a surface protein library was constructed from S. aureus USA300 genome (2564 genes), and screened through a number of parameters as disclosed in FIG. 1, including (i) selection of genes with signal peptides or transmembrane domains 789 genes), (ii) selection of genes with >100 amino acids in the extracellular domain (266 genes), (iii) selection of genes convered in all sequences strains (231 genes), (iv) construction of vectors and and screening of constructs expressing the genes (231 genes), and (v) expression and purification of SA antigens in E. coli (195 genes).

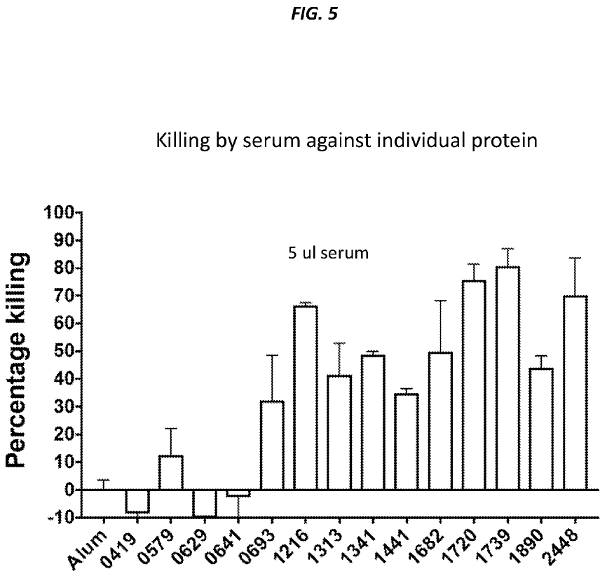

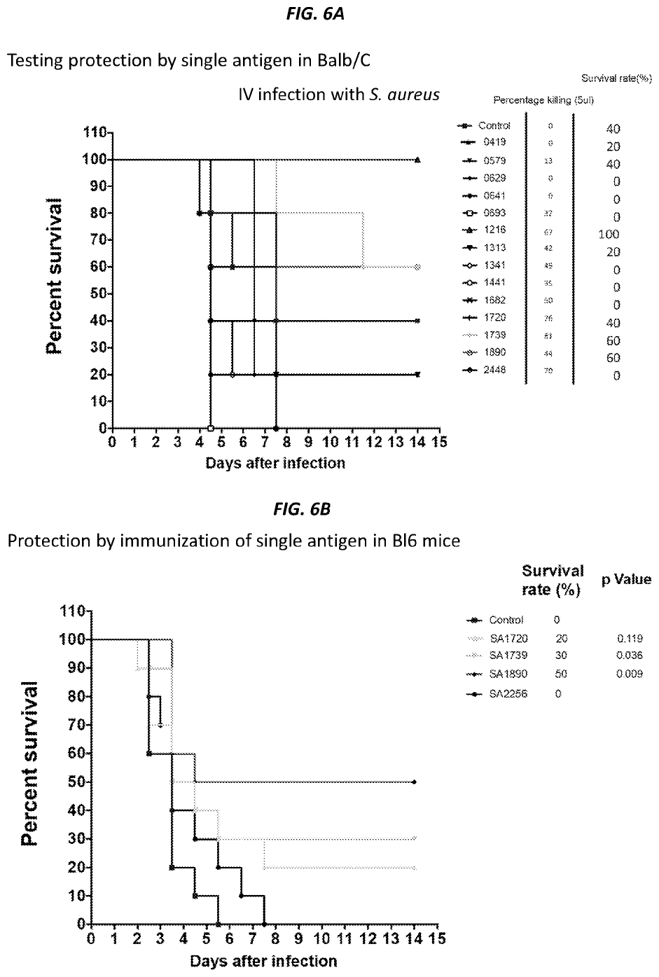

Blood draw was obtained from human subjects (n=31), with FIG. 7 showing the clinical study overview and details of the clinical enrollment and sampling schedules. FIG. 8 shows categorization of subject in the clinical study (n=31).

Results from Cytokine Assessment of Subjects:

The inventors determined that there was a large variability in both cytokine responses, and a broad range of responses particularly for IFN-g in any given individual and also between visits. In the assessment of IL-17A, there were generally low antigen-specific responses, and ⅓ (˜2000) of all protein-stimulated IL-17A values at each Visit 1 and Visit 3 were at the lowest limit of detection (LLD) for IL-17A ELISA. For IFN γ, there as generally higher antigen-specific responses, and far fewer values at the LLD. LPS stimulated some IFN γ (but no IL-17A)-relevant given variable LPS content in proteins used in stimulations. Infact, LPS dose-response curves in subjects demonstrated that the effect of LPS on IFN γ production was maxed out at an LPS concentration below the LPS concentration measured in most of the recombinant proteins-thus LPS effect should have been relatively similar across protein library.

The inventors discovered that the following antigens had higher responses in non carriers than carriers, and enabled informed antigens selection:

IFNγ antigens:
SAUSA300_0775—hypothetical function
SAUSA300_0312—psuG, indigoidine synthase, ?role in pyrimidine metabolism
SAUSA300_0377—putative lipoprotein
SAUSA300_1169—ftsK, DNA translocase

### IL-17A Antigen:

SAUSA300_0815—ear, E. coli ampicillin resistance protein, super-antigen similarity.

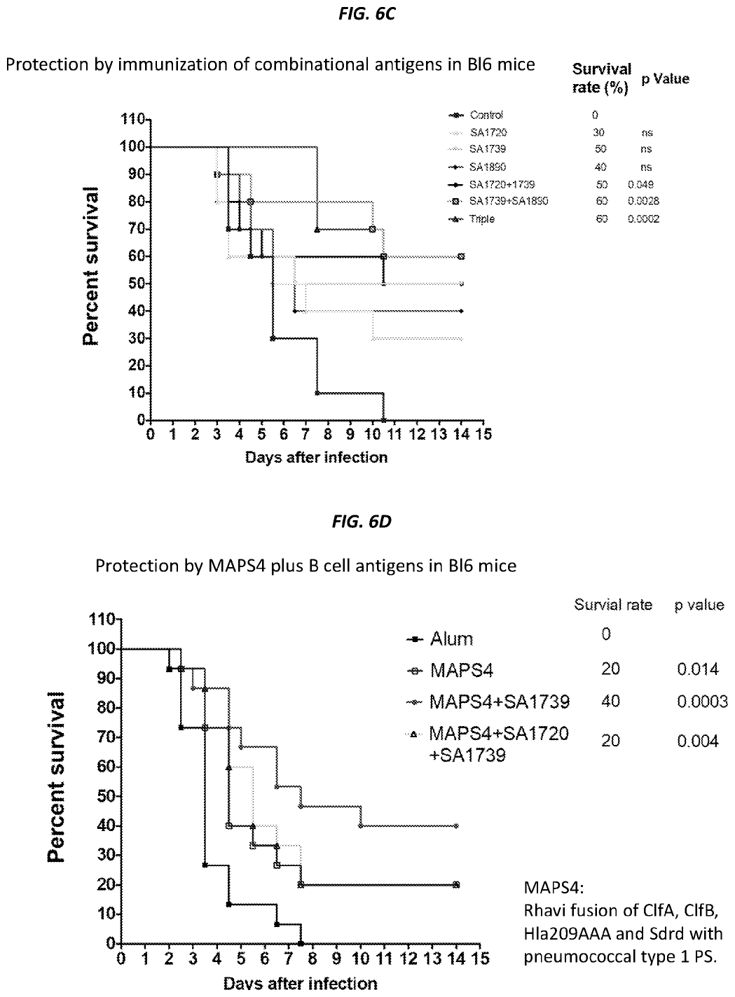

The top T-cell inducing SA antigens assessed are shown in the Table in FIG. 9.

### Rationalle for Selection of T-Cell Inducing SA Proteins.

Toxins and secreted proteins are not good T cell antigens, and thus were not progressed as candidate SA antigens. In particular, the inventors decided not to select antigens that are either secreted or are toxins, as in general, these types of antigens are not good T cell targets and do not contribute significantly to T-cell mediated protection against mucosal colonization or skin infection (e.g. pneumolysin of pneumococcus, Hla of SA). Additionally, the inventors decided not to select antigens that have superantigen potential (e.g. Eap) for potential safety concerns; and also did not select antigens that are already very highly stimulatory in every subject, which makes them unlikely to be good antigen targets (e.g. SA protein 1588).

The inventors selected 10 SA proteins as T-cell inducing SA antigens that fit the following important criteria:
a) SA proteins that express and purify moderately well or well
b) When the three visits in the clinical study are overlapped, these proteins are always in the top 50 (top quartile) for either IFN-g or IL-17A (including both carrier and noncarrier)
c) We also will include the two proteins that were suggested by the clinical study when analyzed with respect to higher cytokine (IFN-g) response in noncarrier vs. carrier (0775 and 0377)
d) assessment of how well these proteins are expressed (at RNA level) in pediatric abscess study and their function in SA pathogenesis
e) The function of the SA proteins related to pathogenesis.

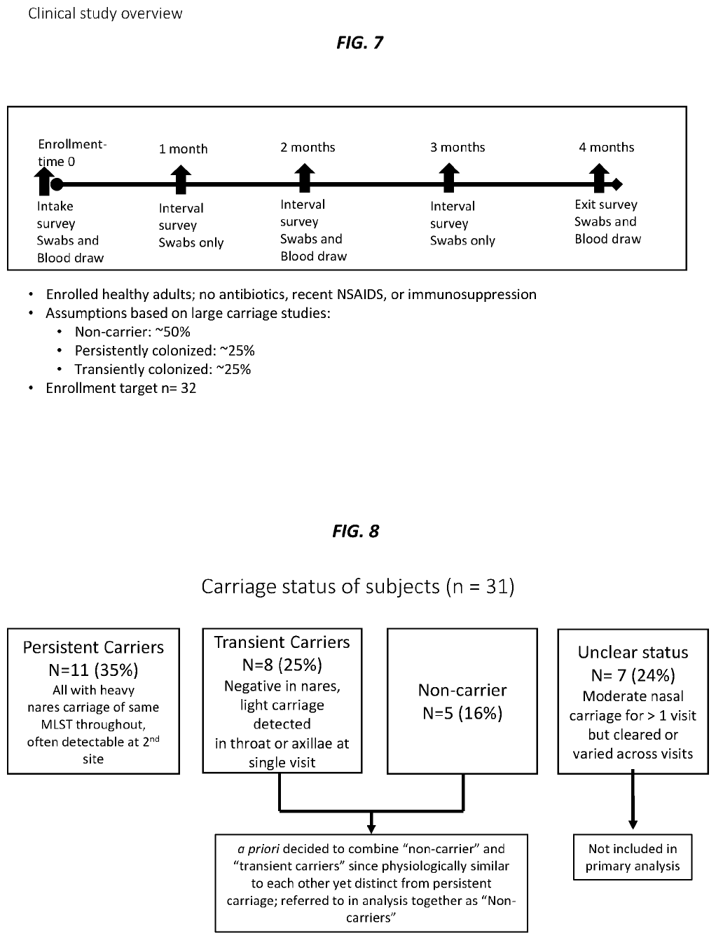

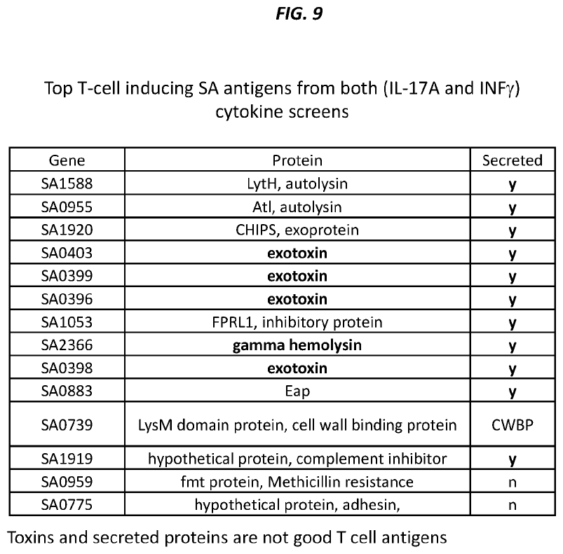

The inventors selected the ten T-cell antigens shown in the Table on FIG. 10 for purification and further analysis, with four T-cell antigens SA0103, SA0377, SA0693 and SA2105 (see FIG. 11) selected for fusion to a biotin-binding protein (e.g., Rhavi) for use in a SA-MAPS complex. The inventors did not have success in purification of the other six antigens from FIG. 10 as Rhavi-fusion proteins.

### Example 5

### In Vivo Protection Against Skin Abscess by Four T-Cell SA Antigens:

The inventors assessed the protection against skin abscess by four T cell antigens. Mice were immunized three times with two-weeks apart and challenged with USA300 TCH959 strain subcutaneously. Bacterial CFU in the abscess was determined 4 days after infection. Antigens used in the experiment were SA0103 (T1), SA0377 (T2), SA0693 (T3) and SA2105 (T4), which were purified as Rhavi fusion proteins and complexed to pneumococcal CPS1 as MAPS. FIG. 12 shows the significant protection against abscess by T cell antigens SA0103 (T1), SA0377 (T2), SA0693 (T3) and SA2105 (T4), when they present as part of a MAPS composition, or when SA0103 (T1) and SA2105 (T4) are both present in a MAPS composition.

### Enhanced In Vivo Protection Against Sepsis by Addition of T-Cell and B-Cell SA Antigens as Part of a MAPS4 Complex:

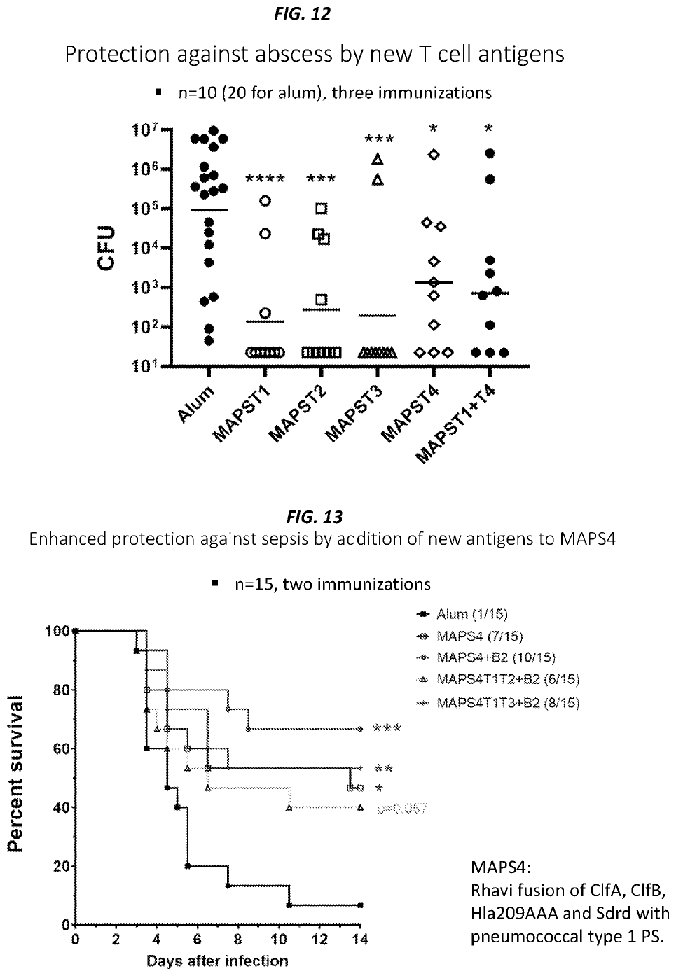

The inventors assessed the protection against sepsis challenge by MAPS4 with B and T antigens. Mice were immunized two times with two-weeks apart and challenged with 29213 strain IV. TIT2 is a fusion of SA0103, SA0377 and Rhavi. TIT3 is a fusion of SA0103, SA0693 and Rhavi. FIG. 13 shows MAPS4 plus SA1739 (B2) shows greatest percent survival against sepsis, and MAPS4 plus three different SA antigens selected from the groups of: (i) SA0103 (T1)+SA0377 (T2)+SA1739 (B2) (i.e., T1-T2 fusion and B2) or (ii) SA0103 (T1), +SA0693 (T3)+SA1739 (B2) (i.e., T1-T3 fusion and B2) results in significant survival benefit from sepsis. MAPS4 is an immunogenic MAPS composition comprising a pneumococcal type 1 polysaccharide, and comprising 4 SA proteins: hemolysin (Hl) (e.g., Hla209AA), Clumping factor A (ClfA), Clumping factor B (ClfB) and serine-aspirate repeat protein D (SdrD).

### Enhanced In Vivo Protection Against Abscess by Addition of T-Cell and B-Cell SA Antigens as Part of a MAPS4 Complex.

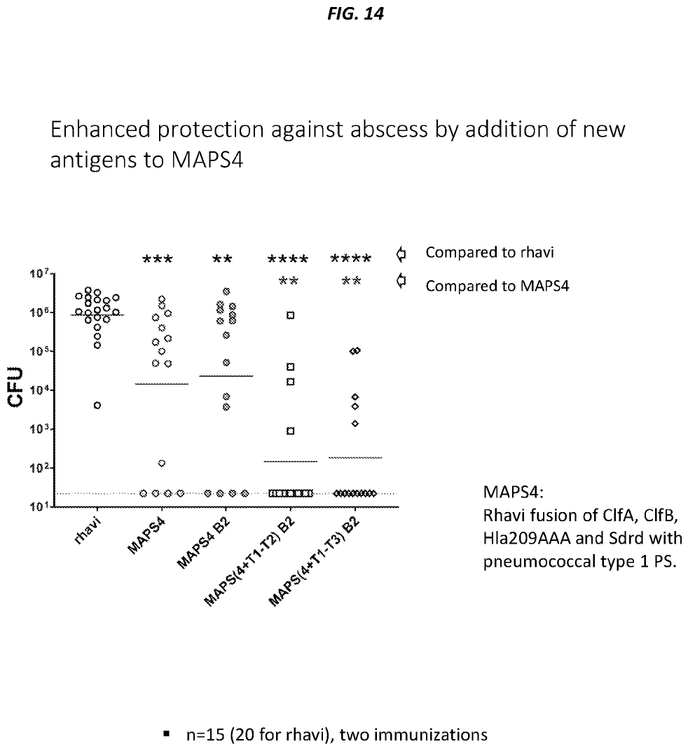

T antigens enhance the protection against skin abscess by MAPS4. Mice were immunized two times with two-weeks apart and challenged with USA300 TCH959 strain subcutaneously. Bacterial CFU in the abscess was determined 4 days after infection. FIG. 14 shows protection of MAPS4 plus B-cell antigens (e.g., SA1739 (B2)) or B-cell and T cell antigens (e.g., SA1739 (B2) and SA0103-SA0377 T-cell antigen fusion protein; or SA1739 (B2) and SA0103-SA0693 T-cell fusion protein). Importantly, the inventors discovered that the combination of both B-cell antigens and T-cell antigens, e.g., in MAPS4 complexes further comprising SA1739 (B2) and SA0103-SA0377 T-cell antigen fusion protein; or SA1739 (B2) and SA0103-SA0693 T-cell fusion protein results in significant protection against sepsis as compared to MAPS4 alone (FIG. 14). Pneumococcal CPS1 was used as backbone of MAPS4. T1T2 is a fusion of SA0103, SA0377 and Rhavi (e.g., Rhavi-SA0103-SA0377 fusion protein). T1T3 is a fusion of SA0103, SA0693 and Rhavi (e.g. Rhavi-SA0103-SA0693 fusion protein).

Accordingly, the inventors have discovered that in prevention of skin abscess, adding T1-T2 or T1-T3 to MAPS4 significantly improved protection against skin abscess. However, adding B2 did not have obvious impact on protection. Additionally, the inventors discovered that in the prevention of sepsis, adding B2 to MAPS4 improved protection against sepsis, and that adding T1-T2 or T1-T3 fusion proteins may have slight interference on protection after two immunizations.

TABLE 4E
Combinations of SA antigens in SA-MAPS compositions useful in the methods and compositions as disclosed herein.

| | Description of | Subgroups with the different SA antigens. [Note: SA antigens in |
|---|---|---|
| GROUP | the subgroup | square brackets indicate the SA antigens in a single subgroup] |
| 1 | 1 SA antigen from | SA1739, SA1720, SA1890, SA0103, SA0377, SA0693, SA2105 |
| | SA antigens in | |
| | Group A selected | |
| | from any of: | |
| 2 | At least 2 “Group | [SA1739, SA1720], [SA1739, SA1890], [SA1739, SA0103], [SA1739, SA0377], |
| | A” SA antigens | [SA1739, SA0693], [SA1739, SA2105], [SA1720, SA1739], [SA1720, SA1890], |
| | selected from the | [SA1720, SA0103], [SA1720, SA0377], [SA1720, SA0693], [SA1720, SA2105], |
| | subgroups of: (42 | [SA1890, SA1739], [SA1890, SA1720], [SA1890, SA0103], [SA1890, SA0377], |
| | combinations) | [SA1890, SA0693], [SA1890, SA2105], [SA0103, SA1739], [SA0103, SA1720], |
| | | [SA0103, SA1890], [SA0103, SA0377], [SA0103, SA0693], [SA0103, SA2105], |
| | | [SA0377, SA1739], [SA0377, SA1720], [SA0377, SA1890], [SA0377, SA0103], |
| | | [SA0377, SA0693], [SA0377, SA2105], [SA0693, SA1739], [SA0693, SA1720], |
| | | [SA0693, SA1890], [SA0693, SA0103], [SA0693, SA0377], [SA0693, SA2105], |
| | | [SA2105, SA1739], [SA2105, SA1720], [SA2105, SA1890], [SA2105, SA0103], |
| | | [SA2105, SA0377], [SA2105, SA0693] |
| 3 | At least 1 “Group | Hla209, ClfA, ClfB, SdrD, IsdA or IsdB. |
| | B” SA antigen | |
| | selected from any | |
| | of: | |
| 4 | at least 2 “Group | [Hla209, ClfA], [Hla209, ClfB], [Hla209, SdrD], [Hla209, IsdA], [Hla209, IsdB], [ClfA, Hla209], |
| | B” SA antigens | [ClfA, ClfB], [ClfA, SdrD], [ClfA, IsdA], [ClfA, IsdB], [ClfB, Hla209], [ClfB, ClfA], [ClfB, |
| | selected from the | SdrD], [ClfB, IsdA], [ClfB, IsdB], [SdrD, Hla209], [SdrD, ClfA], [SdrD, ClfB], [SdrD, IsdA], |
| | subgroups of: | [SdrD, IsdB], [IsdA, Hla209], [IsdA, ClfA], [IsdA, ClfB], [IsdA, SdrD], [IsdA, IsdB], [IsdB, |
| | | Hla209], [IsdB, ClfA], [IsdB, ClfB], [IsdB, SdrD], [IsdB, IsdA] |
| 5 | at least 3 “Group | [Hla209, ClfA, ClfB], [Hla209, ClfA, SdrD], [Hla209, ClfA, IsdA], [Hla209, ClfA, IsdB], [Hla209, |
| | B” SA antigens | ClfB, ClfA], [Hla209, ClfB, SdrD], [Hla209, ClfB, IsdA], [Hla209, ClfB, IsdB], [Hla209, SdrD, |
| | selected from the | ClfA], [Hla209, SdrD, ClfB], [Hla209, SdrD, IsdA], [Hla209, SdrD, IsdB], [Hla209, IsdA, ClfA], |
| | subgroups of: | [Hla209, IsdA, ClfB], [Hla209, IsdA, SdrD], [Hla209, IsdA, IsdB], [Hla209, IsdB, ClfA], [Hla209, |
| | | IsdB, ClfB], [Hla209, IsdB, SdrD], [Hla209, IsdB, IsdA], [ClfA, Hla209, ClfB], [ClfA, Hla209, |
| | | SdrD], [ClfA, Hla209, IsdA], [ClfA, Hla209, IsdB], [ClfA, ClfB, Hla209], [ClfA, ClfB, SdrD], [ClfA, |
| | | ClfB, IsdA], [ClfA, ClfB, IsdB], [ClfA, SdrD, Hla209], [ClfA, SdrD, ClfB], [ClfA, SdrD, IsdA], |
| | | [ClfA, SdrD, IsdB], [ClfA, IsdA, Hla209], [ClfA, IsdA, ClfB], [ClfA, IsdA, SdrD], [ClfA, IsdA, |
| | | IsdB], [ClfA, IsdB, Hla209], [ClfA, IsdB, ClfB], [ClfA, IsdB, SdrD], [ClfA, IsdB, IsdA], [ClfB, |
| | | Hla209, ClfA], [ClfB, Hla209, SdrD], [ClfB, Hla209, IsdA], [ClfB, Hla209, IsdB], [ClfB, ClfA, Hla209], |
| | | [ClfB, ClfA, SdrD], [ClfB, ClfA, IsdA], [ClfB, ClfA, IsdB], [ClfB, SdrD, Hla209], [ClfB, SdrD, |
| | | ClfA], [ClfB, SdrD, IsdA], [ClfB, SdrD, IsdB], [ClfB, IsdA, Hla209], [ClfB, IsdA, ClfA], [ClfB, |
| | | IsdA, SdrD], [ClfB, IsdA, IsdB], [ClfB, IsdB, Hla209], [ClfB, IsdB, ClfA], [ClfB, IsdB, SdrD], |
| | | [ClfB, IsdB, IsdA], [SdrD, Hla209, ClfA], [SdrD, Hla209, ClfB], [SdrD, Hla209, IsdA], [SdrD, Hla209, |
| | | IsdB], [SdrD, ClfA, Hla209], [SdrD, ClfA, ClfB], [SdrD, ClfA, IsdA], [SdrD, ClfA, IsdB], [SdrD, |
| | | ClfB, Hla209], [SdrD, ClfB, ClfA], [SdrD, ClfB, IsdA], [SdrD, ClfB, IsdB], [SdrD, IsdA, Hla209], |
| | | [SdrD, IsdA, ClfA], [SdrD, IsdA, ClfB], [SdrD, IsdA, IsdB], [SdrD, IsdB, Hla209], [SdrD, IsdB, |
| | | ClfA], [SdrD, IsdB, ClfB], [SdrD, IsdB, IsdA], [IsdA, Hla209, ClfA], [IsdA, Hla209, ClfB], [IsdA, |
| | | Hla209, SdrD], [IsdA, Hla209, IsdB], [IsdA, ClfA, Hla209], [IsdA, ClfA, ClfB], [IsdA, ClfA, |
| | | SdrD], [IsdA, ClfA, IsdB], [IsdA, ClfB, Hla209], [IsdA, ClfB, ClfA], [IsdA, ClfB, SdrD], [IsdA, ClfB, |
| | | IsdB], [IsdA, SdrD, Hla209], [IsdA, SdrD, ClfA], [IsdA, SdrD, ClfB], [IsdA, SdrD, IsdB], [IsdA, |
| | | IsdB, Hla209], [IsdA, IsdB, ClfA], [IsdA, IsdB, ClfB], [IsdA, IsdB, SdrD], [IsdB, Hla209, ClfA], |
| | | [IsdB, Hla209, ClfB], [IsdB, Hla209, SdrD], [IsdB, Hla209, IsdA], [IsdB, ClfA, Hla209], [IsdB, |
| | | ClfA, ClfB], [IsdB, ClfA, SdrD], [IsdB, ClfA, IsdA], [IsdB, ClfB, Hla209], [IsdB, ClfB, ClfA], [IsdB, |
| | | ClfB, SdrD], [IsdB, ClfB, IsdA], [IsdB, SdrD, Hla209], [IsdB, SdrD, ClfA], [IsdB, SdrD, ClfB], [IsdB, |
| | | SdrD, IsdA], [IsdB, IsdA, Hla209], [IsdB, IsdA, ClfA], [IsdB, IsdA, ClfB], [IsdB, IsdA, SdrD] |
| 6 | at least 4 “Group | [Hla209, ClfA, ClfB, SdrD], [Hla209, ClfA, ClfB, IsdA], [Hla209, ClfA, ClfB, IsdB], [Hla209, |
| | B” SA antigens | ClfA, SdrD, ClfB], [Hla209, ClfA, SdrD, IsdA], [Hla209, ClfA, SdrD, IsdB], [Hla209, ClfA, IsdA, |
| | selected from the | ClfB], [Hla209, ClfA, IsdA, SdrD], [Hla209, ClfA, IsdA, IsdB], [Hla209, ClfA, IsdB, ClfB], [Hla209, |
| | subgroups of: | ClfA, IsdB, SdrD], [Hla209, ClfA, IsdB, IsdA], [Hla209, ClfB, ClfA, SdrD], [Hla209, ClfB, ClfA, |
| | | IsdA], [Hla209, ClfB, ClfA, IsdB], [Hla209, ClfB, SdrD, ClfA], [Hla209, ClfB, SdrD, IsdA], |
| | | [Hla209, ClfB, SdrD, IsdB], [Hla209, ClfB, IsdA, ClfA], [Hla209, ClfB, IsdA, SdrD], [Hla209, ClfB, |
| | | IsdA, IsdB], [Hla209, ClfB, IsdB, ClfA], [Hla209, ClfB, IsdB, SdrD], [Hla209, ClfB, IsdB, IsdA], |
| | | [Hla209, SdrD, ClfA, ClfB], [Hla209, SdrD, ClfA, IsdA], [Hla209, SdrD, ClfA, IsdB], [Hla209, |
| | | SdrD, ClfB, ClfA], [Hla209, SdrD, ClfB, IsdA], [Hla209, SdrD, ClfB, IsdB], [Hla209, SdrD, IsdA, |
| | | ClfA], [Hla209, SdrD, IsdA, ClfB], [Hla209, SdrD, IsdA, IsdB], [Hla209, SdrD, IsdB, ClfA], |
| | | [Hla209, SdrD, IsdB, ClfB], [Hla209, SdrD, IsdB, IsdA], [Hla209, IsdA, ClfA, ClfB], [Hla209, IsdA, |
| | | ClfA, SdrD], [Hla209, IsdA, ClfA, IsdB], [Hla209, IsdA, ClfB, ClfA], [Hla209, IsdA, ClfB, SdrD], |
| | | [Hla209, IsdA, ClfB, IsdB], [Hla209, IsdA, SdrD, ClfA], [Hla209, IsdA, SdrD, ClfB], [Hla209, IsdA, |
| | | SdrD, IsdB], [Hla209, IsdA, IsdB, ClfA], [Hla209, IsdA, IsdB, ClfB], [Hla209, IsdA, IsdB, SdrD], |
| | | [Hla209, IsdB, ClfA, ClfB], [Hla209, IsdB, ClfA, SdrD], [Hla209, IsdB, ClfA, IsdA], [Hla209, |
| | | IsdB, ClfB, ClfA], [Hla209, IsdB, ClfB, SdrD], [Hla209, IsdB, ClfB, IsdA], [Hla209, IsdB, SdrD, |
| | | ClfA], [Hla209, IsdB, SdrD, ClfB], [Hla209, IsdB, SdrD, IsdA], [Hla209, IsdB, IsdA, ClfA], |
| | | [Hla209, IsdB, IsdA, ClfB], [Hla209, IsdB, IsdA, SdrD], [ClfA, Hla209, ClfB, SdrD], [ClfA, Hla209, |
| | | ClfB, IsdA], [ClfA, Hla209, ClfB, IsdB], [ClfA, Hla209, SdrD, ClfB], [ClfA, Hla209, SdrD, IsdA], |
| | | [ClfA, Hla209, SdrD, IsdB], [ClfA, Hla209, IsdA, ClfB], [ClfA, Hla209, IsdA, SdrD], [ClfA, Hla209, |
| | | IsdA, IsdB], [ClfA, Hla209, IsdB, ClfB], [ClfA, Hla209, IsdB, SdrD], [ClfA, Hla209, IsdB, IsdA], |
| | | [ClfA, ClfB, Hla209, SdrD], [ClfA, ClfB, Hla209, IsdA], [ClfA, ClfB, Hla209, IsdB], [ClfA, |
| | | ClfB, SdrD, Hla209], [ClfA, ClfB, SdrD, IsdA], [ClfA, ClfB, SdrD, IsdB], [ClfA, ClfB, IsdA, Hla209], |
| | | [ClfA, ClfB, IsdA, SdrD], [ClfA, ClfB, IsdA, IsdB], [ClfA, ClfB, IsdB, Hla209], [ClfA, ClfB, |
| | | IsdB, SdrD], [ClfA, ClfB, IsdB, IsdA], [ClfA, SdrD, Hla209, ClfB], [ClfA, SdrD, Hla209, IsdA], [ClfA, |
| | | SdrD, Hla209, IsdB], [ClfA, SdrD, ClfB, Hla209], [ClfA, SdrD, ClfB, IsdA], [ClfA, SdrD, ClfB, |
| | | IsdB], [ClfA, SdrD, IsdA, Hla209], [ClfA, SdrD, IsdA, ClfB], [ClfA, SdrD, IsdA, IsdB], [ClfA, |
| | | SdrD, IsdB, Hla209], [ClfA, SdrD, IsdB, ClfB], [ClfA, SdrD, IsdB, IsdA], [ClfA, IsdA, Hla209, |
| | | ClfB], [ClfA, IsdA, Hla209, SdrD], [ClfA, IsdA, Hla209, IsdB], [ClfA, IsdA, ClfB, Hla209], [ClfA, |
| | | IsdA, ClfB, SdrD], [ClfA, IsdA, ClfB, IsdB], [ClfA, IsdA, SdrD, Hla209], [ClfA, IsdA, SdrD, ClfB], |
| | | [ClfA, IsdA, SdrD, IsdB], [ClfA, IsdA, IsdB, Hla209], [ClfA, IsdA, IsdB, ClfB], [ClfA, IsdA, IsdB, |
| | | SdrD], [ClfA, IsdB, Hla209, ClfB], [ClfA, IsdB, Hla209, SdrD], [ClfA, IsdB, Hla209, IsdA], |
| | | [ClfA, IsdB, ClfB, Hla209], [ClfA, IsdB, ClfB, SdrD], [ClfA, IsdB, ClfB, IsdA], [ClfA, IsdB, SdrD, |
| | | Hla209], [ClfA, IsdB, SdrD, ClfB], [ClfA, IsdB, SdrD, IsdA], [ClfA, IsdB, IsdA, Hla209], [ClfA, IsdB, |
| | | IsdA, ClfB], [ClfA, IsdB, IsdA, SdrD], [ClfB, Hla209, ClfA, SdrD], [ClfB, Hla209, ClfA, IsdA], |
| | | [ClfB, Hla209, ClfA, IsdB], [ClfB, Hla209, SdrD, ClfA], [ClfB, Hla209, SdrD, IsdA], [ClfB, |
| | | Hla209, SdrD, IsdB], [ClfB, Hla209, IsdA, ClfA], [ClfB, Hla209, IsdA, SdrD], [ClfB, Hla209, IsdA, |
| | | IsdB], [ClfB, Hla209, IsdB, ClfA], [ClfB, Hla209, IsdB, SdrD], [ClfB, Hla209, IsdB, IsdA], [ClfB, |
| | | ClfA, Hla209, SdrD], [ClfB, ClfA, Hla209, IsdA], [ClfB, ClfA, Hla209, IsdB], [ClfB, ClfA, SdrD, |
| | | Hla209], [ClfB, ClfA, SdrD, IsdA], [ClfB, ClfA, SdrD, IsdB], [ClfB, ClfA, IsdA, Hla209], [ClfB, |
| | | ClfA, IsdA, SdrD], [ClfB, ClfA, IsdA, IsdB], [ClfB, ClfA, IsdB, Hla209], [ClfB, ClfA, IsdB, SdrD], |
| | | [ClfB, ClfA, IsdB, IsdA], [ClfB, SdrD, Hla209, ClfA], [ClfB, SdrD, Hla209, IsdA], [ClfB, SdrD, |
| | | Hla209, IsdB], [ClfB, SdrD, ClfA, Hla209], [ClfB, SdrD, ClfA, IsdA], [ClfB, SdrD, ClfA, IsdB], [ClfB, |
| | | SdrD, IsdA, Hla209], [ClfB, SdrD, IsdA, ClfA], [ClfB, SdrD, IsdA, IsdB], [ClfB, SdrD, IsdB, |
| | | Hla209], [ClfB, SdrD, IsdB, ClfA], [ClfB, SdrD, IsdB, IsdA], [ClfB, IsdA, Hla209, ClfA], [ClfB, IsdA, |
| | | Hla209, SdrD], [ClfB, IsdA, Hla209, IsdB], [ClfB, IsdA, ClfA, Hla209], [ClfB, IsdA, ClfA, SdrD], |
| | | [ClfB, IsdA, ClfA, IsdB], [ClfB, IsdA, SdrD, Hla209], [ClfB, IsdA, SdrD, ClfA], [ClfB, IsdA, |
| | | SdrD, IsdB], [ClfB, IsdA, IsdB, Hla209], [ClfB, IsdA, IsdB, ClfA], [ClfB, IsdA, IsdB, SdrD], [ClfB, |
| | | IsdB, Hla209, ClfA], [ClfB, IsdB, Hla209, SdrD], [ClfB, IsdB, Hla209, IsdA], [ClfB, IsdB, ClfA, |
| | | Hla209], [ClfB, IsdB, ClfA, SdrD], [ClfB, IsdB, ClfA, IsdA], [ClfB, IsdB, SdrD, Hla209], [ClfB, |
| | | IsdB, SdrD, ClfA], [ClfB, IsdB, SdrD, IsdA], [ClfB, IsdB, IsdA, Hla209], [ClfB, IsdB, IsdA, ClfA], |
| | | [ClfB, IsdB, IsdA, SdrD], [SdrD, Hla209, ClfA, ClfB], [SdrD, Hla209, ClfA, IsdA], [SdrD, Hla209, |
| | | ClfA, IsdB], [SdrD, Hla209, ClfB, ClfA], [SdrD, Hla209, ClfB, IsdA], [SdrD, Hla209, ClfB, IsdB], |
| | | [SdrD, Hla209, IsdA, ClfA], [SdrD, Hla209, IsdA, ClfB], [SdrD, Hla209, IsdA, IsdB], [SdrD, |
| | | Hla209, IsdB, ClfA], [SdrD, Hla209, IsdB, ClfB], [SdrD, Hla209, IsdB, IsdA], [SdrD, ClfA, Hla209, |
| | | ClfB], [SdrD, ClfA, Hla209, IsdA], [SdrD, ClfA, Hla209, IsdB], [SdrD, ClfA, ClfB, Hla209], |
| | | [SdrD, ClfA, ClfB, IsdA], [SdrD, ClfA, ClfB, IsdB], [SdrD, ClfA, IsdA, Hla209], [SdrD, ClfA, IsdA, |
| | | ClfB], [SdrD, ClfA, IsdA, IsdB], [SdrD, ClfA, IsdB, Hla209], [SdrD, ClfA, IsdB, ClfB], [SdrD, |
| | | ClfA, IsdB, IsdA], [SdrD, ClfB, Hla209, ClfA], [SdrD, ClfB, Hla209, IsdA], [SdrD, ClfB, Hla209, |
| | | IsdB], [SdrD, ClfB, ClfA, Hla209], [SdrD, ClfB, ClfA, IsdA], [SdrD, ClfB, ClfA, IsdB], [SdrD, |
| | | ClfB, IsdA, Hla209], [SdrD, ClfB, IsdA, ClfA], [SdrD, ClfB, IsdA, IsdB], [SdrD, ClfB, IsdB, Hla209], |
| | | [SdrD, ClfB, IsdB, ClfA], [SdrD, ClfB, IsdB, IsdA], [SdrD, IsdA, Hla209, ClfA], [SdrD, IsdA, |
| | | Hla209, ClfB], [SdrD, IsdA, Hla209, IsdB], [SdrD, IsdA, ClfA, Hla209], [SdrD, IsdA, ClfA, ClfB], |
| | | [SdrD, IsdA, ClfA, IsdB], [SdrD, IsdA, ClfB, Hla209], [SdrD, IsdA, ClfB, ClfA], [SdrD, IsdA, ClfB, |
| | | IsdB], [SdrD, IsdA, IsdB, Hla209], [SdrD, IsdA, IsdB, ClfA], [SdrD, IsdA, IsdB, ClfB], [SdrD, |
| | | IsdB, Hla209, ClfA], [SdrD, IsdB, Hla209, ClfB], [SdrD, IsdB, Hla209, IsdA], [SdrD, IsdB, ClfA, |
| | | Hla209], [SdrD, IsdB, ClfA, ClfB], [SdrD, IsdB, ClfA, IsdA], [SdrD, IsdB, ClfB, Hla209], [SdrD, |
| | | IsdB, ClfB, ClfA], [SdrD, IsdB, ClfB, IsdA], [SdrD, IsdB, IsdA, Hla209], [SdrD, IsdB, IsdA, ClfA], |
| | | [SdrD, IsdB, IsdA, ClfB], [IsdA, Hla209, ClfA, ClfB], [IsdA, Hla209, ClfA, SdrD], [IsdA, Hla209, |
| | | ClfA, IsdB], [IsdA, Hla209, ClfB, ClfA], [IsdA, Hla209, ClfB, SdrD], [IsdA, Hla209, ClfB, IsdB], |
| | | [IsdA, Hla209, SdrD, ClfA], [IsdA, Hla209, SdrD, ClfB], [IsdA, Hla209, SdrD, IsdB], [IsdA, |
| | | Hla209, IsdB, ClfA], [IsdA, Hla209, IsdB, ClfB], [IsdA, Hla209, IsdB, SdrD], [IsdA, ClfA, Hla209, |
| | | ClfB], [IsdA, ClfA, Hla209, SdrD], [IsdA, ClfA, Hla209, IsdB], [IsdA, ClfA, ClfB, Hla209], [IsdA, |
| | | ClfA, ClfB, SdrD], [IsdA, ClfA, ClfB, IsdB], [IsdA, ClfA, SdrD, Hla209], [IsdA, ClfA, SdrD, ClfB], |
| | | [IsdA, ClfA, SdrD, IsdB], [IsdA, ClfA, IsdB, Hla209], [IsdA, ClfA, IsdB, ClfB], [IsdA, ClfA, IsdB, |
| | | SdrD], [IsdA, ClfB, Hla209, ClfA], [IsdA, ClfB, Hla209, SdrD], [IsdA, ClfB, Hla209, IsdB], [IsdA, |
| | | ClfB, ClfA, Hla209], [IsdA, ClfB, ClfA, SdrD], [IsdA, ClfB, ClfA, IsdB], [IsdA, ClfB, SdrD, |
| | | Hla209], [IsdA, ClfB, SdrD, ClfA], [IsdA, ClfB, SdrD, IsdB], [IsdA, ClfB, IsdB, Hla209], [IsdA, ClfB, |
| | | IsdB, ClfA], [IsdA, ClfB, IsdB, SdrD], [IsdA, SdrD, Hla209, ClfA], [IsdA, SdrD, Hla209, ClfB], |
| | | [IsdA, SdrD, Hla209, IsdB], [IsdA, SdrD, ClfA, Hla209], [IsdA, SdrD, ClfA, ClfB], [IsdA, SdrD, |
| | | ClfA, IsdB], [IsdA, SdrD, ClfB, Hla209], [IsdA, SdrD, ClfB, ClfA], [IsdA, SdrD, ClfB, IsdB], [IsdA, |
| | | SdrD, IsdB, Hla209], [IsdA, SdrD, IsdB, ClfA], [IsdA, SdrD, IsdB, ClfB], [IsdA, IsdB, Hla209, |
| | | ClfA], [IsdA, IsdB, Hla209, ClfB], [IsdA, IsdB, Hla209, SdrD], [IsdA, IsdB, ClfA, Hla209], [IsdA, |
| | | IsdB, ClfA, ClfB], [IsdA, IsdB, ClfA, SdrD], [IsdA, IsdB, ClfB, Hla209], [IsdA, IsdB, ClfB, ClfA], |
| | | [IsdA, IsdB, ClfB, SdrD], [IsdA, IsdB, SdrD, Hla209], [IsdA, IsdB, SdrD, ClfA], [IsdA, IsdB, SdrD, |
| | | ClfB], [IsdB, Hla209, ClfA, ClfB], [IsdB, Hla209, ClfA, SdrD], [IsdB, Hla209, ClfA, IsdA], |
| | | [IsdB, Hla209, ClfB, ClfA], [IsdB, Hla209, ClfB, SdrD], [IsdB, Hla209, ClfB, IsdA], [IsdB, Hla209, |
| | | SdrD, ClfA], [IsdB, Hla209, SdrD, ClfB], [IsdB, Hla209, SdrD, IsdA], [IsdB, Hla209, IsdA, ClfA], |
| | | [IsdB, Hla209, IsdA, ClfB], [IsdB, Hla209, IsdA, SdrD], [IsdB, ClfA, Hla209, ClfB], [IsdB, ClfA, |
| | | Hla209, SdrD], [IsdB, ClfA, Hla209, IsdA], [IsdB, ClfA, ClfB, Hla209], [IsdB, ClfA, ClfB, SdrD], |
| | | [IsdB, ClfA, ClfB, IsdA], [IsdB, ClfA, SdrD, Hla209], [IsdB, ClfA, SdrD, ClfB], [IsdB, ClfA, SdrD, |
| | | IsdA], [IsdB, ClfA, IsdA, Hla209], [IsdB, ClfA, IsdA, ClfB], [IsdB, ClfA, IsdA, SdrD], [IsdB, |
| | | ClfB, Hla209, ClfA], [IsdB, ClfB, Hla209, SdrD], [IsdB, ClfB, Hla209, IsdA], [IsdB, ClfB, ClfA, |
| | | Hla209], [IsdB, ClfB, ClfA, SdrD], [IsdB, ClfB, ClfA, IsdA], [IsdB, ClfB, SdrD, Hla209], [IsdB, ClfB, |
| | | SdrD, ClfA], [IsdB, ClfB, SdrD, IsdA], [IsdB, ClfB, IsdA, Hla209], [IsdB, ClfB, IsdA, ClfA], |
| | | [IsdB, ClfB, IsdA, SdrD], [IsdB, SdrD, Hla209, ClfA], [IsdB, SdrD, Hla209, ClfB], [IsdB, SdrD, |
| | | Hla209, IsdA], [IsdB, SdrD, ClfA, Hla209], [IsdB, SdrD, ClfA, ClfB], [IsdB, SdrD, ClfA, IsdA], [IsdB, |
| | | SdrD, ClfB, Hla209], [IsdB, SdrD, ClfB, ClfA], [IsdB, SdrD, ClfB, IsdA], [IsdB, SdrD, IsdA, |
| | | Hla209], [IsdB, SdrD, IsdA, ClfA], [IsdB, SdrD, IsdA, ClfB], [IsdB, IsdA, Hla209, ClfA], [IsdB, IsdA, |
| | | Hla209, ClfB], [IsdB, IsdA, Hla209, SdrD], [IsdB, IsdA, ClfA, Hla209], [IsdB, IsdA, ClfA, ClfB], |
| | | [IsdB, IsdA, ClfA, SdrD], [IsdB, IsdA, ClfB, Hla209], [IsdB, IsdA, ClfB, ClfA], [IsdB, IsdA, ClfB, |
| | | SdrD], [IsdB, IsdA, SdrD, Hla209], [IsdB, IsdA, SdrD, ClfA], [IsdB, IsdA, SdrD, ClfB] |
| 7 | at least 5 “Group | [Hla209, ClfA, ClfB, SdrD, IsdA], [Hla209, ClfA, ClfB, SdrD, IsdB], [Hla209, ClfA, ClfB, IsdA, |
| | B” SA antigens | SdrD], [Hla209, ClfA, ClfB, IsdA, IsdB], [Hla209, ClfA, ClfB, IsdB, SdrD], [Hla209, ClfA, ClfB, |
| | selected from the | IsdB, IsdA], [Hla209, ClfA, SdrD, ClfB, IsdA], [Hla209, ClfA, SdrD, ClfB, IsdB], [Hla209, ClfA, |
| | subgroups of: | SdrD, IsdA, ClfB], [Hla209, ClfA, SdrD, IsdA, IsdB], [Hla209, ClfA, SdrD, IsdB, ClfB], [Hla209, |
| | | ClfA, SdrD, IsdB, IsdA], [Hla209, ClfA, IsdA, ClfB, SdrD], [Hla209, ClfA, IsdA, ClfB, IsdB], |
| | | [Hla209, ClfA, IsdA, SdrD, ClfB], [Hla209, ClfA, IsdA, SdrD, IsdB], [Hla209, ClfA, IsdA, IsdB, ClfB], |
| | | [Hla209, ClfA, IsdA, IsdB, SdrD], [Hla209, ClfA, IsdB, ClfB, SdrD], [Hla209, ClfA, IsdB, ClfB, |
| | | IsdA], [Hla209, ClfA, IsdB, SdrD, ClfB], [Hla209, ClfA, IsdB, SdrD, IsdA], [Hla209, ClfA, IsdB, |
| | | IsdA, ClfB], [Hla209, ClfA, IsdB, IsdA, SdrD], [Hla209, ClfB, ClfA, SdrD, IsdA], [Hla209, ClfB, |
| | | ClfA, SdrD, IsdB], [Hla209, ClfB, ClfA, IsdA, SdrD], [Hla209, ClfB, ClfA, IsdA, IsdB], [Hla209, |
| | | ClfB, ClfA, IsdB, SdrD], [Hla209, ClfB, ClfA, IsdB, IsdA], [Hla209, ClfB, SdrD, ClfA, IsdA], |
| | | [Hla209, ClfB, SdrD, ClfA, IsdB], [Hla209, ClfB, SdrD, IsdA, ClfA], [Hla209, ClfB, SdrD, IsdA, |
| | | IsdB], [Hla209, ClfB, SdrD, IsdB, ClfA], [Hla209, ClfB, SdrD, IsdB, IsdA], [Hla209, ClfB, IsdA, |
| | | ClfA, SdrD], [Hla209, ClfB, IsdA, ClfA, IsdB], [Hla209, ClfB, IsdA, SdrD, ClfA], [Hla209, ClfB, |
| | | IsdA, SdrD, IsdB], [Hla209, ClfB, IsdA, IsdB, ClfA], [Hla209, ClfB, IsdA, IsdB, SdrD], [Hla209, |
| | | ClfB, IsdB, ClfA, SdrD], [Hla209, ClfB, IsdB, ClfA, IsdA], [Hla209, ClfB, IsdB, SdrD, ClfA], [Hla209, |
| | | ClfB, IsdB, SdrD, IsdA], [Hla209, ClfB, IsdB, IsdA, ClfA], [Hla209, ClfB, IsdB, IsdA, SdrD], |
| | | [Hla209, SdrD, ClfA, ClfB, IsdA], [Hla209, SdrD, ClfA, ClfB, IsdB], [Hla209, SdrD, ClfA, IsdA, |
| | | ClfB], [Hla209, SdrD, ClfA, IsdA, IsdB], [Hla209, SdrD, ClfA, IsdB, ClfB], [Hla209, SdrD, ClfA, |
| | | IsdB, IsdA], [Hla209, SdrD, ClfB, ClfA, IsdA], [Hla209, SdrD, ClfB, ClfA, IsdB], [Hla209, SdrD, |
| | | ClfB, IsdA, ClfA], [Hla209, SdrD, ClfB, IsdA, IsdB], [Hla209, SdrD, ClfB, IsdB, ClfA], [Hla209, |
| | | SdrD, ClfB, IsdB, IsdA], [Hla209, SdrD, IsdA, ClfA, ClfB], [Hla209, SdrD, IsdA, ClfA, IsdB], |
| | | [Hla209, SdrD, IsdA, ClfB, ClfA], [Hla209, SdrD, IsdA, ClfB, IsdB], [Hla209, SdrD, IsdA, IsdB, ClfA], |
| | | [Hla209, SdrD, IsdA, IsdB, ClfB], [Hla209, SdrD, IsdB, ClfA, ClfB], [Hla209, SdrD, IsdB, ClfA, |
| | | IsdA], [Hla209, SdrD, IsdB, ClfB, ClfA], [Hla209, SdrD, IsdB, ClfB, IsdA], [Hla209, SdrD, IsdB, |
| | | IsdA, ClfA], [Hla209, SdrD, IsdB, IsdA, ClfB], [Hla209, IsdA, ClfA, ClfB, SdrD], [Hla209, IsdA, |
| | | ClfA, ClfB, IsdB], [Hla209, IsdA, ClfA, SdrD, ClfB], [Hla209, IsdA, ClfA, SdrD, IsdB], [Hla209, |
| | | IsdA, ClfA, IsdB, ClfB], [Hla209, IsdA, ClfA, IsdB, SdrD], [Hla209, IsdA, ClfB, ClfA, SdrD], |
| | | [Hla209, IsdA, ClfB, ClfA, IsdB], [Hla209, IsdA, ClfB, SdrD, ClfA], [Hla209, IsdA, ClfB, SdrD, IsdB], |
| | | [Hla209, IsdA, ClfB, IsdB, ClfA], [Hla209, IsdA, ClfB, IsdB, SdrD], [Hla209, IsdA, SdrD, ClfA, |
| | | ClfB], [Hla209, IsdA, SdrD, ClfA, IsdB], [Hla209, IsdA, SdrD, ClfB, ClfA], [Hla209, IsdA, SdrD, |
| | | ClfB, IsdB], [Hla209, IsdA, SdrD, IsdB, ClfA], [Hla209, IsdA, SdrD, IsdB, ClfB], [Hla209, IsdA, |
| | | IsdB, ClfA, ClfB], [Hla209, IsdA, IsdB, ClfA, SdrD], [Hla209, IsdA, IsdB, ClfB, ClfA], [Hla209, |
| | | IsdA, IsdB, ClfB, SdrD], [Hla209, IsdA, IsdB, SdrD, ClfA], [Hla209, IsdA, IsdB, SdrD, ClfB], [Hla209, |
| | | IsdB, ClfA, ClfB, SdrD], [Hla209, IsdB, ClfA, ClfB, IsdA], [Hla209, IsdB, ClfA, SdrD, ClfB], |
| | | [Hla209, IsdB, ClfA, SdrD, IsdA], [Hla209, IsdB, ClfA, IsdA, ClfB], [Hla209, IsdB, ClfA, IsdA, |
| | | SdrD], [Hla209, IsdB, ClfB, ClfA, SdrD], [Hla209, IsdB, ClfB, ClfA, IsdA], [Hla209, IsdB, ClfB, SdrD, |
| | | ClfA], [Hla209, IsdB, ClfB, SdrD, IsdA], [Hla209, IsdB, ClfB, IsdA, ClfA], [Hla209, IsdB, ClfB, |
| | | IsdA, SdrD], [Hla209, IsdB, SdrD, ClfA, ClfB], [Hla209, IsdB, SdrD, ClfA, IsdA], [Hla209, IsdB, |
| | | SdrD, ClfB, ClfA], [Hla209, IsdB, SdrD, ClfB, IsdA], [Hla209, IsdB, SdrD, IsdA, ClfA], [Hla209, |
| | | IsdB, SdrD, IsdA, ClfB], [Hla209, IsdB, IsdA, ClfA, ClfB], [Hla209, IsdB, IsdA, ClfA, SdrD], |
| | | [Hla209, IsdB, IsdA, ClfB, ClfA], [Hla209, IsdB, IsdA, ClfB, SdrD], [Hla209, IsdB, IsdA, SdrD, |
| | | ClfA], [Hla209, IsdB, IsdA, SdrD, ClfB], [ClfA, Hla209, ClfB, SdrD, IsdA], [ClfA, Hla209, ClfB, |
| | | SdrD, IsdB], [ClfA, Hla209, ClfB, IsdA, SdrD], [ClfA, Hla209, ClfB, IsdA, IsdB], [ClfA, Hla209, ClfB, |
| | | IsdB, SdrD], [ClfA, Hla209, ClfB, IsdB, IsdA], [ClfA, Hla209, SdrD, ClfB, IsdA], [ClfA, Hla209, |
| | | SdrD, ClfB, IsdB], [ClfA, Hla209, SdrD, IsdA, ClfB], [ClfA, Hla209, SdrD, IsdA, IsdB], [ClfA, |
| | | Hla209, SdrD, IsdB, ClfB], [ClfA, Hla209, SdrD, IsdB, IsdA], [ClfA, Hla209, IsdA, ClfB, SdrD], |
| | | [ClfA, Hla209, IsdA, ClfB, IsdB], [ClfA, Hla209, IsdA, SdrD, ClfB], [ClfA, Hla209, IsdA, SdrD, |
| | | IsdB], [ClfA, Hla209, IsdA, IsdB, ClfB], [ClfA, Hla209, IsdA, IsdB, SdrD], [ClfA, Hla209, IsdB, |
| | | ClfB, SdrD], [ClfA, Hla209, IsdB, ClfB, IsdA], [ClfA, Hla209, IsdB, SdrD, ClfB], [ClfA, Hla209, |
| | | IsdB, SdrD, IsdA], [ClfA, Hla209, IsdB, IsdA, ClfB], [ClfA, Hla209, IsdB, IsdA, SdrD], [ClfA, ClfB, |
| | | Hla209, SdrD, IsdA], [ClfA, ClfB, Hla209, SdrD, IsdB], [ClfA, ClfB, Hla209, IsdA, SdrD], [ClfA, |
| | | ClfB, Hla209, IsdA, IsdB], [ClfA, ClfB, Hla209, IsdB, SdrD], [ClfA, ClfB, Hla209, IsdB, IsdA], |
| | | [ClfA, ClfB, SdrD, Hla209, IsdA], [ClfA, ClfB, SdrD, Hla209, IsdB], [ClfA, ClfB, SdrD, IsdA, |
| | | Hla209], [ClfA, ClfB, SdrD, IsdA, IsdB], [ClfA, ClfB, SdrD, IsdB, Hla209], [ClfA, ClfB, SdrD, IsdB, |
| | | IsdA], [ClfA, ClfB, IsdA, Hla209, SdrD], [ClfA, ClfB, IsdA, Hla209, IsdB], [ClfA, ClfB, IsdA, SdrD, |
| | | Hla209], [ClfA, ClfB, IsdA, SdrD, IsdB], [ClfA, ClfB, IsdA, IsdB, Hla209], [ClfA, ClfB, IsdA, |
| | | IsdB, SdrD], [ClfA, ClfB, IsdB, Hla209, SdrD], [ClfA, ClfB, IsdB, Hla209, IsdA], [ClfA, ClfB, IsdB, |
| | | SdrD, Hla209], [ClfA, ClfB, IsdB, SdrD, IsdA], [ClfA, ClfB, IsdB, IsdA, Hla209], [ClfA, ClfB, |
| | | IsdB, IsdA, SdrD], [ClfA, SdrD, Hla209, ClfB, IsdA], [ClfA, SdrD, Hla209, ClfB, IsdB], [ClfA, SdrD, |
| | | Hla209, IsdA, ClfB], [ClfA, SdrD, Hla209, IsdA, IsdB], [ClfA, SdrD, Hla209, IsdB, ClfB], [ClfA, |
| | | SdrD, Hla209, IsdB, IsdA], [ClfA, SdrD, ClfB, Hla209, IsdA], [ClfA, SdrD, ClfB, Hla209, IsdB], |
| | | [ClfA, SdrD, ClfB, IsdA, Hla209], [ClfA, SdrD, ClfB, IsdA, IsdB], [ClfA, SdrD, ClfB, IsdB, |
| | | Hla209], [ClfA, SdrD, ClfB, IsdB, IsdA], [ClfA, SdrD, IsdA, Hla209, ClfB], [ClfA, SdrD, IsdA, Hla209, |
| | | IsdB], [ClfA, SdrD, IsdA, ClfB, Hla209], [ClfA, SdrD, IsdA, ClfB, IsdB], [ClfA, SdrD, IsdA, IsdB, |
| | | Hla209], [ClfA, SdrD, IsdA, IsdB, ClfB], [ClfA, SdrD, IsdB, Hla209, ClfB], [ClfA, SdrD, IsdB, |
| | | Hla209, IsdA], [ClfA, SdrD, IsdB, ClfB, Hla209], [ClfA, SdrD, IsdB, ClfB, IsdA], [ClfA, SdrD, |
| | | IsdB, IsdA, Hla209], [ClfA, SdrD, IsdB, IsdA, ClfB], [ClfA, IsdA, Hla209, ClfB, SdrD], [ClfA, IsdA, |
| | | Hla209, ClfB, IsdB], [ClfA, IsdA, Hla209, SdrD, ClfB], [ClfA, IsdA, Hla209, SdrD, IsdB], [ClfA, |
| | | IsdA, Hla209, IsdB, ClfB], [ClfA, IsdA, Hla209, IsdB, SdrD], [ClfA, IsdA, ClfB, Hla209, SdrD], |
| | | [ClfA, IsdA, ClfB, Hla209, IsdB], [ClfA, IsdA, ClfB, SdrD, Hla209], [ClfA, IsdA, ClfB, SdrD, IsdB], |
| | | [ClfA, IsdA, ClfB, IsdB, Hla209], [ClfA, IsdA, ClfB, IsdB, SdrD], [ClfA, IsdA, SdrD, Hla209, |
| | | ClfB], [ClfA, IsdA, SdrD, Hla209, IsdB], [ClfA, IsdA, SdrD, ClfB, Hla209], [ClfA, IsdA, SdrD, |
| | | ClfB, IsdB], [ClfA, IsdA, SdrD, IsdB, Hla209], [ClfA, IsdA, SdrD, IsdB, ClfB], [ClfA, IsdA, IsdB, |
| | | Hla209, ClfB], [ClfA, IsdA, IsdB, Hla209, SdrD], [ClfA, IsdA, IsdB, ClfB, Hla209], [ClfA, IsdA, IsdB, |
| | | ClfB, SdrD], [ClfA, IsdA, IsdB, SdrD, Hla209], [ClfA, IsdA, IsdB, SdrD, ClfB], [ClfA, IsdB, |
| | | Hla209, ClfB, SdrD], [ClfA, IsdB, Hla209, ClfB, IsdA], [ClfA, IsdB, Hla209, SdrD, ClfB], [ClfA, IsdB, |
| | | Hla209, SdrD, IsdA], [ClfA, IsdB, Hla209, IsdA, ClfB], [ClfA, IsdB, Hla209, IsdA, SdrD], [ClfA, |
| | | IsdB, ClfB, Hla209, SdrD], [ClfA, IsdB, ClfB, Hla209, IsdA], [ClfA, IsdB, ClfB, SdrD, Hla209], |
| | | [ClfA, IsdB, ClfB, SdrD, IsdA], [ClfA, IsdB, ClfB, IsdA, Hla209], [ClfA, IsdB, ClfB, IsdA, SdrD], |
| | | [ClfA, IsdB, SdrD, Hla209, ClfB], [ClfA, IsdB, SdrD, Hla209, IsdA], [ClfA, IsdB, SdrD, ClfB, |
| | | Hla209], [ClfA, IsdB, SdrD, ClfB, IsdA], [ClfA, IsdB, SdrD, IsdA, Hla209], [ClfA, IsdB, SdrD, IsdA, |
| | | ClfB], [ClfA, IsdB, IsdA, Hla209, ClfB], [ClfA, IsdB, IsdA, Hla209, SdrD], [ClfA, IsdB, IsdA, |
| | | ClfB, Hla209], [ClfA, IsdB, IsdA, ClfB, SdrD], [ClfA, IsdB, IsdA, SdrD, Hla209], [ClfA, IsdB, IsdA, |
| | | SdrD, ClfB], [ClfB, Hla209, ClfA, SdrD, IsdA], [ClfB, Hla209, ClfA, SdrD, IsdB], [ClfB, Hla209, |
| | | ClfA, IsdA, SdrD], [ClfB, Hla209, ClfA, IsdA, IsdB], [ClfB, Hla209, ClfA, IsdB, SdrD], [ClfB, |
| | | Hla209, ClfA, IsdB, IsdA], [ClfB, Hla209, SdrD, ClfA, IsdA], [ClfB, Hla209, SdrD, ClfA, IsdB], |
| | | [ClfB, Hla209, SdrD, IsdA, ClfA], [ClfB, Hla209, SdrD, IsdA, IsdB], [ClfB, Hla209, SdrD, IsdB, |
| | | ClfA], [ClfB, Hla209, SdrD, IsdB, IsdA], [ClfB, Hla209, IsdA, ClfA, SdrD], [ClfB, Hla209, IsdA, |
| | | ClfA, IsdB], [ClfB, Hla209, IsdA, SdrD, ClfA], [ClfB, Hla209, IsdA, SdrD, IsdB], [ClfB, Hla209, |
| | | IsdA, IsdB, ClfA], [ClfB, Hla209, IsdA, IsdB, SdrD], [ClfB, Hla209, IsdB, ClfA, SdrD], [ClfB, Hla209, |
| | | IsdB, ClfA, IsdA], [ClfB, Hla209, IsdB, SdrD, ClfA], [ClfB, Hla209, IsdB, SdrD, IsdA], [ClfB, |
| | | Hla209, IsdB, IsdA, ClfA], [ClfB, Hla209, IsdB, IsdA, SdrD], [ClfB, ClfA, Hla209, SdrD, IsdA], |
| | | [ClfB, ClfA, Hla209, SdrD, IsdB], [ClfB, ClfA, Hla209, IsdA, SdrD], [ClfB, ClfA, Hla209, IsdA, |
| | | IsdB], [ClfB, ClfA, Hla209, IsdB, SdrD], [ClfB, ClfA, Hla209, IsdB, IsdA], [ClfB, ClfA, SdrD, Hla209, |
| | | IsdA], [ClfB, ClfA, SdrD, Hla209, IsdB], [ClfB, ClfA, SdrD, IsdA, Hla209], [ClfB, ClfA, SdrD, |
| | | IsdA, IsdB], [ClfB, ClfA, SdrD, IsdB, Hla209], [ClfB, ClfA, SdrD, IsdB, IsdA], [ClfB, ClfA, IsdA, |
| | | Hla209, SdrD], [ClfB, ClfA, IsdA, Hla209, IsdB], [ClfB, ClfA, IsdA, SdrD, Hla209], [ClfB, ClfA, |
| | | IsdA, SdrD, IsdB], [ClfB, ClfA, IsdA, IsdB, Hla209], [ClfB, ClfA, IsdA, IsdB, SdrD], [ClfB, ClfA, |
| | | IsdB, Hla209, SdrD], [ClfB, ClfA, IsdB, Hla209, IsdA], [ClfB, ClfA, IsdB, SdrD, Hla209], [ClfB, |
| | | ClfA, IsdB, SdrD, IsdA], [ClfB, ClfA, IsdB, IsdA, Hla209], [ClfB, ClfA, IsdB, IsdA, SdrD], [ClfB, |
| | | SdrD, Hla209, ClfA, IsdA], [ClfB, SdrD, Hla209, ClfA, IsdB], [ClfB, SdrD, Hla209, IsdA, ClfA], |
| | | [ClfB, SdrD, Hla209, IsdA, IsdB], [ClfB, SdrD, Hla209, IsdB, ClfA], [ClfB, SdrD, Hla209, IsdB, |
| | | IsdA], [ClfB, SdrD, ClfA, Hla209, IsdA], [ClfB, SdrD, ClfA, Hla209, IsdB], [ClfB, SdrD, ClfA, IsdA, |
| | | Hla209], [ClfB, SdrD, ClfA, IsdA, IsdB], [ClfB, SdrD, ClfA, IsdB, Hla209], [ClfB, SdrD, ClfA, |
| | | IsdB, IsdA], [ClfB, SdrD, IsdA, Hla209, ClfA], [ClfB, SdrD, IsdA, Hla209, IsdB], [ClfB, SdrD, |
| | | IsdA, ClfA, Hla209], [ClfB, SdrD, IsdA, ClfA, IsdB], [ClfB, SdrD, IsdA, IsdB, Hla209], [ClfB, SdrD, |
| | | IsdA, IsdB, ClfA], [ClfB, SdrD, IsdB, Hla209, ClfA], [ClfB, SdrD, IsdB, Hla209, IsdA], [ClfB, |
| | | SdrD, IsdB, ClfA, Hla209], [ClfB, SdrD, IsdB, ClfA, IsdA], [ClfB, SdrD, IsdB, IsdA, Hla209], |
| | | [ClfB, SdrD, IsdB, IsdA, ClfA], [ClfB, IsdA, Hla209, ClfA, SdrD], [ClfB, IsdA, Hla209, ClfA, IsdB], |
| | | [ClfB, IsdA, Hla209, SdrD, ClfA], [ClfB, IsdA, Hla209, SdrD, IsdB], [ClfB, IsdA, Hla209, IsdB, |
| | | ClfA], [ClfB, IsdA, Hla209, IsdB, SdrD], [ClfB, IsdA, ClfA, Hla209, SdrD], [ClfB, IsdA, ClfA, Hla209, |
| | | IsdB], [ClfB, IsdA, ClfA, SdrD, Hla209], [ClfB, IsdA, ClfA, SdrD, IsdB], [ClfB, IsdA, ClfA, |
| | | IsdB, Hla209], [ClfB, IsdA, ClfA, IsdB, SdrD], [ClfB, IsdA, SdrD, Hla209, ClfA], [ClfB, IsdA, SdrD, |
| | | Hla209, IsdB], [ClfB, IsdA, SdrD, ClfA, Hla209], [ClfB, IsdA, SdrD, ClfA, IsdB], [ClfB, IsdA, |
| | | SdrD, IsdB, Hla209], [ClfB, IsdA, SdrD, IsdB, ClfA], [ClfB, IsdA, IsdB, Hla209, ClfA], [ClfB, IsdA, |
| | | IsdB, Hla209, SdrD], [ClfB, IsdA, IsdB, ClfA, Hla209], [ClfB, IsdA, IsdB, ClfA, SdrD], [ClfB, |
| | | IsdA, IsdB, SdrD, Hla209], [ClfB, IsdA, IsdB, SdrD, ClfA], [ClfB, IsdB, Hla209, ClfA, SdrD], [ClfB, |
| | | IsdB, Hla209, ClfA, IsdA], [ClfB, IsdB, Hla209, SdrD, ClfA], [ClfB, IsdB, Hla209, SdrD, IsdA], |
| | | [ClfB, IsdB, Hla209, IsdA, ClfA], [ClfB, IsdB, Hla209, IsdA, SdrD], [ClfB, IsdB, ClfA, Hla209, |
| | | SdrD], [ClfB, IsdB, ClfA, Hla209, IsdA], [ClfB, IsdB, ClfA, SdrD, Hla209], [ClfB, IsdB, ClfA, SdrD, |
| | | IsdA], [ClfB, IsdB, ClfA, IsdA, Hla209], [ClfB, IsdB, ClfA, IsdA, SdrD], [ClfB, IsdB, SdrD, |
| | | Hla209, ClfA], [ClfB, IsdB, SdrD, Hla209, IsdA], [ClfB, IsdB, SdrD, ClfA, Hla209], [ClfB, IsdB, |
| | | SdrD, ClfA, IsdA], [ClfB, IsdB, SdrD, IsdA, Hla209], [ClfB, IsdB, SdrD, IsdA, ClfA], [ClfB, IsdB, |
| | | IsdA, Hla209, ClfA], [ClfB, IsdB, IsdA, Hla209, SdrD], [ClfB, IsdB, IsdA, ClfA, Hla209], [ClfB, |
| | | IsdB, IsdA, ClfA, SdrD], [ClfB, IsdB, IsdA, SdrD, Hla209], [ClfB, IsdB, IsdA, SdrD, ClfA], [SdrD, |
| | | Hla209, ClfA, ClfB, IsdA], [SdrD, Hla209, ClfA, ClfB, IsdB], [SdrD, Hla209, ClfA, IsdA, ClfB], |
| | | [SdrD, Hla209, ClfA, IsdA, IsdB], [SdrD, Hla209, ClfA, IsdB, ClfB], [SdrD, Hla209, ClfA, IsdB, |
| | | IsdA], [SdrD, Hla209, ClfB, ClfA, IsdA], [SdrD, Hla209, ClfB, ClfA, IsdB], [SdrD, Hla209, ClfB, |
| | | IsdA, ClfA], [SdrD, Hla209, ClfB, IsdA, IsdB], [SdrD, Hla209, ClfB, IsdB, ClfA], [SdrD, Hla209, |
| | | ClfB, IsdB, IsdA], [SdrD, Hla209, IsdA, ClfA, ClfB], [SdrD, Hla209, IsdA, ClfA, IsdB], [SdrD, |
| | | Hla209, IsdA, ClfB, ClfA], [SdrD, Hla209, IsdA, ClfB, IsdB], [SdrD, Hla209, IsdA, IsdB, ClfA], [SdrD, |
| | | Hla209, IsdA, IsdB, ClfB], [SdrD, Hla209, IsdB, ClfA, ClfB], [SdrD, Hla209, IsdB, ClfA, IsdA], |
| | | [SdrD, Hla209, IsdB, ClfB, ClfA], [SdrD, Hla209, IsdB, ClfB, IsdA], [SdrD, Hla209, IsdB, IsdA, |
| | | ClfA], [SdrD, Hla209, IsdB, IsdA, ClfB], [SdrD, ClfA, Hla209, ClfB, IsdA], [SdrD, ClfA, Hla209, |
| | | ClfB, IsdB], [SdrD, ClfA, Hla209, IsdA, ClfB], [SdrD, ClfA, Hla209, IsdA, IsdB], [SdrD, ClfA, |
| | | Hla209, IsdB, ClfB], [SdrD, ClfA, Hla209, IsdB, IsdA], [SdrD, ClfA, ClfB, Hla209, IsdA], [SdrD, |
| | | ClfA, ClfB, Hla209, IsdB], [SdrD, ClfA, ClfB, IsdA, Hla209], [SdrD, ClfA, ClfB, IsdA, IsdB], [SdrD, |
| | | ClfA, ClfB, IsdB, Hla209], [SdrD, ClfA, ClfB, IsdB, IsdA], [SdrD, ClfA, IsdA, Hla209, ClfB], |
| | | [SdrD, ClfA, IsdA, Hla209, IsdB], [SdrD, ClfA, IsdA, ClfB, Hla209], [SdrD, ClfA, IsdA, ClfB, IsdB], |
| | | [SdrD, ClfA, IsdA, IsdB, Hla209], [SdrD, ClfA, IsdA, IsdB, ClfB], [SdrD, ClfA, IsdB, Hla209, |
| | | ClfB], [SdrD, ClfA, IsdB, Hla209, IsdA], [SdrD, ClfA, IsdB, ClfB, Hla209], [SdrD, ClfA, IsdB, ClfB, |
| | | IsdA], [SdrD, ClfA, IsdB, IsdA, Hla209], [SdrD, ClfA, IsdB, IsdA, ClfB], [SdrD, ClfB, Hla209, |
| | | ClfA, IsdA], [SdrD, ClfB, Hla209, ClfA, IsdB], [SdrD, ClfB, Hla209, IsdA, ClfA], [SdrD, ClfB, |
| | | Hla209, IsdA, IsdB], [SdrD, ClfB, Hla209, IsdB, ClfA], [SdrD, ClfB, Hla209, IsdB, IsdA], [SdrD, ClfB, |
| | | ClfA, Hla209, IsdA], [SdrD, ClfB, ClfA, Hla209, IsdB], [SdrD, ClfB, ClfA, IsdA, Hla209], [SdrD, |
| | | ClfB, ClfA, IsdA, IsdB], [SdrD, ClfB, ClfA, IsdB, Hla209], [SdrD, ClfB, ClfA, IsdB, IsdA], [SdrD, |
| | | ClfB, IsdA, Hla209, ClfA], [SdrD, ClfB, IsdA, Hla209, IsdB], [SdrD, ClfB, IsdA, ClfA, Hla209], |
| | | [SdrD, ClfB, IsdA, ClfA, IsdB], [SdrD, ClfB, IsdA, IsdB, Hla209], [SdrD, ClfB, IsdA, IsdB, ClfA], |
| | | [SdrD, ClfB, IsdB, Hla209, ClfA], [SdrD, ClfB, IsdB, Hla209, IsdA], [SdrD, ClfB, IsdB, ClfA, |
| | | Hla209], [SdrD, ClfB, IsdB, ClfA, IsdA], [SdrD, ClfB, IsdB, IsdA, Hla209], [SdrD, ClfB, IsdB, IsdA, |
| | | ClfA], [SdrD, IsdA, Hla209, ClfA, ClfB], [SdrD, IsdA, Hla209, ClfA, IsdB], [SdrD, IsdA, Hla209, |
| | | ClfB, ClfA], [SdrD, IsdA, Hla209, ClfB, IsdB], [SdrD, IsdA, Hla209, IsdB, ClfA], [SdrD, IsdA, |
| | | Hla209, IsdB, ClfB], [SdrD, IsdA, ClfA, Hla209, ClfB], [SdrD, IsdA, ClfA, Hla209, IsdB], [SdrD, |
| | | IsdA, ClfA, ClfB, Hla209], [SdrD, IsdA, ClfA, ClfB, IsdB], [SdrD, IsdA, ClfA, IsdB, Hla209], |
| | | [SdrD, IsdA, ClfA, IsdB, ClfB], [SdrD, IsdA, ClfB, Hla209, ClfA], [SdrD, IsdA, ClfB, Hla209, IsdB], |
| | | [SdrD, IsdA, ClfB, ClfA, Hla209], [SdrD, IsdA, ClfB, ClfA, IsdB], [SdrD, IsdA, ClfB, IsdB, Hla209], |
| | | [SdrD, IsdA, ClfB, IsdB, ClfA], [SdrD, IsdA, IsdB, Hla209, ClfA], [SdrD, IsdA, IsdB, Hla209, |
| | | ClfB], [SdrD, IsdA, IsdB, ClfA, Hla209], [SdrD, IsdA, IsdB, ClfA, ClfB], [SdrD, IsdA, IsdB, ClfB, |
| | | Hla209], [SdrD, IsdA, IsdB, ClfB, ClfA], [SdrD, IsdB, Hla209, ClfA, ClfB], [SdrD, IsdB, Hla209, |
| | | ClfA, IsdA], [SdrD, IsdB, Hla209, ClfB, ClfA], [SdrD, IsdB, Hla209, ClfB, IsdA], [SdrD, IsdB, |
| | | Hla209, IsdA, ClfA], [SdrD, IsdB, Hla209, IsdA, ClfB], [SdrD, IsdB, ClfA, Hla209, ClfB], [SdrD, |
| | | IsdB, ClfA, Hla209, IsdA], [SdrD, IsdB, ClfA, ClfB, Hla209], [SdrD, IsdB, ClfA, ClfB, IsdA], [SdrD, |
| | | IsdB, ClfA, IsdA, Hla209], [SdrD, IsdB, ClfA, IsdA, ClfB], [SdrD, IsdB, ClfB, Hla209, ClfA], |
| | | [SdrD, IsdB, ClfB, Hla209, IsdA], [SdrD, IsdB, ClfB, ClfA, Hla209], [SdrD, IsdB, ClfB, ClfA, IsdA], |
| | | [SdrD, IsdB, ClfB, IsdA, Hla209], [SdrD, IsdB, ClfB, IsdA, ClfA], [SdrD, IsdB, IsdA, Hla209, |
| | | ClfA], [SdrD, IsdB, IsdA, Hla209, ClfB], [SdrD, IsdB, IsdA, ClfA, Hla209], [SdrD, IsdB, IsdA, |
| | | ClfA, ClfB], [SdrD, IsdB, IsdA, ClfB, Hla209], [SdrD, IsdB, IsdA, ClfB, ClfA], [IsdA, Hla209, ClfA, |
| | | ClfB, SdrD], [IsdA, Hla209, ClfA, ClfB, IsdB], [IsdA, Hla209, ClfA, SdrD, ClfB], [IsdA, Hla209, |
| | | ClfA, SdrD, IsdB], [IsdA, Hla209, ClfA, IsdB, ClfB], [IsdA, Hla209, ClfA, IsdB, SdrD], [IsdA, |
| | | Hla209, ClfB, ClfA, SdrD], [IsdA, Hla209, ClfB, ClfA, IsdB], [IsdA, Hla209, ClfB, SdrD, ClfA], [IsdA, |
| | | Hla209, ClfB, SdrD, IsdB], [IsdA, Hla209, ClfB, IsdB, ClfA], [IsdA, Hla209, ClfB, IsdB, SdrD], |
| | | [IsdA, Hla209, SdrD, ClfA, ClfB], [IsdA, Hla209, SdrD, ClfA, IsdB], [IsdA, Hla209, SdrD, ClfB, |
| | | ClfA], [IsdA, Hla209, SdrD, ClfB, IsdB], [IsdA, Hla209, SdrD, IsdB, ClfA], [IsdA, Hla209, SdrD, |
| | | IsdB, ClfB], [IsdA, Hla209, IsdB, ClfA, ClfB], [IsdA, Hla209, IsdB, ClfA, SdrD], [IsdA, Hla209, |
| | | IsdB, ClfB, ClfA], [IsdA, Hla209, IsdB, ClfB, SdrD], [IsdA, Hla209, IsdB, SdrD, ClfA], [IsdA, |
| | | Hla209, IsdB, SdrD, ClfB], [IsdA, ClfA, Hla209, ClfB, SdrD], [IsdA, ClfA, Hla209, ClfB, IsdB], |
| | | [IsdA, ClfA, Hla209, SdrD, ClfB], [IsdA, ClfA, Hla209, SdrD, IsdB], [IsdA, ClfA, Hla209, IsdB, |
| | | ClfB], [IsdA, ClfA, Hla209, IsdB, SdrD], [IsdA, ClfA, ClfB, Hla209, SdrD], [IsdA, ClfA, ClfB, |
| | | Hla209, IsdB], [IsdA, ClfA, ClfB, SdrD, Hla209], [IsdA, ClfA, ClfB, SdrD, IsdB], [IsdA, ClfA, ClfB, |
| | | IsdB, Hla209], [IsdA, ClfA, ClfB, IsdB, SdrD], [IsdA, ClfA, SdrD, Hla209, ClfB], [IsdA, ClfA, SdrD, |
| | | Hla209, IsdB], [IsdA, ClfA, SdrD, ClfB, Hla209], [IsdA, ClfA, SdrD, ClfB, IsdB], [IsdA, ClfA, |
| | | SdrD, IsdB, Hla209], [IsdA, ClfA, SdrD, IsdB, ClfB], [IsdA, ClfA, IsdB, Hla209, ClfB], [IsdA, ClfA, |
| | | IsdB, Hla209, SdrD], [IsdA, ClfA, IsdB, ClfB, Hla209], [IsdA, ClfA, IsdB, ClfB, SdrD], [IsdA, |
| | | ClfA, IsdB, SdrD, Hla209], [IsdA, ClfA, IsdB, SdrD, ClfB], [IsdA, ClfB, Hla209, ClfA, SdrD], [IsdA, |
| | | ClfB, Hla209, ClfA, IsdB], [IsdA, ClfB, Hla209, SdrD, ClfA], [IsdA, ClfB, Hla209, SdrD, IsdB], |
| | | [IsdA, ClfB, Hla209, IsdB, ClfA], [IsdA, ClfB, Hla209, IsdB, SdrD], [IsdA, ClfB, ClfA, Hla209, |
| | | SdrD], [IsdA, ClfB, ClfA, Hla209, IsdB], [IsdA, ClfB, ClfA, SdrD, Hla209], [IsdA, ClfB, ClfA, |
| | | SdrD, IsdB], [IsdA, ClfB, ClfA, IsdB, Hla209], [IsdA, ClfB, ClfA, IsdB, SdrD], [IsdA, ClfB, SdrD, |
| | | Hla209, ClfA], [IsdA, ClfB, SdrD, Hla209, IsdB], [IsdA, ClfB, SdrD, ClfA, Hla209], [IsdA, ClfB, |
| | | SdrD, ClfA, IsdB], [IsdA, ClfB, SdrD, IsdB, Hla209], [IsdA, ClfB, SdrD, IsdB, ClfA], [IsdA, ClfB, |
| | | IsdB, Hla209, ClfA], [IsdA, ClfB, IsdB, Hla209, SdrD], [IsdA, ClfB, IsdB, ClfA, Hla209], [IsdA, |
| | | ClfB, IsdB, ClfA, SdrD], [IsdA, ClfB, IsdB, SdrD, Hla209], [IsdA, ClfB, IsdB, SdrD, ClfA], [IsdA, |
| | | SdrD, Hla209, ClfA, ClfB], [IsdA, SdrD, Hla209, ClfA, IsdB], [IsdA, SdrD, Hla209, ClfB, ClfA], |
| | | [IsdA, SdrD, Hla209, ClfB, IsdB], [IsdA, SdrD, Hla209, IsdB, ClfA], [IsdA, SdrD, Hla209, IsdB, |
| | | ClfB], [IsdA, SdrD, ClfA, Hla209, ClfB], [IsdA, SdrD, ClfA, Hla209, IsdB], [IsdA, SdrD, ClfA, ClfB, |
| | | Hla209], [IsdA, SdrD, ClfA, ClfB, IsdB], [IsdA, SdrD, ClfA, IsdB, Hla209], [IsdA, SdrD, ClfA, |
| | | IsdB, ClfB], [IsdA, SdrD, ClfB, Hla209, ClfA], [IsdA, SdrD, ClfB, Hla209, IsdB], [IsdA, SdrD, |
| | | ClfB, ClfA, Hla209], [IsdA, SdrD, ClfB, ClfA, IsdB], [IsdA, SdrD, ClfB, IsdB, Hla209], [IsdA, SdrD, |
| | | ClfB, IsdB, ClfA], [IsdA, SdrD, IsdB, Hla209, ClfA], [IsdA, SdrD, IsdB, Hla209, ClfB], [IsdA, SdrD, |
| | | IsdB, ClfA, Hla209], [IsdA, SdrD, IsdB, ClfA, ClfB], [IsdA, SdrD, IsdB, ClfB, Hla209], [IsdA, |
| | | SdrD, IsdB, ClfB, ClfA], [IsdA, IsdB, Hla209, ClfA, ClfB], [IsdA, IsdB, Hla209, ClfA, SdrD], [IsdA, |
| | | IsdB, Hla209, ClfB, ClfA], [IsdA, IsdB, Hla209, ClfB, SdrD], [IsdA, IsdB, Hla209, SdrD, ClfA], |
| | | [IsdA, IsdB, Hla209, SdrD, ClfB], [IsdA, IsdB, ClfA, Hla209, ClfB], [IsdA, IsdB, ClfA, Hla209, |
| | | SdrD], [IsdA, IsdB, ClfA, ClfB, Hla209], [IsdA, IsdB, ClfA, ClfB, SdrD], [IsdA, IsdB, ClfA, SdrD, |
| | | Hla209], [IsdA, IsdB, ClfA, SdrD, ClfB], [IsdA, IsdB, ClfB, Hla209, ClfA], [IsdA, IsdB, ClfB, |
| | | Hla209, SdrD], [IsdA, IsdB, ClfB, ClfA, Hla209], [IsdA, IsdB, ClfB, ClfA, SdrD], [IsdA, IsdB, ClfB, |
| | | SdrD, Hla209], [IsdA, IsdB, ClfB, SdrD, ClfA], [IsdA, IsdB, SdrD, Hla209, ClfA], [IsdA, IsdB, |
| | | SdrD, Hla209, ClfB], [IsdA, IsdB, SdrD, ClfA, Hla209], [IsdA, IsdB, SdrD, ClfA, ClfB], [IsdA, IsdB, |
| | | SdrD, ClfB, Hla209], [IsdA, IsdB, SdrD, ClfB, ClfA], [IsdB, Hla209, ClfA, ClfB, SdrD], [IsdB, |
| | | Hla209, ClfA, ClfB, IsdA], [IsdB, Hla209, ClfA, SdrD, ClfB], [IsdB, Hla209, ClfA, SdrD, IsdA], [IsdB, |
| | | Hla209, ClfA, IsdA, ClfB], [IsdB, Hla209, ClfA, IsdA, SdrD], [IsdB, Hla209, ClfB, ClfA, SdrD], |
| | | [IsdB, Hla209, ClfB, ClfA, IsdA], [IsdB, Hla209, ClfB, SdrD, ClfA], [IsdB, Hla209, ClfB, SdrD, |
| | | IsdA], [IsdB, Hla209, ClfB, IsdA, ClfA], [IsdB, Hla209, ClfB, IsdA, SdrD], [IsdB, Hla209, SdrD, |
| | | ClfA, ClfB], [IsdB, Hla209, SdrD, ClfA, IsdA], [IsdB, Hla209, SdrD, ClfB, ClfA], [IsdB, Hla209, |
| | | SdrD, ClfB, IsdA], [IsdB, Hla209, SdrD, IsdA, ClfA], [IsdB, Hla209, SdrD, IsdA, ClfB], [IsdB, |
| | | Hla209, IsdA, ClfA, ClfB], [IsdB, Hla209, IsdA, ClfA, SdrD], [IsdB, Hla209, IsdA, ClfB, ClfA], [IsdB, |
| | | Hla209, IsdA, ClfB, SdrD], [IsdB, Hla209, IsdA, SdrD, ClfA], [IsdB, Hla209, IsdA, SdrD, ClfB], |
| | | [IsdB, ClfA, Hla209, ClfB, SdrD], [IsdB, ClfA, Hla209, ClfB, IsdA], [IsdB, ClfA, Hla209, SdrD, |
| | | ClfB], [IsdB, ClfA, Hla209, SdrD, IsdA], [IsdB, ClfA, Hla209, IsdA, ClfB], [IsdB, ClfA, Hla209, |
| | | IsdA, SdrD], [IsdB, ClfA, ClfB, Hla209, SdrD], [IsdB, ClfA, ClfB, Hla209, IsdA], [IsdB, ClfA, ClfB, |
| | | SdrD, Hla209], [IsdB, ClfA, ClfB, SdrD, IsdA], [IsdB, ClfA, ClfB, IsdA, Hla209], [IsdB, ClfA, |
| | | ClfB, IsdA, SdrD], [IsdB, ClfA, SdrD, Hla209, ClfB], [IsdB, ClfA, SdrD, Hla209, IsdA], [IsdB, |
| | | ClfA, SdrD, ClfB, Hla209], [IsdB, ClfA, SdrD, ClfB, IsdA], [IsdB, ClfA, SdrD, IsdA, Hla209], [IsdB, |
| | | ClfA, SdrD, IsdA, ClfB], [IsdB, ClfA, IsdA, Hla209, ClfB], [IsdB, ClfA, IsdA, Hla209, SdrD], |
| | | [IsdB, ClfA, IsdA, ClfB, Hla209], [IsdB, ClfA, IsdA, ClfB, SdrD], [IsdB, ClfA, IsdA, SdrD, Hla209], |
| | | [IsdB, ClfA, IsdA, SdrD, ClfB], [IsdB, ClfB, Hla209, ClfA, SdrD], [IsdB, ClfB, Hla209, ClfA, IsdA], |
| | | [IsdB, ClfB, Hla209, SdrD, ClfA], [IsdB, ClfB, Hla209, SdrD, IsdA], [IsdB, ClfB, Hla209, IsdA, |
| | | ClfA], [IsdB, ClfB, Hla209, IsdA, SdrD], [IsdB, ClfB, ClfA, Hla209, SdrD], [IsdB, ClfB, ClfA, |
| | | Hla209, IsdA], [IsdB, ClfB, ClfA, SdrD, Hla209], [IsdB, ClfB, ClfA, SdrD, IsdA], [IsdB, ClfB, ClfA, |
| | | IsdA, Hla209], [IsdB, ClfB, ClfA, IsdA, SdrD], [IsdB, ClfB, SdrD, Hla209, ClfA], [IsdB, ClfB, |
| | | SdrD, Hla209, IsdA], [IsdB, ClfB, SdrD, ClfA, Hla209], [IsdB, ClfB, SdrD, ClfA, IsdA], [IsdB, |
| | | ClfB, SdrD, IsdA, Hla209], [IsdB, ClfB, SdrD, IsdA, ClfA], [IsdB, ClfB, IsdA, Hla209, ClfA], [IsdB, |
| | | ClfB, IsdA, Hla209, SdrD], [IsdB, ClfB, IsdA, ClfA, Hla209], [IsdB, ClfB, IsdA, ClfA, SdrD], [IsdB, |
| | | ClfB, IsdA, SdrD, Hla209], [IsdB, ClfB, IsdA, SdrD, ClfA], [IsdB, SdrD, Hla209, ClfA, ClfB], |
| | | [IsdB, SdrD, Hla209, ClfA, IsdA], [IsdB, SdrD, Hla209, ClfB, ClfA], [IsdB, SdrD, Hla209, ClfB, |
| | | IsdA], [IsdB, SdrD, Hla209, IsdA, ClfA], [IsdB, SdrD, Hla209, IsdA, ClfB], [IsdB, SdrD, ClfA, |
| | | Hla209, ClfB], [IsdB, SdrD, ClfA, Hla209, IsdA], [IsdB, SdrD, ClfA, ClfB, Hla209], [IsdB, SdrD, ClfA, |
| | | ClfB, IsdA], [IsdB, SdrD, ClfA, IsdA, Hla209], [IsdB, SdrD, ClfA, IsdA, ClfB], [IsdB, SdrD, |
| | | ClfB, Hla209, ClfA], [IsdB, SdrD, ClfB, Hla209, IsdA], [IsdB, SdrD, ClfB, ClfA, Hla209], [IsdB, |
| | | SdrD, ClfB, ClfA, IsdA], [IsdB, SdrD, ClfB, IsdA, Hla209], [IsdB, SdrD, ClfB, IsdA, ClfA], [IsdB, |
| | | SdrD, IsdA, Hla209, ClfA], [IsdB, SdrD, IsdA, Hla209, ClfB], [IsdB, SdrD, IsdA, ClfA, Hla209], |
| | | [IsdB, SdrD, IsdA, ClfA, ClfB], [IsdB, SdrD, IsdA, ClfB, Hla209], [IsdB, SdrD, IsdA, ClfB, ClfA], |
| | | [IsdB, IsdA, Hla209, ClfA, ClfB], [IsdB, IsdA, Hla209, ClfA, SdrD], [IsdB, IsdA, Hla209, ClfB, ClfA], |
| | | [IsdB, IsdA, Hla209, ClfB, SdrD], [IsdB, IsdA, Hla209, SdrD, ClfA], [IsdB, IsdA, Hla209, SdrD, |
| | | ClfB], [IsdB, IsdA, ClfA, Hla209, ClfB], [IsdB, IsdA, ClfA, Hla209, SdrD], [IsdB, IsdA, ClfA, |
| | | ClfB, Hla209], [IsdB, IsdA, ClfA, ClfB, SdrD], [IsdB, IsdA, ClfA, SdrD, Hla209], [IsdB, IsdA, ClfA, |
| | | SdrD, ClfB], [IsdB, IsdA, ClfB, Hla209, ClfA], [IsdB, IsdA, ClfB, Hla209, SdrD], [IsdB, IsdA, |
| | | ClfB, ClfA, Hla209], [IsdB, IsdA, ClfB, ClfA, SdrD], [IsdB, IsdA, ClfB, SdrD, Hla209], [IsdB, IsdA, |
| | | ClfB, SdrD, ClfA], [IsdB, IsdA, SdrD, Hla209, ClfA], [IsdB, IsdA, SdrD, Hla209, ClfB], [IsdB, |
| | | IsdA, SdrD, ClfA, Hla209], [IsdB, IsdA, SdrD, ClfA, ClfB], [IsdB, IsdA, SdrD, ClfB, Hla209], |
| | | [IsdB, IsdA, SdrD, ClfB, ClfA] |
| 8 | all 4 SA antigens | Hla209(27-319), ClfA(221-559), ClfB (203-542), SdrD (246-682) |
| | from Group B(iii) | |
| | of: | |
| 9 | At least 3 “Group | [SA1739, SA1720, SA1890], [SA1739, SA1720, SA0103], [SA1739, SA1720, SA0377], |
| | A” SA antigens | [SA1739, SA1720, SA0693], [SA1739, SA1720, SA2105], [SA1739, SA1890, SA1720], |
| | selected from the | [SA1739, SA1890, SA0103], [SA1739, SA1890, SA0377], [SA1739, SA1890, SA0693], |
| | subgroups of: (210 | [SA1739, SA1890, SA2105], [SA1739, SA0103, SA1720], [SA1739, SA0103, SA1890], |
| | combinations) | [SA1739, SA0103, SA0377], [SA1739, SA0103, SA0693], [SA1739, SA0103, SA2105], |
| | | [SA1739, SA0377, SA1720], [SA1739, SA0377, SA1890], [SA1739, SA0377, SA0103], |
| | | [SA1739, SA0377, SA0693], [SA1739, SA0377, SA2105], [SA1739, SA0693, SA1720], |
| | | [SA1739, SA0693, SA1890], [SA1739, SA0693, SA0103], [SA1739, SA0693, SA0377], |
| | | [SA1739, SA0693, SA2105], [SA1739, SA2105, SA1720], [SA1739, SA2105, SA1890], |
| | | [SA1739, SA2105, SA0103], [SA1739, SA2105, SA0377], [SA1739, SA2105, SA0693], |
| | | [SA1720, SA1739, SA1890], [SA1720, SA1739, SA0103], [SA1720, SA1739, SA0377], |
| | | [SA1720, SA1739, SA0693], [SA1720, SA1739, SA2105], [SA1720, SA1890, SA1739], |
| | | [SA1720, SA1890, SA0103], [SA1720, SA1890, SA0377], [SA1720, SA1890, SA0693], |
| | | [SA1720, SA1890, SA2105], [SA1720, SA0103, SA1739], [SA1720, SA0103, SA1890], |
| | | [SA1720, SA0103, SA0377], [SA1720, SA0103, SA0693], [SA1720, SA0103, SA2105], |
| | | [SA1720, SA0377, SA1739], [SA1720, SA0377, SA1890], [SA1720, SA0377, SA0103], |
| | | [SA1720, SA0377, SA0693], [SA1720, SA0377, SA2105], [SA1720, SA0693, SA1739], |
| | | [SA1720, SA0693, SA1890], [SA1720, SA0693, SA0103], [SA1720, SA0693, SA0377], |
| | | [SA1720, SA0693, SA2105], [SA1720, SA2105, SA1739], [SA1720, SA2105, SA1890], |
| | | [SA1720, SA2105, SA0103], [SA1720, SA2105, SA0377], [SA1720, SA2105, SA0693], |
| | | [SA1890, SA1739, SA1720], [SA1890, SA1739, SA0103], [SA1890, SA1739, SA0377], |
| | | [SA1890, SA1739, SA0693], [SA1890, SA1739, SA2105], [SA1890, SA1720, SA1739], |
| | | [SA1890, SA1720, SA0103], [SA1890, SA1720, SA0377], [SA1890, SA1720, SA0693], |
| | | [SA1890, SA1720, SA2105], [SA1890, SA0103, SA1739], [SA1890, SA0103, SA1720], |
| | | [SA1890, SA0103, SA0377], [SA1890, SA0103, SA0693], [SA1890, SA0103, SA2105], |
| | | [SA1890, SA0377, SA1739], [SA1890, SA0377, SA1720], [SA1890, SA0377, SA0103], |
| | | [SA1890, SA0377, SA0693], [SA1890, SA0377, SA2105], [SA1890, SA0693, SA1739], |
| | | [SA1890, SA0693, SA1720], [SA1890, SA0693, SA0103], [SA1890, SA0693, SA0377], |
| | | [SA1890, SA0693, SA2105], [SA1890, SA2105, SA1739], [SA1890, SA2105, SA1720], |
| | | [SA1890, SA2105, SA0103], [SA1890, SA2105, SA0377], [SA1890, SA2105, SA0693], |
| | | [SA0103, SA1739, SA1720], [SA0103, SA1739, SA1890], [SA0103, SA1739, SA0377], |
| | | [SA0103, SA1739, SA0693], [SA0103, SA1739, SA2105], [SA0103, SA1720, SA1739], |
| | | [SA0103, SA1720, SA1890], [SA0103, SA1720, SA0377], [SA0103, SA1720, SA0693], |
| | | [SA0103, SA1720, SA2105], [SA0103, SA1890, SA1739], [SA0103, SA1890, SA1720], |
| | | [SA0103, SA1890, SA0377], [SA0103, SA1890, SA0693], [SA0103, SA1890, SA2105], |
| | | [SA0103, SA0377, SA1739], [SA0103, SA0377, SA1720], [SA0103, SA0377, SA1890], |
| | | [SA0103, SA0377, SA0693], [SA0103, SA0377, SA2105], [SA0103, SA0693, SA1739], |
| | | [SA0103, SA0693, SA1720], [SA0103, SA0693, SA1890], [SA0103, SA0693, SA0377], |
| | | [SA0103, SA0693, SA2105], [SA0103, SA2105, SA1739], [SA0103, SA2105, SA1720], |
| | | [SA0103, SA2105, SA1890], [SA0103, SA2105, SA0377], [SA0103, SA2105, SA0693], |
| | | [SA0377, SA1739, SA1720], [SA0377, SA1739, SA1890], [SA0377, SA1739, SA0103], |
| | | [SA0377, SA1739, SA0693], [SA0377, SA1739, SA2105], [SA0377, SA1720, SA1739], |
| | | [SA0377, SA1720, SA1890], [SA0377, SA1720, SA0103], [SA0377, SA1720, SA0693], |
| | | [SA0377, SA1720, SA2105], [SA0377, SA1890, SA1739], [SA0377, SA1890, SA1720], |
| | | [SA0377, SA1890, SA0103], [SA0377, SA1890, SA0693], [SA0377, SA1890, SA2105], |
| | | [SA0377, SA0103, SA1739], [SA0377, SA0103, SA1720], [SA0377, SA0103, SA1890], |
| | | [SA0377, SA0103, SA0693], [SA0377, SA0103, SA2105], [SA0377, SA0693, SA1739], |
| | | [SA0377, SA0693, SA1720], [SA0377, SA0693, SA1890], [SA0377, SA0693, SA0103], |
| | | [SA0377, SA0693, SA2105], [SA0377, SA2105, SA1739], [SA0377, SA2105, SA1720], |
| | | [SA0377, SA2105, SA1890], [SA0377, SA2105, SA0103], [SA0377, SA2105, SA0693], |
| | | [SA0693, SA1739, SA1720], [SA0693, SA1739, SA1890], [SA0693, SA1739, SA0103], |
| | | [SA0693, SA1739, SA0377], [SA0693, SA1739, SA2105], [SA0693, SA1720, SA1739], |
| | | [SA0693, SA1720, SA1890], [SA0693, SA1720, SA0103], [SA0693, SA1720, SA0377], |
| | | [SA0693, SA1720, SA2105], [SA0693, SA1890, SA1739], [SA0693, SA1890, SA1720], |
| | | [SA0693, SA1890, SA0103], [SA0693, SA1890, SA0377], [SA0693, SA1890, SA2105], |
| | | [SA0693, SA0103, SA1739], [SA0693, SA0103, SA1720], [SA0693, SA0103, SA1890], |
| | | [SA0693, SA0103, SA0377], [SA0693, SA0103, SA2105], [SA0693, SA0377, SA1739], |
| | | [SA0693, SA0377, SA1720], [SA0693, SA0377, SA1890], [SA0693, SA0377, SA0103], |
| | | [SA0693, SA0377, SA2105], [SA0693, SA2105, SA1739], [SA0693, SA2105, SA1720], |
| | | [SA0693, SA2105, SA1890], [SA0693, SA2105, SA0103], [SA0693, SA2105, SA0377], |
| | | [SA2105, SA1739, SA1720], [SA2105, SA1739, SA1890], [SA2105, SA1739, SA0103], |
| | | [SA2105, SA1739, SA0377], [SA2105, SA1739, SA0693], [SA2105, SA1720, SA1739], |
| | | [SA2105, SA1720, SA1890], [SA2105, SA1720, SA0103], [SA2105, SA1720, SA0377], |
| | | [SA2105, SA1720, SA0693], [SA2105, SA1890, SA1739], [SA2105, SA1890, SA1720], |
| | | [SA2105, SA1890, SA0103], [SA2105, SA1890, SA0377], [SA2105, SA1890, SA0693], |
| | | [SA2105, SA0103, SA1739], [SA2105, SA0103, SA1720], [SA2105, SA0103, SA1890], |
| | | [SA2105, SA0103, SA0377], [SA2105, SA0103, SA0693], [SA2105, SA0377, SA1739], |
| | | [SA2105, SA0377, SA1720], [SA2105, SA0377, SA1890], [SA2105, SA0377, SA0103], |
| | | [SA2105, SA0377, SA0693], [SA2105, SA0693, SA1739], [SA2105, SA0693, SA1720], |
| | | [SA2105, SA0693, SA1890], [SA2105, SA0693, SA0103], [SA2105, SA0693, SA0377] |
| 10 | At least 4 “Group | [SA1739, SA1720, SA1890, SA0103], [SA1739, SA1720, SA1890, SA0377], [SA1739, SA1720, SA1890, SA0693], |
| | A” SA antigens | [SA1739, SA1720, SA1890, SA2105], [SA1739, SA1720, SA0103, SA1890], [SA1739, SA1720, SA0103, SA0377], |
| | selected from the | [SA1739, SA1720, SA0103, SA0693], [SA1739, SA1720, SA0103, SA2105], [SA1739, SA1720, SA0377, SA1890], |
| | combinations) | [SA1739, SA1720, SA0377, SA0103], [SA1739, SA1720, SA0377, SA0693], [SA1739, SA1720, SA0377, SA2105], |
| | | [SA1739, SA1720, SA0693, SA1890], [SA1739, SA1720, SA0693, SA0103], [SA1739, SA1720, SA0693, SA0377], |
| | | [SA1739, SA1720, SA0693, SA2105], [SA1739, SA1720, SA2105, SA1890], [SA1739, SA1720, SA2105, SA0103], |
| | | [SA1739, SA1720, SA2105, SA0377], [SA1739, SA1720, SA2105, SA0693], [SA1739, SA1890, SA1720, SA0103], |
| | | [SA1739, SA1890, SA1720, SA0377], [SA1739, SA1890, SA1720, SA0693], [SA1739, SA1890, SA1720, SA2105], |
| | | [SA1739, SA1890, SA0103, SA1720], [SA1739, SA1890, SA0103, SA0377], [SA1739, SA1890, SA0103, SA0693], |
| | | [SA1739, SA1890, SA0103, SA2105], [SA1739, SA1890, SA0377, SA1720], [SA1739, SA1890, SA0377, SA0103], |
| | | [SA1739, SA1890, SA0377, SA0693], [SA1739, SA1890, SA0377, SA2105], [SA1739, SA1890, SA0693, SA1720], |
| | | [SA1739, SA1890, SA0693, SA0103], [SA1739, SA1890, SA0693, SA0377], [SA1739, SA1890, SA0693, SA2105], |
| | | [SA1739, SA1890, SA2105, SA1720], [SA1739, SA1890, SA2105, SA0103], [SA1739, SA1890, SA2105, SA0377], |
| | | [SA1739, SA1890, SA2105, SA0693], [SA1739, SA0103, SA1720, SA1890], [SA1739, SA0103, SA1720, SA0377], |
| | | [SA1739, SA0103, SA1720, SA0693], [SA1739, SA0103, SA1720, SA2105], [SA1739, SA0103, SA1890, SA1720], |
| | | [SA1739, SA0103, SA1890, SA0377], [SA1739, SA0103, SA1890, SA0693], [SA1739, SA0103, SA1890, SA2105], |
| | | [SA1739, SA0103, SA0377, SA1720], [SA1739, SA0103, SA0377, SA1890], [SA1739, SA0103, SA0377, SA0693], |
| | | [SA1739, SA0103, SA0377, SA2105], [SA1739, SA0103, SA0693, SA1720], [SA1739, SA0103, SA0693, SA1890], |
| | | [SA1739, SA0103, SA0693, SA0377], [SA1739, SA0103, SA0693, SA2105], [SA1739, SA0103, SA2105, SA1720], |
| | | [SA1739, SA0103, SA2105, SA1890], [SA1739, SA0103, SA2105, SA0377], [SA1739, SA0103, SA2105, SA0693], |
| | | [SA1739, SA0377, SA1720, SA1890], [SA1739, SA0377, SA1720, SA0103], [SA1739, SA0377, SA1720, SA0693], |
| | | [SA1739, SA0377, SA1720, SA2105], [SA1739, SA0377, SA1890, SA1720], [SA1739, SA0377, SA1890, SA0103], |
| | | [SA1739, SA0377, SA1890, SA0693], [SA1739, SA0377, SA1890, SA2105], [SA1739, SA0377, SA0103, SA1720], |
| | | [SA1739, SA0377, SA0103, SA1890], [SA1739, SA0377, SA0103, SA0693], [SA1739, SA0377, SA0103, SA2105], |
| | | [SA1739, SA0377, SA0693, SA1720], [SA1739, SA0377, SA0693, SA1890], [SA1739, SA0377, SA0693, SA0103], |
| | | [SA1739, SA0377, SA0693, SA2105], [SA1739, SA0377, SA2105, SA1720], [SA1739, SA0377, SA2105, SA1890], |
| | | [SA1739, SA0377, SA2105, SA0103], [SA1739, SA0377, SA2105, SA0693], [SA1739, SA0693, SA1720, SA1890], |
| | | [SA1739, SA0693, SA1720, SA0103], [SA1739, SA0693, SA1720, SA0377], [SA1739, SA0693, SA1720, SA2105], |
| | | [SA1739, SA0693, SA1890, SA1720], [SA1739, SA0693, SA1890, SA0103], [SA1739, SA0693, SA1890, SA0377], |
| | | [SA1739, SA0693, SA1890, SA2105], [SA1739, SA0693, SA0103, SA1720], [SA1739, SA0693, SA0103, SA1890], |
| | | [SA1739, SA0693, SA0103, SA0377], [SA1739, SA0693, SA0103, SA2105], [SA1739, SA0693, SA0377, SA1720], |
| | | [SA1739, SA0693, SA0377, SA1890], [SA1739, SA0693, SA0377, SA0103], [SA1739, SA0693, SA0377, SA2105], |
| | | [SA1739, SA0693, SA2105, SA1720], [SA1739, SA0693, SA2105, SA1890], [SA1739, SA0693, SA2105, SA0103], |
| | | [SA1739, SA0693, SA2105, SA0377], [SA1739, SA2105, SA1720, SA1890], [SA1739, SA2105, SA1720, SA0103], |
| | | [SA1739, SA2105, SA1720, SA0377], [SA1739, SA2105, SA1720, SA0693], [SA1739, SA2105, SA1890, SA1720], |
| | | [SA1739, SA2105, SA1890, SA0103], [SA1739, SA2105, SA1890, SA0377], [SA1739, SA2105, SA1890, SA0693], |
| | | [SA1739, SA2105, SA0103, SA1720], [SA1739, SA2105, SA0103, SA1890], [SA1739, SA2105, SA0103, SA0377], |
| | | [SA1739, SA2105, SA0103, SA0693], [SA1739, SA2105, SA0377, SA1720], [SA1739, SA2105, SA0377, SA1890], |
| | | [SA1739, SA2105, SA0377, SA0103], [SA1739, SA2105, SA0377, SA0693], [SA1739, SA2105, SA0693, SA1720], |
| | | [SA1739, SA2105, SA0693, SA1890], [SA1739, SA2105, SA0693, SA0103], [SA1739, SA2105, SA0693, SA0377], |
| | | [SA1720, SA1739, SA1890, SA0103], [SA1720, SA1739, SA1890, SA0377], [SA1720, SA1739, SA1890, SA0693], |
| | | [SA1720, SA1739, SA1890, SA2105], [SA1720, SA1739, SA0103, SA1890], [SA1720, SA1739, SA0103, SA0377], |
| | | [SA1720, SA1739, SA0103, SA0693], [SA1720, SA1739, SA0103, SA2105], [SA1720, SA1739, SA0377, SA1890], |
| | | [SA1720, SA1739, SA0377, SA0103], [SA1720, SA1739, SA0377, SA0693], [SA1720, SA1739, SA0377, SA2105], |
| | | [SA1720, SA1739, SA0693, SA1890], [SA1720, SA1739, SA0693, SA0103], [SA1720, SA1739, SA0693, SA0377], |
| | | [SA1720, SA1739, SA0693, SA2105], [SA1720, SA1739, SA2105, SA1890], [SA1720, SA1739, SA2105, SA0103], |
| | | [SA1720, SA1739, SA2105, SA0377], [SA1720, SA1739, SA2105, SA0693], [SA1720, SA1890, SA1739, SA0103], |
| | | [SA1720, SA1890, SA1739, SA0377], [SA1720, SA1890, SA1739, SA0693], [SA1720, SA1890, SA1739, SA2105], |
| | | [SA1720, SA1890, SA0103, SA1739], [SA1720, SA1890, SA0103, SA0377], [SA1720, SA1890, SA0103, SA0693], |
| | | [SA1720, SA1890, SA0103, SA2105], [SA1720, SA1890, SA0377, SA1739], [SA1720, SA1890, SA0377, SA0103], |
| | | [SA1720, SA1890, SA0377, SA0693], [SA1720, SA1890, SA0377, SA2105], [SA1720, SA1890, SA0693, SA1739], |
| | | [SA1720, SA1890, SA0693, SA0103], [SA1720, SA1890, SA0693, SA0377], [SA1720, SA1890, SA0693, SA2105], |
| | | [SA1720, SA1890, SA2105, SA1739], [SA1720, SA1890, SA2105, SA0103], [SA1720, SA1890, SA2105, SA0377], |
| | | [SA1720, SA1890, SA2105, SA0693], [SA1720, SA0103, SA1739, SA1890], [SA1720, SA0103, SA1739, SA0377], |
| | | [SA1720, SA0103, SA1739, SA0693], [SA1720, SA0103, SA1739, SA2105], [SA1720, SA0103, SA1890, SA1739], |
| | | [SA1720, SA0103, SA1890, SA0377], [SA1720, SA0103, SA1890, SA0693], [SA1720, SA0103, SA1890, SA2105], |
| | | [SA1720, SA0103, SA0377, SA1739], [SA1720, SA0103, SA0377, SA1890], [SA1720, SA0103, SA0377, SA0693], |
| | | [SA1720, SA0103, SA0377, SA2105], [SA1720, SA0103, SA0693, SA1739], [SA1720, SA0103, SA0693, SA1890], |
| | | [SA1720, SA0103, SA0693, SA0377], [SA1720, SA0103, SA0693, SA2105], [SA1720, SA0103, SA2105, SA1739], |
| | | [SA1720, SA0103, SA2105, SA1890], [SA1720, SA0103, SA2105, SA0377], [SA1720, SA0103, SA2105, SA0693], |
| | | [SA1720, SA0377, SA1739, SA1890], [SA1720, SA0377, SA1739, SA0103], [SA1720, SA0377, SA1739, SA0693], |
| | | [SA1720, SA0377, SA1739, SA2105], [SA1720, SA0377, SA1890, SA1739], [SA1720, SA0377, SA1890, SA0103], |
| | | [SA1720, SA0377, SA1890, SA0693], [SA1720, SA0377, SA1890, SA2105], [SA1720, SA0377, SA0103, SA1739], |
| | | [SA1720, SA0377, SA0103, SA1890], [SA1720, SA0377, SA0103, SA0693], [SA1720, SA0377, SA0103, SA2105], |
| | | [SA1720, SA0377, SA0693, SA1739], [SA1720, SA0377, SA0693, SA1890], [SA1720, SA0377, SA0693, SA0103], |
| | | [SA1720, SA0377, SA0693, SA2105], [SA1720, SA0377, SA2105, SA1739], [SA1720, SA0377, SA2105, SA1890], |
| | | [SA1720, SA0377, SA2105, SA0103], [SA1720, SA0377, SA2105, SA0693], [SA1720, SA0693, SA1739, SA1890], |
| | | [SA1720, SA0693, SA1739, SA0103], [SA1720, SA0693, SA1739, SA0377], [SA1720, SA0693, SA1739, SA2105], |
| | | [SA1720, SA0693, SA1890, SA1739], [SA1720, SA0693, SA1890, SA0103], [SA1720, SA0693, SA1890, SA0377], |
| | | [SA1720, SA0693, SA1890, SA2105], [SA1720, SA0693, SA0103, SA1739], [SA1720, SA0693, SA0103, SA1890], |
| | | [SA1720, SA0693, SA0103, SA0377], [SA1720, SA0693, SA0103, SA2105], [SA1720, SA0693, SA0377, SA1739], |
| | | [SA1720, SA0693, SA0377, SA1890], [SA1720, SA0693, SA0377, SA0103], [SA1720, SA0693, SA0377, SA2105], |
| | | [SA1720, SA0693, SA2105, SA1739], [SA1720, SA0693, SA2105, SA1890], [SA1720, SA0693, SA2105, SA0103], |
| | | [SA1720, SA0693, SA2105, SA0377], [SA1720, SA2105, SA1739, SA1890], [SA1720, SA2105, SA1739, SA0103], |
| | | [SA1720, SA2105, SA1739, SA0377], [SA1720, SA2105, SA1739, SA0693], [SA1720, SA2105, SA1890, SA1739], |
| | | [SA1720, SA2105, SA1890, SA0103], [SA1720, SA2105, SA1890, SA0377], [SA1720, SA2105, SA1890, SA0693], |
| | | [SA1720, SA2105, SA0103, SA1739], [SA1720, SA2105, SA0103, SA1890], [SA1720, SA2105, SA0103, SA0377], |
| | | [SA1720, SA2105, SA0103, SA0693], [SA1720, SA2105, SA0377, SA1739], [SA1720, SA2105, SA0377, SA1890], |
| | | [SA1720, SA2105, SA0377, SA0103], [SA1720, SA2105, SA0377, SA0693], [SA1720, SA2105, SA0693, SA1739], |
| | | [SA1720, SA2105, SA0693, SA1890], [SA1720, SA2105, SA0693, SA0103], [SA1720, SA2105, SA0693, SA0377], |
| | | [SA1890, SA1739, SA1720, SA0103], [SA1890, SA1739, SA1720, SA0377], [SA1890, SA1739, SA1720, SA0693], |
| | | [SA1890, SA1739, SA1720, SA2105], [SA1890, SA1739, SA0103, SA1720], [SA1890, SA1739, SA0103, SA0377], |
| | | [SA1890, SA1739, SA0103, SA6093], [SA1890, SA1739, SA0103, SA2105], [SA1890, SA1739, SA0377, SA1720], |
| | | [SA1890, SA1739, SA0377, SA0103], [SA1890, SA1739, SA0377, SA0693], [SA1890, SA1739, SA0377, SA2105], |
| | | [SA1890, SA1739, SA0693, SA1720], [SA1890, SA1739, SA0693, SA0103], [SA1890, SA1739, SA0693, SA0377], |
| | | [SA1890, SA1739, SA0693, SA2105], [SA1890, SA1739, SA2105, SA1720], [SA1890, SA1739, SA2105, SA0103], |
| | | [SA1890, SA1739, SA2105, SA0377], [SA1890, SA1739, SA2105, SA0693], [SA1890, SA1720, SA1739, SA0103], |
| | | [SA1890, SA1720, SA1739, SA0377], [SA1890, SA1720, SA1739, SA0693], [SA1890, SA1720, SA1739, SA2105], |
| | | [SA1890, SA1720, SA0103, SA1739], [SA1890, SA1720, SA0103, SA0377], [SA1890, SA1720, SA0103, SA0693], |
| | | [SA1890, SA1720, SA0103, SA2105], [SA1890, SA1720, SA0377, SA1739], [SA1890, SA1720, SA0377, SA0103], |
| | | [SA1890, SA1720, SA0377, SA0693], [SA1890, SA1720, SA0377, SA2105], [SA1890, SA1720, SA0693, SA1739], |
| | | [SA1890, SA1720, SA0693, SA0103], [SA1890, SA1720, SA0693, SA0377], [SA1890, SA1720, SA0693, SA2105], |
| | | [SA1890, SA1720, SA2105, SA1739], [SA1890, SA1720, SA2105, SA0103], [SA1890, SA1720, SA2105, SA0377], |
| | | [SA1890, SA1720, SA2105, SA0693], [SA1890, SA0103, SA1739, SA1720], [SA1890, SA0103, SA1739, SA0377], |
| | | [SA1890, SA0103, SA1739, SA0693], [SA1890, SA0103, SA1739, SA2105], [SA1890, SA0103, SA1720, SA1739], |
| | | [SA1890, SA0103, SA1720, SA0377], [SA1890, SA0103, SA1720, SA0693], [SA1890, SA0103, SA1720, SA2105], |
| | | [SA1890, SA0103, SA0377, SA1739], [SA1890, SA0103, SA0377, SA1720], [SA1890, SA0103, SA0377, SA0693], |
| | | [SA1890, SA0103, SA0377, SA2105], [SA1890, SA0103, SA0693, SA1739], [SA1890, SA0103, SA0693, SA1720], |
| | | [SA1890, SA0103, SA0693, SA0377], [SA1890, SA0103, SA0693, SA2105], [SA1890, SA0103, SA2105, SA1739], |
| | | [SA1890, SA0103, SA2105, SA1720], [SA1890, SA0103, SA2105, SA0377], [SA1890, SA0103, SA2105, SA0693], |
| | | [SA1890, SA0377, SA1739, SA1720], [SA1890, SA0377, SA1739, SA0103], [SA1890, SA0377, SA1739, SA0693], |
| | | [SA1890, SA0377, SA1739, SA2105], [SA1890, SA0377, SA1720, SA1739], [SA1890, SA0377, SA1720, SA0103], |
| | | [SA1890, SA0377, SA1720, SA0693], [SA1890, SA0377, SA1720, SA2105], [SA1890, SA0377, SA0103, SA1739], |
| | | [SA1890, SA0377, SA0103, SA1720], [SA1890, SA0377, SA0103, SA0693], [SA1890, SA0377, SA0103, SA2105], |
| | | [SA1890, SA0377, SA0693, SA1739], [SA1890, SA0377, SA0693, SA1720], [SA1890, SA0377, SA0693, SA0103], |
| | | [SA1890, SA0377, SA0693, SA2105], [SA1890, SA0377, SA2105, SA1739], [SA1890, SA0377, SA2105, SA1720], |
| | | [SA1890, SA0377, SA2105, SA0103], [SA1890, SA0377, SA2105, SA0693], [SA1890, SA0693, SA1739, SA1720], |
| | | [SA1890, SA0693, SA1739, SA0103], [SA1890, SA0693, SA1739, SA0377], [SA1890, SA0693, SA1739, SA2105], |
| | | [SA1890, SA0693, SA1720, SA1739], [SA1890, SA0693, SA1720, SA0103], [SA1890, SA0693, SA1720, SA0377], |
| | | [SA1890, SA0693, SA1720, SA2105], [SA1890, SA0693, SA0103, SA1739], [SA1890, SA0693, SA0103, SA1720], |
| | | [SA1890, SA0693, SA0103, SA0377], [SA1890, SA0693, SA0103, SA2105], [SA1890, SA0693, SA0377, SA1739], |
| | | [SA1890, SA0693, SA0377, SA1720], [SA1890, SA0693, SA0377, SA0103], [SA1890, SA0693, SA0377, SA2105], |
| | | [SA1890, SA0693, SA2105, SA1739], [SA1890, SA0693, SA2105, SA1720], [SA1890, SA0693, SA2105, SA0103], |
| | | [SA1890, SA0693, SA2105, SA0377], [SA1890, SA2105, SA1739, SA1720], [SA1890, SA2105, SA1739, SA0103], |
| | | [SA1890, SA2105, SA1739, SA0377], [SA1890, SA2105, SA1739, SA0693], [SA1890, SA2105, SA1720, SA1739], |
| | | [SA1890, SA2105, SA1720, SA0103], [SA1890, SA2105, SA1720, SA0377], [SA1890, SA2105, SA1720, SA0693], |
| | | [SA1890, SA2105, SA0103, SA1739], [SA1890, SA2105, SA0103, SA1720], [SA1890, SA2105, SA0103, SA0377], |
| | | [SA1890, SA2105, SA0103, SA0693], [SA1890, SA2105, SA0377, SA1739], [SA1890, SA2105, SA0377, SA1720], |
| | | [SA1890, SA2105, SA0377, SA0103], [SA1890, SA2105, SA0377, SA0693], [SA1890, SA2105, SA0693, SA1739], |
| | | [SA1890, SA2105, SA0693, SA1720], [SA1890, SA2105, SA0693, SA0103], [SA1890, SA2105, SA0693, SA0377], |
| | | [SA0103, SA1739, SA1720, SA1890], [SA0103, SA1739, SA1720, SA0377], [SA0103, SA1739, SA1720, SA0693], |
| | | [SA0103, SA1739, SA1720, SA2105], [SA0103, SA1739, SA1890, SA1720], [SA0103, SA1739, SA1890, SA0377], |
| | | [SA0103, SA1739, SA1890, SA0693], [SA0103, SA1739, SA1890, SA2105], [SA0103, SA1739, SA0377, SA1720], |
| | | [SA0103, SA1739, SA0377, SA1890], [SA0103, SA1739, SA0377, SA0693], [SA0103, SA1739, SA0377, SA2105], |
| | | [SA0103, SA1739, SA0693, SA1720], [SA0103, SA1739, SA0693, SA1890], [SA0103, SA1739, SA0693, SA0377], |
| | | [SA0103, SA1739, SA0693, SA2105], [SA0103, SA1739, SA2105, SA1720], [SA0103, SA1739, SA2105, SA1890], |
| | | [SA0103, SA1739, SA2105, SA0377], [SA0103, SA1739, SA2105, SA0693], [SA0103, SA1720, SA1739, SA1890], |
| | | [SA0103, SA1720, SA1739, SA0377], [SA0103, SA1720, SA1739, SA0693], [SA0103, SA1720, SA1739, SA2105], |
| | | [SA0103, SA1720, SA1890, SA1739], [SA0103, SA1720, SA1890, SA0377], [SA0103, SA1720, SA1890, SA0693], |
| | | [SA0103, SA1720, SA1890, SA2105], [SA0103, SA1720, SA0377, SA1739], [SA0103, SA1720, SA0377, SA1890], |
| | | [SA0103, SA1720, SA0377, SA0693], [SA0103, SA1720, SA0377, SA2105], [SA0103, SA1720, SA0693, SA1739], |
| | | [SA0103, SA1720, SA0693, SA1890], [SA0103, SA1720, SA0693, SA0377], [SA0103, SA1720, SA0693, SA2105], |
| | | [SA0103, SA1720, SA2105, SA1739], [SA0103, SA1720, SA2105, SA1890], [SA0103, SA1720, SA2105, SA0377], |
| | | [SA0103, SA1720, SA2105, SA0693], [SA0103, SA1890, SA1739, SA1720], [SA0103, SA1890, SA1739, SA0377], |
| | | [SA0103, SA1890, SA1739, SA0693], [SA0103, SA1890, SA1739, SA2105], [SA0103, SA1890, SA1720, SA1739], |
| | | [SA0103, SA1890, SA1720, SA0377], [SA0103, SA1890, SA1720, SA0693], [SA0103, SA1890, SA1720, SA2105], |
| | | [SA0103, SA1890, SA0377, SA1739], [SA0103, SA1890, SA0377, SA1720], [SA0103, SA1890, SA0377, SA0693], |
| | | [SA0103, SA1890, SA0377, SA2105], [SA0103, SA1890, SA0693, SA1739], [SA0103, SA1890, SA0693, SA1720], |
| | | [SA0103, SA1890, SA0693, SA0377], [SA0103, SA1890, SA0693, SA2105], [SA0103, SA1890, SA2105, SA1739], |
| | | [SA0103, SA1890, SA2105, SA1720], [SA0103, SA1890, SA2105, SA0377], [SA0103, SA1890, SA2105, SA0693], |
| | | [SA0103, SA0377, SA1739, SA1720], [SA0103, SA0377, SA1739, SA1890], [SA0103, SA0377, SA1739, SA0693], |
| | | [SA0103, SA0377, SA1739, SA2105], [SA0103, SA0377, SA1720, SA1739], [SA0103, SA0377, SA1720, SA1890], |
| | | [SA0103, SA0377, SA1720, SA0693], [SA0103, SA0377, SA1720, SA2105], [SA0103, SA0377, SA1890, SA1739], |
| | | [SA0103, SA0377, SA1890, SA1720], [SA0103, SA0377, SA1890, SA0693], [SA0103, SA0377, SA1890, SA2105], |
| | | [SA0103, SA0377, SA0693, SA1739], [SA0103, SA0377, SA0693, SA1720], [SA0103, SA0377, SA0693, SA1890], |
| | | [SA0103, SA0377, SA0693, SA2105], [SA0103, SA0377, SA2105, SA1739], [SA0103, SA0377, SA2105, SA1720], |
| | | [SA0103, SA0377, SA2105, SA1890], [SA0103, SA0377, SA2105, SA0693], [SA0103, SA0693, SA1739, SA1720], |
| | | [SA0103, SA0693, SA1739, SA1890], [SA0103, SA0693, SA1739, SA0377], [SA0103, SA0693, SA1739, SA2105], |
| | | [SA0103, SA0693, SA1720, SA1739], [SA0103, SA0693, SA1720, SA1890], [SA0103, SA0693, SA1720, SA0377], |
| | | [SA0103, SA0693, SA1720, SA2105], [SA0103, SA0693, SA1890, SA1739], [SA0103, SA0693, SA1890, SA1720], |
| | | [SA0103, SA0693, SA1890, SA0377], [SA0103, SA0693, SA1890, SA2105], [SA0103, SA0693, SA0377, SA1739], |
| | | [SA0103, SA0693, SA0377, SA1720], [SA0103, SA0693, SA0377, SA1890], [SA0103, SA0693, SA0377, SA2105], |
| | | [SA0103, SA0693, SA2105, SA1739], [SA0103, SA0693, SA2105, SA1720], [SA0103, SA0693, SA2105, SA1890], |
| | | [SA0103, SA0693, SA2105, SA0377], [SA0103, SA2105, SA1739, SA1720], [SA0103, SA2105, SA1739, SA1890], |
| | | [SA0103, SA2105, SA1739, SA0377], [SA0103, SA2105, SA1739, SA0693], [SA0103, SA2105, SA1720, SA1739], |
| | | [SA0103, SA2105, SA1720, SA1890], [SA0103, SA2105, SA1720, SA0377], [SA0103, SA2105, SA1720, SA0693], |
| | | [SA0103, SA2105, SA1890, SA1739], [SA0103, SA2105, SA1890, SA1720], [SA0103, SA2105, SA1890, SA0377], |
| | | [SA0103, SA2105, SA1890, SA0693], [SA0103, SA2105, SA0377, SA1739], [SA0103, SA2105, SA0377, SA1720], |
| | | [SA0103, SA2105, SA0377, SA1890], [SA0103, SA2105, SA0377, SA0693], [SA0103, SA2105, SA0693, SA1739], |
| | | [SA0103, SA2105, SA0693, SA1720], [SA0103, SA2105, SA0693, SA1890], [SA0103, SA2105, SA0693, SA0377], |
| | | [SA0377, SA1739, SA1720, SA1890], [SA0377, SA1739, SA1720, SA0103], [SA0377, SA1739, SA1720, SA0693], |
| | | [SA0377, SA1739, SA1720, SA2105], [SA0377, SA1739, SA1890, SA1720], [SA0377, SA1739, SA1890, SA0103], |
| | | [SA0377, SA1739, SA1890, SA0693], [SA0377, SA1739, SA1890, SA2105], [SA0377, SA1739, SA0103, SA1720], |
| | | [SA0377, SA1739, SA0103, SA1890], [SA0377, SA1739, SA0103, SA0693], [SA0377, SA1739, SA0103, SA2105], |
| | | [SA0377, SA1739, SA0693, SA1720], [SA0377, SA1739, SA0693, SA1890], [SA0377, SA1739, SA0693, SA0103], |
| | | [SA0377, SA1739, SA0693, SA2105], [SA0377, SA1739, SA2105, SA1720], [SA0377, SA1739, SA2105, SA1890], |
| | | [SA0377, SA1739, SA2105, SA0103], [SA0377, SA1739, SA2105, SA0693], [SA0377, SA1720, SA1739, SA1890], |
| | | [SA0377, SA1720, SA1739, SA0103], [SA0377, SA1720, SA1739, SA0693], [SA0377, SA1720, SA1739, SA2105], |
| | | [SA0377, SA1720, SA1890, SA1739], [SA0377, SA1720, SA1890, SA0103], [SA0377, SA1720, SA1890, SA0693], |
| | | [SA0377, SA1720, SA1890, SA2105], [SA0377, SA1720, SA0103, SA1739], [SA0377, SA1720, SA0103, SA1890], |
| | | [SA0377, SA1720, SA0103, SA0693], [SA0377, SA1720, SA0103, SA2105], [SA0377, SA1720, SA0693, SA1739], |
| | | [SA0377, SA1720, SA0693, SA1890], [SA0377, SA1720, SA0693, SA0103], [SA0377, SA1720, SA0693, SA2105], |
| | | [SA0377, SA1720, SA2105, SA1739], [SA0377, SA1720, SA2105, SA1890], [SA0377, SA1720, SA2105, SA0103], |
| | | [SA0377, SA1720, SA2105, SA0693], [SA0377, SA1890, SA1739, SA1720], [SA0377, SA1890, SA1739, SA0103], |
| | | [SA0377, SA1890, SA1739, SA0693], [SA0377, SA1890, SA1739, SA2105], [SA0377, SA1890, SA1720, SA1739], |
| | | [SA0377, SA1890, SA1720, SA0103], [SA0377, SA1890, SA1720, SA0693], [SA0377, SA1890, SA1720, SA2105], |
| | | [SA0377, SA1890, SA0103, SA1739], [SA0377, SA1890, SA0103, SA1720], [SA0377, SA1890, SA0103, SA0693], |
| | | [SA0377, SA1890, SA0103, SA2105], [SA0377, SA1890, SA0693, SA1739], [SA0377, SA1890, SA0693, SA1720], |
| | | [SA0377, SA1890, SA0693, SA0103], [SA0377, SA1890, SA0693, SA2105], [SA0377, SA1890, SA2105, SA1739], |
| | | [SA0377, SA1890, SA2105, SA1720], [SA0377, SA1890, SA2105, SA0103], [SA0377, SA1890, SA2105, SA0693], |
| | | [SA0377, SA0103, SA1739, SA1720], [SA0377, SA0103, SA1739, SA1890], [SA0377, SA0103, SA1739, SA0693], |
| | | [SA0377, SA0103, SA1739, SA2105], [SA0377, SA0103, SA1720, SA1739], [SA0377, SA0103, SA1720, SA1890], |
| | | [SA0377, SA0103, SA1720, SA0693], [SA0377, SA0103, SA1720, SA2105], [SA0377, SA0103, SA1890, SA1739], |
| | | [SA0377, SA0103, SA1890, SA1720], [SA0377, SA0103, SA1890, SA0693], [SA0377, SA0103, SA1890, SA2105], |
| | | [SA0377, SA0103, SA0693, SA1739], [SA0377, SA0103, SA0693, SA1720], [SA0377, SA0103, SA0693, SA1890], |
| | | [SA0377, SA0103, SA0693, SA2105], [SA0377, SA0103, SA2105, SA1739], [SA0377, SA0103, SA2105, SA1720], |
| | | [SA0377, SA0103, SA2105, SA1890], [SA0377, SA0103, SA2105, SA0693], [SA0377, SA0693, SA1739, SA1720], |
| | | [SA0377, SA0693, SA1739, SA1890], [SA0377, SA0693, SA1739, SA0103], [SA0377, SA0693, SA1739, SA2105], |
| | | [SA0377, SA0693, SA1720, SA1739], [SA0377, SA0693, SA1720, SA1890], [SA0377, SA0693, SA1720, SA0103], |
| | | [SA0377, SA0693, SA1720, SA2105], [SA0377, SA0693, SA1890, SA1739], [SA0377, SA0693, SA1890, SA1720], |
| | | [SA0377, SA0693, SA1890, SA0103], [SA0377, SA0693, SA1890, SA2105], [SA0377, SA0693, SA0103, SA1739], |
| | | [SA0377, SA0693, SA0103, SA1720], [SA0377, SA0693, SA0103, SA1890], [SA0377, SA0693, SA0103, SA2105], |
| | | [SA0377, SA0693, SA2105, SA1739], [SA0377, SA0693, SA2105, SA1720], [SA0377, SA0693, SA2105, SA1890], |
| | | [SA0377, SA0693, SA2105, SA0103], [SA0377, SA2105, SA1739, SA1720], [SA0377, SA2105, SA1739, SA1890], |
| | | [SA0377, SA2105, SA1739, SA0103], [SA0377, SA2105, SA1739, SA0693], [SA0377, SA2105, SA1720, SA1739], |
| | | [SA0377, SA2105, SA1720, SA1890], [SA0377, SA2105, SA1720, SA0103], [SA0377, SA2105, SA1720, SA0693], |
| | | [SA0377, SA2105, SA1890, SA1739], [SA0377, SA2105, SA1890, SA1720], [SA0377, SA2105, SA1890, SA0103], |
| | | [SA0377, SA2105, SA1890, SA0693], [SA0377, SA2105, SA0103, SA1739], [SA0377, SA2105, SA0103, SA1720], |
| | | [SA0377, SA2105, SA0103, SA1890], [SA0377, SA2105, SA0103, SA0693], [SA0377, SA2105, SA0693, SA1739], |
| | | [SA0377, SA2105, SA0693, SA1720], [SA0377, SA2105, SA0693, SA1890], [SA0377, SA2105, SA0693, SA0103], |
| | | [SA0693, SA1739, SA1720, SA1890], [SA0693, SA1739, SA1720, SA0103], [SA0693, SA1739, SA1720, SA0377], |
| | | [SA0693, SA1739, SA1720, SA2105], [SA0693, SA1739, SA1890, SA1720], [SA0693, SA1739, SA1890, SA0103], |
| | | [SA0693, SA1739, SA1890, SA0377], [SA0693, SA1739, SA1890, SA2105], [SA0693, SA1739, SA0103, SA1720], |
| | | [SA0693, SA1739, SA0103, SA1890], [SA0693, SA1739, SA0103, SA0377], [SA0693, SA1739, SA0103, SA2105], |
| | | [SA0693, SA1739, SA0377, SA1720], [SA0693, SA1739, SA0377, SA1890], [SA0693, SA1739, SA0377, SA0103], |
| | | [SA0693, SA1739, SA0377, SA2105], [SA0693, SA1739, SA2105, SA1720], [SA0693, SA1739, SA2105, SA1890], |
| | | [SA0693, SA1739, SA2105, SA0103], [SA0693, SA1739, SA2105, SA0377], [SA0693, SA1720, SA1739, SA1890], |
| | | [SA0693, SA1720, SA1739, SA0103], [SA0693, SA1720, SA1739, SA0377], [SA0693, SA1720, SA1739, SA2105], |
| | | [SA0693, SA1720, SA1890, SA1739], [SA0693, SA1720, SA1890, SA0103], [SA0693, SA1720, SA1890, SA0377], |
| | | [SA0693, SA1720, SA1890, SA2105], [SA0693, SA1720, SA0103, SA1739], [SA0693, SA1720, SA0103, SA1890], |
| | | [SA0693, SA1720, SA0103, SA0377], [SA0693, SA1720, SA0103, SA2105], [SA0693, SA1720, SA0377, SA1739], |
| | | [SA0693, SA1720, SA0377, SA1890], [SA0693, SA1720, SA0377, SA0103], [SA0693, SA1720, SA0377, SA2105], |
| | | [SA0693, SA1720, SA2105, SA1739], [SA0693, SA1720, SA2105, SA1890], [SA0693, SA1720, SA2105, SA0103], |
| | | [SA0693, SA1720, SA2105, SA0377], [SA0693, SA1890, SA1739, SA1720], [SA0693, SA1890, SA1739, SA0103], |
| | | [SA0693, SA1890, SA1739, SA0377], [SA0693, SA1890, SA1739, SA2105], [SA0693, SA1890, SA1720, SA1739], |
| | | [SA0693, SA1890, SA1720, SA0103], [SA0693, SA1890, SA1720, SA0377], [SA0693, SA1890, SA1720, SA2105], |
| | | [SA0693, SA1890, SA0103, SA1739], [SA0693, SA1890, SA0103, SA1720], [SA0693, SA1890, SA0103, SA0377], |
| | | [SA0693, SA1890, SA0103, SA2105], [SA0693, SA1890, SA0377, SA1739], [SA0693, SA1890, SA0377, SA1720], |
| | | [SA0693, SA1890, SA0377, SA0103], [SA0693, SA1890, SA0377, SA2105], [SA0693, SA1890, SA2105, SA1739], |
| | | [SA0693, SA1890, SA2105, SA1720], [SA0693, SA1890, SA2105, SA0103], [SA0693, SA1890, SA2105, SA0377], |
| | | [SA0693, SA0103, SA1739, SA1720], [SA0693, SA0103, SA1739, SA1890], [SA0693, SA0103, SA1739, SA0377], |
| | | [SA0693, SA0103, SA1739, SA2105], [SA0693, SA0103, SA1720, SA1739], [SA0693, SA0103, SA1720, SA1890], |
| | | [SA0693, SA0103, SA1720, SA0377], [SA0693, SA0103, SA1720, SA2105], [SA0693, SA0103, SA1890, SA1739], |
| | | [SA0693, SA0103, SA1890, SA1720], [SA0693, SA0103, SA1890, SA0377], [SA0693, SA0103, SA1890, SA2105], |
| | | [SA0693, SA0103, SA0377, SA1739], [SA0693, SA0103, SA0377, SA1720], [SA0693, SA0103, SA0377, SA1890], |
| | | [SA0693, SA0103, SA0377, SA2105], [SA0693, SA0103, SA2105, SA1739], [SA0693, SA0103, SA2105, SA1720], |
| | | [SA0693, SA0103, SA2105, SA1890], [SA0693, SA0103, SA2105, SA0377], [SA0693, SA0377, SA1739, SA1720], |
| | | [SA0693, SA0377, SA1739, SA1890], [SA0693, SA0377, SA1739, SA0103], [SA0693, SA0377, SA1739, SA2105], |
| | | [SA0693, SA0377, SA1720, SA1739], [SA0693, SA0377, SA1720, SA1890], [SA0693, SA0377, SA1720, SA0103], |
| | | [SA0693, SA0377, SA1720, SA2105], [SA0693, SA0377, SA1890, SA1739], [SA0693, SA0377, SA1890, SA1720], |
| | | [SA0693, SA0377, SA1890, SA0103], [SA0693, SA0377, SA1890, SA2105], [SA0693, SA0377, SA0103, SA1739], |
| | | [SA0693, SA0377, SA0103, SA1720], [SA0693, SA0377, SA0103, SA1890], [SA0693, SA0377, SA0103, SA2105], |
| | | [SA0693, SA0377, SA2105, SA1739], [SA0693, SA0377, SA2105, SA1720], [SA0693, SA0377, SA2105, SA1890], |
| | | [SA0693, SA0377, SA2105, SA0103], [SA0693, SA2105, SA1739, SA1720], [SA0693, SA2105, SA1739, SA1890], |
| | | [SA0693, SA2105, SA1739, SA0103], [SA0693, SA2105, SA1739, SA0377], [SA0693, SA2105, SA1720, SA1739], |
| | | [SA0693, SA2105, SA1720, SA1890], [SA0693, SA2105, SA1720, SA0103], [SA0693, SA2105, SA1720, SA0377], |
| | | [SA0693, SA2105, SA1890, SA1739], [SA0693, SA2105, SA1890, SA1720], [SA0693, SA2105, SA1890, SA0103], |
| | | [SA0693, SA2105, SA1890, SA0377], [SA0693, SA2105, SA0103, SA1739], [SA0693, SA2105, SA0103, SA1720], |
| | | [SA0693, SA2105, SA0103, SA1890], [SA0693, SA2105, SA0103, SA0377], [SA0693, SA2105, SA0377, SA1739], |
| | | [SA0693, SA2105, SA0377, SA1720], [SA0693, SA2105, SA0377, SA1890], [SA0693, SA2105, SA0377, SA0103], |
| | | [SA2105, SA1739, SA1720, SA1890], [SA2105, SA1739, SA1720, SA0103], [SA2105, SA1739, SA1720, SA0377], |
| | | [SA2105, SA1739, SA1720, SA0693], [SA2105, SA1739, SA1890, SA1720], [SA2105, SA1739, SA1890, SA0103], |
| | | [SA2105, SA1739, SA1890, SA0377], [SA2105, SA1739, SA1890, SA0693], [SA2105, SA1739, SA0103, SA1720], |
| | | [SA2105, SA1739, SA0103, SA1890], [SA2105, SA1739, SA0103, SA0377], [SA2105, SA1739, SA0103, SA0693], |
| | | [SA2105, SA1739, SA0377, SA1720], [SA2105, SA1739, SA0377, SA1890], [SA2105, SA1739, SA0377, SA0103], |
| | | [SA2105, SA1739, SA0377, SA0693], [SA2105, SA1739, SA0693, SA1720], [SA2105, SA1739, SA0693, SA1890], |
| | | [SA2105, SA1739, SA0693, SA0103], [SA2105, SA1739, SA0693, SA0377], [SA2105, SA1720, SA1739, SA1890], |
| | | [SA2105, SA1720, SA1739, SA0103], [SA2105, SA1720, SA1739, SA0377], [SA2105, SA1720, SA1739, SA0693], |
| | | [SA2105, SA1720, SA1890, SA1739], [SA2105, SA1720, SA1890, SA0103], [SA2105, SA1720, SA1890, SA0377], |
| | | [SA2105, SA1720, SA1890, SA0693], [SA2105, SA1720, SA0103, SA1739], [SA2105, SA1720, SA0103, SA1890], |
| | | [SA2105, SA1720, SA0103, SA0377], [SA2105, SA1720, SA0103, SA0693], [SA2105, SA1720, SA0377, SA1739], |
| | | [SA2105, SA1720, SA0377, SA1890], [SA2105, SA1720, SA0377, SA0103], [SA2105, SA1720, SA0377, SA0693], |
| | | [SA2105, SA1720, SA0693, SA1739], [SA2105, SA1720, SA0693, SA1890], [SA2105, SA1720, SA0693, SA0103], |
| | | [SA2105, SA1720, SA0693, SA0377], [SA2105, SA1890, SA1739, SA1720], [SA2105, SA1890, SA1739, SA0103], |
| | | [SA2105, SA1890, SA1739, SA0377], [SA2105, SA1890, SA1739, SA0693], [SA2105, SA1890, SA1720, SA1739], |
| | | [SA2105, SA1890, SA1720, SA0103], [SA2105, SA1890, SA1720, SA0377], [SA2105, SA1890, SA1720, SA0693], |
| | | [SA2105, SA1890, SA0103, SA1739], [SA2105, SA1890, SA0103, SA1720], [SA2105, SA1890, SA0103, SA0377], |
| | | [SA2105, SA1890, SA0103, SA0693], [SA2105, SA1890, SA0377, SA1739], [SA2105, SA1890, SA0377, SA1720], |
| | | [SA2105, SA1890, SA0377, SA0103], [SA2105, SA1890, SA0377, SA0693], [SA2105, SA1890, SA0693, SA1739], |
| | | [SA2105, SA1890, SA0693, SA1720], [SA2105, SA1890, SA0693, SA0103], [SA2105, SA1890, SA0693, SA0377], |
| | | [SA2105, SA0103, SA1739, SA1720], [SA2105, SA0103, SA1739, SA1890], [SA2105, SA0103, SA1739, SA0377], |
| | | [SA2105, SA0103, SA1739, SA0693], [SA2105, SA0103, SA1720, SA1739], [SA2105, SA0103, SA1720, SA1890], |
| | | [SA2105, SA0103, SA1720, SA0377], [SA2105, SA0103, SA1720, SA0693], [SA2105, SA0103, SA1890, SA1739], |
| | | [SA2105, SA0103, SA1890, SA1720], [SA2105, SA0103, SA1890, SA0377], [SA2105, SA0103, SA1890, SA0693], |
| | | [SA2105, SA0103, SA0377, SA1739], [SA2105, SA0103, SA0377, SA1720], [SA2105, SA0103, SA0377, SA1890], |
| | | [SA2105, SA0103, SA0377, SA0693], [SA2105, SA0103, SA0693, SA1739], [SA2105, SA0103, SA0693, SA1720], |
| | | [SA2105, SA0103, SA0693, SA1890], [SA2105, SA0103, SA0693, SA0377], [SA2105, SA0377, SA1739, SA1720], |
| | | [SA2105, SA0377, SA1739, SA1890], [SA2105, SA0377, SA1739, SA0103], [SA2105, SA0377, SA1739, SA0693], |
| | | [SA2105, SA0377, SA1720, SA1739], [SA2105, SA0377, SA1720, SA1890], [SA2105, SA0377, SA1720, SA0103], |
| | | [SA2105, SA0377, SA1720, SA0693], [SA2105, SA0377, SA1890, SA1739], [SA2105, SA0377, SA1890, SA1720], |
| | | [SA2105, SA0377, SA1890, SA0103], [SA2105, SA0377, SA1890, SA0693], [SA2105, SA0377, SA0103, SA1739], |
| | | [SA2105, SA0377, SA0103, SA1720], [SA2105, SA0377, SA0103, SA1890], [SA2105, SA0377, SA0103, SA0693], |
| | | [SA2105, SA0377, SA0693, SA1739], [SA2105, SA0377, SA0693, SA1720], [SA2105, SA0377, SA0693, SA1890], |
| | | [SA2105, SA0377, SA0693, SA0103], [SA2105, SA0693, SA1739, SA1720], [SA2105, SA0693, SA1739, SA1890], |
| | | [SA2105, SA0693, SA1739, SA0103], [SA2105, SA0693, SA1739, SA0377], [SA2105, SA0693, SA1720, SA1739], |
| | | [SA2105, SA0693, SA1720, SA1890], [SA2105, SA0693, SA1720, SA0103], [SA2105, SA0693, SA1720, SA0377], |
| | | [SA2105, SA0693, SA1890, SA1739], [SA2105, SA0693, SA1890, SA1720], [SA2105, SA0693, SA1890, SA0103], |
| | | [SA2105, SA0693, SA1890, SA0377], [SA2105, SA0693, SA0103, SA1739], [SA2105, SA0693, SA0103, SA1720], |
| | | [SA2105, SA0693, SA0103, SA1890], [SA2105, SA0693, SA0103, SA0377], [SA2105, SA0693, SA0377, SA1739], |
| | | [SA2105, SA0693, SA0377, SA1720], [SA2105, SA0693, SA0377, SA1890], [SA2105, SA0693, SA0377, SA0103] |

### EQUIVALENTS AND SCOPE

Those skilled in the art will recognize, or be able to ascertain using no more than routine experimentation, many equivalents of the embodiments described herein. The scope of the present disclosure is not intended to be limited to the above description, but rather is as set forth in the appended claims.

Articles such as “a,” “an,” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which more than one members of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the disclosure encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any element or subgroup of elements can be removed from the group. It is also noted that the term “comprising” is intended to be open and permits the inclusion of additional elements or steps. It should be understood that, in general, where an embodiment, product, or method is referred to as comprising particular elements, features, or steps, embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

Where websites are provided, URL addresses are provided as non-browser-executable codes, with periods of the respective web address in parentheses. The actual web addresses do not contain the parentheses.

In addition, it is to be understood that any particular embodiment of the present disclosure may be explicitly excluded from any one or more of the claims. Where ranges are given, any value within the range may explicitly be excluded from any one or more of the claims. Any embodiment, element, feature, application, or aspect of the compositions and/or methods of the disclosure, can be excluded from any one or more claims. For purposes of brevity, all of the embodiments in which one or more elements, features, purposes, or aspects is excluded are not set forth explicitly herein.

SEQUENCES:

| S. | | |
|---|---|---|
| aureus | | |
| antigen | | |
| name | Protein SEQ ID NO: | DNA SEQ ID NO: |
| SA1720 | SEQ ID NO: 50 | SEQ ID NO: 57 |
| (B1) | ISDQIFFKHVKSDIKIEKLNVTLNDAAKKQIN | ATATCCGATCAGATATTTTTCAAACATGTTAAATCCGACATTAA |
| | NYTSQQVSNKKNDAWRDASATEIKSAMDSGTF | GATTGAAAAGTTAAATGTTACATTAAACGATGCAGCAAAGAAAC |
| | IDNEKQKYQFLDLSKYQGIDKNRIKRMLVDRP | AAATAAATAATTATACGAGTCAACAGGTATCAAATAAAAAGAAT |
| | TLLKHTDDFLKAAKDKHVNEVYLISHALLETG | GATGCATGGAGAGATGCATCTGCAACTGAAATTAAAAGTGCAAT |
| | AVKSELANGVEIDGKKYYNFYGVGALDKDPIK | GGATAGCGGTACTTTTATCGATAATGAAAAGCAAAAATATCAAT |
| | TGAEYAKKHGWDTPEKAISGGADFIHKHFLSS | TTTTAGATTTATCAAAGTATCAAGGGATTGATAAAAATAGAATT |
| | TDQNTLYSMRWNPKNPGEHQYATDIKWAESNA | AAACGTATGTTAGTAGATAGACCAACGTTATTGAAACATACGGA |
| | TIIADFYKNMKTEGKYFKYFVYKDDSKHLNK | TGATTTCTTAAAAGCTGCTAAAGATAAGCACGTTAACGAAGTTT |
| | | ATTTAATTTCACATGCATTATTAGAAACTGGCGCAGTTAAAAGT |
| | | GAATTAGCTAATGGAGTCGAAATTGATGGCAAAAAGTACTACAA |
| | | TTTCTATGGAGTAGGAGCCCTTGATAAAGACCCAATTAAAACAG |
| | | GTGCAGAATATGCTAAAAAGCATGGTTGGGATACACCTGAAAAA |
| | | GCTATTTCAGGCGGTGCTGATTTCATTCATAAGCACTTCTTATC |
| | | AAGCACAGATCAAAATACATTGTATAGTATGAGATGGAATCCAA |
| | | AAAATCCAGGAGAACATCAATATGCTACAGATATTAAGTGGGCA |
| | | GAAAGTAATGCAACAATTATCGCTGACTTTTATAAGAACATGAA |
| | | GACTGAAGGAAAATACTTCAAATACTTTGTGTATAAAGATGACA |
| | | GTAAACATTTGAATAAGTAA |
| SA1739 | SEQ ID NO: 51 | SEQ ID NO: 58 |
| (B2) | NNDNSTNNQSSKTQLTQKDENKNEDKPEEKSE | AATAATGACAATTCCACTAATAATCAATCATCAAAAACGCAATT |
| | TATDEDLQSTEEVPANENTENNQHEIDEITTK | AACTCAAAAAGATGAAAATAAAAATGAAGATAAGCCTGAGGAAA |
| | DQSDDDINTPNVAEDKSQDDLKDDLKEKQQSS | AATCAGAAACAGCAACAGATGAGGATTTACAATCAACCGAAGAA |
| | NHHQSTQPKTSPSTETNTQQSFANCKQLRQVY | GTACCTGCAAATGAAAATACTGAAAATAATCAACATGAAATTGA |
| | PNGVTADHPAYRPHLDRDKDKRACEPDKY | TGAAATAACAACAAAAGATCAATCAGACGATGATATTAACACAC |
| | | CAAACGTTGCAGAAGATAAATCACAAGACGACTTGAAAGATGAT |
| | | TTAAAAGAAAAGCAACAATCAAGTAACCATCATCAATCCACGCA |
| | | ACCTAAGACCTCACCATCAACTGAAACAAACACGCAACAATCAT |
| | | TTGCTAATTGTAAGCAACTTAGACAAGTATATCCGAATGGTGTC |
| | | ACTGCCGATCATCCAGCATATCGACCACATTTAGATAGAGATAA |
| | | AGATAAACGTGCATGTGAACCTGATAAATATTAA |
| SA1890 | SEQ ID NO: 52 | SEQ ID NO: 59 |
| (B3) | ESNSNIKAKDKKHVQVNVEDKSVPTDVRNLAQ | GAGAGCAATTCAAATATCAAAGCAAAAGATAAGAAGCATGTGCA |
| | KDYLSYVTSLDKIYNKEKASYTLGEPFKIYKF | AGTTAATGTCGAGGACAAGAGTGTTCCTACTGACGTACGTAATT |
| | NKKSDGNYYFPVLNTEGNIDYIVTISPKITKY | TAGCGCAAAAAGACTATCTTTCTTATGTTACTTCACTTGATAAA |
| | SSSSSKYTINVSPFLSKVLNQYKDQQITILTN | ATCTATAATAAAGAGAAAGCCTCGTATACACTTGGTGAACCTTT |
| | SKGYYVVTQNHKAKLVLKTPRLEDKKLKKTES | TAAAATTTATAAATTCAACAAAAAGAGTGATGGTAATTATTATT |
| | IPTGNNVTQLKQKASVTMPTSQFKSNNYTYNE | TTCCAGTTTTGAACACTGAAGGTAATATCGATTATATAGTTACA |
| | QYINKLENFKIRETQGNNGWCAGYTMSELLNA | ATTTCACCAAAAATCACTAAGTATTCTAGTTCATCTTCTAAATA |
| | TYNTNKYHAEAVMRFLHPNLQGQRFQFTGLTP | CACAATCAATGTTTCTCCATTTTTATCTAAAGTCTTAAATCAAT |
| | REMIYFGQTQGRSPQLLNRMTTYNEVDNLTKN | ATAAAGATCAACAAATTACTATTTTGACAAACTCAAAAGGTTAC |
| | NKGIAVLGSRVESRNGMHAGHAMAVVGNAKLD | TATGTGGTTACTCAAAATCATAAAGCTAAACTAGTACTAAAAAC |
| | NGQEVIIIWNPWDNGFMTQDAKNNVIPVSNGD | ACCACGTCTAGAAGATAAAAAGTTAAAAAAGACTGAGAGTATAC |
| | HYRWYSSIYGY | CTACTGGCAATAATGTAACGCAACTAAAGCAAAAAGCTTCTGTC |
| | | ACTATGCCAACTAGTCAATTTAAAAGTAATAATTATACTTACAA |
| | | CGAGCAATATATAAATAAACTTGAGAATTTCAAGATAAGAGAAA |
| | | CTCAAGGTAACAATGGTTGGTGTGCAGGCTATACGATGTCTGAA |
| | | TTACTAAATGCGACTTATAATACTAACAAATATCATGCAGAAGC |
| | | TGTGATGAGATTTTTACACCCAAACTTACAAGGACAACGATTCC |
| | | AATTTACTGGTTTAACGCCAAGAGAGATGATTTATTTTGGACAA |
| | | ACTCAGGGAAGAAGTCCACAACTTCTTAATAGAATGACTACATA |
| | | TAATGAAGTTGACAATCTTACTAAAAATAATAAAGGAATTGCTG |
| | | TTTTAGGTAGTAGAGTAGAATCAAGAAATGGTATGCACGCAGGG |
| | | CATGCCATGGCAGTTGTTGGGAACGCTAAATTAGATAATGGACA |
| | | AGAGGTTATAATTATATGGAATCCATGGGATAATGGTTTTATGA |
| | | CTCAAGATGCTAAAAACAATGTTATTCCAGTATCCAATGGCGAC |
| | | CATTATCGATGGTATTCATCTATTTATGGTTATTAA |
| SA0103 | SEQ ID NO: 53 | SEQ ID NO: 60 |
| (T1) | GKEAEVKKSFEKTLSMYPIKNLEDLYDKEGYR | GGTAAAGAAGCGGAAGTTAAGAAAAGCTTTGAAAAAACATTGAG |
| | DDEFDKNDKGTWIIGSEMVVQPKGERMKSKGM | TATGTATCCGATTAAAAATCTAGAGGATTTATACGATAAAGAAG |
| | VLYMNRNTKTTTGKYIVSETLHDEDGRPKSKD | GTTATCGTGATGACGAATTTGATAAAAATGATAAAGGTACATGG |
| | KEYPVKMVDNKIIPTKGIKDENIKKEIENFKF | ATTATTGGTTCTGAAATGGTTGTTCAACCTAAAGGGGAACGTAT |
| | FAQYGSFKDLSKYKDGDISYNPEVPSYSAKYQ | GAAATCAAAAGGTATGGTTCTATATATGAATAGAAACACTAAGA |
| | LTNDDYNVKQLRKRYKIPTNKAPKLLLKGSGD | CTACAACCGGGAAGTATATAGTGAGTGAAACACTTCATGATGAA |
| | LKGSSVGYKDIEFTFVEKKGENTFFTDSLHLE | GATGGTAGACCTAAGAGTAAAGATAAAGAATATCCCGTTAAAAT |
| | PSEDK | GGTAGACAATAAAATCATTCCAACTAAAGGTATCAAAGACGAAA |
| | | ATATAAAAAAAGAAATCGAGAATTTTAAGTTCTTCGCGCAATAT |
| | | GGCAGCTTTAAAGATTTGTCGAAGTACAAAGACGGCGATATATC |
| | | ATATAATCCAGAGGTACCGAGTTATTCAGCAAAATATCAATTAA |
| | | CTAATGATGACTATAATGTAAAACAATTACGAAAAAGATATAAA |
| | | ATACCAACGAATAAAGCACCAAAGTTATTGTTGAAAGGTTCGGG |
| | | AGACTTAAAAGGTTCATCAGTTGGATATAAAGACATTGAATTTA |
| | | CTTTTGTGGAGAAAAAAGGTGAAAATACTTTTTTTACTGATAGT |
| | | CTACATCTTGAACCGAGTGAGGATAAATAA |
| SA0377 | SEQ ID NO: 54 | SEQ ID NO: 61 |
| (T2) | SDQSDNEDHHTSTGIHAPKSAKKLETKDIFNS | TCCGATCAATCTGATAACGAAGATCATCATACAAGTACTGGCAT |
| | DKKNSDISDAEMKQAIEKYLSVNSDILDNKYI | CCATGCACCTAAAAGCGCTAAAAAATTAGAAACAAAAGATATCT |
| | MQHKLDKQIDSQTKVTEKQAETLSHLSNLAVK | TTAACTCAGATAAGAAAAATAGTGATATTAGTGATGCAGAGATG |
| | NDLHFKKFVTENNIPKEYKKPVELMMNYFKAL | AAGCAAGCTATAGAAAAGTATTTGTCTGTAAATAGCGATATACT |
| | NSTIANVDEDIEKLSYQPQNKINVVDVPTKYA | TGATAACAAATATATTATGCAACATAAACTAGATAAACAAATTG |
| | GDVNKKQQDKIKDFLKSKGIKSDVIDK | ACAGTCAAACAAAAGTGACTGAAAAACAAGCAGAAACACTTAGC |
| | | CACTTATCTAACTTAGCTGTTAAAAACGATTTGCATTTCAAGAA |
| | | ATTTGTCACTGAAAACAATATTCCTAAAGAATATAAGAAACCCG |
| | | TTGAATTAATGATGAATTACTTCAAAGCTTTAAATAGTACAATC |
| | | GCCAATGTTGATGAAGATATTGAAAAGCTTAGCTATCAACCGCA |
| | | AAATAAAATCAATGTTGTCGACGTGCCAACAAAATATGCCGGTG |
| | | ATGTTAATAAAAAGCAACAAGATAAAATTAAAGATTTCTTAAAA |
| | | TCTAAAGGTATCAAGAGTGATGTTATCGATAAATAA |
| SA0693 | SEQ ID NO: 55 | SEQ ID NO: 62 |
| (T3) | QDQGNKTEQKTKSEDSNVKTDKTKHLTGTESS | CAAGATCAAGGTAACAAAACTGAACAAAAAACAAAGTCAGAAGA |
| | KNGETVEGKAEIKNGKLMLTNYKSSKGPDLYV | TAGCAATGTTAAAACTGATAAAACAAAACACCTAACAGGTACAT |
| | YLTKNGDIKNGKEIAMVDYDKEKQTFDLKNVD | TCAGTTCTAAAAACGGTGAAACTGTTGAAGGTAAAGCTGAGATT |
| | LSKYDEVTIYCKKAHVIFGGAKLK | AAAAATGGTAAATTAATGCTTACTAACTACAAATCATCAAAAGG |
| | | TCCAGATTTATACGTCTACCTAACAAAAAATGGCGACATTAAAA |
| | | ACGGTAAAGAAATCGCAATGGTTGACTACGATAAAGAAAAACAA |
| | | ACATTTGATCTTAAAAATGTAGATTTAAGCAAATATGATGAAGT |
| | | AACAATCTATTGTAAAAAAGCTCACGTCATCTTCGGCGGCGCTA |
| | | AATTAAAATAA |
| SA2105 | SEQ ID NO: 56 | SEQ ID NO: 63 |
| (T4) | KFTREPKQDLEAATAQMENTKGKKSSVASKLV | AAGTTCACTAGAGAACCAAAGCAGGATTTAGAAGCTGCGACAGC |
| | SSDKNVNTEENASGNVSETSSSDDDPEALLDN | TCAAATGGAAAATACTAAAGGGAAAAAATCAAGCGTTGCTTCTA |
| | YNTEDVDAHNYNNINHVIFACDAGMGSSAMGA | AGTTAGTATCTTCTGATAAAAATGTTAATACAGAAGAAAATGCT |
| | SMLRNKFKKAGINDITVTNTAINQLPKDAQLV | AGTGGTAATGTTAGTGAAACATCTTCATCAGATGATGATCCTGA |
| | ITQKKLTDRAIKQTPNAIHISVDNFLNSPRYE | AGCGCTATTGGATAATTACAACACTGAAGATGTTGATGCACACA |
| | ELLNNLKKDDQA | ATTACAATAATATAAATCATGTTATTTTTGCCTGCGATGCGGGT |
| | | ATGGGTTCTAGTGCAATGGGTGCAAGCATGTTACGTAATAAATT |
| | | TAAAAAGGCGGGCATTAATGATATTACAGTTACAAATACTGCGA |
| | | TTAATCAATTGCCAAAAGATGCTCAATTAGTTATCACTCAGAAA |
| | | AAACTAACTGATCGTGCTATTAAACAAACACCAAATGCCATCCA |
| | | TATTTCAGTGGATAATTTCCTTAATTCACCAAGATATGAAGAAC |
| | | TTTTAAATAATCTAAAAAAAGATGATCAAGCATAA |
| Rhizav | SEQ ID NO: 1 | SEQ ID NO: 81 |
| idin | FDASNFKDFSSIASASSSWQNQSGSTMIIQVD | TTTGATGCGAGTAACTTTAAAGATTTCAGCTCTATTGCGAGCGC |
| (rhavi) | SFGNVSGQYVNRAQGTGCQNSPYPLTGRVNGT | CAGTAGCTCTTGGCAGAATCAGAGTGGCAGCACCATGATTATCC |
| | FIAFSVGWNNSTENCNSATGWTGYAQVNGNNT | AGGTGGATTCTTTCGGCAACGTTAGTGGCCAGTATGTTAATCGT |
| | EIVTSWNLAYEGGSGPAIEQGQDTFQYVPTTE | GCGCAGGGCACGGGTTGCCAGAACTCTCCGTACCCGCTGACCGG |
| | NKSLLKD | CCGCGTGAATGGCACGTTTATCGCCTTCAGCGTTGGCTGGAACA |
| | | ATTCTACCGAAAACTGTAATAGTGCAACCGGCTGGACGGGTTAT |
| | | GCGCAGGTGAACGGTAACAATACCGAAATTGTTACGAGCTGGAA |
| | | TCTGGCCTATGAAGGCGGTTCTGGCCCGGCAATCGAACAGGGTC |
| | | AGGATACCTTTCAGTACGTTCCGACCACGGAAAACAAAAGCCTG |
| | | CTGAAAGAT |

1. An immunogenic composition comprising at least one immunogenic complex, wherein the immunogenic complex comprises:
at least one biotinylated immunogenic polysaccharide antigen, and
at least one fusion protein, the fusion protein comprising:
a biotin-binding protein or biotin-binding moiety, and at least one S. aureus polypeptide antigen,
wherein the biotinylated polysaccharide antigen is non-covalently associated with the biotin-binding moiety of the fusion protein to form an immunogenic complex,
wherein the at least one immunogenic polysaccharide is selected from: type 1 capsular polysaccharide of Streptococcus pneumoniae, a type 5 capsular polysaccharide of S. aureus, and/or a type 8 capsular polysaccharide of S. aureus; and
wherein the at least one of the S. aureus polypeptide antigen is selected from any of: a SA1739 (B2) antigen comprising the amino acid sequence of SEQ ID NO: 51 or at least 85% sequence identity to SEQ ID NO: 51; a SA1720 (B1) antigen comprising the amino acid sequence of SEQ ID NO: 50 or at least 85% sequence identity to SEQ ID NO: 50; a SA1890 (B3) antigen comprising the amino acid sequence of SEQ ID NO: 52 or at least 85% sequence identity to SEQ ID NO: 52, a SA0103 (T1) antigen comprising the amino acid sequence of SEQ ID NO: 53 or at least 85% sequence identity to SEQ ID NO: 53; a SA0377 (T2) antigen comprising the amino acid sequence of SEQ ID NO: 54 or at least 85% sequence identity to SEQ ID NO: 54; a SA0693 (T3) antigen comprising the amino acid sequence of SEQ ID NO: 55 or at least 85% sequence identity to SEQ ID NO: 55, and a SA2105 (T4) antigen comprising the amino acid sequence of SEQ ID NO: 56 or at least 85% sequence identity to SEQ ID NO: 56.

2. The immunogenic composition of claim 1, wherein the immunogenic complex comprises at least two S. aureus polypeptide antigens, comprising (i) a first S. aureus polypeptide antigens comprising a SA 1739 (B2) antigen comprising the amino acid sequence of SEQ ID NO: 51 or a polypeptide that has at least 85% sequence identity to SEQ ID NO: 51, and (ii) a second polypeptide antigen selected from any of the group comprising: hemolysin (Hl), Clumping factor A (ClfA), Clumping factor B (ClfB), serine-aspirate repeat protein D (SdrD), serine-aspirate repeat protein E (SdrE), Iron regulator surface protein A (IsdA), Iron regulator surface protein B (IsdB), Leukotoxin D (LukD), Leukotoxin E (LukE), SA1720 (B1), SA1890 (B3), SA0103 (T1), SA0377 (T2), SA0693 (T3) or SA2105 (T4).

3. The immunogenic composition of claim 2, wherein the immunogenic complex comprises at least three S. aureus polypeptide antigens, comprising (i) a first S. aureus polypeptide antigens comprising a SA 1739 (B2) antigen comprising the amino acid sequence of SEQ ID NO: 51 or a polypeptide that has at least 85% sequence identity to SEQ ID NO: 51, and (ii) a second and third S. aureus antigen selected from any of the group comprising: hemolysin (Hl), Clumping Clumping factor A (ClfA), Clumping factor B (ClfB), serine-aspirate repeat protein D (SdrD), serine-aspirate repeat protein E (SdrE), Iron regulator surface protein A (IsdA), Iron regulator surface protein B (IsdB), Leukotoxin D (LukD), Leukotoxin E (LukE), SA1720 (B1), SA1890 (B3), SA0103 (T1), SA0377 (T2), SA0693 (T3) or SA2105 (T4).

4. The immunogenic composition of claim 3, wherein the immunogenic complex comprises at least four S. aureus polypeptide antigens, comprising (i) a first S. aureus polypeptide antigens comprising a SA 1739 (B2) antigen comprising the amino acid sequence of SEQ ID NO: 51 or a polypeptide that has at least 85% sequence identity to SEQ ID NO: 51, and (ii) at least three additional S. aureus antigen selected from any of the group comprising: hemolysin (Hl), Clumping Clumping factor A (ClfA), Clumping factor B (ClfB), serine-aspirate repeat protein D (SdrD), serine-aspirate repeat protein E (SdrE), Iron regulator surface protein A (IsdA), Iron regulator surface protein B (IsdB), Leukotoxin D (LukD), Leukotoxin E (LukE), SA1720 (B1), SA1890 (B3), SA0103 (T1), SA0377 (T2), SA0693 (T3) or SA2105 (T4).

5. The immunogenic composition of claim 4, wherein the immunogenic complex comprises at least five S. aureus polypeptide antigens, comprising (i) a first S. aureus polypeptide antigens comprising a SA 1739 (B2) antigen comprising the amino acid sequence of SEQ ID NO: 51 or a polypeptide that has at least 85% sequence identity to SEQ ID NO: 51, and (ii) at least four additional S. aureus antigen selected from any of the group comprising: hemolysin (Hl), Clumping Clumping factor A (ClfA), Clumping factor B (ClfB), serine-aspirate repeat protein D (SdrD), serine-aspirate repeat protein E (SdrE), Iron regulator surface protein A (IsdA), Iron regulator surface protein B (IsdB), Leukotoxin D (LukD), Leukotoxin E (LukE), SA1720 (B1), SA1890 (B3), SA0103 (T1), SA0377 (T2), SA0693 (T3) or SA2105 (T4).

6. The immunogenic composition of claim 5, wherein the immunogenic complex comprises (i) a first S. aureus polypeptide antigens comprising a SA1739 (B2) antigen comprising the amino acid sequence of SEQ ID NO: 51 or a polypeptide that has at least 85% sequence identity to SEQ ID NO: 51, and (ii) at least S. aureus antigens hemolysin (Hl), Clumping Clumping factor A (ClfA), Clumping factor B (ClfB), serine-aspirate repeat protein D (SdrD).

7. The immunogenic composition of claim 5, wherein the immunogenic complex comprises (i) a first S. aureus polypeptide antigens comprising a SA1739 (B2) antigen comprising the amino acid sequence of SEQ ID NO: 51 or a polypeptide that has at least 85% sequence identity to SEQ ID NO: 51, and (ii) a second S. aureus polypeptide antigen selected from SA0103 (T1), SA0377 (T2) or SA0693 (T3), and (iii) a third S. aureus polypeptide antigen selected from: hemolysin (Hl), Clumping Clumping factor A (ClfA), Clumping factor B (ClfB), serine-aspirate repeat protein D (SdrD).

8. The immunogenic composition of claim 7, wherein the immunogenic complex comprises (i) a first S. aureus polypeptide antigens comprising a SA1739 (B2) antigen comprising the amino acid sequence of SEQ ID NO: 51 or a polypeptide that has at least 85% sequence identity to SEQ ID NO: 51 and (ii) a second S. aureus polypeptide antigen comprising SA0103 (T1), and (iii) a third S. aureus polypeptide antigen selected from: hemolysin (Hi), Clumping Clumping factor A (ClfA), Clumping factor B (ClfB), serine-aspirate repeat protein D (SdrD).

9. The immunogenic composition of claim 7, wherein the immunogenic complex comprises (i) a first S. aureus polypeptide antigen comprising a SA1739 (B2) antigen comprising the amino acid sequence of SEQ ID NO: 51 or a polypeptide that has at least 85% sequence identity to SEQ ID NO: 51 and (ii) a second S. aureus polypeptide antigen comprising SA0103 (T1) and SA0693 (T3), and (iii) a third S. aureus polypeptide antigen selected from; hemolysin (Hl), Clumping Clumping factor A (ClfA), Clumping factor B (ClfB), serine-aspirate repeat protein D (SdrD).

10. The immunogenic composition of claim 7, wherein the immunogenic complex comprises (i) a first S. aureus polypeptide antigens comprising a SA1739 (B2) antigen comprising the amino acid sequence of SEQ ID NO: 51 or a polypeptide that has at least 85% sequence identity to SEQ ID NO: 51 and (ii) a second S. aureus polypeptide antigen comprising SA0103 (T1) and SA0377 (T2), and (iii)) a third S. aureus polypeptide antigen selected from; hemolysin (Hl), Clumping Clumping factor A (ClfA), Clumping factor B (ClfB), serine-aspirate repeat protein D (SdrD).

11. The immunogenic composition of claim 9 or 10, wherein the second S. aureus polypeptide antigen is a S. aureus antigen fusion protein, comprising SA0103 (T1) fused to SA0377 (T2), or SA0103 (T1) fused to SA0693 (T3).

12. The immunogenic composition of claim 1, wherein the immunogenic complex comprises at least two S. aureus polypeptide antigens, which are (i) a S. aureus antigen selected from SA0103 (T1), SA0377 (T2) or SA0693 (T3), or SA2105 (T4) and (ii) a polypeptide antigen selected from any of the group comprising: SA1720 (B1), SA1739 (B2), SA1890 (B3), hemolysin (Hl), Clumping factor A (ClfA), Clumping factor B (ClfB), serine-aspirate repeat protein D (SdrD), serine-aspirate repeat protein E (SdrE), Iron regulator surface protein A (IsdA), Iron regulator surface protein B (IsdB), Leukotoxin D (LukD), or Leukotoxin E (LukE)

13. The immunogenic composition of claim 12, wherein the immunogenic complex comprises at least three S. aureus polypeptide antigens, which are (i) a S. aureus antigen selected from SA0103 (T1), SA0377 (T2) or SA0693 (T3), or SA2105 (T4) and (ii) at least two additional S. aureus antigen selected from any of the group comprising: SA1720 (B1), SA1739 (B2), SA1890 (B3), hemolysin (Hl), Clumping Clumping factor A (ClfA), Clumping factor B (ClfB), serine-aspirate repeat protein D (SdrD), serine-aspirate repeat protein E (SdrE), Iron regulator surface protein A (IsdA), Iron regulator surface protein B (IsdB), Leukotoxin D (LukD), or Leukotoxin E (LukE).

14. The immunogenic composition of claim 13, wherein the immunogenic complex comprises at least four S. aureus polypeptide antigens, which are (i) a S. aureus antigen selected from SA0103 (T1), SA0377 (T2) or SA0693 (T3) or SA2105 (T4) and, (ii) at least three additional S. aureus antigen selected from any of the group comprising: SA1720 (B1), SA1739 (B2), SA1890 (B3), hemolysin (Hl), Clumping Clumping factor A (ClfA), Clumping factor B (ClfB), serine-aspirate repeat protein D (SdrD), serine-aspirate repeat protein E (SdrE), Iron regulator surface protein A (IsdA), Iron regulator surface protein B (IsdB), Leukotoxin D (LukD), or Leukotoxin E (LukE).

15. The immunogenic composition of claim 14, wherein the immunogenic complex comprises at least five S. aureus polypeptide antigens, which are (i) a S. aureus antigen selected from SA0103 (T1), SA0377 (T2) or SA0693 (T3) or SA2105 (T4) and, (ii) at least four additional S. aureus antigen selected from any of the group comprising: SA1720 (B1), SA1739 (B2), SA1890 (B3), hemolysin (Hl), Clumping Clumping factor A (ClfA), Clumping factor B (ClfB), serine-aspirate repeat protein D (SdrD), serine-aspirate repeat protein E (SdrE), Iron regulator surface protein A (IsdA), Iron regulator surface protein B (IsdB), Leukotoxin D (LukD), or Leukotoxin E (LukE).

16. The immunogenic composition of claim 15, wherein the immunogenic complex comprises (i) a SA0103 (T1) S. aureus antigen and (ii) at least S. aureus antigens hemolysin (Hl), Clumping Clumping factor A (ClfA), Clumping factor B (ClfB), serine-aspirate repeat protein D (SdrD).

17. The immunogenic composition of claim 16, wherein the immunogenic composition comprises (i) S. aureus antigens SA0103 (T1) and SA0377 (T2), or SA0103 (T1) and SA0693 (T3), or SA0103 (T1) and SA2105 (T4) and (ii) at least S. aureus antigens hemolysin (Hl), Clumping Clumping factor A (ClfA), Clumping factor B (ClfB), serine-aspirate repeat protein D (SdrD).

18. The immunogenic composition of claim 17, wherein the immunogenic complex comprises (i) S. aureus antigens: SA0103 (T1) and SA0377 (T2), or SA0103 (T1) and SA0693 (T3) or SA0103 (T1) and SA2105 (T4) and (ii) a S. aureus antigen selected from: SA1720 (B1), SA1739 (B2), SA1890 (B3), and (iii) at least S. aureus antigens hemolysin (Hl), Clumping Clumping factor A (ClfA), Clumping factor B (ClfB), serine-aspirate repeat protein D (SdrD).

19. The immunogenic composition of claim 17 or 18, wherein the immunogenic complex comprises a S. aureus antigen fusion protein, comprising SA0103 (T1) fused to any one of: SA0377 (T2), SA0693 (T3) or SA2105 (T4).

20. The immunogenic composition of claim 12, wherein the immunogenic complex comprises (i) a SA0377 (T2) S. aureus antigen and (ii) at least S. aureus antigens hemolysin (Hl), Clumping Clumping factor A (ClfA), Clumping factor B (ClfB), serine-aspirate repeat protein D (SdrD).

21. The immunogenic composition of claim 12, wherein the immunogenic complex comprises (i) a SA0693 (T3) S. aureus antigen and (ii) at least S. aureus antigens hemolysin (Hl), Clumping Clumping factor A (ClfA), Clumping factor B (ClfB), serine-aspirate repeat protein D (SdrD).

22. The immunogenic composition of claim 12, wherein the immunogenic complex comprises (i) a SA2015 (T4) S. aureus antigen and (ii) at least S. aureus antigens hemolysin (Hl), Clumping Clumping factor A (ClfA), Clumping factor B (ClfB), serine-aspirate repeat protein D (SdrD).

23. The immunogenic composition of any of claims 1-19, wherein the immunogenic complex comprises S. aureus antigens Hla209(27-319), ClfA(221-559), ClfB (203-542), SdrD (246-682), IsdA (47-324) and IsdB (48-447), or functional modifications thereof.

24. The immunogenic composition of any of claims 1 to 20, wherein Hl antigen is a α-hemolysin (Hla), a β-hemolysin (Hlb) or a γ-hemolysin (Hl-gamma) from S. aureus.

25. The immunogenic composition of any of claims 1 to 21, wherein Hl is wildtype Hla (WT Hla) or a Hla with a reduced hemolytic activity or is a non-hemolytic Hla protein.

26. The immunogenic composition of any of claims 1 to 22, wherein the Hla antigen with a reduced hemolytic activity comprises amino acids of SEQ ID NO: 14, SEQ ID NO: 15, SEQ ID NO: 17 or SEQ ID NO: 18 or a polypeptide with at least 85% sequence identity thereto.

27. The immunogenic composition of any of claims 1 to 24, wherein the Hla antigen with a reduced hemolytic activity is amino acids of SEQ ID NO: 16 or a polypeptide with at least 85% sequence identity thereto.

28. The immunogenic composition of any of claims 1 to 20, wherein the ClfA antigen comprises at least SEQ ID NO: 3 or a polypeptide with at least 85% sequence identity to SEQ ID NO: 3.

29. The immunogenic composition of any of claims 1 to 20, wherein the ClfA antigen comprises a fragment of at least 30 amino acids of SEQ ID NO: 2 or a polypeptide of at least 30 amino acids that has at least 85% sequence identity to a portion of SEQ ID NO: 2.

30. The immunogenic composition of any of claims 1 to 20, wherein the ClfB antigen comprises at least SEQ ID NO: 5 or a polypeptide with at least 85% sequence identity to SEQ ID NO: 5.

31. The immunogenic composition of any of claims 1 to 20, wherein the ClfB antigen comprises a fragment of at least 30 amino acids of SEQ ID NO: 4 or a polypeptide of at least 30 amino acids that has at least 85% sequence identity to a portion of SEQ ID NO: 4.

32. The immunogenic composition of any of claims 1 to 20, wherein the SdrD antigen comprises at least SEQ ID NO: 7 or a polypeptide with at least 85% sequence identity to SEQ ID NO: 7.

33. The immunogenic composition of any of claims 1 to 20, wherein the SdrD antigen comprises a fragment of at least 30 amino acids of SEQ ID NO:6 or a polypeptide of at least 30 amino acids that has at least 85% sequence identity to a portion of SEQ ID NO: 6.

34. The immunogenic composition of any of claims 1-33, wherein the biotin-binding is selected from the group consisting of: rhizavidin, avidin, streptavidin, or a homologue or derivative thereof.

35. The immunogenic composition of claim 34, wherein the rhizavidin is amino acids of SEQ ID NO: 1, or 85% sequence identity to amino acids of SEQ ID NO: 1.

36. The immunogenic composition of any of claims 1 to 35, wherein the fusion protein comprises at least one S. aureus polypeptide antigen fused to the biotin-binding molecule.

37. The immunogenic composition of any of claims 1 to 36, wherein the fusion protein comprises at least two S. aureus polypeptide antigens, wherein a first S. aureus polypeptide antigen fused to the biotin-binding molecule and a second S. aureus polypeptide antigen is fused to the first S. aureus polypeptide antigen, or wherein the first S. aureus polypeptide antigen, and the second S. aureus polypeptide antigen is fused to the biotin-binding molecule.

38. The immunogenic composition of claim 37, wherein the first S. aureus polypeptide antigen is a SA0103 (T1) S. aureus polypeptide antigen, which is fused to a second S. aureus polypeptide antigen selected from any of: SA0377 (T2), SA0693 (T3), SA2104 (T4), wherein SA0103 (T1) is fused to the second affinity molecule.

39. The immunogenic composition of claim 38, wherein the the first S. aureus polypeptide is a SA0103 (T1) S. aureus polypeptide antigen which is fused to to a second S. aureus polypeptide antigen selected from any of: SA0377 (T2), SA0693 (T3) or SA2104 (T4), wherein SA0377 (T2) or SA0693 (T3) or SA2104 (T4) is fused to the second affinity molecule.

40. The immunogenic composition of claim 1, the fusion protein further comprising at least one linker peptide located between the at least one of the S. aureus polypeptide antigen and the biotin-binding protein or biotin-binding moiety.

41. The immunogenic composition of any of claims 1 to 40, further comprising at least one adjuvant or co-stimulation factor.

42. The immunogenic composition of claim 1 for use in any one or more of:
(i) as a diagnostic for exposure to a S. aureus pathogen or immune threat,
(ii) to prevent or treat an infection by S. aureus,
(iii) to prevent colonization of a subject by S. aureus,
(iv) to elicit an immune response to S. aureus in a subject, wherein the immune response is selected from any of:
i. an antibody or B-cell response,
ii. an antibody or B-cell response and T-cell response,
iii. an immune response to the at least one immunogenic polysaccharide and at least one of the S. aureus polypeptide antigens,
iv. an immune response that is a CD4+ T cell response, including Th1, Th2, or Th17 or Th22 response, or a CD8+ T cell response, or CD4+ and CD8+ T cell response,
v. an antibody or B cell response to the at least one immunogenic polysaccharide and a CD4+ T cell response, including Th1, Th2, or Th17 or Th22 response, or a CD8+ T cell response, or CD4+/CD8+ T cell response to at least one of the S. aureus polypeptide antigens,
vi. an antibody or B cell response to the at least one immunogenic polysaccharide, and an antibody or B cell response and a CD4+ T cell response, including Th1, Th2, Th17 or Th22 responses, or a CD8+ T cell response, or CD4+/CD8+ T cell response to at least one of the S. aureus polypeptide antigens,
vii. an immune response results in activation of INF-γ, IL-17A or IL-22 producing cells, or INF-γ, IL-17A and IL-22 producing cells, or
viii. an antibody or B-cell response against at least one of the S. aureus polypeptide antigens which associates with the at least one immunogenic polysaccharide.

43. A method for inducing an immune response in a subject to S. aureus, comprising administering to the subject a immunogenic composition of claim 1 to 42.

44. A method of vaccinating a mammal against S. aureus infection, the method comprising administering an immunogenic composition of claim 1 to 42.

45. The method of any of claim 43 or 44, wherein the subject is a human.

46. The method of any of claim 43 or 44, wherein the subject is an agricultural or non-domestic animal.

47. The method of any of claim 43 or 44, wherein the subject is a domestic animal.

48. The method of any of claim 43 or 44, wherein administration is via subcutaneous, intranasal, intradermal, or intra muscular injection, or via transdermal skin patch.

49. The method of claim 43, wherein the immune response is selected from any of:
i. an antibody or B-cell response,
ii. an antibody or B-cell response and T-cell response,
iii. an immune response to the at least one immunogenic polysaccharide and at least one of the S. aureus polypeptide antigens,
iv. an immune response that is a CD4+ T cell response, including Th1, Th2, or Th17 or Th22 response, or a CD8+ T cell response, or CD4+ and CD8+ T cell response,
v. an antibody or B cell response to the at least one immunogenic polysaccharide and a CD4+ T cell response, including Th1, Th2, or Th17 or Th22 response, or a CD8+ T cell response, or CD4+/CD8+ T cell response to at least one of the S. aureus polypeptide antigens,
vi. an antibody or B cell response to the at least one immunogenic polysaccharide, and an antibody or B cell response and a CD4+ T cell response, including Th1, Th2, Th17 or Th22 responses, or a CD8+ T cell response, or CD4+/CD8+ T cell response to at least one of the S. aureus polypeptide antigens,
vii. an immune response results in activation of INF-γ, IL-17A or IL-22 producing cells, or INF-γ, IL-17A and IL-22 producing cells, or
viii. an antibody or B-cell response against at least one of the S. aureus polypeptide antigens which associates with the at least one immunogenic polysaccharide.

50. A fusion protein comprising, in any order, a rhizavidin protein and at least one S. aureus polypeptide antigen, wherein the rhizavidin protein comprises amino acids of SEQ ID NO: 1, or 85% sequence identity to amino acids of SEQ ID NO: 1, and the S. aureus polypeptide antigen comprises a polypeptide comprising the amino acids selected from the group of SEQ ID NO: 50 (SA1739; B1), SEQ ID NO: 51 (SA1720; B2), SEQ ID NO: 52 (SA1890; B3), SEQ ID NO: 53 (SA0103; T1), SEQ ID NO: 54 (SA0377; T2), SEQ ID NO: 55 (SA0693; T3), SEQ ID NO: 56 (SA2105; T4), or a fragment of at least 20 amino acids thereof, or a a polypeptide having at least 80%, or at least 85% or 90% sequence identity to any of SEQ ID NO: 51-56.

51. The fusion protein of claim 50, where the fusion protein comprises, in the following order:
(a) (i) a rhizavidin protein and at least one S. aureus polypeptide antigen, wherein the rhizavidin protein comprises amino acids of SEQ ID NO: 1, or 85% sequence identity to amino acids of SEQ ID NO: 1, and (ii) a S. aureus polypeptide antigen comprises a polypeptide comprising the amino acids selected from the group of: SEQ ID NO: 50 (SA1739; B1), SEQ ID NO: 51 (SA1720; B2), SEQ ID NO: 52 (SA1890; B3), SEQ ID NO: 53 (SA0103; T1), SEQ ID NO: 54 (SA0377; T2), SEQ ID NO: 55 (SA0693; T3), SEQ ID NO: 56 (SA2105; T4), or a fragment of at least 20 amino acids thereof, or a a polypeptide having at least 80%, or at least 85% or 90% sequence identity to any of SEQ ID NO: 51-56, or
(b) (i) a S. aureus polypeptide antigen comprises a polypeptide comprising the amino acids selected from the group of: SEQ ID NO: 50 (SA1739; B1), SEQ ID NO: 51 (SA1720; B2), SEQ ID NO: 52 (SA1890; B3), SEQ ID NO: 53 (SA0103; T1), SEQ ID NO: 54 (SA0377; T2), SEQ ID NO: 55 (SA0693; T3), SEQ ID NO: 56 (SA2105; T4), or a fragment of at least 20 amino acids thereof, or a a polypeptide having at least 80%, or at least 85% or 90% sequence identity to any of SEQ ID NO: 51-56, and (ii) a rhizavidin protein and at least one S. aureus polypeptide antigen, wherein the rhizavidin protein comprises amino acids of SEQ ID NO: 1, or 85% sequence identity to amino acids of SEQ ID NO: 1.

52. A fusion protein comprising, in any order, a rhizavidin protein and an S. aureus polypeptide antigen, wherein the rhizavidin protein comprises amino acids of SEQ ID NO: 1, or a polypeptide having at least 85% sequence identity to amino acids of SEQ ID NO: 1, and the S. aureus polypeptide comprises a SA1739 (B2) S. aureus polypeptide antigen comprising the amino acid sequence of at least 80%, or at least 85%, or at least 90% sequence identity to SEQ ID NO: 51.

53. The fusion protein of claim 52, wherein the SA1739 (B2) S. aureus polypeptide antigen comprises at least SEQ ID NO: 51 or a protein of at least 20 amino acids that has at least 85% sequence identity to SEQ ID NO: 51.

54. The fusion protein of claim 53, wherein the fusion protein further comprises, at least one additional S. aureus polypeptide antigen selected from any of: hemolysin (Hl), Clumping factor A (ClfA), Clumping factor B (ClfB), serine-aspirate repeat protein D (SdrD), serine-aspirate repeat protein E (SdrE), Iron regulator surface protein A (IsdA), Iron regulator surface protein B (IsdB), Leukotoxin D (LukD), Leukotoxin E (LukE), SA1720 (B1), SA1890 (B3), SA0103 (T1), SA0377 (T2), SA0693 (T3) or SA2105 (T4).

55. The fusion protein of claim 53, wherein the fusion protein further comprises (ii) at least one additional S. aureus polypeptide antigen selected from any of: SEQ ID NO: 50 (SA1739; B1), SEQ ID NO: 52 (SA1890; B3), SEQ ID NO: 53 (SA0103; T1), SEQ ID NO: 54 (SA0377; T2), SEQ ID NO: 55 (SA0693; T3) and SEQ ID NO: 56 (SA2105; T4), or a polypeptide having at least 80%, or at least 85% or 90% sequence identity to any of SEQ ID NO: 50, 52-56.

56. The fusion protein of any of claims 52-55, wherein the fusion protein is selected from any of:
(a) a fusion protein comprising (i) a rhizavidin protein or biotin-binding protein comprising the amino acid sequence of SEQ ID NO: 1, or a polypeptide having at least 85% sequence identity to amino acids of SEQ ID NO: 1, and (ii) a SA1739 (B2) S. aureus polypeptide antigen comprising the amino acid sequence having at least 80%, or at least 85%, or at least 90% sequence identity to SEQ ID NO: 51,
(b) a fusion protein comprising (i) a SA1739 (B2) S. aureus polypeptide antigen comprising the amino acid sequence having at least 80%, or at least 85%, or at least 90% sequence identity to SEQ ID NO: 51, and (ii) a rhizavidin protein or biotin-binding protein comprising the amino acid sequence of SEQ ID NO: 1, or a polypeptide having at least 85% sequence identity to amino acids of SEQ ID NO: 1.

57. A fusion protein comprising, in any order, a rhizavidin protein and an S. aureus polypeptide antigen, wherein the rhizavidin protein comprises amino acids of SEQ ID NO: 1, or a polypeptide having at least 85% sequence identity to amino acids of SEQ ID NO: 1, and the S. aureus polypeptide selected from SA1720 (B1) or SA1890 (B3) S. aureus polypeptide antigen, wherein the SA1720 (B1) S. aureus polypeptide antigen comprises the amino acid sequence of at least 80%, or at least 85%, or at least 90% sequence identity to SEQ ID NO: 50, and wherein the SA1890 (B3) S. aureus polypeptide antigen comprises the amino acid sequence of at least 80%, or at least 85%, or at least 90% sequence identity to SEQ ID NO: 52.

58. The fusion protein of claim 57, wherein the SA1720 (B1) S. aureus polypeptide antigen comprises at least SEQ ID NO: 50 or a protein of at least 20 amino acids that has at least 85% sequence identity to SEQ ID NO: 50, and wherein the SA1890 (B3) S. aureus polypeptide antigen comprises at least SEQ ID NO: 52 or a protein of at least 20 amino acids that has at least 85% sequence identity to SEQ ID NO: 52.

59. The fusion protein of claim 57, wherein the fusion protein is selected from any of:
(a) a fusion protein comprising, in the following order, (i) a rhizavidin protein or biotin-binding protein comprising the amino acids of SEQ ID NO: 1, or a polypeptide having at least 85% sequence identity to amino acids of SEQ ID NO: 1, and (ii) a SA1720 (B1) S. aureus polypeptide comprising the amino acid sequence of at least 80%, or at least 85%, or at least 90% sequence identity to SEQ ID NO: 50;
(b) a fusion protein comprising, in the following order, (i) a SA1720 (B1) S. aureus polypeptide comprising the amino acid sequence of at least 80%, or at least 85%, or at least 90% sequence identity to SEQ ID NO: 50, and (ii) a rhizavidin protein or biotin-binding protein comprising the amino acids of SEQ ID NO: 1, or a polypeptide having at least 85% sequence identity to amino acids of SEQ ID NO: 1,
(c) a fusion protein comprising, in the following order, (i) a rhizavidin protein or biotin-binding protein comprising the amino acids of SEQ ID NO: 1, or a polypeptide having at least 85% sequence identity to amino acids of SEQ ID NO: 1, and (ii) a or SA1890 (B3) S. aureus polypeptide comprising the amino acid sequence of at least 80%, or at least 85%, or at least 90% sequence identity to SEQ ID NO: 52; and
(d) a fusion protein comprising, in the following order, (i) a SA1890 (B3) S. aureus polypeptide comprising the amino acid sequence of at least 80%, or at least 85%, or at least 90% sequence identity to SEQ ID NO: 52, and (ii) a rhizavidin protein or biotin-binding protein comprising the amino acids of SEQ ID NO: 1, or a polypeptide having at least 85% sequence identity to amino acids of SEQ ID NO: 1.

60. The fusion protein of claim 57, wherein the fusion protein further comprises, fused to the SA1720 (B1) or SA1890 (B3) S. aureus polypeptide, at least one additional S. aureus polypeptide antigen selected from any of: hemolysin (Hl), Clumping factor A (ClfA), Clumping factor B (ClfB), serine-aspirate repeat protein D (SdrD), serine-aspirate repeat protein E (SdrE), Iron regulator surface protein A (IsdA), Iron regulator surface protein B (IsdB), Leukotoxin D (LukD), Leukotoxin E (LukE), SA1720 (B1), SA1739 (B2), SA1890 (B3), SA0103 (T1), SA0377 (T2), SA0693 (T3) or SA2105 (T4).

61. The fusion protein of any of claims 57-60, wherein the fusion protein comprises (i) a SA1720 (B1) S. aureus antigen and further comprises (ii) at least one additional S. aureus polypeptide antigen selected from any of: SEQ ID NO: 50 (SA1739; B1), SEQ ID NO: 51 (SA1720; B2), SEQ ID NO: 52 (SA1890; B3), SEQ ID NO: 53 (SA0103; T1), SEQ ID NO: 54 (SA0377; T2), SEQ ID NO: 55 (SA0693; T3), SEQ ID NO: 56 (SA2105; T4), or a fragment of at least 20 amino acids thereof, or a a polypeptide having at least 80%, or at least 85% or 90% sequence identity to any of SEQ ID NO: 51-56.

62. The fusion protein ofany of claims 57-60, wherein the fusion protein comprises (i) a SA1890 (B3) S. aureus antigen and further comprises (ii) at least one additional S. aureus polypeptide antigen selected from any of: SEQ ID NO: 50 (SA1739; B1), SEQ ID NO: 51 (SA1720; B2), SEQ ID NO: 52 (SA1890; B3), SEQ ID NO: 53 (SA0103; T1), SEQ ID NO: 54 (SA0377; T2), SEQ ID NO: 55 (SA0693; T3), SEQ ID NO: 56 (SA2105; T4), or a fragment of at least 20 amino acids thereof, or a polypeptide having at least 80%, or at least 85% or 90% sequence identity to any of SEQ ID NO: 51-56.

63. A fusion protein comprising, in any order, a rhizavidin protein and an S. aureus polypeptide antigen, wherein the rhizavidin protein comprises amino acids of SEQ ID NO: 1, or a polypeptide having at least 85% sequence identity to amino acids of SEQ ID NO: 1, and the S. aureus polypeptide comprises a SA0103 (T1) S. aureus polypeptide antigen comprising the amino acid sequence of at least 80%, or at least 85%, or at least 90% sequence identity to SEQ ID NO: 53.

64. The fusion protein of claim 63, wherein the fusion protein is selected from any of:
(a) a fusion protein comprising (i) a rhizavidin protein or biotin-binding protein comprising the amino acid sequence of SEQ ID NO: 1, or a polypeptide having at least 85% sequence identity to amino acids of SEQ ID NO: 1, and (ii) SA0103 (T1) S. aureus polypeptide antigen comprising the amino acid sequence having at least 80%, or at least 85%, or at least 90% sequence identity to SEQ ID NO: 53,
(b) a fusion protein comprising (i) a SA0103 (T1) S. aureus polypeptide antigen comprising the amino acid sequence having at least 80%, or at least 85%, or at least 90% sequence identity to SEQ ID NO: 53, and (ii) a rhizavidin protein or biotin-binding protein comprising the amino acid sequence of SEQ ID NO: 1, or a polypeptide having at least 85% sequence identity to amino acids of SEQ ID NO: 1.

65. The fusion protein of claim 63 or 64, wherein the SA0103 (T1) S. aureus polypeptide antigen comprises at least SEQ ID NO: 53 or a protein of at least 20 amino acids that has at least 85% sequence identity to SEQ ID NO: 53.

66. The fusion protein of any of claims 63-65, wherein the fusion protein comprises (i) a SA0103 (T1) S. aureus antigen, and further comprises (ii) at least one additional S. aureus polypeptide antigen selected from any of: SA1720 (B1), SA1739 (B2), SA1890 (B3), hemolysin (Hl), Clumping factor A (ClfA), Clumping factor B (ClfB), serine-aspirate repeat protein D (SdrD), serine-aspirate repeat protein E (SdrE), Iron regulator surface protein A (IsdA), Iron regulator surface protein B (IsdB), Leukotoxin D (LukD) or Leukotoxin E (LukE).

67. The fusion protein of any of claims 63-67, wherein the fusion protein further comprises (ii) at least one additional S. aureus polypeptide antigen selected from: SEQ ID NO: 54 (SA0377; T2), SEQ ID NO: 55 (SA0693; T3), SEQ ID NO: 56 (SA2105; T4), or T4) or a polypeptide having at least 80%, or at least 85% or 90% sequence identity to any of SEQ ID NO: 54-56.

68. The fusion protein of any of claims 63-67, wherein the fusion protein comprises (i) a SA0103 (T1) S. aureus polypeptide antigen comprising the amino acid sequence of at least 80%, or at least 85%, or at least 90% sequence identity to SEQ ID NO: 53 and further comprises (ii) at least a SA0377 (T2) S. aureus polypeptide antigen comprising the amino acid sequence of at least 80%, or at least 85%, or at least 90% sequence identity to SEQ ID NO: 54.

69. The fusion protein of any of claims 63-67, wherein the fusion protein comprises (i) SA0103 (T1) S. aureus polypeptide antigen comprising the amino acid sequence of at least 80%, or at least 85%, or at least 90% sequence identity to SEQ ID NO: 53 (T1) S. aureus antigen and further comprises (ii) at least a SA0693 (T3) S. aureus polypeptide antigen comprising the amino acid sequence of at least 80%, or at least 85%, or at least 90% sequence identity to SEQ ID NO: 55.

70. The fusion protein of any of claims 63-67, wherein the fusion protein comprises (i) SA0103 (T1) S. aureus polypeptide antigen comprising the amino acid sequence of at least 80%, or at least 85%, or at least 90% sequence identity to SEQ ID NO: 53 (T1) S. aureus antigen and further comprises (ii) at least a SA2105 (T4) S. aureus polypeptide antigen comprising the amino acid sequence of at least 80%, or at least 85%, or at least 90% sequence identity to SEQ ID NO: 56.

71. A fusion protein comprising, in any order, (i) a rhizavidin protein or biotin-binding protein, (ii) a first S. aureus polypeptide antigen and (iii) a second S. aureus polypeptide antigen, wherein the fusion protein is selected from any of:
(a) a fusion protein comprising, in any order, a rhizavidin protein or biotin-binding protein comprises amino acids of SEQ ID NO: 1 or a polypeptide having at least 85% sequence identity to amino acids of SEQ ID NO: 1, a first S. aureus polypeptide antigen is selected from any of: SA1720 (B1), SA1739 (B2) or SA1890 (B3), and a second S. aureus polypeptide antigen is selected from any of: SA0103 (T1), SA0377 (T2), SA0693 (T3) or SA2105 (T4);
(b) a fusion protein comprising, in any order, a rhizavidin protein or biotin-binding protein comprises amino acids of SEQ ID NO: 1 or a polypeptide having at least 85% sequence identity to amino acids of SEQ ID NO: 1, a first S. aureus polypeptide antigen is selected from any of: SA0103 (T1), SA0377 (T2), SA0693 (T3) or SA2105 (T4); and a second S. aureus polypeptide antigen is selected from any of: SA0103 (T1), SA0377 (T2), SA0693 (T3) or SA2105 (T4); and
(c) a fusion protein comprising, in any order, a rhizavidin protein or biotin-binding protein comprises amino acids of SEQ ID NO: 1 or a polypeptide having at least 85% sequence identity to amino acids of SEQ ID NO: 1, a first S. aureus polypeptide antigen is selected from any of: SA1720 (B1), SA1739 (B2) or SA1890 (B3), and a second S. aureus polypeptide antigen is selected from any of: SA1720 (B1), SA1739 (B2) or SA1890 (B3),
and wherein, the S. aureus polypeptide antigens comprise the amino acid sequence selected from from the group of: SEQ ID NO: 50 (SA1739; B1), SEQ ID NO: 51 (SA1720; B2), SEQ ID NO: 52 (SA1890; B3), SEQ ID NO: 53 (SA0103; T1), SEQ ID NO: 54 (SA0377; T2), SEQ ID NO: 55 (SA0693; T3), SEQ ID NO: 56 (SA2105; T4), or a fragment of at least 20 amino acids thereof, or a polypeptide having at least 80%, or at least 85% or 90% sequence identity to any of SEQ ID NO: 51-56.

72. The fusion protein of claim 71, wherein the fusion protein is selected from any of the fusion proteins disclosed in Table 5B.

73. The fusion protein of claim 71 or 72, wherein the fusion protein comprises a rhizavidin protein or biotin-binding protein and a first S. aureus polypeptide antigen and second S. aureus polypeptide antigen, in any order, selected from the groups of:
SA1720 (B1) and SA0103 (T1),
SA1720 (B1) and SA0377 (T2),
SA1720 (B1) and SA0693 (T3),
SA1720 (B1) and SA2105 (T4),
SA1739 (B2) and SA0103 (T1),
SA1739 (B2) and SA0377 (T2),
SA1739 (B2) and SA0693 (T3),
SA1739 (B2) and SA2105 (T4),
SA1890 (B3) and SA0103 (T1),
SA1890 (B3) and SA0377 (T2),
SA1890 (B3) and SA0693 (T3),
SA1890 (B3) and SA2105 (T4),
SA0103 (T1) and SA0377 (T2),
SA0103 (T1) and SA0693 (T3),
SA0103 (T1) and SA2105 (T4),
SA0377 (T2) and SA0693 (T3),
SA0377 (T2) and SA2105 (T4), and
SA0693 (T3) and SA2105 (T4).

74. The fusion protein of any of claims 71-73, wherein the SA1720 (B1) S. aureus polypeptide antigen comprises the amino acid sequence of SEQ ID NO: 50 or a polypeptide with at least 80%, or at least 85%, or at least 90% amino acid sequence identity to SEQ ID NO: 50, or a protein of at least 20 amino acids that has at least 85% sequence identity to SEQ ID NO: 50.

75. The fusion protein of any of claims 71-73, wherein the SA1739 (B2) S. aureus polypeptide antigen comprises the amino acid sequence of SEQ ID NO: 51 or a polypeptide with at least 80%, or at least 85%, or at least 90% amino acid sequence identity to SEQ ID NO: 51, or a protein of at least 20 amino acids that has at least 85% sequence identity to SEQ ID NO: 51.

76. The fusion protein of any of claims 71-73, wherein the SA1809(B3) S. aureus polypeptide antigen comprises the amino acid sequence of SEQ ID NO: 52 or a polypeptide with at least 80%, or at least 85%, or at least 90% amino acid sequence identity to SEQ ID NO: 52, or or a protein of at least 20 amino acids that has at least 85% sequence identity to SEQ ID NO: 52.

77. The fusion protein of any of claims 71-73, wherein the SA0103 (T1) S. aureus polypeptide antigen comprises the amino acid sequence of SEQ ID NO: 53 or a polypeptide with at least 80%, or at least 85%, or at least 90% amino acid sequence identity to SEQ ID NO: 53, or a protein of at least 20 amino acids that has at least 85% sequence identity to SEQ ID NO: 53.

78. The fusion protein of any of claims 71-73, wherein the SA0377 (T2) S. aureus polypeptide antigen comprises the amino acid sequence of SEQ ID NO: 54 or a polypeptide with at least 80%, or at least 85%, or at least 90% amino acid sequence identity to SEQ ID NO: 54, or a protein of at least 20 amino acids that has at least 85% sequence identity to SEQ ID NO: 54.

79. The fusion protein of any of claims 71-73, wherein the SA0693 (T3) S. aureus polypeptide antigen comprises the amino acid sequence of SEQ ID NO: 55 or a polypeptide with at least 80%, or at least 85%, or at least 90% amino acid sequence identity to SEQ ID NO: 55, or a protein of at least 20 amino acids that has at least 85% sequence identity to SEQ ID NO: 55.

80. The fusion protein of any of claims 71-73, wherein the SA2105 (T4) S. aureus polypeptide antigen comprises the amino acid sequence of SEQ ID NO: 56 or a polypeptide with at least 80%, or at least 85%, or at least 90% amino acid sequence identity to SEQ ID NO: 56, or a protein of at least 20 amino acids that has at least 85% sequence identity to SEQ ID NO: 56.

81. A vaccine composition comprising any of the fusion proteins of claims 50-80.

82. A vaccine composition comprising the immunogenic composition of any of claims 1-42.

83. A pharmaceutical composition comprising the immunogenic composition of any of claims 1-42 and a pharmaceutically acceptable carrier.

84. The pharmaceutical composition of claim 83, comprising any of the fusion proteins of claims 50-82.

85. The pharmaceutical composition of claim 83, further comprising one or more adjuvants.

86. The pharmaceutical composition of claim 85, wherein the one or more adjuvants is or comprises a co-stimulation factor.

87. The pharmaceutical composition of claim 86, wherein the one or more adjuvants are selected from the group consisting of aluminum phosphate, aluminum hydroxide, and phosphated aluminum hydroxide.

88. The pharmaceutical composition of any one of claims 83-87, wherein upon administration to a subject, the pharmaceutical composition induces an opsonic/bactericidal response against one or more serotypes of S. aureus.

89. The pharmaceutical composition of any one of claims 83-87, wherein upon administration to a subject, the pharmaceutical composition reduces or inhibits transmission of one or more serotypes of S. aureus. from the subject to another subject.

90. The pharmaceutical composition of any one of claims 83-87, wherein upon administration to a subject, the pharmaceutical composition inhibits, or reduces the rate of occurrence of, or reduced the severity of any of the diseases or disorders selected from: sepsis, Pneumonia (including ventilator-associated pneumonia), sinusitis, skin infections/abscess/cellulitis/furuncles/impetigo, bloodstream infection, meningitis, endocarditis (heart infection), osteomyelitis, septic arthritis (or bone/joint infections), myositis (muscle infection), mastitis, mastoiditis, toxic shock syndrome, necrotizing fasciitis catheter/central line infection, prosthetic material infection including joint prostheses, surgical site infections associated with or induced by one or more serotypes of S. aureus.

91. The pharmaceutical composition of any one of claims 83-87, wherein upon administration to a subject, the pharmaceutical composition inhibits, or reduces the rate of occurrence of, or reduced the severity of pneumonia associated with or induced by one or more serotypes of S. aureus.

92. The pharmaceutical composition of any one of claims 83-87, wherein upon administration to a subject, the pharmaceutical composition inhibits, or reduces the rate of, colonization of mucosal surfaces by one or more serotypes of S. aureus.

93. The pharmaceutical composition of any one of claims 83-87, wherein upon administration to a subject, the pharmaceutical composition inhibits, or reduces the rate of, colonization of the nasopharynx by one or more serotypes of S. aureus.

94. The pharmaceutical composition of any one of claims 83-93, wherein the pharmaceutical composition is formulated for injection.

95. The pharmaceutical composition of any one of claims 83-94, wherein upon administration to a subject, the pharmaceutical composition induces an immune response.

96. The pharmaceutical composition of claim 90, wherein the immune response comprises an antibody and/or B cell response.

97. The pharmaceutical composition of claim 95 or claim 96, wherein the immune response comprises a CD4+ T cell response (e.g., TH1, TH2, or TH17 response); a CD8+ T cell response; a CD4+ and CD8+ T cell response; or a CD4−/CD8− T cell response.

98. The pharmaceutical composition of any one of claims 95-97, wherein the immune response comprises (i) an antibody or B cell response and (ii) a T cell response.

99. The pharmaceutical composition of any one of claims 95-98, wherein the immune response is to (i) at least one polysaccharide antigen of the immunogenic complex, and/or (ii) at least one polypeptide antigen of the immunogenic complex.

100. The pharmaceutical composition of any one of claims 95-99, wherein the immune response comprises (i) an antibody or B cell response to at least one polysaccharide antigen of the immunogenic complex, and (ii) a CD4+ T cell response (e.g., TH1, TH2, or TH17 response), a CD8+ T cell response, a CD4+ and CD8+ T cell response, or a CD4−/CD8− T cell response to at least one polypeptide antigen of the immunogenic complex.

101. The pharmaceutical composition of any one of claims 95-100, wherein the immune response comprises (i) an antibody or B cell response to at least one polysaccharide antigen of the immunogenic complex, and (ii) an antibody or B cell response to at least one polypeptide antigen of the vaccine or immunogenic complex.

102. The pharmaceutical composition of any one of claims 95-96, wherein the immune response comprises (i) an antibody or B cell response to at least one polysaccharide antigen of the immunogenic complex, and (ii) an antibody or B cell response; and a CD4+ T cell response (including TH1, TH2, or TH17 response), a CD8+ T cell response, a CD4+ and CD8+ T cell response, or a CD4−/CD8− T cell response to at least one polypeptide antigen of the immunogenic complex.

103. A heterologous nucleic acid sequence, comprising, in any order, (i) a nucleic acid sequence encoding a biotin-binding moiety having an amino acid sequence of at least 80%, or 90%, or 95% sequence identity to SEQ ID NO: 1, and (ii) a nucleic acid sequence encoding a S. aureus polypeptide antigen comprises a polypeptide comprising the amino acids selected from the group of: SEQ ID NO: 50 (SA1739; B1), SEQ ID NO: 51 (SA1720; B2), SEQ ID NO: 52 (SA1890; B3), SEQ ID NO: 53 (SA0103; T1), SEQ ID NO: 54 (SA0377; T2), SEQ ID NO: 55 (SA0693; T3), SEQ ID NO: 56 (SA2105; T4), or a fragment of at least 20 amino acids thereof, or a polypeptide having at least 80%, or at least 85% or 90% sequence identity to any of SEQ ID NO: 51-56.

104. The heterologous nucleic acid sequence of claim 103, wherein the nucleic acid sequence comprises, in the following order:
(a) a nucleic acid sequence encoding a biotin-binding moiety having an amino acid sequence of at least 80%, or 90%, or 95% sequence identity to SEQ ID NO: 1, and a nucleic acid sequence encoding a S. aureus polypeptide antigen comprises a polypeptide comprising the amino acids selected from the group of: SEQ ID NO: 50 (SA1739; B1), SEQ ID NO: 51 (SA1720; B2), SEQ ID NO: 52 (SA1890; B3), SEQ ID NO: 53 (SA0103; T1), SEQ ID NO: 54 (SA0377; T2), SEQ ID NO: 55 (SA0693; T3), SEQ ID NO: 56 (SA2105; T4), or a fragment of at least 20 amino acids thereof, or a polypeptide having at least 80%, or at least 85% or 90% sequence identity to any of SEQ ID NO: 51-56, or
(b) (i) a nucleic acid sequence encoding a S. aureus polypeptide antigen, the S. aureus polypeptide antigen comprising the amino acids selected from the group of: SEQ ID NO: 50 (SA1739; B1), SEQ ID NO: 51 (SA1720; B2), SEQ ID NO: 52 (SA1890; B3), SEQ ID NO: 53 (SA0103; T1), SEQ ID NO: 54 (SA0377; T2), SEQ ID NO: 55 (SA0693; T3), SEQ ID NO: 56 (SA2105; T4), or a fragment of at least 20 amino acids thereof, or a polypeptide having at least 80%, or at least 85% or 90% sequence identity to any of SEQ ID NO: 51-56, and (ii) a nucleic acid sequence encoding a biotin-binding moiety having an amino acid sequence of at least 80%, or 90%, or 95% sequence identity to SEQ ID NO: 1.

105. The heterologous nucleic acid sequence of claim 104 or 105, wherein the nucleic acid sequence comprises, in the following order:
(a) a nucleic acid sequence comprising (i) a nucleic acid sequence comprising SEQ ID NO: 81, or a nucleic acid sequence having 80%, or 90%, or 95% sequence identity to SEQ ID NO: 81 that encodes a biotin-binding moiety having an amino acid sequence of at least 80%, or 90%, or 95% sequence identity to SEQ ID NO: 1, and (ii) a nucleic acid sequence selected from the group of: SEQ ID NO: 57 (SA1739; B1), SEQ ID NO: 58 (SA1720; B2), SEQ ID NO: 59 (SA1890; B3), SEQ ID NO: 60 (SA0103; T1), SEQ ID NO: 67 (SA0377;
T2), SEQ ID NO: 62 (SA0693; T3), SEQ ID NO: 63 (SA2105; T4), or a nucleic acid sequence having at least 80%, or at least 85% or 90% sequence identity to any of SEQ ID NO: 57-63 that encodes a polypeptide selected from SEQ ID NO: 51-56 or a polypeptide having at least 85% amino acid sequence identity to SEQ ID NO: 51-56,
(b) a nucleic acid sequence comprising (i) a nucleic acid sequence selected from the group of SEQ ID NO: 57 (SA1739; B1), SEQ ID NO: 58 (SA1720; B2), SEQ ID NO: 59 (SA1890; B3), SEQ ID NO: 60 (SA0103; T1), SEQ ID NO: 67 (SA0377; T2), SEQ ID NO: 62 (SA0693; T3), SEQ ID NO: 63 (SA2105; T4), or a nucleic acid sequence having at least 80%, or at least 85% or 90% sequence identity to any of SEQ ID NO: 57-63 that encodes a polypeptide selected from SEQ ID NO: 51-56 or a polypeptide having at least 85% amino acid sequence identity to SEQ ID NO: 51-56, and (ii) a nucleic acid sequence comprising (i) a nucleic acid sequence comprising SEQ ID NO: 81, or a nucleic acid sequence having 80%, or 90%, or 95% sequence identity to SEQ ID NO: 81 that encodes a biotin-binding moiety having an amino acid sequence of at least 80%, or 90%, or 95% sequence identity to SEQ ID NO: 1.

106. The heterologous nucleic acid sequence of any of claims 103-105, further comprising (iii) a third nucleic acid sequence selected from the group of: SEQ ID NO: 57 (SA1739; B1), SEQ ID NO: 58 (SA1720; B2), SEQ ID NO: 59 (SA1890; B3), SEQ ID NO: 60 (SA0103; T1), SEQ ID NO: 67 (SA0377; T2), SEQ ID NO: 62 (SA0693; T3), SEQ ID NO: 63 (SA2105; T4), or a nucleic acid sequence having at least 80%, or at least 85% or 90% sequence identity to any of SEQ ID NO: 57-63 that encodes a polypeptide selected from SEQ ID NO: 51-56 or a polypeptide having at least 85% amino acid sequence identity to SEQ ID NO: 51-56.

107. The heterologous nucleic acid sequence of any of claims 103-106, wherein the nucleic acid encodes a fusion protein comprising a biotin-binding moiety having an amino acid sequence of at least 80%, or 90%, or 95% sequence identity to SEQ ID NO: 1 that has any one or more of the amino acid modifications: N80, T108, N118, S119A, N138A.

108. The heterologous nucleic acid sequence of any of claims 103-106, operatively linked to a promoter.

109. The heterologous nucleic acid sequence of any of claims 103-108, wherein the heterologous nucleic acid further comprises any one or more of: a nucleic acid that encodes at least one linker, or a nucleic acid that encodes an expression tag, or a nucleic acid that encodes a lipidation sequence.

110. The heterologous nucleic acid sequence of any of claims 103-109, wherein the nucleic acid sequence encoding at least one linker is located between the nucleic acid sequence of SEQ ID NO: 81 and the nucleic acid sequence selected from the group of: SEQ ID NO: 57 (SA1739; B1), SEQ ID NO: 58 (SA1720; B2), SEQ ID NO: 59 (SA1890; B3), SEQ ID NO: 60 (SA0103; T1), SEQ ID NO: 67 (SA0377; T2), SEQ ID NO: 62 (SA0693; T3), SEQ ID NO: 63 (SA2105; T4).

111. The heterologous nucleic acid sequence of claim 109 or 110, wherein the nucleic acid sequence encodes at least one linker having an amino acid sequence selected from any of: GGGGSSS (SEQ ID NO: 71) or AAA (SEQ ID NO: 72) and any of: SEQ ID NOS: 30-37 or SEQ ID NO: 44 or 45.

112. An expression vector comprising a nucleic acid sequence of any of claims 103-110.

113. The expression vector of claim 112, wherein the vector is an expression vector.

114. A cell comprising the heterologous nucleic acid of sequence of any of claims 103-110, or an expression vector of any of claim 112 or 113.

115. The cell of claim 114, where the cell is an expression host cell.

116. The cell of claim 115, wherein the expression host cell is selected from the group consisting of: E. coli, an insect cell line or a mammalian cell line.

117. The cell of claim 116, wherein the insect cell line is baculovirus expression system.

118. The cell of claim 116, wherein the mammalian cell line is a human cell line or Chinese Hamster ovary (CHO) cell line.

119. The cell of any of claims 114-118, wherein the heterologous nucleic acid sequence is codon-optimized to improve expression in the host cell.

120. The cell of any of claims 114-118, wherein the heterologous nucleic acid sequence further comprises a nucleic acid sequence encoding one or more of: polyadenylation sequence or termination sequence, a signal sequence.

121. The fusion protein of any of claims 50-80, encoded by the heterologous nucleic acid sequence of claims 103-110, or expressed by the expression vector of any of claim 112 or 113, or produced in a cell of any of claims 114-120.


SEQUENCE LISTING (81 sequences)

SEQ ID NO: 1 - protein, 135 aa, synthetic construct
  source 1..135: note = Artificial Sequence: Synthetic polypeptide
FDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKD

SEQ ID NO: 2 - protein, 894 aa, Staphylococcus aureus
SENSVTQSDSASNESKSNDSSSVSAAPKTDDTNVSDTKTSSNTNNGETSVAQNPAQQETTQSSSTNATTEETPVTGEATTTTTNQANTPATTQSSNTNAEELVNQTSNETTFNDTNTVSSVNSPQNSTNAENVSTTQDTSTEATPSNNESAPQSTDASNKDVVNQAVNTSAPRMRAFSLAAVAADAPAAGTDITNQLTNVTVGIDSGTTVYPHQAGYVKLNYGFSVPNSAVKGDTFKITVPKELNLNGVTSTAKVPPIMAGDQVLANGVIDSDGNVIYTFTDYVNTKDDVKATLTMPAYIDPENVKKTGNVTLATGIGSTTANKTVLVDYEKYGKFYNLSIKGTIDQIDKTNNTYRQTIYVNPSGDNVIAPVLTGNLKPNTDSNALIDQQNTSIKVYKVDNAADLSESYFVNPENFEDVTNSVNITFPNPNQYKVEFNTPDDQITTPYIVVVNGHIDPNSKGDLALRSTLYGYNSNIIWRSMSWDNEVAFNNGSGSGDGIDKPVVPEQPDEPGEIEPIPEDSDSDPGSDSGSDSNSDSGSDSGSDSTSDSGSDSASDSDSASDSDSASDSDSASDSDSASDSDSDNDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSASDSDSDSDSDSDSDSDSDSDSDSDSDSDSDSDSDSDSDSESDSDSESDSDSDSDSDSDSDSDSDSDSDSASDSDSGSDSDSSSDSDSESDSNSDSESGSNNNVVPPNSPKNGTNASNKNEAKDSKEPLPDTGSEDEANTSLIWGLLASIGSLLLFRRKKENKDKK

SEQ ID NO: 3 - protein, 289 aa, Staphylococcus aureus
VAADAPAAGTDITNQLTNVTVGIDSGTTVYPHQAGYVKLNYGFSVPNSAVKGDTFKITVPKELNLNGVTSTAKVPPIMAGDQVLANGVIDSDGNVIYTFTDYVNTKDDVKATLTMPAYIDPENVKKTGNVTLATGIGSTTANKTVLVDYEKYGKFYNLSIKGTIDQIDKTNNTYRQTIYVNPENFEDVTNSVNITFPNPNQYKVEFNTPDDQITTPYIVVVNGHIDPNSKGDLALRSTLYGYNSNIIWRSMSWDNEVAFNNGSGSGDGIDKPVVPEQPDEPGEIEPIPE

SEQ ID NO: 4 - protein, 855 aa, Staphylococcus aureus
SEQSNDTTQSSKNNASADSEKNNMIETPQLNTTANDTSDISANTNSANVDSTTKPMSTQTSNTTTTEPASTNETPQPTAIKNQATAAKMQDQTVPQEANSQVDNKTTNDANSIATNSELKNSQTLDLPQSSPQTISNAQGTSKPSVRTRAVRSLAVAEPVVNAADAKGTNVNDKVTASNFKLEKTTFDPNQSGNTFMAANFTVTDKVKSGDYFTAKLPDSLTGNGDVDYSNSNNTMPIADIKSTNGDVVAKATYDILTKTYTFVFTDYVNNKENINGQFSLPLFTDRAKAPKSGTYDANINIADEMFNNKITYNYSSPIAGIDKPNGANISSQIIGVDTASGQNTYKQTVFVNPKQRVLGNTWVYIKGYQDKIEESSGKVSATDTKLRIFEVNDISKLSDSYYADPNDSNLKEVTDQFKNRIYYEHPNVASIKFGDITKTYVVLVEGHYDNTGKNLKTQVIQENVDPVTNRDYSIFGWNNENVVRYGGGSADGDSAVNPKDPTPGPPVDPEPSPDPEPEPSPDPDPDSDSDSDSGSDSDSGSDSDSESDSDSDSDSDSDSDSDSESDSDSESDSDSDSDSDSDSDSDSDSDSDSDSDSDSDSDSDSESDSDSESDSESDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSRVTPPNNEQKAPSNPKGEVNHSNKVSKQHKTDALPETGDKSENTNATLFGAMMALLGSLLLFRKRKQDHKEKA

SEQ ID NO: 5 - protein, 340 aa, Staphylococcus aureus
PVVNAADAKGTNVNDKVTASNFKLEKTTFDPNQSGNTFMAANFTVTDKVKSGDYFTAKLPDSLTGNGDVDYSNSNNTMPIADIKSTNGDVVAKATYDILTKTYTFVFTDYVNNKENINGQFSLPLFTDRAKAPKSGTYDANINIADEMFNNKITYNYSSPIAGIDKPNGANISSQIIGVDTASGQNTYKQTVFVNPKQRVLGNTWVYIKGYQDKIEESSGKVSATDTKLRIFEVNDISKLSDSYYADPNDSNLKEVTDQFKNRIYYEHPNVASIKFGDITKTYVVLVEGHYDNTGKNLKTQVIQENVDPVTNRDYSIFGWNNENVVRYGGGSADGDSAVN

SEQ ID NO: 6 - protein, 1329 aa, Staphylococcus aureus
AESTNKELNEATTSASDNQSSDKVDMQQLNQEDNTKNDNQKEMVSSQGNETTSNGNKLIEKESVQSTTGNKVEVSTAKSDEQASPKSTNEDLNTKQTISNQEALQPDLQENKSVVNVQPTNEENKKVDAKTESTTLNVKSDAIKSNDETLVDNNSNSNNENNADIILPKSTAPKRLNTRMRIAAVQPSSTEAKNVNDLITSNTTLTVVDADKNNKIVPAQDYLSLKSQITVDDKVKSGDYFTIKYSDTVQVYGLNPEDIKNIGDIKDPNNGETIATAKHDTANNLITYTFTDYVDRFNSVQMGINYSIYMDADTIPVSKNDVEFNVTIGNTTTKTTANIQYPDYVVNEKNSIGSAFTETVSHVGNKENPGYYKQTIYVNPSENSLTNAKLKVQAYHSSYPNNIGQINKDVTDIKIYQVPKGYTLNKGYDVNTKELTDVTNQYLQKITYGDNNSAVIDFGNADSAYVVMVNTKFQYTNSESPTLVQMATLSSTGNKSVSTGNALGFTNNQSGGAGQEVYKIGNYVWEDTNKNGVQELGEKGVGNVTVTVFDNNTNTKVGEAVTKEDGSYLIPNLPNGDYRVEFSNLPKGYEVTPSKQGNNEELDSNGLSSVITVNGKDNLSADLGIYKPKYNLGDYVWEDTNKNGIQDQDEKGISGVTVTLKDENGNVLKTVTTDADGKYKFTDLDNGNYKVEFTTPEGYTPTTVTSGSDIEKDSNGLTTTGVINGADNMTLDSGFYKTPKYNLGNYVWEDTNKDGKQDSTEKGISGVTVTLKNENGEVLQTTKTDKDGKYQFTGLENGTYKVEFETPSGYTPTQVGSGTDEGIDSNGTSTTGVIKDKDNDTIDSGFYKPTYNLGDYVWEDTNKNGVQDKDEKGISGVTVTLKDENDKVLKTVTTDENGKYQFTDLNNGTYKVEFETPSGYTPTSVTSGNDTEKDSNGLTTTGVIKDADNMTLDSGFYKTPKYSLGDYVWYDSNKDGKQDSTEKGIKDVKVTLLNEKGEVIGTTKTDENGKYCFDNLDSGKYKVIFEKPAGLTQTGTNTTEDDKDADGGEVDVTITDHDDFTLDNGYYEEETSDSDSDSDSDSDSDRDSDSDSDSDSDSDSDSDSDSDSDSDSDSDRDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDAGKHTPVKPMSTTKDHHNKAKALPETGNENSGSNNATLFGGLFAALGSLLLFGRRKKQNK

SEQ ID NO: 7 - protein, 437 aa, Staphylococcus aureus
NVNDLITSNTTLTVVDADKNNKIVPAQDYLSLKSQITVDDKVKSGDYFTIKYSDTVQVYGLNPEDIKNIGDIKDPNNGETIATAKHDTANNLITYTFTDYVDRFNSVQMGINYSIYMDADTIPVSKNDVEFNVTIGNTTTKTTANIQYPDYVVNEKNSIGSAFTETVSHVGNKENPGYYKQTIYVNPSENSLTNAKLKVQAYHSSYPNNIGQINKDVTDIKIYQVPKGYTLNKGYDVNTKELTDVTNQYLQKITYGDNNSAVIDFGNADSAYVVMVNTKFQYTNSESPTLVQMATLSSTGNKSVSTGNALGFTNNQSGGAGQEVYKIGNYVWEDTNKNGVQELGEKGVGNVTVTVFDNNTNTKVGEAVTKEDGSYLIPNLPNGDYRVEFSNLPKGYEVTPSKQGNNEELDSNGLSSVITVNGKDNLSADLGIYKPKY

SEQ ID NO: 8 - protein, 1068 aa, Staphylococcus aureus
AENTSTENAKQDDATTSDNKEVVSETENNSTTENNSTNPIKKETNTDSQPEAKKESTSSSTQKQQNNVTATTETKPQNIEKENVKPSTDKTATEDTSVILEEKKAPNNTNNDVTTKPSTSEPSTSEIQTKPTTPQESTNIENSQPQPTPSKVDNQVTDATNPKEPVNVSKEELKNNPEKLKELVRNDSNTDHSTKPVATAPTSVAPKRVNAKMRFAVAQPAAVASNNVNDLIKVTKQTIKVGDGKDNVAAAHDGKDIEYDTEFTIDNKVKKGDTMTINYDKNVIPSDLTDKNDPIDITDPSGEVIAKGTFDKATKQITYTFTDYVDKYEDIKSRLTLYSYIDKKTVPNETSLNLTFATAGKETSQNVTVDYQDPMVHGDSNIQSIFTKLDEDKQTIEQQIYVNPLKKSATNTKVDIAGSQVDDYGNIKLGNGSTIIDQNTEIKVYKVNSDQQLPQSNRIYDFSQYEDVTSQFDNKKSFSNNVATLDFGDINSAYIIKVVSKYTPTSDGELDIAQGTSMRTTDKYGYYNYAGYSNFIVTSNDTGGGDGTVKPEEKLYKIGDYVWEDVDKDGVQGTDSKEKPMANVLVTLTYPDGTTKSVRTDANGHYEFGGLKDGETYTVKFETPTGYLPTKVNGTTDGEKDSNGSSVTVKINGKDDMSLDTGFYKEPKYNLGDYVWEDTNKDGIQDANEPGIKDVKVTLKDSTGKVIGTTTTDASGKYKFTDLDNGNYTVEFETPAGYTPTVKNTTADDKDSNGLTTTGVIKDADNMTLDSGFYKTPKYSLGDYVWYDSNKDGKQDSTEKGIKDVTVTLQNEKGEVIGTTKTDENGKYRFDNLDSGKYKVIFEKPAGLTQTVTNTTEDDKDADGGEVDVTITDHDDFTLDNGYFEEDTSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDAGKHTPVKPMSTTKDHHNKAKALPET

SEQ ID NO: 9 - skipped

SEQ ID NO: 10 - protein, 304 aa, Staphylococcus aureus
ATEATNATNNQSTQVSQATSQPINFQVQKDGSSEKSHMDDYMQHPGKVIKQNNKYYFQTVLNNASFWKEYKFYNANNQELATTVVNDNKKADTRTINVAVEPGYKSLTTKVHIVVPQINYNHRYTTHLEFEKAIPTLADAAKPNNVKPVQPKPAQPKTPTEQTKPVQPKVEKVKPTVTTTSKVEDNHSTKVVSTDTTKDQTKTQTAHTVKTAQTAQEQNKVQTPVKDVATAKSESNNQAVSDNKSQQTNKVTKHNETPKQASKAKELPKTGLTSVDNFISTVAFATLALLGSLSLLLFKRKESK

SEQ ID NO: 11 - protein, 278 aa, Staphylococcus aureus
ATEATNATNNQSTQVSQATSQPINFQVQKDGSSEKSHMDDYMQHPGKVIKQNNKYYFQTVLNNASFWKEYKFYNANNQELATTVVNDNKKADTRTINVAVEPGYKSLTTKVHIVVPQINYNHRYTTHLEFEKAIPTLADAAKPNNVKPVQPKPAQPKTPTEQTKPVQPKVEKVKPTVTTTSKVEDNHSTKVVSTDTTKDQTKTQTAHTVKTAQTAQEQNKVQTPVKDVATAKSESNNQAVSDNKSQQTNKVTKHNETPKQASKAKELPKTGLTSVDNF

SEQ ID NO: 12 - protein, 605 aa, Staphylococcus aureus
AAEETGGTNTEAQPKTEAVASPTTTSEKAPETKPVANAVSVSNKEVEAPTSETKEAKEVKEVKAPKETKEVKPAAKATNNTYPILNQELREAIKNPAIKDKDHSAPNSRPIDFEMKKKDGTQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKN

SEQ ID NO: 13 - protein, 430 aa, Staphylococcus aureus
TNTEAQPKTEAVASPTTTSEKAPETKPVANAVSVSNKEVEAPTSETKEAKEVKEVKAPKETKEVKPAAKATNNTYPILNQELREAIKNPAIKDKDHSAPNSRPIDFEMKKKDGTQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEAT

SEQ ID NO: 14-15 - skipped (2 entries)

SEQ ID NO: 16 - protein, 293 aa, synthetic construct
  source 1..293: note = Artificial Sequence
ADSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYAAASWNPVYGNQLFMKTRNGSMKAADNFLDPNKASSLLSSGFSPDFATVITMDRKASKQQTNIDVIYERVRDDYQLHWTSTNWKGTNTKDKWIDRSSERYKIDWEKEEMTN

SEQ ID NO: 17 - protein, 293 aa, synthetic construct
  source 1..293: note = Artificial Sequence
ADSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNAGPYDRDSWNPVYGNQLFMKTRNGSMKAADNFLDPNKASSLLSSGFSPDFATVITMDRKASKQQTNIDVIYERVRDDYQLHWTSTNWKGTNTKDKWIDRSSERYKIDWEKEEMTN

SEQ ID NO: 18 - protein, 293 aa, synthetic construct
  source 1..293: note = Artificial Sequence: Synthetic polypeptide
ADSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSANPVYGNQLFMKTRNGSMKAADNFLDPNKASSLLSSGFSPDFATVITMDRKASKQQTNIDVIYERVRDDYQLHWTSTNWKGTNTKDKWIDRSSERYKIDWEKEEMTN

SEQ ID NO: 19 - protein, 5 aa, synthetic construct
  source 1..5: note = Artificial Sequence
  VARIANT 3: note = Any amino acid
LPXTG

SEQ ID NO: 20 - protein, 9 aa, synthetic construct
TYTFTDYVD

SEQ ID NO: 21 - protein, 179 aa, synthetic construct
MIITSLYATFGTIADGRRTSGGKTMIRTNAVAALVFAVATSALAFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKD

SEQ ID NO: 22 - protein, 44 aa, synthetic construct
MIITSLYATFGTIADGRRTSGGKTMIRTNAVAALVFAVATSALA

SEQ ID NO: 23 - protein, 19 aa, Escherichia coli
MKKIWLALAGLVLAFSASA

SEQ ID NO: 24 - protein, 23 aa, Escherichia coli
MKKIWLALAGLVLAFSASAAQDP

SEQ ID NO: 25 - protein, 17 aa, unidentified
  source 1..17: note = Description unknown: signal sequence
MKKVAAFVALSLLMAGC

SEQ ID NO: 26 - protein, 19 aa, unidentified
  source 1..19: note = Description unknown: signal sequence
MKKIWLALAGLVLAFSASA

SEQ ID NO: 27 - protein, 23 aa, unidentified
  source 1..23: note = Description unknown: signal sequence
MAPFEPLASGILLLLWLIAPSRA

SEQ ID NO: 28 - protein, 17 aa, unidentified
  source 1..17: note = Description unknown: signal sequence
MKKVAAFVALSLLMAGC

SEQ ID NO: 29 - protein, 17 aa, synthetic construct
MKKVAAFVALSLLMAGC

SEQ ID NO: 30 - protein, 4 aa, synthetic construct
AQDP

SEQ ID NO: 31 - protein, 4 aa, synthetic construct
VSDP

SEQ ID NO: 32 - protein, 6 aa, synthetic construct
HHHHHH

SEQ ID NO: 33 - protein, 22 aa, synthetic construct
GGGGSSSVDKLAAALEHHHHHH

SEQ ID NO: 34 - protein, 9 aa, synthetic construct
VDKLAAALE

SEQ ID NO: 35 - protein, 16 aa, synthetic construct
GGGGSSSVDKLAAALE

SEQ ID NO: 36 - protein, 14 aa, synthetic construct
VDKLAAALEHHHHH

SEQ ID NO: 37 - protein, 22 aa, synthetic construct
GGGGSSSVDKLAAALEHHHHHH

SEQ ID NO: 38 - protein, 14 aa, synthetic construct
GSPGISGGGGGILE

SEQ ID NO: 39 - protein, 17 aa, synthetic construct
  source 1..17: note = Artificial Sequence: Synthetic polypeptide
MKKVAAFVALSLLMAGC

SEQ ID NO: 40 - protein, 21 aa, synthetic construct
  source 1..21: note = Artificial Sequence
MNSKKLCCICVLFSLLAGCAS

SEQ ID NO: 41 - protein, 19 aa, synthetic construct
  source 1..19: note = Artificial Sequence
MRYSKLTMLIPCALLLSAC

SEQ ID NO: 42 - protein, 24 aa, synthetic construct
  source 1..24: note = Artificial Sequence
MFVTSKKMTAAVLAITLAMSLSAC

SEQ ID NO: 43 - protein, 18 aa, synthetic construct
  source 1..18: note = Artificial Sequence
MIKRVLVVSMVGLSLVGC

SEQ ID NO: 44 - protein, 4 aa, synthetic construct
VSDP

SEQ ID NO: 45 - protein, 4 aa, synthetic construct
AQDP

SEQ ID NO: 46 - skipped

SEQ ID NO: 47 - protein, 9 aa, synthetic construct
  VARIANT 3: note = R or L
  VARIANT 5: note = S or T
  VARIANT 7: note = Y or W
CDXAXPXCG

SEQ ID NO: 48 - protein, 23 aa, unidentified
  source 1..23: note = Description unknown: signal sequence
MAPFEPLASGILLLLWLIAPSRA

SEQ ID NO: 49 - skipped

SEQ ID NO: 50 - protein, 255 aa, Staphylococcus aureus
ISDQIFFKHVKSDIKIEKLNVTLNDAAKKQINNYTSQQVSNKKNDAWRDASATEIKSAMDSGTFIDNEKQKYQFLDLSKYQGIDKNRIKRMLVDRPTLLKHTDDFLKAAKDKHVNEVYLISHALLETGAVKSELANGVEIDGKKYYNFYGVGALDKDPIKTGAEYAKKHGWDTPEKAISGGADFIHKHFLSSTDQNTLYSMRWNPKNPGEHQYATDIKWAESNATIIADFYKNMKTEGKYFKYFVYKDDSKHLNK

SEQ ID NO: 51 - protein, 157 aa, Staphylococcus aureus
NNDNSTNNQSSKTQLTQKDENKNEDKPEEKSETATDEDLQSTEEVPANENTENNQHEIDEITTKDQSDDDINTPNVAEDKSQDDLKDDLKEKQQSSNHHQSTQPKTSPSTETNTQQSFANCKQLRQVYPNGVTADHPAYRPHLDRDKDKRACEPDKY

SEQ ID NO: 52 - protein, 363 aa, Staphylococcus aureus
ESNSNIKAKDKKHVQVNVEDKSVPTDVRNLAQKDYLSYVTSLDKIYNKEKASYTLGEPFKIYKFNKKSDGNYYFPVLNTEGNIDYIVTISPKITKYSSSSSKYTINVSPFLSKVLNQYKDQQITILTNSKGYYVVTQNHKAKLVLKTPRLEDKKLKKTESIPTGNNVTQLKQKASVTMPTSQFKSNNYTYNEQYINKLENFKIRETQGNNGWCAGYTMSELLNATYNTNKYHAEAVMRFLHPNLQGQRFQFTGLTPREMIYFGQTQGRSPQLLNRMTTYNEVDNLTKNNKGIAVLGSRVESRNGMHAGHAMAVVGNAKLDNGQEVIIIWNPWDNGFMTQDAKNNVIPVSNGDHYRWYSSIYGY

SEQ ID NO: 53 - protein, 229 aa, Staphylococcus aureus
GKEAEVKKSFEKTLSMYPIKNLEDLYDKEGYRDDEFDKNDKGTWIIGSEMVVQPKGERMKSKGMVLYMNRNTKTTTGKYIVSETLHDEDGRPKSKDKEYPVKMVDNKIIPTKGIKDENIKKEIENFKFFAQYGSFKDLSKYKDGDISYNPEVPSYSAKYQLTNDDYNVKQLRKRYKIPTNKAPKLLLKGSGDLKGSSVGYKDIEFTFVEKKGENTFFTDSLHLEPSEDK

SEQ ID NO: 54 - protein, 187 aa, Staphylococcus aureus
SDQSDNEDHHTSTGIHAPKSAKKLETKDIFNSDKKNSDISDAEMKQAIEKYLSVNSDILDNKYIMQHKLDKQIDSQTKVTEKQAETLSHLSNLAVKNDLHFKKFVTENNIPKEYKKPVELMMNYFKALNSTIANVDEDIEKLSYQPQNKINVVDVPTKYAGDVNKKQQDKIKDFLKSKGIKSDVIDK

SEQ ID NO: 55 - protein, 120 aa, Staphylococcus aureus
QDQGNKTEQKTKSEDSNVKTDKTKHLTGTFSSKNGETVEGKAEIKNGKLMLTNYKSSKGPDLYVYLTKNGDIKNGKEIAMVDYDKEKQTFDLKNVDLSKYDEVTIYCKKAHVIFGGAKLK

SEQ ID NO: 56 - protein, 172 aa, Staphylococcus aureus
KFTREPKQDLEAATAQMENTKGKKSSVASKLVSSDKNVNTEENASGNVSETSSSDDDPEALLDNYNTEDVDAHNYNNINHVIFACDAGMGSSAMGASMLRNKFKKAGINDITVTNTAINQLPKDAQLVITQKKLTDRAIKQTPNAIHISVDNFLNSPRYEELLNNLKKDDQA

SEQ ID NO: 57 - DNA, 768 bp, Staphylococcus aureus
  source 1..768: mol_type = genomic DNA
atatccgatcagatatttttcaaacatgttaaatccgacattaagattgaaaagttaaatgttacattaaacgatgcagcaaagaaacaaataaataattatacgagtcaacaggtatcaaataaaaagaatgatgcatggagagatgcatctgcaactgaaattaaaagtgcaatggatagcggtacttttatcgataatgaaaagcaaaaatatcaatttttagatttatcaaagtatcaagggattgataaaaatagaattaaacgtatgttagtagatagaccaacgttattgaaacatacggatgatttcttaaaagctgctaaagataagcacgttaacgaagtttatttaatttcacatgcattattagaaactggcgcagttaaaagtgaattagctaatggagtcgaaattgatggcaaaaagtactacaatttctatggagtaggagcccttgataaagacccaattaaaacaggtgcagaatatgctaaaaagcatggttgggatacacctgaaaaagctatttcaggcggtgctgatttcattcataagcacttcttatcaagcacagatcaaaatacattgtatagtatgagatggaatccaaaaaatccaggagaacatcaatatgctacagatattaagtgggcagaaagtaatgcaacaattatcgctgacttttataagaacatgaagactgaaggaaaatacttcaaatactttgtgtataaagatgacagtaaacatttgaataagtaa

SEQ ID NO: 58 - DNA, 474 bp, Staphylococcus aureus
  source 1..474: mol_type = genomic DNA
aataatgacaattccactaataatcaatcatcaaaaacgcaattaactcaaaaagatgaaaataaaaatgaagataagcctgaggaaaaatcagaaacagcaacagatgaggatttacaatcaaccgaagaagtacctgcaaatgaaaatactgaaaataatcaacatgaaattgatgaaataacaacaaaagatcaatcagacgatgatattaacacaccaaacgttgcagaagataaatcacaagacgacttgaaagatgatttaaaagaaaagcaacaatcaagtaaccatcatcaatccacgcaacctaagacctcaccatcaactgaaacaaacacgcaacaatcatttgctaattgtaagcaacttagacaagtatatccgaatggtgtcactgccgatcatccagcatatcgaccacatttagatagagataaagataaacgtgcatgtgaacctgataaatattaa

SEQ ID NO: 59 - DNA, 1092 bp, Staphylococcus aureus
  source 1..1092: mol_type = genomic DNA
gagagcaattcaaatatcaaagcaaaagataagaagcatgtgcaagttaatgtcgaggacaagagtgttcctactgacgtacgtaatttagcgcaaaaagactatctttcttatgttacttcacttgataaaatctataataaagagaaagcctcgtatacacttggtgaaccttttaaaatttataaattcaacaaaaagagtgatggtaattattattttccagttttgaacactgaaggtaatatcgattatatagttacaatttcaccaaaaatcactaagtattctagttcatcttctaaatacacaatcaatgtttctccatttttatctaaagtcttaaatcaatataaagatcaacaaattactattttgacaaactcaaaaggttactatgtggttactcaaaatcataaagctaaactagtactaaaaacaccacgtctagaagataaaaagttaaaaaagactgagagtatacctactggcaataatgtaacgcaactaaagcaaaaagcttctgtcactatgccaactagtcaatttaaaagtaataattatacttacaacgagcaatatataaataaacttgagaatttcaagataagagaaactcaaggtaacaatggttggtgtgcaggctatacgatgtctgaattactaaatgcgacttataatactaacaaatatcatgcagaagctgtgatgagatttttacacccaaacttacaaggacaacgattccaatttactggtttaacgccaagagagatgatttattttggacaaactcagggaagaagtccacaacttcttaatagaatgactacatataatgaagttgacaatcttactaaaaataataaaggaattgctgttttaggtagtagagtagaatcaagaaatggtatgcacgcagggcatgccatggcagttgttgggaacgctaaattagataatggacaagaggttataattatatggaatccatgggataatggttttatgactcaagatgctaaaaacaatgttattccagtatccaatggcgaccattatcgatggtattcatctatttatggttattaa

SEQ ID NO: 60 - DNA, 690 bp, Staphylococcus aureus
  source 1..690: mol_type = genomic DNA
ggtaaagaagcggaagttaagaaaagctttgaaaaaacattgagtatgtatccgattaaaaatctagaggatttatacgataaagaaggttatcgtgatgacgaatttgataaaaatgataaaggtacatggattattggttctgaaatggttgttcaacctaaaggggaacgtatgaaatcaaaaggtatggttctatatatgaatagaaacactaagactacaaccgggaagtatatagtgagtgaaacacttcatgatgaagatggtagacctaagagtaaagataaagaatatcccgttaaaatggtagacaataaaatcattccaactaaaggtatcaaagacgaaaatataaaaaaagaaatcgagaattttaagttcttcgcgcaatatggcagctttaaagatttgtcgaagtacaaagacggcgatatatcatataatccagaggtaccgagttattcagcaaaatatcaattaactaatgatgactataatgtaaaacaattacgaaaaagatataaaataccaacgaataaagcaccaaagttattgttgaaaggttcgggagacttaaaaggttcatcagttggatataaagacattgaatttacttttgtggagaaaaaaggtgaaaatactttttttactgatagtctacatcttgaaccgagtgaggataaataa

SEQ ID NO: 61 - DNA, 564 bp, Staphylococcus aureus
  source 1..564: mol_type = genomic DNA
tccgatcaatctgataacgaagatcatcatacaagtactggcatccatgcacctaaaagcgctaaaaaattagaaacaaaagatatctttaactcagataagaaaaatagtgatattagtgatgcagagatgaagcaagctatagaaaagtatttgtctgtaaatagcgatatacttgataacaaatatattatgcaacataaactagataaacaaattgacagtcaaacaaaagtgactgaaaaacaagcagaaacacttagccacttatctaacttagctgttaaaaacgatttgcatttcaagaaatttgtcactgaaaacaatattcctaaagaatataagaaacccgttgaattaatgatgaattacttcaaagctttaaatagtacaatcgccaatgttgatgaagatattgaaaagcttagctatcaaccgcaaaataaaatcaatgttgtcgacgtgccaacaaaatatgccggtgatgttaataaaaagcaacaagataaaattaaagatttcttaaaatctaaaggtatcaagagtgatgttatcgataaataa

SEQ ID NO: 62 - DNA, 363 bp, Staphylococcus aureus
  source 1..363: mol_type = genomic DNA
caagatcaaggtaacaaaactgaacaaaaaacaaagtcagaagatagcaatgttaaaactgataaaacaaaacacctaacaggtacattcagttctaaaaacggtgaaactgttgaaggtaaagctgagattaaaaatggtaaattaatgcttactaactacaaatcatcaaaaggtccagatttatacgtctacctaacaaaaaatggcgacattaaaaacggtaaagaaatcgcaatggttgactacgataaagaaaaacaaacatttgatcttaaaaatgtagatttaagcaaatatgatgaagtaacaatctattgtaaaaaagctcacgtcatcttcggcggcgctaaattaaaataa

SEQ ID NO: 63 - DNA, 519 bp, Staphylococcus aureus
  source 1..519: mol_type = genomic DNA
aagttcactagagaaccaaagcaggatttagaagctgcgacagctcaaatggaaaatactaaagggaaaaaatcaagcgttgcttctaagttagtatcttctgataaaaatgttaatacagaagaaaatgctagtggtaatgttagtgaaacatcttcatcagatgatgatcctgaagcgctattggataattacaacactgaagatgttgatgcacacaattacaataatataaatcatgttatttttgcctgcgatgcgggtatgggttctagtgcaatgggtgcaagcatgttacgtaataaatttaaaaaggcgggcattaatgatattacagttacaaatactgcgattaatcaattgccaaaagatgctcaattagttatcactcagaaaaaactaactgatcgtgctattaaacaaacaccaaatgccatccatatttcagtggataatttccttaattcaccaagatatgaagaacttttaaataatctaaaaaaagatgatcaagcataa

SEQ ID NO: 64 - protein, 24 aa, Listeria monocytogenes
MKKIMLVITLILVSPIAQQTEAKD

SEQ ID NO: 65 - protein, 27 aa, Staphylococcus aureus
MLKGCGGCLISFILLIILLSACSMMFS

SEQ ID NO: 66 - protein, 25 aa, Staphylococcus aureus
MKRNFPKLIALSLIFSLSVTPIANA

SEQ ID NO: 67 - protein, 26 aa, Staphylococcus aureus
MKRLNKLVLYISFLILVISFTAGCGI

SEQ ID NO: 68 - protein, 24 aa, Staphylococcus aureus
MKKRLLLSTFLASTLILTGCASDQ

SEQ ID NO: 69 - protein, 26 aa, Staphylococcus aureus
MNTKYFLAVGAVASVLTLGACSNSNS

SEQ ID NO: 70 - protein, 138 aa, Staphylococcus aureus
VFTPLGADQAAKAGQSILYTIESNPGPGLGILLAYMIFGKGTAKATSYGAGIIHFLGGIHEIYFPYVLMRPLLFIAVILGGMTGVATYQATGFGFKSPASPGSFIVYCLNAPRGEFLHMLLGVFLAALVSFVVATLIM

SEQ ID NO: 71 - protein, 7 aa, synthetic construct
GGGGSSS

SEQ ID NO: 72 - skipped

SEQ ID NO: 73 - protein, 29 aa, Lactococcus lactis
MKKKIISAILMSTVILSAAAPLSGVYADT

SEQ ID NO: 74 - protein, 31 aa, Bacillus anthracis
MKKRKVLIPLMALSTILVSSTGNLEVIQAEV

SEQ ID NO: 75 - protein, 29 aa, Listeria monocytogenes
MNMKKATIAATAGIAVTAFAAPTIASAST

SEQ ID NO: 76 - protein, 54 aa, Listeria monocytogenes
MQKTRKERILEALQEEKKNKKSKKFKTGATIAGVTAIATSITVPGIEVIVSADE

SEQ ID NO: 77 - protein, 28 aa, Bacillus anthracis
MKKLKMASCALVAGLMFSGLTPNAFAED

SEQ ID NO: 78 - protein, 31 aa, Staphylococcus aureus
MAKKFNYKLPSMVALTLVGSAVTAHQVQAAE

SEQ ID NO: 79 - protein, 59 aa, Listeria monocytogenes
MTDKKSENQTEKTETKENKGMTRREMLKLSAVAGTGIAVGATGLGTILNVVDQVDKALT

SEQ ID NO: 80 - protein, 53 aa, Bacillus subtilis
MAYDSRFDEWVQKLKEESFQNNTFDRRKFIQGAGKIAGLGLGLTIAQSVGAFG

SEQ ID NO: 81 - DNA, 405 bp, synthetic construct
  source 1..405: mol_type = other DNA; note = Artificial Sequence: Synthetic polypeptide
tttgatgcgagtaactttaaagatttcagctctattgcgagcgccagtagctcttggcagaatcagagtggcagcaccatgattatccaggtggattctttcggcaacgttagtggccagtatgttaatcgtgcgcagggcacgggttgccagaactctccgtacccgctgaccggccgcgtgaatggcacgtttatcgccttcagcgttggctggaacaattctaccgaaaactgtaatagtgcaaccggctggacgggttatgcgcaggtgaacggtaacaataccgaaattgttacgagctggaatctggcctatgaaggcggttctggcccggcaatcgaacagggtcaggatacctttcagtacgttccgaccacggaaaacaaaagcctgctgaaagat